(19) United States
(12) Patent Application Publication
(10) Pub. No.: US 2025/0066358 A1
(43) Pub. Date: Feb. 27, 2025

(54) Inhibitors of Menin-MLL Interaction

(71) Applicant: Bala Therapeutics, Inc., San Diego, CA (US)

(72) Inventors: Ruben Abagyan, La Jolla, CA (US); Vladislav Zenonovich Parchinsky, Moscow (RU); Alexander Khvat, San Diego, CA (US); Alexandre Vasilievich Ivachtchenko, Hallandale Beach, FL (US); Nikolay Savchuk, San Diego, CA (US)

(21) Appl. No.: 18/718,012

(22) PCT Filed: Dec. 9, 2022
(86) PCT No.: PCT/US2022/052378

§ 371 Date: Jun. 7, 2024

Related U.S. Application Data
(60) Provisional application No. 63/287,716, filed on Dec. 9, 2021.

(51) Int. Cl. C07D 471/10; A61K 31/517; A61K 31/519; A61K 31/5377; A61K 31/538; A61K 31/5415; A61K 31/554; C07D 401/12; C07D 403/12; C07D 519/00

(52) U.S. Cl. CPC C07D 471/10; A61K 31/517; A61K 31/519; A61K 31/5377; A61K 31/538; A61K 31/5415; A61K 31/554; C07D 401/12; C07D 403/12; C07D 519/00

(57) ABSTRACT

The present invention is directed to the compounds of Formula (I)—inhibitors of the interaction of menin and MLL. The inhibitors described herein can be useful in the treatment of diseases or disorders associated with menin-MLL interaction, such as cancer. In particular, the invention is concerned with compounds and pharmaceutical compositions inhibiting/blocking menin-MLL interaction, methods of treating diseases or disorders associated with menin-MLL interaction, and methods of synthesizing these compounds.

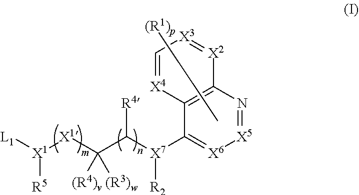

### CROSS-REFERENCE TO RELATED APPLICATIONS

This application claims priority to, and the benefit of, U.S. Provisional Patent Application Ser. No. 63/287,716 filed on Dec. 9, 2021 and entitled “INHIBITORS OF MENIN-MLL INTERACTION,” the disclosure of which is incorporated herein by reference in its entirety for all purposes.

### FIELD OF INVENTION

The present invention is directed to inhibitors of the interaction of menin and MLL. The inhibitors described herein can be useful in the treatment of diseases or disorders associated with menin-MLL interaction, such as cancer. In particular, the invention is concerned with compounds and pharmaceutical compositions inhibiting/blocking menin-MLL interaction, methods of treating diseases or disorders associated with menin-MLL interaction, and methods of synthesizing these compounds.

### BACKGROUND

Translocations of the MLL (mixed lineage leukemia) gene frequently occur in aggressive human acute myeloid and lymphoid leukemias in both children and adults. Fusion of MLL with 1 of more than 60 different genes results in chimeric MLL fusion proteins that enhance proliferation and block hematopoietic differentiation, ultimately leading to acute leukemia. Patients with leukemias harboring MLL translocations have very unfavorable prognoses and respond poorly to currently available treatments. The relapse risk is very high using conventional chemotherapy and stem cell transplantation, leading to an overall 5-year survival rate of only approximately 35% of patients.

Menin is an essential co-factor of oncogenic MLL fusion proteins and the menin-MLL interaction is critical for development of acute leukemia in vivo. Targeting the menin-MLL interaction with small molecules represents an attractive strategy to develop new anticancer agents. Recent developments, including determination of menin crystal structure and development of potent small molecule and peptidomimetic inhibitors, demonstrate feasibility of targeting the menin-MLL interaction. On the other hand, biochemical and structural studies revealed that MLL binds to menin in a complex bivalent mode engaging two MLL motifs, and therefore inhibition of this protein-protein interaction represents a challenge.

Chromosomal rearrangements of the MLL gene located at chromosome band 11q23 are found in patients with de novo acute myeloid (AML) and acute lymphoblastic (ALL) leukemias, and in therapy related leukemias or myelodysplastic syndrome (MDS). As a consequence of chromosomal translocations, the MLL gene is fused with one of over 60 different protein partners, such as the most frequent AF4, AF9, ENL, AF6, ELL, and AF10. Disruption of MLL by gene fusions upregulates expression of HOXA9 and MEIS1 genes that are critical to leukemogenesis. The role of HOXA genes in leukemic transformation has been verified in both, in vitro and in vivo models, demonstrating that MLL fusion protein mediated upregulation of HOXA9 and MEIS1 genes results in enhanced proliferation and blockage of hematopoietic differentiation, ultimately leading to acute leukemia. Patients with leukemias harboring MLL translocations have very unfavorable prognosis (20% event free survival at 3 years) and respond poorly to available treatments, demonstrating a clear need for new therapies.

The oncogenic function of MLL fusion proteins is critically dependent on their direct interaction with menin. Menin is a 67 kDa protein encoded by the MENI (Multiple Endocrine Neoplasia I) gene localized on chromosome 11q13. Menin is a ubiquitously expressed protein, predominantly localized in the nucleus. Menin directly binds to the N-terminus of MLL that is retained in all MLL fusion proteins and plays an important role in recruitment of MLL and MLL fusions to target genes, including HOXA9. Loss of menin binding by MLL fusion proteins abolishes their oncogenic properties in vitro and in vivo. Mutations within the N-terminus of MLL-ENL oncoprotein, resulting in protein unable to associate with menin, abolish its potential to upregulate Hox gene expression and induce leukemia in mice. Expression of a dominant-negative inhibitor composed of the amino terminal MLL sequence inhibits growth of the MLL-AF9 transformed bone marrow cells and blocks leukemogenic transformation.

Inhibiting the interaction of menin with the histone methyltransferase MLL1 (KMT2A) has recently emerged as a novel therapeutic strategy. Beneficial therapeutic effects have been postulated in leukemia, prostate, breast, liver and in synovial sarcoma models. In those indications, MLL1 recruitment by menin was described to critically regulate the expression of disease associated genes.

Blocking the menin-MLL interaction might represent a viable approach to reverse the oncogenic activity of MLL fusion proteins in leukemia and may lead to novel therapeutics.

### SUMMARY

A first aspect of the invention relates to compounds of Formula (I):

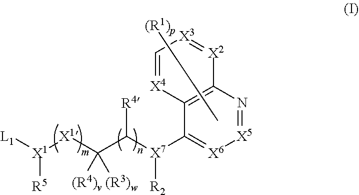

or a pharmaceutically acceptable salt, stereoisomer, solvate, or tautomer thereof, wherein
X¹ is N;
each X¹′ is independently selected from CH₂ and N;
each from X², X³, X⁴, X⁵, and X⁶ is independently selected from CH or N;
and at least one from X², X³, X⁴, X⁵, and X⁶ is N;
X⁷ is N;
each R¹ is independently selected from halogen, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, and NR¹²R¹³ wherein the alkyl, alkoxy, alkenyl, alkynyl, heterocyclyl, cycloalkyl, aryl, or heteroaryl is optionally substituted with one or more substituents independently selected from halogen, OH, NH₂, C₁-C₆ alkyl, C₁-C₆ alkoxy, NR¹²R¹³, C₃-C₁₀ cycloalkyl, C₂-C₆ alkenyl, C₂-C₆ alkynyl, heterocyclyl, aryl, heteroaryl;
R² and R³ are each independently selected from H or C₁-C₆ alkyl; or R² and R³, together with the atoms to which they are bound and any intervening atoms, form a 3-8 membered heterocycle, wherein the heterocycle is optionally substituted with one or more substituents independently selected from halogen, OH, NH₂, C₁-C₆ alkyl, C₁-C₆ alkoxy, and NR¹²R¹³;
R⁴, R⁴′, and R⁵ are each independently selected from H and C₁-C₆ alkyl; or R⁴ and R⁵, together with the atoms to which they are bound and any intervening atoms, form a 3-8 membered heterocycle or heteroaryl, wherein the heterocycle and heteroaryl are optionally substituted with one or more substituents independently selected from halogen, OH, NH₂, C₁-C₆ alkyl, C₁-C₆ alkoxy, and NR¹²R¹³; or R⁴′ and R⁵, together with the atoms to which they are bound and any intervening atoms, form a 3-8 membered heterocycle or heteroaryl, wherein the heterocycle and heteroaryl are optionally substituted with one or more substituents independently selected from halogen, OH, NH₂, C₁-C₆ alkyl, C₁-C₆ alkoxy, and NR¹²R¹³;
wherein at least (a) R² and R³, together with the atoms to which they are bound and any intervening atoms, form said 3-8 membered heterocycle; (b) R⁴ and R⁵, together with the atoms to which they are bound and any intervening atoms, form said 3-8 membered heterocycle or heteroaryl; or (c) R⁴′ and R⁵, together with the atoms to which they are bound and any intervening atoms, form said 3-8 membered heterocycle or heteroaryl;
L¹ is H or

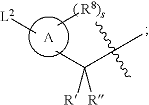

wherein
R′ and R″ are both H;
Ring A is selected from aryl and C₃-C₁₄ cycloalkyl;
L² is independently selected from halogen, C₁-C₆ alkyl, NR¹²R¹³, —N(R⁶)S(O)₂R⁷, —N(R⁶)CH₂R⁷, —N(R⁶)C(O)R⁷, —C(O)NR⁶R⁷, —S(O)₂NR⁶R⁷, wherein the alkyl is optionally substituted with one or more substituents independently selected from halogen, oxo, OH, C₁-C₆ alkyl, C₁-C₆ alkoxy, NR¹²R¹³, cycloalkyl, and heterocyclyl;
R⁶ is selected from H, C₁-C₆ alkyl;
R⁷ is selected from NR¹²R¹³, C₁-C₆ alkyl, C₂-C₆ alkenyl, C₂-C₆ alkynyl, C₃-C₁₀ cycloalkyl, aryl, heteroaryl, or heterocyclyl, wherein the alkyl, alkenyl, alkynyl, cycloalkyl, aryl, heteroaryl, or heterocyclyl is optionally substituted with one or more substituents independently selected from halogen, OH, NH₂, C₁-C₆ alkyl, C₁-C₆ alkoxy, —S(O)₂R⁷, N(R⁶)S(O)₂R⁷, and NR¹²R¹³;
each R⁸ is independently selected from halogen, OH, oxo, CN, C₁-C₆ alkyl, C₂-C₆ alkenyl, C₁-C₆ alkoxy, C₁-C₆ alkyl-NR¹²R¹³, —NR¹²R¹³, wherein the alkyl, or alkoxy is optionally substituted with one or more substituents independently selected from halogen, oxo, OH, NH₂, C₁-C₆ alkyl, C₁-C₆ alkoxy, NR¹²R¹³, cycloalkyl, and heterocyclyl; or
L² and R⁸ together with the atoms to which they are attached and any intervening atoms, form a 3-14 membered cycloalkyl, 5-14 membered heterocycle, or 5-6 membered heteroaryl, wherein the cycloalkyl, heterocycle or heteroaryl is optionally substituted with one or more substituents independently selected from halogen, OH, NH₂, C₁-C₆ alkyl, C₁-C₆ alkoxy, C₁-C₆ alkyl-C₁-C₆ alkoxy, C₁-C₆ alkyl-NHC₁-C₆ alkyl, and NR¹²R¹³; or
each R¹² and R¹³ is independently H, C₁-C₆ alkyl, C₁-C₆ alkoxy, C₃-C₁₀ cycloalkyl, C₂-C₆ alkenyl, —S(O)₂R⁷, C₁-C₆ acyl, heterocyclyl, aryl, or heteroaryl;
or R¹² and R¹³ together with the atoms to which they are attached and any intervening atoms, form a 5-14 membered heterocycle optionally substituted with one or more substituents independently selected from —S(O)₂R⁷;
m is an integer selected from 0, 1, 2, and 3;
n is an integer selected from 0, 1, 2, and 3;
p is an integer selected from 0, 1 and 2;
s is an integer selected from 0, 1 and 2;
v is 1;
w is an integer selected from 0 and 1, w is 0 when R⁴ is a part of aromatic ring; wherein,
aryl is cyclic, aromatic hydrocarbon groups that have 1 to 3 aromatic rings;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

Another aspect of the invention is directed to pharmaceutical compositions comprising a compound of Formula (I), or a pharmaceutically acceptable salt, hydrate, solvate, prodrug, stereoisomer, or tautomer thereof and a pharmaceutically acceptable carrier. The pharmaceutical acceptable carrier may further include an excipient, diluent, or surfactant.

Another aspect of the invention relates to a method of treating a disease or disorder associated with interaction of menin and MLL. The method comprises administering to a patient in need of a treatment for diseases or disorders associated with interaction of menin and MLL an effective amount of a compound of Formula (I), or a pharmaceutically acceptable salt, hydrate, solvate, prodrug, stereoisomer, tautomer, or pharmaceutical composition thereof.

Another aspect of the invention is directed to a method of inhibiting of interaction of menin and MLL. The method involves administering to a patient in need thereof an effective amount of a compound of Formula (I), or a pharmaceutically acceptable salt, hydrate, solvate, prodrug, stereoisomer, tautomer, or pharmaceutical composition thereof.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for inhibiting interaction of menin and MLL.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease associated with inhibiting of interaction of menin and MLL.

Another aspect of the present invention relates to compounds of Formula (I), or pharmaceutically acceptable salts, hydrates, solvates, prodrugs, stereoisomers, tautomers, or pharmaceutical compositions thereof, for use in the manufacture of a medicament for treating or preventing a disease or disorder disclosed herein.

Another aspect of the invention is directed to a method of treating or preventing a disease or disorder disclosed herein in a subject in need thereof. The method involves administering to a patient in need of the treatment an effective amount of a compound of Formula (I), or a pharmaceutically acceptable salt, hydrate, solvate, prodrug, stereoisomer, tautomer, or pharmaceutical composition thereof.

Another aspect of the present invention relates to the use of compounds of Formula (I), or pharmaceutically acceptable salts, hydrates, solvates, prodrugs, stereoisomers, tautomers, or pharmaceutical compositions thereof, in the treatment of a disease or disorder disclosed herein.

The present invention further provides methods of treating a disease or disorder associated with interaction of menin and MLL, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of interaction of menin and MLL that are therapeutic agents in the treatment of diseases and disorders.

The present invention further provides compounds and compositions with an improved efficacy and safety profile relative to known inhibitors of menin and MLL interaction. The present disclosure also provides agents with novel mechanisms of action toward interaction of menin and MLL in the treatment of various types of diseases.

The present invention further provides methods of treating a disease or disorder associated with interaction of menin and MLL, comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

The present invention provides inhibitors of interaction of menin and MLL that are therapeutic agents in the treatment of diseases and disorders.

The present invention further provides methods of treating a disease, disorder, or condition selected from cancer, acute myeloid (AML) and acute lymphoblastic (ALL) leukemias, or myelodysplastic syndrome (MDS), comprising administering to a patient suffering from at least one of said diseases or disorders a compound of Formula (I), or a pharmaceutically acceptable salt, hydrate, solvate, prodrug, stereoisomer, tautomer, or pharmaceutical composition thereof.

In some aspects, the present disclosure provides a compound obtainable by, or obtained by, a method for preparing compounds described herein (e.g., a method comprising one or more steps described in General Procedure A or B).

In some aspects, the present disclosure provides an intermediate as described herein, being suitable for use in a method for preparing a compound as described herein (e.g., the intermediate is selected from the intermediates described in Preparative part-P1-P175).

In some aspects, the present disclosure provides a method of preparing compounds of the present disclosure.

In some aspects, the present disclosure provides a method of preparing compounds of the present disclosure, comprising one or more steps described herein.

Unless otherwise defined, all technical and scientific terms used herein have the same meaning as commonly understood by one of ordinary skill in the art to which this disclosure belongs. In the specification, the singular forms also include the plural unless the context clearly dictates otherwise. Although methods and materials similar or equivalent to those described herein can be used in the practice or testing of the present disclosure, suitable methods and materials are described below. In addition, the materials, methods and examples are illustrative only and are not intended to be limiting. In the case of conflict between the chemical structures and names of the compounds disclosed herein, the chemical structures will control.

Other features and advantages of the disclosure will be apparent from the following detailed description and claims

### DETAILED DESCRIPTION

The present disclosure provides methods of treating, preventing, or ameliorating a disease or disorder in which associated with the inhibition of the interaction of menin and MILLI by administering to a subject in need thereof a therapeutically effective amount of a compound as disclosed herein.

The details of the disclosure are set forth in the accompanying description below. Although methods and materials similar or equivalent to those described herein can be used in the practice or testing of the present disclosure, illustrative methods and materials are now described. Other features, objects, and advantages of the disclosure will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this disclosure belongs. All patents and publications cited in this specification are incorporated herein by reference in their entireties.

### Definitions

The articles “a” and “an” are used in this disclosure to refer to one or more than one (i.e., to at least one) of the grammatical object of the article. By way of example, “an element” means one element or more than one element.

The term “and/or” is used in this disclosure to mean either “and” or “or” unless indicated otherwise.

The term “optionally substituted” is understood to mean that a given chemical moiety (e.g., an alkyl group) can (but is not required to) be bonded other substituents (e.g., heteroatoms). For instance, an alkyl group that is optionally substituted can be a fully saturated alkyl chain (i.e., a pure hydrocarbon). Alternatively, the same optionally substituted alkyl group can have one or more substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups but does not necessarily have any further functional groups. Suitable substituents used in the optional substitution of the described groups include, without limitation, halogen, oxo, —OH, —CN, —COOH, —CH₂CN, —O—(C₁-C₆) alkyl, (C₁-C₆)alkyl, (C₁-C₆) alkoxy, (C₁-C₆) haloalkyl, (C₁-C₆) haloalkoxy, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —NHC(O)(C₁-C₆)alkyl, —C(O)NH(C₁-C₆)alkyl, —S(O)₂(C₁-C₆)alkyl, —S(O)NH(C₁-C₆)alkyl, and —S(O)N((C₁-C₆)alkyl)₂. The substituents can themselves be optionally substituted. “Optionally substituted” as used herein also refers to substituted or unsubstituted whose meaning is described below.

As used herein, the term “substituted” means that the specified group or moiety bears one or more suitable substituents wherein the substituents may connect to the specified group or moiety at one or more positions. For example, an aryl substituted with a cycloalkyl may indicate that the cycloalkyl connects to one atom of the aryl with a bond or by fusing with the aryl and sharing two or more common atoms.

As used herein, the term “unsubstituted” means that the specified group bears no substituents.

Unless otherwise specifically defined, the term “aryl” refers to cyclic, aromatic hydrocarbon groups that have 1 to 3 aromatic rings, including monocyclic or bicyclic groups such as phenyl, biphenyl or naphthyl. Where containing two aromatic rings (bicyclic, etc.), the aromatic rings of the aryl group may be joined at a single point (e.g., biphenyl), or fused (e.g., naphthyl). The aryl group may be optionally substituted by one or more substituents, e.g., 1 to 5 substituents, at any point of attachment. Exemplary substituents include, but are not limited to, —H, -halogen, —O—(C₁-C₆)alkyl, (C₁-C₆)alkyl, —O—(C₂-C₆) alkenyl, —O—(C₂-C₆) alkynyl, (C₂-C₆) alkenyl, (C₂-C₆) alkynyl, —OH, —OP(O)(OH)₂, —OC(O)(C₁-C₆)alkyl, —C(O)(C₁-C₆)alkyl, —OC(O)O(C₁-C₆)alkyl, —NH₂, —NH((C₁-C₆)alkyl), —N((C₁-C₆)alkyl)₂, —S(O)₂—(C₁-C₆)alkyl, —S(O)NH(C₁-C₆)alkyl, and —S(O)N((C₁-C₆)alkyl)₂. The substituents can themselves be optionally substituted. Furthermore, when containing two fused rings the aryl groups herein defined may have one or more saturated or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, tetrahydrobenzoannulenyl, and the like.

Unless otherwise specifically defined, “heteroaryl” means a monovalent monocyclic or a polycyclic aromatic radical of 5 to 24 ring atoms, containing one or more ring heteroatoms selected from N, O, S, P, or B, the remaining ring atoms being C. A polycyclic aromatic radical includes two or more fused rings and may further include two or more spiro-fused rings, e.g., bicyclic, tricyclic, tetracyclic, and the like. Unless otherwise specifically defined, “fused” means two rings sharing two ring atoms. Unless otherwise specifically defined, “spiro-fused” means two rings sharing one ring atom. Heteroaryl as herein defined also means a bicyclic heteroaromatic group wherein the heteroatom is selected from N, O, S, P, or B. Heteroaryl as herein defined also means a tricyclic heteroaromatic group containing one or more ring heteroatoms selected from N, O, S, P, or B. Heteroaryl as herein defined also means a tetracyclic heteroaromatic group containing one or more ring heteroatoms selected from N, O, S, P, or B. The aromatic radical is optionally substituted independently with one or more substituents described herein. Examples include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo (1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuran, chromanyl, thiochromanyl, tetrahydroquinolinyl, dihydrobenzothiazine, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridiny], [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, 3H-indolyl, and derivatives thereof. Furthermore, when containing two or more fused rings, the heteroaryl groups defined herein may have one or more saturated or partially unsaturated ring fused with one or more fully unsaturated aromatic ring. In heteroaryl ring systems containing more than two fused rings, a saturated or partially unsaturated ring may further be fused with a saturated or partially unsaturated ring described herein. Furthermore, when containing three or more fused rings, the heteroaryl groups defined herein may have one or more saturated or partially unsaturated ring spiro-fused. Any saturated or partially unsaturated ring described herein is optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, indolinyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizine, pyrazolo[1,5-a]pyrimidin-7 (4H)-only, 3,4-dihydropyrazino[1,2-a]indol-1(2H)-onyl, benzo[c][1,2]oxaborol-1(3H)-olyl, 6,6a,7,8-tetrahydro-9H-pyrido[2,3-b]puyrrolo[1,2-d][1,4]oxazin-9-onyl, or 6a′,7′-dihydro-6′H,9′H-spiro[cyclopropane-1,8′-pyrido[2,3-b]pyrrolo[1,2-d][1,4]oxazin]-9′-onyl.

Halogen or “halo” refers to fluorine, chlorine, bromine, or iodine.

Alkyl refers to a straight or branched chain saturated hydrocarbon containing 1-12 carbon atoms. Examples of a (C₁-C₆)alkyl group include, but are not limited to, methyl, ethyl, propyl, butyl, pentyl, hexyl, isopropyl, isobutyl, sec-butyl, tert-butyl, isopentyl, neopentyl, and isohexyl.

“Alkoxy” refers to a straight or branched chain saturated hydrocarbon containing 1-12 carbon atoms containing a terminal “O” in the chain, i.e., —O(alkyl). Examples of alkoxy groups include without limitation, methoxy, ethoxy, propoxy, butoxy, t-butoxy, or pentoxy groups.

“Alkenyl” refers to a straight or branched chain unsaturated hydrocarbon containing 2-12 carbon atoms. The “alkenyl” group contains at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl. An alkenyl group can be unsubstituted or substituted. Alkenyl, as herein defined, may be straight or branched.

“Alkynyl” refers to a straight or branched chain unsaturated hydrocarbon containing 2-12 carbon atoms. The “alkynyl” group contains at least one triple bond in the chain. Examples of alkenyl groups include ethynyl, propargyl, n-butynyl, iso-butynyl, pentynyl, or hexynyl. An alkynyl group can be unsubstituted or substituted.

The term “alkylene” or “alkylenyl” refers to a divalent alkyl radical. Any of the above-mentioned monovalent alkyl groups may be an alkylene by abstraction of a second hydrogen atom from the alkyl. As herein defined, alkylene may also be a C₁-C₆ alkylene. An alkylene may further be a C₁-C₄ alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

“Cycloalkyl” means mono or polycyclic saturated carbon rings containing 3-18 carbon atoms. Polycyclic cycloalkyl may be fused bicyclic cycloalkyl, bridged bicyclic cycloalkyl, or spiro-fused bicyclic cycloalkyl. A polycyclic cycloalkyl comprises at least one non-aromatic ring. Examples of cycloalkyl groups include, without limitations, cyclopropyl, cyclobutyl, cyclopentyl, cyclohexyl, cycloheptanyl, cyclooctanyl, norbornyl, norborenyl, 1,2,3,4-tetrahydronaphthyl, 2,3-dihydro-1H-indenyl, spiro[3.5]nonyl, spiro[5.5]undecyl, bicyclo[1.1.1]pentanyl, bicyclo[2.2.2]octanyl, or bicyclo[2.2.2]octenyl.

“Heterocyclyl”, “heterocycle” or “heterocycloalkyl” mono or polycyclic rings containing 3-24 atoms which include carbon and one or more heteroatoms selected from N, O, S, P, or B and wherein the rings are not aromatic. The heterocycloalkyl ring structure may be substituted by one or more substituents. The substituents can themselves be optionally substituted. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl.

The term “aromatic” means a planar ring having 4n+2 electrons in a conjugated system. As used herein, “conjugated system” means a system of connected p-orbitals with delocalized electrons, and the system may include lone electron pairs.

The term “haloalkyl” as used herein refers to an alkyl group, as defined herein, which is substituted one or more halogen. Examples of haloalkyl groups include, but are not limited to, trifluoromethyl, difluoromethyl, pentafluoroethyl, trichloromethyl, etc.

The term “haloalkoxy” as used herein refers to an alkoxy group, as defined herein, which is substituted with one or more halogen. Examples of haloalkyl groups include, but are not limited to, trifluoromethoxy, difluoromethoxy, pentafluoroethoxy, trichloromethoxy, etc.

The term “cyano” as used herein means a substituent having a carbon atom joined to a nitrogen atom by a triple bond, i.e., C═N.

“Spirocycloalkyl” or “spirocyclyl” means carbogenic bicyclic ring systems with both rings connected through a single atom. The ring can be different in size and nature, or identical in size and nature. Examples include spiropentane, spriohexane, spiroheptane, spirooctane, spirononane, or spirodecane. One or both of the rings in a spirocycle can be fused to another ring carbocyclic, heterocyclic, aromatic, or heteroaromatic ring. One or more of the carbon atoms in the spirocycle can be substituted with a heteroatom (e.g., O, N, S, or P). A (C₃-C₁₂) spirocycloalkyl is a spirocycle containing between 3 and 12 carbon atoms. One or more of the carbon atoms can be substituted with a heteroatom.

The term “spiroheterocycloalkyl”, “spiroheterocycle”, or “spiroheterocyclyl” is understood to mean a spirocycle wherein at least one of the rings is a heterocycle (e.g., at least one of the rings is furanyl, morpholinyl, or piperidinyl).

The term “solvate” refers to a complex of variable stoichiometry formed by a solute and solvent. Such solvents for the purpose of the disclosure may not interfere with the biological activity of the solute. Examples of suitable solvents include, but are not limited to, water, MeOH, EtOH, and AcOH. Solvates wherein water is the solvent molecule are typically referred to as hydrates. Hydrates include compositions containing stoichiometric amounts of water, as well as compositions containing variable amounts of water.

The term “isomer” refers to compounds that have the same composition and molecular weight but differ in physical and/or chemical properties. The structural difference may be in constitution (geometric isomers) or in the ability to rotate the plane of polarized light (stereoisomers). With regard to stereoisomers, the compounds of Formula (I) may have one or more asymmetric carbon atom and may occur as racemates, racemic mixtures and as individual enantiomers or diasteromers.

The present disclosure also contemplates isotopically labelled compounds of Formula I (e.g., those labeled with ²H and ¹⁴C). Deuterated (i.e., ²H or D) and carbon-14 (i.e., ¹⁴C) isotopes are particularly preferred for their ease of preparation and detectability. Further, substitution with heavier isotopes such as deuterium may afford certain therapeutic advantages resulting from greater metabolic stability (e.g., increased in vivo half-life or reduced dosage requirements) and hence may be preferred in some circumstances. Isotopically labelled compounds of Formula I can generally be prepared by following procedures analogous to those disclosed in the Schemes and/or in the Examples herein below, by substituting an appropriate isotopically labelled reagent for a non-isotopically labelled reagent.

The disclosure also includes pharmaceutical compositions comprising a therapeutically effective amount of a disclosed compound and a pharmaceutically acceptable carrier. Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate, pantothenate, phosphate/diphosphate, picrate, polygalacturonate, propionate, p-toluenesulfonate, salicylate, stearate, subacetate, succinate, sulfate, sulfosalicylate, tannate, tartrate, teoclate, tosylate, triethiodide, and valerate salts.

A “patient” or “subject” is a mammal, e.g., a human, mouse, rat, guinea pig, dog, cat, horse, cow, pig, or non-human primate, such as a monkey, chimpanzee, baboon, or rhesus.

An “effective amount” when used in connection with a compound is an amount effective for treating or preventing a disease in a subject as described herein.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body of a subject.

The term “treating” with regard to a subject, refers to improving at least one symptom of the subject's disorder. Treating includes curing, improving, or at least partially ameliorating the disorder.

The term “disorder” is used in this disclosure to mean, and is used interchangeably with, the terms disease, condition, or illness, unless otherwise indicated.

The term “administer”, “administering”, or “administration” as used in this disclosure refers to either directly administering a disclosed compound or pharmaceutically acceptable salt of the disclosed compound or a composition to a subject or administering a prodrug derivative or analog of the compound or pharmaceutically acceptable salt of the compound or composition to the subject, which can form an equivalent amount of active compound within the subject's body.

The term “prodrug,” as used in this disclosure, means a compound which is convertible in vivo by metabolic means (e.g., by hydrolysis) to a disclosed compound

The term “salt” refers to pharmaceutically acceptable salts

The term “pharmaceutically acceptable salt” also refers to a salt of the compositions of the present disclosure having an acidic functional group, such as a carboxylic acid functional group, and a base.

“Menin/MLL interaction inhibitor” as used herein refer to compounds of Formula I and/or compositions comprising a compound of Formula I which inhibits the interaction of menin and MLL.

The amount of compound of composition described herein needed for achieving a therapeutic effect may be determined empirically in accordance with conventional procedures for the particular purpose. Generally, for administering therapeutic agents (e.g., compounds or compositions of Formula I (and/or additional agents) described herein) for therapeutic purposes, the therapeutic agents are given at a pharmacologically effective dose. A “pharmacologically effective amount,” “pharmacologically effective dose,” “therapeutically effective amount,” or “effective amount” refers to an amount sufficient to produce the desired physiological effect or amount capable of achieving the desired result, particularly for treating the disorder or disease. An effective amount as used herein would include an amount sufficient to, for example, delay the development of a symptom of the disorder or disease, alter the course of a symptom of the disorder or disease (e.g., slow the progression of a symptom of the disease), reduce or eliminate one or more symptoms or manifestations of the disorder or disease, and reverse a symptom of a disorder or disease. For example, administration of therapeutic agents to a subject suffering from cancer provides a therapeutic benefit not only when the underlying condition is eradicated or ameliorated, but also when the subject reports a decrease in the severity or duration of the symptoms associated with the disease, e.g., a decrease in tumor burden, a decrease in circulating tumor cells, an increase in progression free survival. Therapeutic benefit also includes halting or slowing the progression of the underlying disease or disorder, regardless of whether improvement is realized.

### Compounds of the Present Disclosure

In various aspects of the present disclosure, inhibitors of menin/MLL interaction comprise compounds having a structure represented by Formula (I):

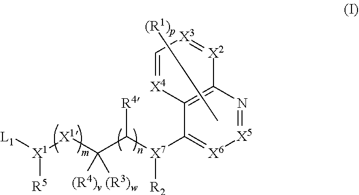

or a pharmaceutically acceptable salt, stereoisomer, solvate, or tautomer thereof, wherein,
X¹ is N;
each X¹′ is independently selected from CH₂ and N;
each from X², X³, X⁴, X⁵, and X⁶ is independently selected from CH or N;
and at least one from X², X³, X⁴, X⁵, and X⁶ is N;
X⁷ is N;
each R¹ is independently selected from halogen, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂—Co alkynyl-aryl, C₂-C₆ alkynyl-heteroaryl, and NR¹²R¹³ wherein the alkyl, alkoxy, alkenyl, alkynyl, heterocyclyl, cycloalkyl, aryl, or heteroaryl is optionally substituted with one or more substituents independently selected from halogen, OH, NH₂, C₁-C₆ alkyl, C₁-C₆ alkoxy, NR¹²R¹³, C₃-C₁₀ cycloalkyl, C₂-C₆ alkenyl, C₂-C₆ alkynyl, heterocyclyl, aryl, heteroaryl;
R² and R³ are each independently selected from H or C₁-C₆alkyl; or R² and R³, together with the atoms to which they are bound and any intervening atoms, form a 3-8 membered heterocycle, wherein the heterocycle is optionally substituted with one or more substituents independently selected from halogen, OH, NH₂, C₁-C₆ alkyl, C₁-C₆ alkoxy, and NR¹²R¹³;
R⁴, R⁴′, and R⁵ are each independently selected from H and C₁-C₆ alkyl; or R⁴ and R⁵, together with the atoms to which they are bound and any intervening atoms, form a 3-8 membered heterocycle or heteroaryl, wherein the heterocycle and heteroaryl are optionally substituted with one or more substituents independently selected from halogen, OH, NH₂, C₁-C₆ alkyl, C₁-C₆ alkoxy, and NR¹²R¹³; or R⁴′ and R⁵, together with the atoms to which they are bound and any intervening atoms, form a 3-8 membered heterocycle or heteroaryl, wherein the heterocycle and heteroaryl are optionally substituted with one or more substituents independently selected from halogen, OH, NH₂, C₁-C₆ alkyl, C₁-C₆ alkoxy, and NR¹²R¹³;
wherein at least (a) R² and R³, together with the atoms to which they are bound and any intervening atoms, form said 3-8 membered heterocycle; (b) R⁴ and R⁵, together with the atoms to which they are bound and any intervening atoms, form said 3-8 membered heterocycle or heteroaryl; or (c) R⁴′ and R⁵, together with the atoms to which they are bound and any intervening atoms, form said 3-8 membered heterocycle or heteroaryl;
L¹ is H or

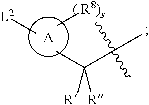

wherein
R′ and R″ are both H;
Ring A is selected from aryl and C₃-C₁₄ cycloalkyl;
L² is independently selected from halogen, C₁-C₆ alkyl, NR¹²R¹³, —N(R⁶)S(O)₂R⁷, —N(R⁶)CH₂R⁷, —N(R⁶)C(O)R⁷, —C(O)NR⁶R⁷, —S(O)₂NR⁶R⁷, wherein the alkyl is optionally substituted with one or more substituents independently selected from halogen, oxo, OH, C₁-C₆ alkyl, C₁-C₆ alkoxy, NR¹²R¹³, cycloalkyl, and heterocyclyl;
R⁶ is selected from H, C₁-C₆ alkyl;
R⁷ is selected from NR¹²R¹³, C₁-C₆ alkyl, C₂-C₆ alkenyl, C₂-C₆ alkynyl, C₃-C₁₀ cycloalkyl, aryl, heteroaryl, or heterocyclyl, wherein the alkyl, alkenyl, alkynyl, cycloalkyl, aryl, heteroaryl, or heterocyclyl is optionally substituted with one or more substituents independently selected from halogen, OH, NH₂, C₁-C₆ alkyl, C₁-C₆ alkoxy, —S(O)₂R⁷, N(R⁶)S(O))₂R⁷, and NR¹²R¹³;
each R⁸ is independently selected from halogen, OH, oxo, CN, C₁-C₆ alkyl, C₂-C₆ alkenyl, C₁-C₆ alkoxy, C₁-C₆ alkyl-NR¹²R¹³, —NR¹²R¹³, wherein the alkyl, or alkoxy is optionally substituted with one or more substituents independently selected from halogen, oxo, OH, NH₂, C₁-C₆ alkyl, C₁-C₆ alkoxy, NR¹²R¹³, cycloalkyl, and heterocyclyl; or
L² and R⁸ together with the atoms to which they are attached and any intervening atoms, form a 3-14 membered cycloalkyl, 5-14 membered heterocycle, or 5-6 membered heteroaryl, wherein the cycloalkyl, heterocycle or heteroaryl is optionally substituted with one or more substituents independently selected from halogen, OH, NH₂, C₁-C₆ alkyl, C₁-C₆ alkoxy, C₁-C₆ alkyl-C₁-C₆ alkoxy, C₁-C₆ alkyl-NHC₁-C₆ alkyl, and NR¹²R¹³; or
each R¹² and R¹³ is independently H, C₁-C₆ alkyl, C₁-C₆ alkoxy, C₃-C₁₀ cycloalkyl, C₂-C₁₆ alkenyl, —S(O)₂R⁷, C₁-C₆ acyl, heterocyclyl, aryl, or heteroaryl;
or R¹² and R¹³ together with the atoms to which they are attached and any intervening atoms, form a 5-14 membered heterocycle optionally substituted with one or more substituents independently selected from —S(O)₂R⁷;
m is an integer selected from 0, 1, 2, and 3;
n is an integer selected from 0, 1, 2, and 3;
p is an integer selected from 0, 1 and 2;
s is an integer selected from 0, 1 and 2;
vis 1; and
w is an integer selected from 0 and 1, w is 0 when R⁴ is a part of aromatic ring.

For the compounds according to Formula (I), heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B; and heteroaryl is a monovalent monocyclic or a polycyclic aromatic radical of 5 to 24 ring atoms, containing one or more ring heteroatoms selected from N, O, S, P, or B, the remaining ring atoms being C.

In various aspects, the present disclosure provides compounds of Formula (I) and salts, stereoisomers, solvates, prodrugs, isotopic derivatives, and tautomers thereof:

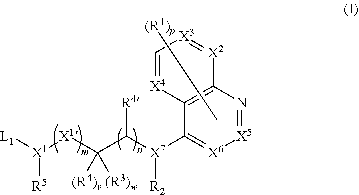

wherein R¹, R², R³, R⁴, R⁴′, R⁵, X¹, X¹′, X², X³, X⁴, X⁵, X⁶, X⁷, L¹, m, n, p, s, v, and w are as described herein.

It is understood that, for a compound of Formula (I), R¹, R², R³, R⁴, R⁴′, R⁵, R⁶, R⁷, R⁸, R⁹, R¹⁰, R¹¹, R¹², R¹³, R′, R″, A, X¹, X¹′, X², X³, X⁴, X⁵, X⁶, X⁷, L¹, L², m, n, p, s, v and w can each be, where applicable, selected from the groups described herein, and any group described herein for any of R¹, R², R³, R⁴, R⁴′, R⁵, R⁶, R⁷, R⁸, R⁹, R¹⁰, R¹¹, R¹², R¹³, R′, R″, A, X¹, X¹′, X², X³, X⁴, X⁵, X⁶, X⁷, L¹, L², m, n, p, s, v and w can be combined, where applicable, with any group described herein for one or more of the remainder of R¹, R², R³, R⁴, R⁴′, R⁵, R⁶, R⁷, R⁸, R⁹, R¹⁰, R¹¹, R¹², R¹³, R′, R″, A, X¹, X¹′, X², X³, X⁴, X⁵, X⁶, X⁷, L¹, L², m, n, p, s, v and w.

In some embodiments,
X¹ is N;
each X¹′ is independently selected from CH₂ and N;
each from X², X³, X⁴, X⁵, and X⁶ is independently selected from CH or N;
and at least one from X², X³, X⁴, X⁵, and X⁶ is N;
X⁷ is N;
each R¹ is independently selected from halogen, C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, and NR¹²R¹³ wherein the alkyl, alkoxy, alkenyl, alkynyl, heterocyclyl, cycloalkyl, aryl, or heteroaryl is optionally substituted with one or more substituents independently selected from halogen, OH, NH₂, C₁-C₆ alkyl, C₁-C₆ alkoxy, NR¹²R¹³, C₃-C₁₀ cycloalkyl, C₂-C₆ alkenyl, C₂-C₆ alkynyl, heterocyclyl, aryl, heteroaryl;
R² and R³ are each independently selected from H or C₁-C₆alkyl; or R² and R³, together with the atoms to which they are bound and any intervening atoms, form a 3-8 membered heterocycle, wherein the heterocycle is optionally substituted with one or more substituents independently selected from halogen, OH, NH₂, C₁-C₆ alkyl, C₁-C₆ alkoxy, and NR¹²R¹³;
R⁴, R⁴′, and R⁵ are each independently selected from H and C₁-C₆ alkyl; or R⁴ and R⁵, together with the atoms to which they are bound and any intervening atoms, form a 3-8 membered heterocycle or heteroaryl, wherein the heterocycle and heteroaryl are optionally substituted with one or more substituents independently selected from halogen, OH, NH₂, C₁-C₆ alkyl, C₁-C₆ alkoxy, and NR¹²R¹³; or R⁴′ and R⁵, together with the atoms to which they are bound and any intervening atoms, form a 3-8 membered heterocycle or heteroaryl, wherein the heterocycle and heteroaryl are optionally substituted with one or more substituents independently selected from halogen, OH, NH₂, C₁-C₆ alkyl, C₁-C₆ alkoxy, and NR¹²R¹³;
wherein at least (a) R² and R³, together with the atoms to which they are bound and any intervening atoms, form said 3-8 membered heterocycle; (b) R⁴ and R⁵, together with the atoms to which they are bound and any intervening atoms, form said 3-8 membered heterocycle or heteroaryl; or (c) R⁴′ and R⁵, together with the atoms to which they are bound and any intervening atoms, form said 3-8 membered heterocycle or heteroaryl;
L¹ is H or

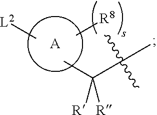

wherein
R′ and R″ are both H;
Ring A is selected from aryl and C₃-C₁₄ cycloalkyl;
L² is independently selected from halogen, C₁-C₆ alkyl, NR¹²R¹³, —N(R⁶)S(O)₂R⁷, —N(R⁶)CH₂R⁷, —N(R⁶)C(O)R⁷, —C(O)NR⁶R⁷, —S(O)₂NR⁶R⁷, wherein the alkyl is optionally substituted with one or more substituents independently selected from halogen, oxo, OH, C₁-C₆ alkyl, C₁-C₆ alkoxy, NR¹²R¹³, cycloalkyl, and heterocyclyl;
R⁶ is selected from H, C₁-C₆ alkyl;
R⁷ is selected from NR¹²R¹³, C₁-C₆ alkyl, C₂-C₆ alkenyl, C₂-C₆ alkynyl, C₃-C₁₀ cycloalkyl, aryl, heteroaryl, or heterocyclyl, wherein the alkyl, alkenyl, alkynyl, cycloalkyl, aryl, heteroaryl, or heterocyclyl is optionally substituted with one or more substituents independently selected from halogen, OH, NH₂, C₁-C₆ alkyl, C₁-C₆ alkoxy, —S(O)₂R⁷, N(R⁶)S(O)₂R⁷, and NR¹²R¹³;
each R⁸ is independently selected from halogen, OH, oxo, CN, C₁-C₆ alkyl, C₂-C₆ alkenyl, C₁-C₆ alkoxy, C₁-C₆ alkyl-NR¹²R¹³, —NR¹²R¹³, wherein the alkyl, or alkoxy is optionally substituted with one or more substituents independently selected from halogen, oxo, OH, NH₂, C₁-C₆ alkyl, C₁-C₆ alkoxy, NR¹²R¹³, cycloalkyl, and heterocyclyl; or
L² and R⁸ together with the atoms to which they are attached and any intervening atoms, form a 3-14 membered cycloalkyl, 5-14 membered heterocycle, or 5-6 membered heteroaryl, wherein the cycloalkyl, heterocycle or heteroaryl is optionally substituted with one or more substituents independently selected from halogen, OH, NH₂, C₁-C₆ alkyl, C₁-C₆ alkoxy, C₁-C₆ alkyl-C₁-C₆ alkoxy, C₁-C₆ alkyl-NHC₁-C₆ alkyl, and NR¹²R¹³; or
each R¹² and R¹³ is independently H, C₁-C₆ alkyl, C₁-C₆ alkoxy, C₃-C₁₀ cycloalkyl, C₂-C₆ alkenyl, —S(O)₂R⁷, C₁-C₆ acyl, heterocyclyl, aryl, or heteroaryl;
or R¹² and R¹³ together with the atoms to which they are attached and any intervening atoms, form a 5-14 membered heterocycle optionally substituted with one or more substituents independently selected from —S(O)₂R⁷;
m is an integer selected from 0, 1, 2, and 3;
n is an integer selected from 0, 1, 2, and 3;
p is an integer selected from 0, 1 and 2;
s is an integer selected from 0, 1 and 2;
v is 1;
w is an integer selected from 0 and 1, w is 0 when R⁴ is a part of aromatic ring.

In some embodiments, L¹ is H.

In some embodiments, L¹ is

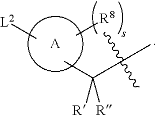

In some embodiments, X¹ is CH or N. In some embodiments, X¹ is CH. In some embodiments, X¹ is N.

In some embodiments, m is 0.

In some embodiments, m is 1. In some embodiments, m is 1 and X¹′ is CH or N. In some embodiments, m is 1 and X″ is CH. In some embodiments, m is 1 and X¹′ is N.

In some embodiments, m is 2. In some embodiments, m is 2 and each X¹′ is independently selected from CH or N. In some embodiments, m is 2 and both X¹′ are CH. In some embodiments, m is 2 and both X¹′ are N. In some embodiments, m is 2, the X¹′ adjacent to X¹ is CH, and the other X¹′ is N. In some embodiments, m is 2, the X¹′ adjacent to X¹ is N, and the other X¹′ is CH.

In some embodiments, X² is CH or N. In some embodiments, X² is CH. In some embodiments, X² is N.

In some embodiments, X³ is CH or N. In some embodiments, X³ is CH. In some embodiments, X³ is N.

In some embodiments, X⁴ is CH or N. In some embodiments, X⁴ is CH. In some embodiments, X⁴ is N.

In some embodiments, X⁵ is CH or N. In some embodiments, X⁵ is CH. In some embodiments, X⁵ is N.

In some embodiments, X⁶ is CH or N. In some embodiments, X⁶ is CH. In some embodiments, X⁶ is N.

In some embodiments, X⁷ is C, CH, or N. In some embodiments, X⁷ is C and X⁷ is part of aromatic ring. In some embodiments, X⁷ is CH. In some embodiments, X⁷ is N.

In some embodiments, each R¹ is independently selected from halogen, C₁-C₆ alkyl, C₁-C₆ alkoxy, C₂-C₆ alkenyl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, and C₂-C₆ alkynyl-heteroaryl. In some embodiments, each R¹ is independently selected from C₁-C₆ alkyl, C₁-C₆ alkoxy, C₂-C₆ alkenyl, and C₂-C₆ alkynyl.

In some embodiments, R¹ is halogen.

In some embodiments, each R¹ is independently selected from C₃-C₁₀ cycloalkyl, heterocyclyl, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, and C₂-C₆ alkynyl-heteroaryl. In some embodiments, each R¹ is independently selected from C₃-C₁₀ cycloalkyl, heterocyclyl, aryl, and heteroaryl.

In some embodiments, each R¹ is independently selected from C₁-C₆ alkyl and C₁-C₆ alkoxy. In some embodiments, each R¹ is independently selected from C₃-C₁₀ cycloalkyl and aryl. In some embodiments, each R¹ is independently selected from heterocyclyl and heteroaryl.

In some embodiments, R¹ is C₁-C₆ alkyl.

In some embodiments, R¹ is methyl. In some embodiments, R¹ is ethyl. In some embodiments, R¹ is propyl. In some embodiments, R¹ is n-propyl. In some embodiments, R¹ is isopropyl. In some embodiments, R¹ is butyl. In some embodiments, R¹ is n-butyl. In some embodiments, R¹ is isobutyl. In some embodiments, R¹ is sec-butyl. In some embodiments, R¹ is tert-butyl. In some embodiments, R¹ is pentyl. In some embodiments, R¹ is hexyl.

In some embodiments, R¹ is C₁-C₆ alkoxy.

In some embodiments, R¹ is methoxy. In some embodiments, R¹ is ethoxy. In some embodiments, R¹ is propoxy. In some embodiments, R¹ is butoxy. In some embodiments, R¹ is pentoxy. In some embodiments, one R¹ is hexoxy.

In some embodiments, R¹ is C₃-C₁₀ cycloalkyl.

In some embodiments, R¹ is a monocyclic C₃-C₁₀ cycloalkyl. In some embodiments, R¹ is a polycyclic C₃-C₁₀ cycloalkyl.

In some embodiments, R¹ is C₅-C₆ cycloalkyl.

In some embodiments, R¹ is cyclopropyl, cyclobutyl, cyclopentyl, cyclohexyl, cycloheptyl, cyclooctyl, cyclononyl, or cyclodecyl. In some embodiments, R¹ is cyclopropyl. In some embodiments, R¹ is cyclobutyl. In some embodiments, R¹ is cyclopentyl. In some embodiments, R¹ is cyclohexyl. In some embodiments, R¹ is cycloheptyl. In some embodiments, R¹ is cyclooctyl. In some embodiments, R¹ is cyclononyl. In some embodiments, R¹ is cyclodecyl.

In some embodiments, R¹ is a fused polycyclic C₃-C₁₀ cycloalkyl. In some embodiments, R¹ is a bridged polycyclic C₃-C₁₀ cycloalkyl. In some embodiments, R¹ is a C₃-C₁₀ spirocycloalkyl.

In some embodiments, R¹ is C₂-C₆ alkenyl.

In some embodiments, R¹ is C₂ alkenyl. In some embodiments, R¹ is C₃ alkenyl. In some embodiments, R¹ is C₄ alkenyl. In some embodiments, R¹ is C₅ alkenyl. In some embodiments, R¹ is C₆ alkenyl.

In some embodiments, R¹ is C₂-C₆ alkynyl.

In some embodiments, R¹ is C₂ alkynyl. In some embodiments, R¹ is C₅ alkynyl. In some embodiments, R¹ is C₄ alkynyl. In some embodiments, R¹ is C₅ alkynyl. In some embodiments, R¹ is C₆ alkynyl.

In some embodiments, R¹ is heterocyclyl. In some embodiments, R¹ is 3-10 membered heterocycle. In some embodiments, R¹ is heterocycle comprising one, two, or three heteroatoms. In some embodiments, R¹ is 3-10 membered heterocycle comprising one, two, or three heteroatoms.

In some embodiments, R¹ is a monocyclic heterocycle. In some embodiments, R¹ is a polycyclic heterocycle.

In some embodiments, R¹ is 3-membered heterocycle. In some embodiments, R¹ is 4-membered heterocycle. In some embodiments, R¹ is 5-membered heterocycle. In some embodiments, R¹ is 6-membered heterocycle. In some embodiments, R¹ is 7-membered heterocycle. In some embodiments, R¹ is 8-membered heterocycle. In some embodiments, R¹ is 9-membered heterocycle. In some embodiments, R¹ is 10-membered heterocycle.

In some embodiments, R¹ is 5- to 6-membered heterocycle.

In some embodiments, R¹ is aryl.

In some embodiments, R¹ is C₆ aryl (e.g., phenyl).

In some embodiments, R¹ is heteroaryl. In some embodiments, R¹ is 5- to 6-membered heteroaryl.

In some embodiments, R¹ is C₁-C₆ alkyl-aryl.

In some embodiments, R¹ is methyl-aryl. In some embodiments, R¹ is ethyl-aryl. In some embodiments, R¹ is propyl-aryl. In some embodiments, R¹ is n-propyl-aryl. In some embodiments, R¹ is isopropyl-aryl. In some embodiments, R¹ is butyl-aryl. In some embodiments, R¹ is n-butyl-aryl. In some embodiments, R¹ is isobutyl-aryl. In some embodiments, R¹ is sec-butyl-aryl. In some embodiments, R¹ is tert-butyl-aryl. In some embodiments, R¹ is pentyl-aryl. In some embodiments, R¹ is hexyl-aryl.

In some embodiments, R¹ is C₁-C₆ alkyl-heteroaryl.

In some embodiments, R¹ is methyl-heteroaryl. In some embodiments, R¹ is ethyl-heteroaryl. In some embodiments, R¹ is propyl-heteroaryl. In some embodiments, R¹ is n-propyl-heteroaryl. In some embodiments, R¹ is isopropyl-heteroaryl. In some embodiments, R¹ is butyl-heteroaryl. In some embodiments, R¹ is n-butyl-heteroaryl. In some embodiments, R¹ is isobutyl-heteroaryl. In some embodiments, R¹ is sec-butyl-heteroaryl. In some embodiments, R¹ is tert-butyl-heteroaryl. In some embodiments, R¹ is pentyl-heteroaryl. In some embodiments, R¹ is hexyl-heteroaryl.

In some embodiments, R¹ is C₂-C₆ alkenyl-aryl.

In some embodiments, R¹ is C₂ alkenyl-aryl. In some embodiments, R¹ is C₅ alkenyl-aryl. In some embodiments, R¹ is C₄ alkenyl-aryl. In some embodiments, R¹ is C₅ alkenyl-aryl. In some embodiments, R¹ is C₆ alkenyl-aryl.

In some embodiments, R¹ is C₂-C₆ alkenyl-heteroaryl.

In some embodiments, R¹ is C₂ alkenyl-heteroaryl. In some embodiments, R¹ is C₃ alkenyl-heteroaryl. In some embodiments, R¹ is C₄ alkenyl-heteroaryl. In some embodiments, R¹ is C₅ alkenyl-heteroaryl. In some embodiments, R¹ is C₆ alkenyl-heteroaryl.

In some embodiments, R is C₂-C₆ alkynyl-aryl.

In some embodiments, R¹ is C₂ alkynyl-aryl. In some embodiments, R¹ is C₃ alkynyl-aryl. In some embodiments, R¹ is C₄ alkynyl-aryl. In some embodiments, R¹ is C₅ alkynyl-aryl. In some embodiments, R¹ is C₆ alkynyl-aryl.

In some embodiments, R¹ is and C₂-C₆ alkynyl-heteroaryl.

In some embodiments, R¹ is C₂ alkynyl-heteroaryl. In some embodiments, R¹ is C₃ alkynyl-heteroaryl. In some embodiments, R¹ is C₄ alkynyl-heteroaryl. In some embodiments, R¹ is C₅ alkynyl-heteroaryl. In some embodiments, R¹ is Co alkynyl-heteroaryl.

In some embodiments, R¹ is NR¹²R¹³. In some embodiments, R¹ is NHR¹³. In some embodiments, R¹ is NHCH₃.

In some embodiments, p is 2, one R¹ is NR¹²R¹³, and the other R¹ is selected from C₁-C₆ alkyl, C₁-C₆ alkoxy, C₃-C₁₀ cycloalkyl, C₂-C₆ alkenyl, C₂-C₆ alkynyl, heterocyclyl, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, and NR¹²R¹³ wherein the alkyl, alkoxy, alkenyl, alkynyl, heterocyclyl, cycloalkyl, aryl, or heteroaryl is optionally substituted with one or more substituents independently selected from halogen, OH, NH₂, C₁-C₆ alkyl, C₁-C₆ alkoxy, NR¹²R¹³, C₃-C₁₀ cycloalkyl, C₂-C₆ alkenyl, C₂-C₆ alkynyl, heterocyclyl, aryl, heteroaryl.

In some embodiments, p is 2, one R¹ is NHCH₃, and the other R¹ is C₁-C₆ alkyl optionally substituted with one or more halogen. In some embodiments, p is 2, one R¹ is NHCH₃, and the other R¹ is C₁-C₆ alkyl optionally substituted with one or more fluoro. In some embodiments, one R¹ is NHCH₃, and the other R¹ is CH₂CF₃.

In some embodiments, R¹ is C₁-C₆ alkyl substituted with one or more halogen. In some embodiments, R¹ is C₁-C₆ alkyl substituted with one or more F. In some embodiments, R¹ is C₁-C₆ alkyl substituted with one or more Cl. In some embodiments, R¹ is C₁-C₆ alkyl substituted with one or more Br. In some embodiments, R¹ is C₁-C₆ alkyl substituted with one or more I. In some embodiments, R¹ is C₁-C₆ alkyl substituted with one or more OH. In some embodiments, R¹ is C₁-C₆ alkyl substituted with one or more NH₂. In some embodiments, R¹ is C₁-C₆ alkyl substituted with one or more C₁-C₆ alkyl. In some embodiments, R¹ is C₁-C₆ alkyl substituted with one or more C₁-C₆ alkoxy. In some embodiments, R¹ is C₁-C₆ alkyl substituted with one or more NR¹²R¹³. In some embodiments, R¹ is C₁-C₆ alkyl substituted with one or more C₃-C₁₀ cycloalkyl. In some embodiments, R¹ is C₁-C₆ alkyl substituted with one or more C₂-C₆ alkenyl. In some embodiments, R¹ is C₁-C₆ alkyl substituted with one or more C₂-C₆ alkynyl. In some embodiments, R¹ is C₁-C₆ alkyl substituted with one or more heterocyclyl. In some embodiments, R¹ is C₁-C₆ alkyl substituted with one or more aryl. In some embodiments, R¹ is C₁-C₆ alkyl substituted with one or more heteroaryl.

In some embodiments, R¹ is C₁-C₆ alkyl substituted with one or more F atoms. In some embodiments, R¹ is methyl substituted with one or more F. In some embodiments, R¹ is ethyl substituted with one or more F. In some embodiments, R¹ is propyl substituted with one or more F. In some embodiments, R¹ is n-propyl substituted with one or more F. In some embodiments, R¹ is isopropyl substituted with one or more F. In some embodiments, R¹ is butyl substituted with one or more F. In some embodiments, R¹ is n-butyl substituted with one or more F. In some embodiments, R¹ is isobutyl substituted with one or more F. In some embodiments, R¹ is sec-butyl substituted with one or more F. In some embodiments, R¹ is tert-butyl substituted with one or more F. In some embodiments, R¹ is pentyl substituted with one or more F. In some embodiments, R¹ is hexyl substituted with one or more F.

In some embodiments, R¹ is (CH₂)₀₋₅CF₃. In some embodiments, R¹ is CF₃. In some embodiments, R¹ is CH₂CF₃.

In some embodiments, p is 1 and R¹ is (CH₂)₀₋₅CF₃. In some embodiments, p is 1 and R¹ is CF₃. In some embodiments, p is 1 and R¹ is CH₂CF₃.

In some embodiments, p is 2 and at least one and R¹ is (CH₂)₀₋₅CF₃. In some embodiments, p is 2 and at least one and R¹ is CF₃. In some embodiments, p is 2 and at least one and R¹ is CH₂CF₃.

In some embodiments, R¹ is C₁-C₆ alkoxy substituted with one or more halogen. In some embodiments, R¹ is C₁-C₆ alkoxy substituted with one or more F. In some embodiments, R¹ is C₁-C₆ alkoxy substituted with one or more Cl. In some embodiments, R¹ is C₁-C₆ alkoxy substituted with one or more Br. In some embodiments, R¹ is C₁-C₆ alkoxy substituted with one or more I. In some embodiments, R¹ is C₁-C₆ alkoxy substituted with one or more OH. In some embodiments, R¹ is C₁-C₆ alkoxy substituted with one or more NH₂. In some embodiments, R¹ is C₁-C₆ alkoxy substituted with one or more C₁-C₆ alkyl. In some embodiments, R¹ is C₁-C₆ alkoxy substituted with one or more C₁-C₆ alkoxy. In some embodiments, R¹ is C₁-C₆ alkoxy substituted with one or more NR¹2 R¹3. In some embodiments, R¹ is C₁-C₆ alkoxy substituted with one or more C₃-C₁₀ cycloalkyl. In some embodiments, R¹ is C₁-C₆ alkoxy substituted with one or more C₂-C₆ alkenyl. In some embodiments, R¹ is C₁-C₆ alkoxy substituted with one or more C₂-C₆ alkynyl. In some embodiments, R¹ is C₁-C₆ alkoxy substituted with one or more heterocyclyl. In some embodiments, R¹ is C₁-C₆ alkoxy substituted with one or more aryl. In some embodiments, R¹ is C₁-C₆ alkoxy substituted with one or more heteroaryl.

In some embodiments, R¹ is C₃-C₁₀ cycloalkyl substituted with one or more halogen. In some embodiments, R¹ is C₃-C₁₀ cycloalkyl substituted with one or more F. In some embodiments, R¹ is C₃-C₁₀ cycloalkyl substituted with one or more Cl. In some embodiments, R¹ is C₃-C₁₀ cycloalkyl substituted with one or more Br. In some embodiments, R¹ is C₃-C₁₀ cycloalkyl substituted with one or more I. In some embodiments, R¹ is C₃-C₁₀ cycloalkyl substituted with one or more OH. In some embodiments, R¹ is C₃-C₁₀ cycloalkyl substituted with one or more NH₂. In some embodiments, R¹ is C₃-C₁₀ cycloalkyl substituted with one or more C₁-C₆ alkyl. In some embodiments, R¹ is C₃-C₁₀ cycloalkyl substituted with one or more C₁-C₆ alkoxy. In some embodiments, R¹ is C₃-C₁₀ cycloalkyl substituted with one or more NR¹²R¹³. In some embodiments, R¹ is C₃-C₁₀ cycloalkyl substituted with one or more C₃-C₁₀ cycloalkyl. In some embodiments, R¹ is C₁-C₁₀ cycloalkyl substituted with one or more C₂-C₆ alkenyl. In some embodiments, R¹ is C₃-C₁₀ cycloalkyl substituted with one or more C₂-C₆ alkynyl. In some embodiments, R¹ is C₃-C₁₀ cycloalkyl substituted with one or more heterocyclyl. In some embodiments, R¹ is C₃-C₁₀ cycloalkyl substituted with one or more aryl. In some embodiments, R¹ is C₃-C₁₀ cycloalkyl substituted with one or more heteroaryl.

In some embodiments, R¹ is C₂-C₆ alkenyl substituted with one or more halogen. In some embodiments, R¹ is C₂-C₆ alkenyl substituted with one or more F. In some embodiments, R¹ is C₂-C₆ alkenyl substituted with one or more Cl. In some embodiments, R¹ is C₂-C₆ alkenyl substituted with one or more Br. In some embodiments, R¹ is C₂-C₆ alkenyl substituted with one or more I. In some embodiments, R¹ is C₂-C₆ alkenyl substituted with one or more OH. In some embodiments, R¹ is C₂-C₆ alkenyl substituted with one or more NH₂. In some embodiments, R¹ is C₂-C₆ alkenyl substituted with one or more C₁-C₆ alkyl. In some embodiments, R¹ is C₂-C₆ alkenyl substituted with one or more C₁-C₆ alkoxy. In some embodiments, R¹ is C₂-C₆ alkenyl substituted with one or more NR¹²R¹³. In some embodiments, R¹ is C₂-C₆ alkenyl substituted with one or more C₃-C₁₀ cycloalkyl. In some embodiments, R¹ is C₂-C₆ alkenyl substituted with one or more C₂-C₆ alkenyl. In some embodiments, R¹ is C₂-C₆ alkenyl substituted with one or more C₂-C₆ alkynyl. In some embodiments, R¹ is C₂-C₆ alkenyl substituted with one or more heterocyclyl. In some embodiments, R¹ is C₂-C₆ alkenyl substituted with one or more aryl. In some embodiments, R¹ is C₂-C₆ alkenyl substituted with one or more heteroaryl.

In some embodiments, R¹ is C₂-C₆ alkynyl substituted with one or more halogen. In some embodiments, R¹ is C₂-C₆ alkynyl substituted with one or more heteroaryl.

In some embodiments, R¹ is heterocyclyl substituted with one or more halogen. In some embodiments, R¹ is heterocyclyl substituted with one or more heteroaryl.

In some embodiments, R¹ is aryl substituted with one or more halogen. In some embodiments, R¹ is aryl substituted with one or more heteroaryl.

In some embodiments, R¹ is heteroaryl substituted with one or more halogen. In some embodiments, R¹ is heteroaryl substituted with one or more heteroaryl.

In some embodiments, R¹ is C₁-C₆ alkyl-aryl substituted with one or more halogen. In some embodiments, R¹ is C₁-C₆ alkyl-aryl substituted with one or more heteroaryl.

In some embodiments, R¹ is C₁-C₆ alkyl-heteroaryl substituted with one or more halogen. In some embodiments, R¹ is C₁-C₆ alkyl-heteroaryl substituted with one or more heteroaryl.

In some embodiments, R¹ is C₂-C₆ alkenyl-aryl substituted with one or more halogen. In some embodiments, R¹ is C₂-C₆ alkenyl-aryl substituted with one or more heteroaryl.

In some embodiments, R¹ is C₂-C₆ alkenyl-heteroaryl substituted with one or more halogen. In some embodiments, R¹ is C₂-C₆ alkenyl-heteroaryl substituted with one or more heteroaryl.

In some embodiments, R¹ is C₂-C₆ alkynyl-aryl substituted with one or more halogen. In some embodiments, R¹ is C₂-C₆ alkynyl-aryl substituted with one or more heteroaryl.

In some embodiments, R¹ is C₂-C₆ alkynyl-heteroaryl substituted with one or more halogen. In some embodiments, R¹ is C₂-C₆ alkynyl-heteroaryl substituted with one or more heteroaryl.

In some embodiments, R¹ is unsubstituted C₁-C₆ alkyl. In some embodiments, R¹ is unsubstituted C₁-C₆ alkoxy. In some embodiments, R¹ is unsubstituted C₃-C₁₀ cycloalkyl. In some embodiments, R¹ is unsubstituted C₂-C₆ alkenyl. In some embodiments, R¹ is unsubstituted C₂-C₆ alkynyl. In some embodiments, R¹ is unsubstituted heterocyclyl. In some embodiments, R¹ is unsubstituted aryl. In some embodiments, R¹ is unsubstituted heteroaryl. In some embodiments, R¹ is unsubstituted C₁-C₆ alkyl-aryl. In some embodiments, R¹ is unsubstituted C₁-C₆ alkyl-heteroaryl. In some embodiments, R¹ is unsubstituted C₂-C₆ alkenyl-aryl. In some embodiments, R¹ is unsubstituted C₂-C₆ alkenyl-heteroaryl. In some embodiments, R¹ is unsubstituted C₂-C₆ alkynyl-aryl. In some embodiments, R¹ is unsubstituted C₂-C₆ alkynyl-heteroaryl.

In some embodiments, R⁴′ is H. In some embodiments, R⁴′ is C₁-C₆ alkyl. In some embodiment, each R⁴′ is H. In some embodiments, at least one R⁴′ is C₁-C₆ alkyl.

In some embodiments, at least one R⁴′ is methyl. In some embodiments, at least one R⁴′ is ethyl. In some embodiments, at least one R⁴′ is propyl. In some embodiments, at least one R⁴′ is n-propyl. In some embodiments, at least one R⁴′ is isopropyl. In some embodiments, at least one R⁴′ is butyl. In some embodiments, at least one R⁴′ is n-butyl. In some embodiments, at least one R⁴′ is isobutyl. In some embodiments, at least one R⁴′ is sec-butyl. In some embodiments, at least one R⁴ is tert-butyl. In some embodiments, at least one R⁴′ is pentyl. In some embodiments, at least one R⁴′ is hexyl.

In some embodiments, R² is H. In some embodiments, R² is C₁-C₆ alkyl.

In some embodiments, R² is methyl. In some embodiments, R² is ethyl. In some embodiments, R² is propyl. In some embodiments, R² is n-propyl. In some embodiments, R² is isopropyl. In some embodiments, R² is butyl. In some embodiments, R² is n-butyl. In some embodiments, R² is isobutyl. In some embodiments, R² is sec-butyl. In some embodiments, R² is tert-butyl. In some embodiments, R² is pentyl. In some embodiments, R² is hexyl.

In some embodiments, R³ is H. In some embodiments, R³ is C₁-C₆ alkyl.

In some embodiments, R³ is methyl. In some embodiments, R³ is ethyl. In some embodiments, R³ is propyl. In some embodiments, R³ is n-propyl. In some embodiments, R³ is isopropyl. In some embodiments, R³ is butyl. In some embodiments, R³ is n-butyl. In some embodiments, R³ is isobutyl. In some embodiments, R³ is sec-butyl. In some embodiments, R³ is tert-butyl. In some embodiments, R³ is pentyl. In some embodiments, R³ is hexyl.

In some embodiments, R² and R³, together with the atoms to which they are bound and any intervening atoms, form a 3-8 membered heterocycle, wherein the heterocycle is optionally substituted with one or more substituents independently selected from halogen, OH, NH₂, C₁-C₆ alkyl, C₁-C₆ alkoxy, and NR¹²R¹³.

In some embodiments, R² and R³, together with the atoms to which they are bound and any intervening atoms, form a 3-membered heterocycle that is unsubstituted. In some embodiments, R² and R³, together with the atoms to which they are bound and any intervening atoms, form a 3-membered heterocycle substituted by one or more halogen. In some embodiments, R² and R³, together with the atoms to which they are bound and any intervening atoms, form a 3-membered heterocycle substituted by one or more F. In some embodiments, R² and R³, together with the atoms to which they are bound and any intervening atoms, form a 3-membered heterocycle substituted by one or more Cl. In some embodiments, R² and R³, together with the atoms to which they are bound and any intervening atoms, form a 3-membered heterocycle substituted by one or more Br. In some embodiments, R² and R³, together with the atoms to which they are bound and any intervening atoms, form a 3-membered heterocycle substituted by one or more I. In some embodiments, R² and R³, together with the atoms to which they are bound and any intervening atoms, form a 3-membered heterocycle substituted by one or more OH. In some embodiments, R² and R³, together with the atoms to which they are bound and any intervening atoms, form a 3-membered heterocycle substituted by one or more NH₂. In some embodiments, R² and R³, together with the atoms to which they are bound and any intervening atoms, form a 3-membered heterocycle substituted by one or more C₁-C₆ alkyl. In some embodiments, R² and R³, together with the atoms to which they are bound and any intervening atoms, form a 3-membered heterocycle substituted by one or more C₁-C₆ alkoxy. In some embodiments, R² and R³, together with the atoms to which they are bound and any intervening atoms, form a 3-membered heterocycle substituted by one or more NR¹²R¹³.

In some embodiments, R² and R³, together with the atoms to which they are bound and any intervening atoms, form a 4-membered heterocycle that is unsubstituted. In some embodiments, R² and R³, together with the atoms to which they are bound and any intervening atoms, form a 4-membered heterocycle substituted by one or more halogen. In some embodiments, R² and R³, together with the atoms to which they are bound and any intervening atoms, form a 4-membered heterocycle substituted by one or more F. In some embodiments, R² and R³, together with the atoms to which they are bound and any intervening atoms, form a 4-membered heterocycle substituted by one or more Cl. In some embodiments, R² and R³, together with the atoms to which they are bound and any intervening atoms, form a 4-membered heterocycle substituted by one or more Br. In some embodiments, R² and R³, together with the atoms to which they are bound and any intervening atoms, form a 4-membered heterocycle substituted by one or more I. In some embodiments, R² and R³, together with the atoms to which they are bound and any intervening atoms, form a 4-membered heterocycle substituted by one or more OH. In some embodiments, R² and R³, together with the atoms to which they are bound and any intervening atoms, form a 4-membered heterocycle substituted by one or more NH2. In some embodiments, R² and R³, together with the atoms to which they are bound and any intervening atoms, form a 4-membered heterocycle substituted by one or more C₁-C₆ alkyl. In some embodiments, R² and R³, together with the atoms to which they are bound and any intervening atoms, form a 4-membered heterocycle substituted by one or more C₁-C₆ alkoxy. In some embodiments, R² and R³, together with the atoms to which they are bound and any intervening atoms, form a 4-membered heterocycle substituted by one or more NR¹²R¹³.

In some embodiments, R² and R³, together with the atoms to which they are bound and any intervening atoms, form a 5-membered heterocycle that is unsubstituted. In some embodiments, R² and R³, together with the atoms to which they are bound and any intervening atoms, form a 5-membered heterocycle substituted by one or more halogen. In some embodiments, R² and R³, together with the atoms to which they are bound and any intervening atoms, form a 5-membered heterocycle substituted by one or more F. In some embodiments, R² and R³, together with the atoms to which they are bound and any intervening atoms, form a 5-membered heterocycle substituted by one or more Cl. In some embodiments, R² and R³, together with the atoms to which they are bound and any intervening atoms, form a 5-membered heterocycle substituted by one or more Br. In some embodiments, R² and R³, together with the atoms to which they are bound and any intervening atoms, form a 5-membered heterocycle substituted by one or more 1. In some embodiments, R² and R³, together with the atoms to which they are bound and any intervening atoms, form a 5-membered heterocycle substituted by one or more OH. In some embodiments, R² and R³, together with the atoms to which they are bound and any intervening atoms, form a 5-membered heterocycle substituted by one or more NH2. In some embodiments, R² and R³, together with the atoms to which they are bound and any intervening atoms, form a 5-membered heterocycle substituted by one or more C₁-C₆ alkyl. In some embodiments, R² and R³, together with the atoms to which they are bound and any intervening atoms, form a 5-membered heterocycle substituted by one or more C₁-C₆ alkoxy. In some embodiments, R² and R³, together with the atoms to which they are bound and any intervening atoms, form a 5-membered heterocycle substituted by one or more NR¹²R¹³.

In some embodiments, R² and R³, together with the atoms to which they are bound and any intervening atoms, form a 6-membered heterocycle that is unsubstituted. In some embodiments, R² and R³, together with the atoms to which they are bound and any intervening atoms, form a 6-membered heterocycle substituted by one or more halogen. In some embodiments, R² and R³, together with the atoms to which they are bound and any intervening atoms, form a 6-membered heterocycle substituted by one or more F. In some embodiments, R² and R³, together with the atoms to which they are bound and any intervening atoms, form a 6-membered heterocycle substituted by one or more Cl. In some embodiments, R² and R³, together with the atoms to which they are bound and any intervening atoms, form a 6-membered heterocycle substituted by one or more Br. In some embodiments, R² and R³, together with the atoms to which they are bound and any intervening atoms, form a 6-membered heterocycle substituted by one or more I. In some embodiments, R² and R³, together with the atoms to which they are bound and any intervening atoms, form a 6-membered heterocycle substituted by one or more OH. In some embodiments, R² and R³, together with the atoms to which they are bound and any intervening atoms, form a 6-membered heterocycle substituted by one or more NH₂. In some embodiments, R² and R³, together with the atoms to which they are bound and any intervening atoms, form a 6-membered heterocycle substituted by one or more C₁-C₆ alkyl. In some embodiments, R² and R³, together with the atoms to which they are bound and any intervening atoms, form a 6-membered heterocycle substituted by one or more C₁-C₆ alkoxy. In some embodiments, R² and R³, together with the atoms to which they are bound and any intervening atoms, form a 6-membered heterocycle substituted by one or more NR¹²R¹³.

In some embodiments, R² and R³, together with the atoms to which they are bound and any intervening atoms, form a 7-membered heterocycle that is unsubstituted. In some embodiments, R² and R³, together with the atoms to which they are bound and any intervening atoms, form a 7-membered heterocycle substituted by one or more halogen. In some embodiments, R² and R³, together with the atoms to which they are bound and any intervening atoms, form a 7-membered heterocycle substituted by one or more F. In some embodiments, R² and R³, together with the atoms to which they are bound and any intervening atoms, form a 7-membered heterocycle substituted by one or more Cl. In some embodiments, R² and R³, together with the atoms to which they are bound and any intervening atoms, form a 7-membered heterocycle substituted by one or more Br. In some embodiments, R² and R³, together with the atoms to which they are bound and any intervening atoms, form a 7-membered heterocycle substituted by one or more I. In some embodiments, R² and R³, together with the atoms to which they are bound and any intervening atoms, form a 7-membered heterocycle substituted by one or more OH. In some embodiments, R² and R³, together with the atoms to which they are bound and any intervening atoms, form a 7-membered heterocycle substituted by one or more NH₂. In some embodiments, R² and R³, together with the atoms to which they are bound and any intervening atoms, form a 7-membered heterocycle substituted by one or more C₁-C₆ alkyl. In some embodiments, R² and R³, together with the atoms to which they are bound and any intervening atoms, form a 7-membered heterocycle substituted by one or more Ct-Ce alkoxy. In some embodiments, R² and R³, together with the atoms to which they are bound and any intervening atoms, form a 7-membered heterocycle substituted by one or more NR¹²R¹³.

some embodiments, R⁴ and R⁵, together with the atoms to which they are bound and any intervening atoms, form a 3-membered heterocycle that is unsubstituted. In some embodiments, R⁴ and R⁵, together with the atoms to which they are bound and any intervening atoms, form a 3-membered heterocycle substituted by one or more halogen. In some embodiments, R⁴ and R⁵, together with the atoms to which they are bound and any intervening atoms, form a 3-membered heterocycle substituted by one or more F. In some embodiments, R⁴ and R⁵, together with the atoms to which they are bound and any intervening atoms, form a 3-membered heterocycle substituted by one or more Cl. In some embodiments, R⁴ and R⁵, together with the atoms to which they are bound and any intervening atoms, form a 3-membered heterocycle substituted by one or more Br. In some embodiments, R⁴ and R⁵, together with the atoms to which they are bound and any intervening atoms, form a 3-membered heterocycle substituted by one or more I. In some embodiments, R⁴ and R⁵, together with the atoms to which they are bound and any intervening atoms, form a 3-membered heterocycle substituted by one or more OH. In some embodiments, R⁴ and R⁵, together with the atoms to which they are bound and any intervening atoms, form a 3-membered heterocycle substituted by one or more NH2. In some embodiments, R⁴ and R⁵, together with the atoms to which they are bound and any intervening atoms, form a 3-membered heterocycle substituted by one or more C₁-C₆ alkyl. In some embodiments, R⁴ and R⁵, together with the atoms to which they are bound and any intervening atoms, form a 3-membered heterocycle substituted by one or more C₁-C₆ alkoxy. In some embodiments, R⁴ and R⁵, together with the atoms to which they are bound and any intervening atoms, form a 3-membered heterocycle substituted by one or more NR¹²R¹³.

In some embodiments, R⁴ and R⁵, together with the atoms to which they are bound and any intervening atoms, form a 4-membered heterocycle that is unsubstituted. In some embodiments, R⁴ and R⁵, together with the atoms to which they are bound and any intervening atoms, form a 4-membered heterocycle substituted by one or more halogen. In some embodiments, R⁴ and R⁵, together with the atoms to which they are bound and any intervening atoms, form a 4-membered heterocycle substituted by one or more F. In some embodiments, R⁴ and R⁵, together with the atoms to which they are bound and any intervening atoms, form a 4-membered heterocycle substituted by one or more Cl. In some embodiments, R⁴ and R⁵, together with the atoms to which they are bound and any intervening atoms, form a 4-membered heterocycle substituted by one or more Br. In some embodiments, R⁴ and R⁵, together with the atoms to which they are bound and any intervening atoms, form a 4-membered heterocycle substituted by one or more I. In some embodiments, R⁴ and R⁵, together with the atoms to which they are bound and any intervening atoms, form a 4-membered heterocycle substituted by one or more OH. In some embodiments, R⁴ and R⁵, together with the atoms to which they are bound and any intervening atoms, form a 4-membered heterocycle substituted by one or more NH2. In some embodiments, R⁴ and R⁵, together with the atoms to which they are bound and any intervening atoms, form a 4-membered heterocycle substituted by one or more C₁-C₆ alkyl. In some embodiments, R⁴ and R⁵, together with the atoms to which they are bound and any intervening atoms, form a 4-membered heterocycle substituted by one or more C₁-C₆ alkoxy. In some embodiments, R⁴ and R⁵, together with the atoms to which they are bound and any intervening atoms, form a 4-membered heterocycle substituted by one or more NR¹²R¹³.

In some embodiments, R⁴ and R⁵, together with the atoms to which they are bound and any intervening atoms, form a S-membered heterocycle that is unsubstituted. In some embodiments, R⁴ and R⁵, together with the atoms to which they are bound and any intervening atoms, form a 5-membered heterocycle substituted by one or more halogen. In some embodiments, R⁴ and R⁵, together with the atoms to which they are bound and any intervening atoms, form a 5-membered heterocycle substituted by one or more F. In some embodiments, R⁴ and R⁵, together with the atoms to which they are bound and any intervening atoms, form a 5-membered heterocycle substituted by one or more Cl. In some embodiments, R⁴ and R⁵, together with the atoms to which they are bound and any intervening atoms, form a 5-membered heterocycle substituted by one or more Br. In some embodiments, R⁴ and R⁵, together with the atoms to which they are bound and any intervening atoms, form a 5-membered heterocycle substituted by one or more I. In some embodiments, R⁴ and R⁵, together with the atoms to which they are bound and any intervening atoms, form a 5-membered heterocycle substituted by one or more OH. In some embodiments, R⁴ and R⁵, together with the atoms to which they are bound and any intervening atoms, form a 5-membered heterocycle substituted by one or more NH2. In some embodiments, R⁴ and R⁵, together with the atoms to which they are bound and any intervening atoms, form a 5-membered heterocycle substituted by one or more C₁-C₆ alkyl. In some embodiments, R⁴ and R⁵, together with the atoms to which they are bound and any intervening atoms, form a 5-membered heterocycle substituted by one or more C₁-C₆ alkoxy. In some embodiments, R⁴ and R⁵, together with the atoms to which they are bound and any intervening atoms, form a 5-membered heterocycle substituted by one or more NR¹²R¹³.

In some embodiments, R⁴ and R⁵, together with the atoms to which they are bound and any intervening atoms, form a 6-membered heterocycle that is unsubstituted. In some embodiments, R⁴ and R⁵, together with the atoms to which they are bound and any intervening atoms, form a 6-membered heterocycle substituted by one or more halogen. In some embodiments, R⁴ and R⁵, together with the atoms to which they are bound and any intervening atoms, form a 6-membered heterocycle substituted by one or more F. In some embodiments, R⁴ and R⁵, together with the atoms to which they are bound and any intervening atoms, form a 6-membered heterocycle substituted by one or more Cl. In some embodiments, R⁴ and R⁵, together with the atoms to which they are bound and any intervening atoms, form a 6-membered heterocycle substituted by one or more Br. In some embodiments, R⁴ and R⁵, together with the atoms to which they are bound and any intervening atoms, form a 6-membered heterocycle substituted by one or more I. In some embodiments, R⁴ and R⁵, together with the atoms to which they are bound and any intervening atoms, form a 6-membered heterocycle substituted by one or more OH. In some embodiments, R⁴ and R⁵, together with the atoms to which they are bound and any intervening atoms, form a 6-membered heterocycle substituted by one or more NH2. In some embodiments, R⁴ and R⁵, together with the atoms to which they are bound and any intervening atoms, form a 6-membered heterocycle substituted by one or more C₁-C₆ alkyl. In some embodiments, R⁴ and R⁵, together with the atoms to which they are bound and any intervening atoms, form a 6-membered heterocycle substituted by one or more C₁-C₆ alkoxy. In some embodiments, R⁴ and R⁵, together with the atoms to which they are bound and any intervening atoms, form a 6-membered heterocycle substituted by one or more NR¹²R¹³.

In some embodiments, R⁴ and R⁵, together with the atoms to which they are bound and any intervening atoms, form a 7-membered heterocycle that is unsubstituted. In some embodiments, R⁴ and R⁵, together with the atoms to which they are bound and any intervening atoms, form a 7-membered heterocycle substituted by one or more halogen. In some embodiments, R⁴ and R⁵, together with the atoms to which they are bound and any intervening atoms, form a 7-membered heterocycle substituted by one or more F. In some embodiments, R⁴ and R⁵, together with the atoms to which they are bound and any intervening atoms, form a 7-membered heterocycle substituted by one or more Cl. In some embodiments, R⁴ and R⁵, together with the atoms to which they are bound and any intervening atoms, form a 7-membered heterocycle substituted by one or more Br. In some embodiments, R⁴ and R⁵, together with the atoms to which they are bound and any intervening atoms, form a 7-membered heterocycle substituted by one or more I. In some embodiments, R⁴ and R⁵, together with the atoms to which they are bound and any intervening atoms, form a 7-membered heterocycle substituted by one or more OH. In some embodiments, R⁴ and R⁵, together with the atoms to which they are bound and any intervening atoms, form a 7-membered heterocycle substituted by one or more NH₂. In some embodiments, R⁴ and R⁵, together with the atoms to which they are bound and any intervening atoms, form a 7-membered heterocycle substituted by one or more C₁-C₆ alkyl. In some embodiments, R⁴ and R⁵, together with the atoms to which they are bound and any intervening atoms, form a 7-membered heterocycle substituted by one or more C₁-C₆ alkoxy. In some embodiments, R⁴ and R⁵, together with the atoms to which they are bound and any intervening atoms, form a 7-membered heterocycle substituted by one or more NR¹²R¹³.

In some embodiments, R′ and R″ are both H.

In some embodiments Ring A is aryl or C₃-C₁₄ cycloalkyl.

In some embodiments Ring A is aryl.

In some embodiments Ring A is C₆ aryl (e.g., phenyl).

In some embodiments Ring A is C₃-C₁₄ cycloalkyl.

In some embodiments, Ring A is a monocyclic C₃-C₁₄ cycloalkyl. In some embodiments, Ring A is a polycyclic C₃-C₁₄ cycloalkyl.

In some embodiments, Ring A is C₅-C₆ cycloalkyl.

In some embodiments, Ring A is cyclopropyl, cyclobutyl, cyclopentyl, cyclohexyl, cycloheptyl, cyclooctyl, cyclononyl, cyclodecyl, cycloundecyl, cyclododecyl, cyclotridecyl, or cyclotetradecyl. In some embodiments, Ring A is cyclopropyl. In some embodiments, Ring A is cyclobutyl. In some embodiments, Ring A is cyclopentyl. In some embodiments, Ring A is cyclohexyl. In some embodiments, Ring A is cycloheptyl. In some embodiments, Ring A is cyclooctyl. In some embodiments, Ring A is cyclononyl. In some embodiments, Ring A is cyclodecyl.

In some embodiments, Ring A is a fused polycyclic C₃-C₁₀ cycloalkyl. In some embodiments, Ring A is a bridged polycyclic C₃-C₁₀ cycloalkyl. In some embodiments, Ring A is a C₃-C₁₀ spirocycloalkyl.

In some embodiments, Ring A is:

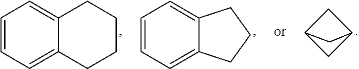

In some embodiments, Ring A is

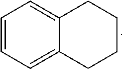

In some embodiments, Ring A is

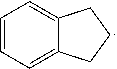

In some embodiments, Ring A is

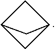

In some embodiments, L² is —N(R⁶)S(O)₂R⁷, —N(R⁶)CH₂R⁷, —N(R⁶)C(O)R⁷, —C(O)NR⁶R⁷, C(O)C₁₋₃ alkenyl, C(O)C₁₋₃ alkenyl-NR⁶R⁷, —S(O)₂R⁶, or —S(O)₂NR⁶R⁷.

In some embodiments, L¹ is

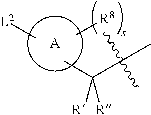

and L² is —N(R⁶)S(O)₂R⁷, —N(R⁶)CH₂R⁷, —N(R⁶)C(O)R⁷, —C(O)NR⁶R⁷, C(O)C₁₋₃ alkenyl, C(O)C₁₋₃ alkenyl-NR⁶R⁷, —S(O)₂R⁶, or —S(O)₂NR⁶R⁷.

In some embodiments, L² is —N(R⁶)S(O)₂R⁷, —N(R⁶)CH₂R⁷, —N(R⁶)C(O)R⁷, —C(O)NR⁶R⁷, —S(O)₂R⁶, or —S(O)₂NR⁶R⁷.

In some embodiments, L¹ is

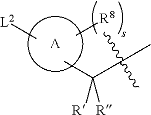

and L² is —N(R⁶)S(O)₂R⁷, —N(R⁶)CH₂R², —N(R⁶)C(O)R⁷, —C(O)NR⁶R⁷, —S(O)₂R⁶, or —S(O)₂NR⁶R⁷.

In some embodiments, L² is —N(R⁶)S(O)₂R⁷. In some embodiments, L¹ is

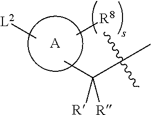

and L² is —N(R⁶)S(O)₂R⁷.

In some embodiments, L² is —N(R⁶)CH₂R⁷. In some embodiments, L¹ is

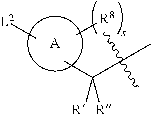

and L² is —N(R⁶)CH₂R⁷.

In some embodiments, L² is —N(R⁶)C(O)R⁷. In some embodiments, L¹ is

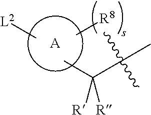

and L² is —N(R⁶)C(O)R⁷.

In some embodiments, L² is —C(O)NR⁶R⁷.

In some embodiments, L¹ is

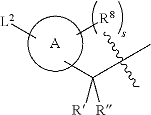

and L² is —C(O)NR⁶R⁷.

In some embodiments, L² is —C(O)C₁₋₃ alkenyl. In some embodiments, L¹ is

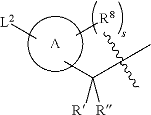

and L² is —C(O)C₁₋₃ alkenyl.

In some embodiments, L² is —C(O)C₁₃ alkenyl-NR⁶R⁷. In some embodiments, L¹ is

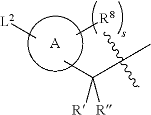

and L² is —C(O)C₁₋₃ alkenyl-NR⁶R⁷.

In some embodiments, L² is —S(O)₂R⁶.

In some embodiments, L¹ is

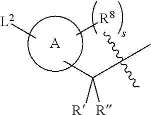

and L² is —S(O)₂R⁶.

In some embodiments, L² is —S(O)₂NR⁶R⁷.

In some embodiments, L¹ is

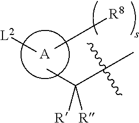

and L² is —S(O)₂NR⁶R⁷.

In some embodiments, L¹ is selected from:

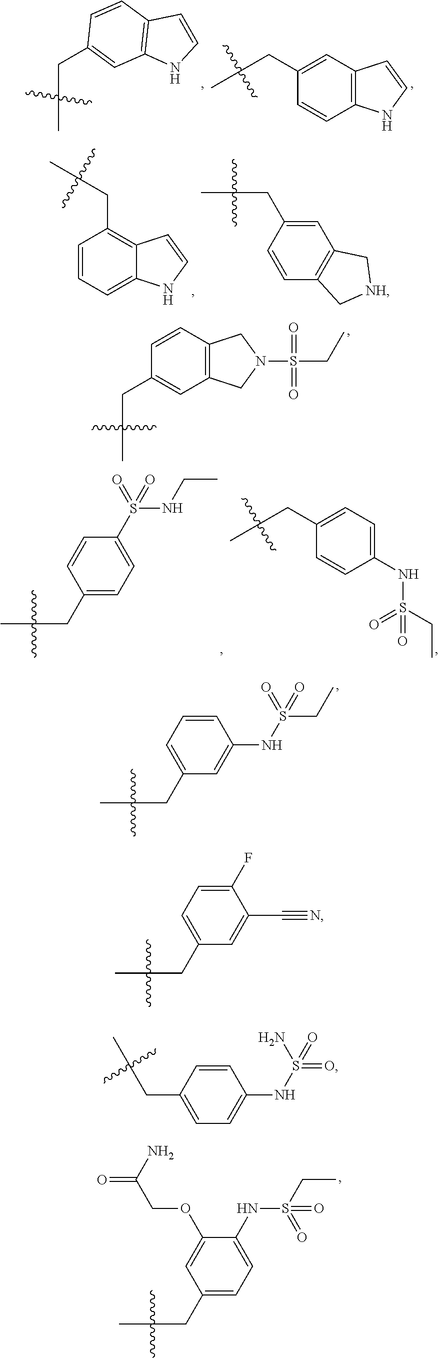

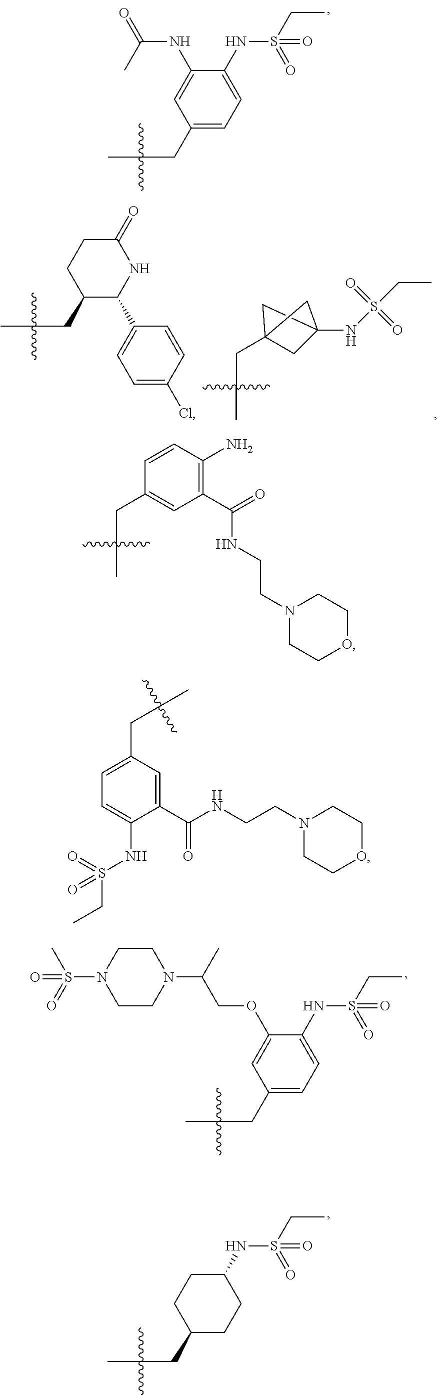

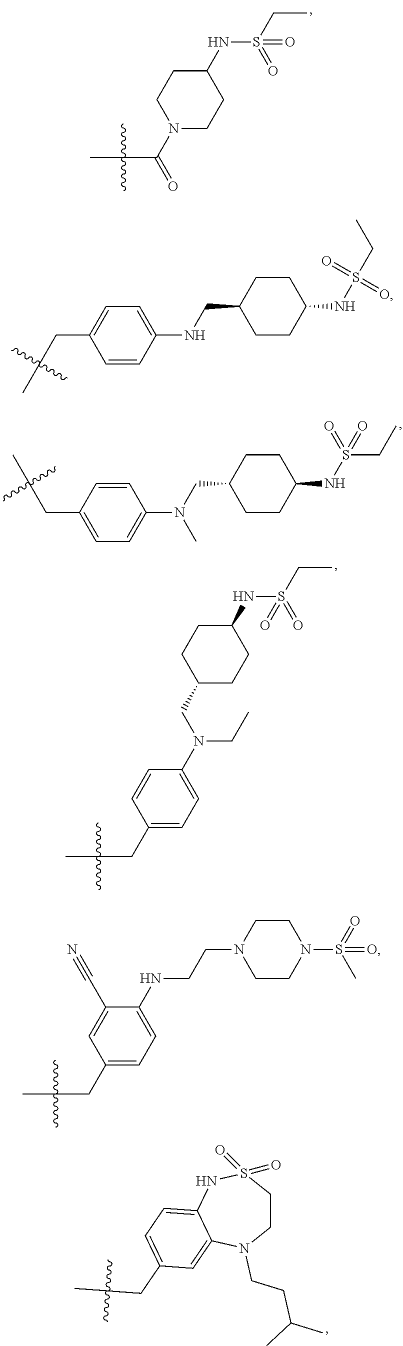 [SMILES: *C(=O)N1CCC(NS(=O)(=O)CC)CC1 ; *Cc1ccc(NC[C@H]2CC[C@H](NS(=O)(=O)CC)CC2)cc1 ; *Cc1ccc(N(C)C[C@H]2CC[C@H](NS(=O)(=O)CC)CC2)cc1 ; *Cc1ccc(N(CC)C[C@H]2CC[C@H](NS(=O)(=O)CC)CC2)cc1 ; *Cc1ccc(NCCN2CCN(S(C)(=O)=O)CC2)c(C#N)c1 ; *Cc1ccc2c(c1)N(CCC(C)C)CCS(=O)(=O)N2]

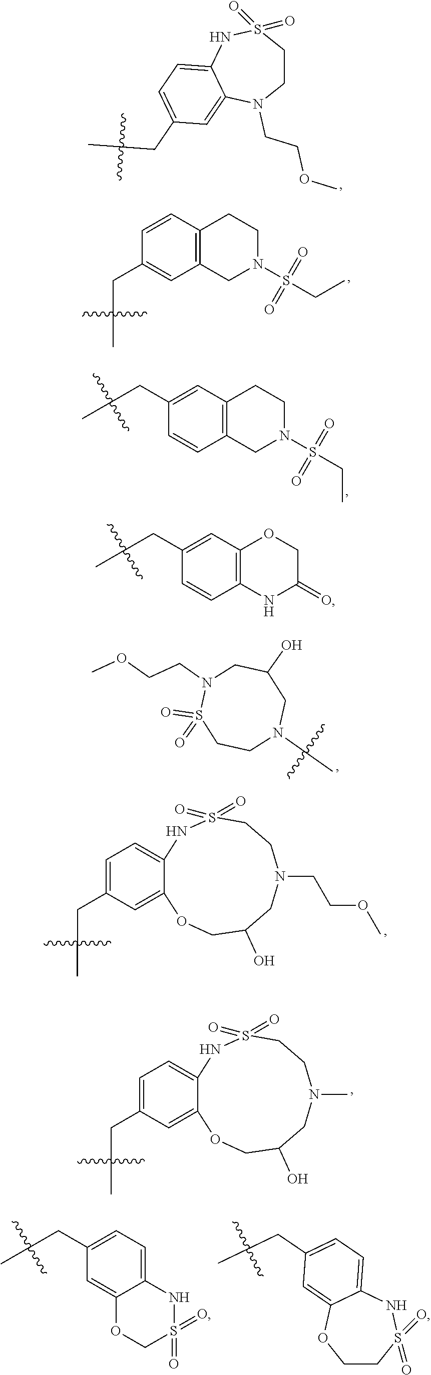

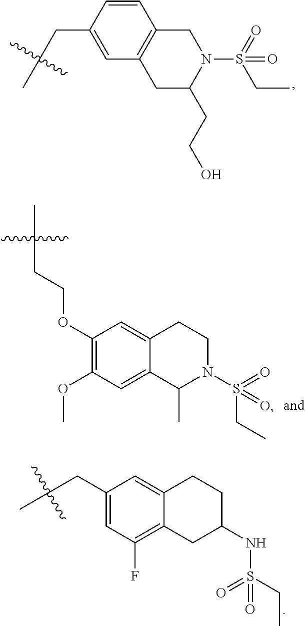 [SMILES: *Cc1ccc2c(c1)CC(CCO)N(S(=O)(=O)CC)C2 ; *CCOc1cc2c(cc1OC)C(C)N(S(=O)(=O)CC)CC2 ; *Cc1cc(F)c2c(c1)CCC(NS(=O)(=O)CC)C2]

In some embodiments, R⁶ is NH₂. In some embodiments, R⁶ is NR₁₂R₁₃. In some embodiments, R⁶ is C₁-C₆ alkyl. In some embodiments, R⁶ is C₂-C₆ alkenyl. In some embodiments, R⁶ is C₂-C₆ alkynyl. In some embodiments, R⁶ is C₃-C₁₀ cycloalkyl. In some embodiments, R⁶ is aryl. In some embodiments, R⁶ is heterocycle. In some embodiments, R⁶ is heteroaryl.

In some embodiments, R⁶ is NH₂ or NR₁₂R₁₃. In some embodiments, R⁶ is C₁-C₆ alkyl, C₂-C₆ alkenyl, or C₂-C₆ alkynyl. In some embodiments, R⁶ is C₃-C₁₀ cycloalkyl, aryl, heterocyclyl, or heteroaryl.

In some embodiments, R⁶ is NH₂. In some embodiments, R⁶ is NR₁₂R₁₃. In some embodiments, R⁶ is unsubstituted C₁-C₆ alkyl. In some embodiments, R⁶ is unsubstituted C₂-C₆ alkenyl. In some embodiments, R⁶ is unsubstituted C₂-C₆ alkynyl. In some embodiments, R⁶ is unsubstituted C₃-C₁₀ cycloalkyl. In some embodiments, R⁶ is unsubstituted aryl. In some embodiments, R⁶ is unsubstituted heterocyclyl. In some embodiments, R⁶ is unsubstituted heteroaryl.

In some embodiments, R⁶ is methyl. In some embodiments, R⁶ is ethyl. In some embodiments, R⁶ is propyl. In some embodiments, R⁶ is n-propyl. In some embodiments, R⁶ is isopropyl. In some embodiments, R⁶ is butyl. In some embodiments, R⁶ is n-butyl. In some embodiments, R⁶ is isobutyl. In some embodiments, R⁶ is sec-butyl. In some embodiments, R⁶ is tert-butyl. In some embodiments, R⁶ is pentyl. In some embodiments, R⁶ is hexyl.

In some embodiments, R⁶ is C₁-C₆ alkyl substituted with one or more halogen. In some embodiments, R⁶ is C₁-C₆ alkyl substituted with one or more F. In some embodiments, Re is C₁-C₆ alkyl substituted with one or more Cl. In some embodiments, R⁶ is C₁-C₆ alkyl substituted with one or more Br. In some embodiments, R⁶ is C₁-C₆ alkyl substituted with one or more I. In some embodiments, R⁶ is C₁-C₆ alkyl substituted with one or more OH. In some embodiments, R⁶ is C₁-C₆ alkyl substituted with one or more NH₂. In some embodiments, R⁶ is C₁-C₆ alkyl substituted with one or more C₁-C₆ alkyl. In some embodiments, R⁶ is C₁-C₆ alkyl substituted with one or more C₁-C₆ alkoxy. In some embodiments, R⁶ is C₁-C₆ alkyl substituted with one or more NR₁₂R₁₃.

In some embodiments, R⁶ is C₂ alkenyl. In some embodiments, R⁶ is C₃ alkenyl. In some embodiments, R⁶ is C₄ alkenyl. In some embodiments, R⁶ is C₅ alkenyl. In some embodiments, R⁶ is C₆ alkenyl.

In some embodiments, R⁶ is C₂-C₆ alkenyl substituted with one or more halogen. In some embodiments, R⁶ is C₂-C₆ alkenyl substituted with one or more F. In some embodiments, R⁶ is C₂-C₆ alkenyl substituted with one or more Cl. In some embodiments, R⁶ is C₂-C₆ alkenyl substituted with one or more Br. In some embodiments, R⁶ is C₂-C₆ alkenyl substituted with one or more I. In some embodiments, R⁶ is C₂-C₆ alkenyl substituted with one or more OH. In some embodiments, R⁶ is C₂-C₆ alkenyl substituted with one or more NH₂. In some embodiments, R⁶ is C₂-C₆ alkenyl substituted with one or more C₁-C₆ alkyl. In some embodiments, R⁶ is C₂-C₆ alkenyl substituted with one or more C₁-C₆ alkoxy. In some embodiments, R⁶ is C₂-C₆ alkenyl substituted with one or more NR¹²R¹³.

In some embodiments, R⁶ is C₂ alkynyl. In some embodiments, R⁶ is C₃ alkynyl. In some embodiments, R⁶ is C₄ alkynyl. In some embodiments, Reis C₅ alkynyl. In some embodiments, R⁶ is C₆ alkynyl.

In some embodiments, R⁶ is C₂-C₆ alkynyl substituted with one or more halogen. In some embodiments, R⁶ is C₂-C₆ alkynyl substituted with one or more F. In some embodiments, R⁶ is C₂-C₆ alkynyl substituted with one or more Cl. In some embodiments, R⁶ is C₂-C₆ alkynyl substituted with one or more Br. In some embodiments, R⁶ is C₂-C₆ alkynyl substituted with one or more I. In some embodiments, R⁶ is C₂-C₆ alkynyl substituted with one or more OH. In some embodiments, R⁶ is C₂-C₆ alkynyl substituted with one or more NH₂. In some embodiments, R⁶ is C₂-C₆ alkynyl substituted with one or more C₁-C₆ alkyl. In some embodiments, R⁶ is C₂-C₆ alkynyl substituted with one or more C₁-C₆ alkoxy. In some embodiments, R⁶ is C₂-C₆ alkynyl substituted with one or more NR¹²R¹³.

In some embodiments, R⁶ is cyclopropyl. In some embodiments, R⁶ is cyclobutyl. In some embodiments, R⁶ is cyclopentyl. In some embodiments, R⁶ is cyclohexyl. In some embodiments, R⁶ is cycloheptyl. In some embodiments, R⁶ is cyclooctyl. In some embodiments, R⁶ is cyclononyl. In some embodiments, R⁶ is cyclodecyl.

In some embodiments, R⁶ is C₃-C₁₀ cycloalkyl substituted with one or more halogen. In some embodiments, R⁶ is C₃-C₁₀ cycloalkyl substituted with one or more F. In some embodiments, R⁶ is C₃-C₁₀ cycloalkyl substituted with one or more Cl. In some embodiments, R⁶ is C₃-C₁₀ cycloalkyl substituted with one or more Br. In some embodiments, R⁶ is C₃-C₁₀ cycloalkyl substituted with one or more I. In some embodiments, R⁶ is C₃-C₁₀ cycloalkyl substituted with one or more OH. In some embodiments, R⁶ is C₃-C₁₀ cycloalkyl substituted with one or more NH₂. In some embodiments, R⁶ is C₃-C₁₀ cycloalkyl substituted with one or more C₁-C₆ alkyl. In some embodiments, R⁶ is C₃-C₁₀ cycloalkyl substituted with one or more C₁-C₆ alkoxy. In some embodiments, R⁶ is C₃-C₁₀ cycloalkyl substituted with one or more NR¹²R¹³.

In some embodiments, R⁶ is C₆ aryl (e.g., phenyl).

In some embodiments, R⁶ is aryl substituted with one or more halogen. In some embodiments, R⁶ is aryl substituted with one or more F. In some embodiments, R⁶ is aryl substituted with one or more Cl. In some embodiments, R⁶ is aryl substituted with one or more Br. In some embodiments, R⁶ is aryl substituted with one or more I. In some embodiments, R⁶ is aryl substituted with one or more OH. In some embodiments, R⁶ is aryl substituted with one or more NH₂. In some embodiments, R⁶ is aryl substituted with one or more C₁-C₆ alkyl. In some embodiments, R⁶ is aryl substituted with one or more C₁-C₆ alkoxy. In some embodiments, R⁶ is aryl substituted with one or more NR¹²R¹³.

In some embodiments R⁶ is 3- to 8-membered heterocycle. In some embodiments R⁶ is 3- to 10-membered heterocycle.

In some embodiments, R⁶ is a monocyclic heterocycle. In some embodiments, R⁶ is a polycyclic heterocycle.

In some embodiments, R⁶ is 3-membered heterocycle. In some embodiments, R⁶ is 4-membered heterocycle. In some embodiments, R⁶ is 5-membered heterocycle. In some embodiments, R⁶ is 6-membered heterocycle. In some embodiments, R⁶ is 7-membered heterocycle. In some embodiments, R⁶ is 8-membered heterocycle. In some embodiments, R⁶ is 9-membered heterocycle. In some embodiments, R⁶ is 10-membered heterocycle.

In some embodiments, R⁶ is heterocyclyl substituted with one or more halogen. In some embodiments, R⁶ is heterocyclyl substituted with one or more F. In some embodiments, R⁶ is heterocyclyl substituted with one or more Cl. In some embodiments, R⁶ is heterocyclyl substituted with one or more Br. In some embodiments, R⁶ is heterocyclyl substituted with one or more I. In some embodiments, R⁶ is heterocyclyl substituted with one or more OH. In some embodiments, R⁶ is heterocyclyl substituted with one or more NH₂. In some embodiments, R⁶ is heterocyclyl substituted with one or more C₁-C₆ alkyl. In some embodiments, R⁶ is heterocyclyl substituted with one or more C₁-C₆ alkoxy. In some embodiments, R⁶ is heterocyclyl substituted with one or more NR₁₂R₁₃.

In some embodiments R⁶ is heteroaryl. In some embodiments, R⁶ is 5- to 6-membered heteroaryl. In some embodiments, R⁶ is 5- to 10-membered heteroaryl.

In some embodiments, R⁶ is heteroaryl comprising one, two, or three heteroatoms.

In some embodiments, R⁶ is heteroaryl comprising one, two, or three heteroatoms selected from N, O, and S.

In some embodiments, R⁶ is heteroaryl comprising one, two, or three heteroatoms selected from N and O.

In some embodiments, R⁶ is S-membered heteroaryl comprising 1-4 heteroatoms selected from O, N, and S. In some embodiments, R⁶ is 6-membered heteroaryl comprising 1-4 heteroatoms selected from O, N, and S. In some embodiments, R⁶ is 7-membered heteroaryl comprising 1-4 heteroatoms selected from O, N, and S. In some embodiments, R⁶ is 8-membered heteroaryl comprising 1-4 heteroatoms selected from O, N, and S. In some embodiments, R⁶ is 9-membered heteroaryl comprising 1-4 heteroatoms selected from O, N, and S. In some embodiments, R⁶ is 10-membered heteroaryl comprising 1-4 heteroatoms selected from O, N, and S.

In some embodiments, R⁶ is heteroaryl substituted with one or more halogen. In some embodiments, R⁶ is heteroaryl substituted with one or more F. In some embodiments, R⁶ is heteroaryl substituted with one or more Cl. In some embodiments, R⁶ is heteroaryl substituted with one or more Br. In some embodiments, R⁶ is heteroaryl substituted with one or more I. In some embodiments, R⁶ is heteroaryl substituted with one or more OH. In some embodiments, R⁶ is heteroaryl substituted with one or more NH₂. In some embodiments, R⁶ is heteroaryl substituted with one or more C₁-C₆ alkyl. In some embodiments, R⁶ is heteroaryl substituted with one or more C₁-C₆ alkoxy. In some embodiments, R⁶ is heteroaryl substituted with one or more NR₁₂R₁₃.

In some embodiments, R⁷ is NH₂. In some embodiments, R⁷ is NR₁₂R₁₃. In some embodiments, R⁷ is C₁-C₆ alkyl. In some embodiments, R⁷ is C₂-C₆ alkenyl. In some embodiments, R⁷ is C₂-C₆ alkynyl. In some embodiments, R⁷ is C₃-C₁₀ cycloalkyl. In some embodiments, R⁷ is aryl. In some embodiments, R⁷ is heterocyclyl. In some embodiments, R⁷ is heteroaryl.

In some embodiments, R⁷ is NH₂ or NR₁₂R₁₃. In some embodiments, R⁷ is C₁-C₆ alkyl, C₂-C₆ alkenyl, or C₂-C₆ alkynyl. In some embodiments, R⁷ is C₃-C₁₀ cycloalkyl, aryl, heterocyclyl, or heteroaryl.

In some embodiments, R⁷ is NH₂. In some embodiments, R⁷ is NR₁₂R₁₃. In some embodiments, R⁷ is unsubstituted C₁-C₆ alkyl. In some embodiments, R⁷ is unsubstituted C₂-C₆ alkenyl. In some embodiments, R⁷ is unsubstituted C₂-C₆ alkynyl. In some embodiments, R⁷ is unsubstituted C₃-C₁₀ cycloalkyl. In some embodiments, R⁷ is unsubstituted aryl. In some embodiments, R⁷ is unsubstituted heterocyclyl. In some embodiments, R⁷ is unsubstituted heteroaryl.

In some embodiments, R⁷ is methyl. In some embodiments, R⁷ is ethyl. In some embodiments, R⁷ is propyl. In some embodiments, R⁷ is n-propyl. In some embodiments, R⁷ is isopropyl. In some embodiments, R⁷ is butyl. In some embodiments, R⁷ is n-butyl. In some embodiments, R⁷ is isobutyl. In some embodiments, R⁷ is sec-butyl. In some embodiments, R⁷ is tert-butyl. In some embodiments, R⁷ is pentyl. In some embodiments, R⁷ is hexyl.

In some embodiments, R⁷ is C₁-C₆ alkyl substituted with one or more halogen. In some embodiments, R⁷ is C₁-C₆ alkyl substituted with one or more F. In some embodiments, R⁷ is C₁-C₆ alkyl substituted with one or more Cl. In some embodiments, R⁷ is C₁-C₆ alkyl substituted with one or more Br. In some embodiments, R⁷ is C₁-C₆ alkyl substituted with one or more I. In some embodiments, R⁷ is C₁-C₆ alkyl substituted with one or more OH. In some embodiments, R⁷ is C₁-C₆ alkyl substituted with one or more NH₂. In some embodiments, R⁷ is C₁-C₆ alkyl substituted with one or more C₁-C₆ alkyl. In some embodiments, R⁷ is C₁-C₆ alkyl substituted with one or more C₁-C₆ alkoxy. In some embodiments, R⁷ is C₁-C₆ alkyl substituted with one or more NR₁₂R₁₃.

In some embodiments, R⁷ is C₂ alkenyl. In some embodiments, R⁷ is C₃ alkenyl. In some embodiments, R⁷ is C₄ alkenyl. In some embodiments, R⁷ is C₅ alkenyl. In some embodiments, R⁷ is C₆ alkenyl.

In some embodiments, R⁷ is C₂-C₆ alkenyl substituted with one or more halogen. In some embodiments, R⁷ is C₂-C₆ alkenyl substituted with one or more F. In some embodiments, R⁷ is C₂-C₆ alkenyl substituted with one or more Cl. In some embodiments, R⁷ is C₂-C₆ alkenyl substituted with one or more Br. In some embodiments, R⁷ is C₂-C₆ alkenyl substituted with one or more I. In some embodiments, R⁷ is C₂-C₆ alkenyl substituted with one or more OH. In some embodiments, R⁷ is C₂-C₆ alkenyl substituted with one or more NH₂. In some embodiments, R⁷ is C₂-C₆ alkenyl substituted with one or more C₁-C₆ alkyl. In some embodiments, R⁷ is C₂-C₆ alkenyl substituted with one or more C₁-C₆ alkoxy. In some embodiments, R⁷ is C₂-C₆ alkenyl substituted with one or more NR₁₂R₁₃.

In some embodiments, R⁷ is C₂ alkynyl. In some embodiments, R⁷ is C₃ alkynyl. In some embodiments, R⁷ is C₄ alkynyl. In some embodiments, R⁷ is C₅ alkynyl. In some embodiments, R⁷ is C₆ alkynyl.

In some embodiments, R⁷ is C₂-C₆ alkynyl substituted with one or more halogen. In some embodiments, R⁷ is C₂-C₆ alkynyl substituted with one or more F. In some embodiments, R⁷ is C₂-C₆ alkynyl substituted with one or more Cl. In some embodiments, R⁷ is C₂-C₆ alkynyl substituted with one or more Br. In some embodiments, R⁶ is C₂-C₆ alkynyl substituted with one or more I. In some embodiments, R⁷ is C₂-C₆ alkynyl substituted with one or more OH. In some embodiments, R⁷ is C₂-C₆ alkynyl substituted with one or more NH₂. In some embodiments, R⁷ is C₂-C₆ alkynyl substituted with one or more C₁-C₆ alkyl. In some embodiments, R⁷ is C₂-C₆ alkynyl substituted with one or more C₁-C₆ alkoxy. In some embodiments, R⁷ is C₂-C₆ alkynyl substituted with one or more NR₁₂R₁₃.

In some embodiments, R⁷ is cyclopropyl. In some embodiments, R⁷ is cyclobutyl. In some embodiments, R⁷ is cyclopentyl. In some embodiments, R⁷ is cyclohexyl. In some embodiments, R⁷ is cycloheptyl. In some embodiments, R⁷ is cyclooctyl. In some embodiments, R⁷ is cyclononyl. In some embodiments, R⁷ is cyclodecyl.

In some embodiments, R⁷ is C₃-C₁₀ cycloalkyl substituted with one or more halogen. In some embodiments, R⁷ is C₃-C₁₀ cycloalkyl substituted with one or more F. In some embodiments, R⁷ is C₃-C₁₀ cycloalkyl substituted with one or more Cl. In some embodiments, R⁷ is C₃-C₁₀ cycloalkyl substituted with one or more Br. In some embodiments, R⁷ is C₃-C₁₀ cycloalkyl substituted with one or more I. In some embodiments, R⁷ is C₃-C₁₀ cycloalkyl substituted with one or more OH. In some embodiments, R⁷ is C₃-C₁₀ cycloalkyl substituted with one or more NH₂. In some embodiments, R⁷ is C₃-C₁₀ cycloalkyl substituted with one or more C₁-C₆ alkyl. In some embodiments, R⁷ is C₃-C₁₀ cycloalkyl substituted with one or more C₁-C₆ alkoxy. In some embodiments, R⁷ is C₃-C₁₀ cycloalkyl substituted with one or more NR₁₂R₁₃.

In some embodiments, R⁷ is C₆ aryl (e.g., phenyl).

In some embodiments, R⁷ is aryl substituted with one or more halogen. In some embodiments, R⁷ is aryl substituted with one or more F. In some embodiments, R⁷ is aryl substituted with one or more Cl. In some embodiments, R⁷ is aryl substituted with one or more Br. In some embodiments, R⁷ is aryl substituted with one or more I. In some embodiments, R⁷ is aryl substituted with one or more OH. In some embodiments, R⁷ is aryl substituted with one or more NH₂. In some embodiments, R⁷ is aryl substituted with one or more C₁-C₆ alkyl. In some embodiments, R⁷ is aryl substituted with one or more C₁-C₆ alkoxy. In some embodiments, R⁷ is aryl substituted with one or more NR₁₂R₁₃.

In some embodiments R⁷ is 3- to 8-membered heterocycle. In some embodiments R⁷ is 3- to 10-membered heterocycle.

In some embodiments, R⁷ is a monocyclic heterocyclyl. In some embodiments, R⁷ is a polycyclic heterocyclyl.

In some embodiments, R⁷ is 3-membered heterocycle. In some embodiments, R⁷ is 4-membered heterocycle. In some embodiments, R⁷ is 5-membered heterocycle. In some embodiments, R⁷ is 6-membered heterocycle. In some embodiments, R⁷ is 7-membered heterocycle. In some embodiments, R⁷ is 8-membered heterocycle. In some embodiments, R⁷ is 9-membered heterocycle. In some embodiments, R⁷ is 10-membered heterocycle.

In some embodiments, R⁷ is heterocycle substituted with one or more halogen. In some embodiments, R⁷ is heterocycle substituted with one or more F. In some embodiments, R⁷ is heterocycle substituted with one or more Cl. In some embodiments, R⁷ is heterocycle substituted with one or more Br. In some embodiments, R⁷ is heterocycle substituted with one or more I. In some embodiments, R⁷ is heterocycle substituted with one or more OH. In some embodiments, R⁷ is heterocycle substituted with one or more NH₂. In some embodiments, R⁷ is heterocycle substituted with one or more C₁-C₆ alkyl. In some embodiments, R⁷ is heterocycle substituted with one or more C₁-C₆ alkoxy. In some embodiments, R⁷ is heterocycle substituted with one or more NR₁₂R₁₃.

In some embodiments R⁷ is heteroaryl. In some embodiments, R⁷ is 5- to 6-membered heteroaryl. In some embodiments, R⁷ is 5- to 10-membered heteroaryl.

In some embodiments, R⁷ is heteroaryl comprising one, two, or three heteroatoms.

In some embodiments, R⁷ is heteroaryl comprising one, two, or three heteroatoms selected from N, O, and S.

In some embodiments, R⁷ is heteroaryl comprising one, two, or three heteroatoms selected from N and O.

In some embodiments, R⁷ is 5-membered heteroaryl comprising 1-4 heteroatoms selected from O, N, and S. In some embodiments, R⁷ is 6-membered heteroaryl comprising 1-4 heteroatoms selected from O, N, and S. In some embodiments, R⁷ is 7-membered heteroaryl comprising 1-4 heteroatoms selected from O, N, and S. In some embodiments, R⁷ is 8-membered heteroaryl comprising 1-4 heteroatoms selected from O, N, and S. In some embodiments, R⁷ is 9-membered heteroaryl comprising 1-4 heteroatoms selected from O, N, and S. In some embodiments, R⁷ is 10-membered heteroaryl comprising 1-4 heteroatoms selected from O, N, and S.

In some embodiments, R⁷ is heteroaryl substituted with one or more halogen. In some embodiments, R⁷ is heteroaryl substituted with one or more F. In some embodiments, R⁷ is heteroaryl substituted with one or more Cl. In some embodiments, R⁷ is heteroaryl substituted with one or more Br. In some embodiments, R⁷ is heteroaryl substituted with one or more 1. In some embodiments, R⁷ is heteroaryl substituted with one or more OH. In some embodiments, R⁷ is heteroaryl substituted with one or more NH₂. In some embodiments, R⁷ is heteroaryl substituted with one or more C₁-C₆ alkyl. In some embodiments, R⁷ is heteroaryl substituted with one or more C₁-C₆ alkoxy. In some embodiments, R⁷ is heteroaryl substituted with one or more NR₁₂R₁₃.

In some embodiments, R⁸ is halogen. In some embodiments, R⁸ is F. In some embodiments, R⁸ is Cl. In some embodiments, R⁸ is Br. In some embodiments, R⁸ is I. In some embodiments, R⁸ is oxo. In some embodiments, R⁸ is CN. In some embodiments, R⁸ is C₁-C₆ alkyl. In some embodiments, R⁸ is C₁-C₆ alkoxy. In some embodiments, R⁸ is C₁-C₆ alkyl-C₁-C₆ alkoxy. In some embodiments, R⁸ is C₁-C₆ alkyl-NR₁₂R₁₃.

In some embodiments, R⁸ is halogen. In some embodiments, R⁸ is F. In some embodiments, R⁸ is Cl. In some embodiments, R⁸ is Br. In some embodiments, R⁸ is I. In some embodiments, R⁸ is oxo. In some embodiments, R⁸ is CN. In some embodiments, R⁸ is unsubstituted C₁-C₆ alkyl. In some embodiments, R⁸ is unsubstituted C₁-C₆ alkoxy. In some embodiments, R⁸ is unsubstituted C₁-C₆ alkyl-C₁-C₆ alkoxy. In some embodiments, R⁸ is unsubstituted C₁-C₆ alkyl-NR₁₂R₁₃.

In some embodiments, R⁸ is C₁-C₆ alkyl substituted with one or more halogen. In some embodiments, R⁸ is C₁-C₆ alkyl substituted with one or more F. In some embodiments, R⁸ is C₁-C₆ alkyl substituted with one or more Cl. In some embodiments, R⁸ is C₁-C₆ alkyl substituted with one or more Br. In some embodiments, R⁸ is C₁-C₆ alkyl substituted with one or more I. In some embodiments, R⁸ is C₁-C₆ alkyl substituted with one or more oxo. In some embodiments, R⁸ is C₁-C₆ alkyl substituted with one or more OH. In some embodiments, R⁸ is C₁-C₆ alkyl substituted with one or more NH₂. In some embodiments, R⁸ is C₁-C₆ alkyl substituted with one or more C₁-C₆ alkyl. In some embodiments, R⁸ is C₁-C₆ alkyl substituted with one or more C₁-C₆ alkoxy. In some embodiments, R⁸ is C₁-C₆ alkyl substituted with one or more NR₁₂R₁₃. In some embodiments, R⁸ is C₁-C₆ alkyl substituted with one or more C₃-C₁₀ cycloalkyl. In some embodiments, R⁸ is C₁-C₆ alkyl substituted with one or more heterocycloalkyl.

In some embodiments, R⁸ is C₁-C₆ alkoxy substituted with one or more halogen. In some embodiments, R⁸ is C₁-C₆ alkoxy substituted with one or more F. In some embodiments, R⁸ is C₁-C₆ alkoxy substituted with one or more Cl. In some embodiments, R⁸ is C₁-C₆ alkoxy substituted with one or more Br. In some embodiments, R⁸ is C₁-C₆ alkoxy substituted with one or more I. In some embodiments, R⁸ is C₁-C₆ alkoxy substituted with one or more oxo. In some embodiments, R⁸ is C₁-C₆ alkoxy substituted with one or more OH. In some embodiments, R⁸ is C₁-C₆ alkoxy substituted with one or more NH₂. In some embodiments, R⁸ is C₁-C₆ alkoxy substituted with one or more C₁-C₆ alkyl. In some embodiments, R⁸ is C₁-C₆ alkoxy substituted with one or more C₁-C₆ alkoxy. In some embodiments, R⁸ is C₁-C₆ alkoxy substituted with one or more NR₁₂R₁₃. In some embodiments, R⁸ is C₁-C₆ alkoxy substituted with one or more C₃-C₁₀ cycloalkyl. In some embodiments, R⁸ is C₁-C₆ alkoxy substituted with one or more heterocycloalkyl.

In some embodiments, R⁸ is C₁-C₆ alkyl-C₁-C₆ alkoxy substituted with one or more halogen. In some embodiments, R⁸ is C₁-C₆ alkyl-C₁-C₆ alkoxy substituted with one or more F. In some embodiments, R⁸ is C₁-C₆ alkyl-C₁-C₆ alkoxy substituted with one or more Cl. In some embodiments, R⁸ is C₁-C₆ alkyl-C₁-C₆ alkoxy substituted with one or more Br. In some embodiments, R⁸ is C₁-C₆ alkyl-C₁-C₆ alkoxy substituted with one or more I. In some embodiments, R⁸ is C₁-C₆ alkyl-C₁-C₆ alkoxy substituted with one or more oxo. In some embodiments, R⁸ is C₁-C₆ alkyl-C₁-C₆ alkoxy substituted with one or more OH. In some embodiments, R⁸ is C₁-C₆ alkyl-C₁-C₆ alkoxy NH₂. In some embodiments, R⁸ is C₁-C₆ alkyl-C₁-C₆ alkoxy substituted with one or more C₁-C₆ alkyl. In some embodiments, R⁸ is C₁-C₆ alkyl-C₁-C₆ alkoxy substituted with one or more C₁-C₆ alkoxy. In some embodiments, R⁸ is C₁-C₆ alkyl-C₁-C₆ alkoxy substituted with one or more NR₁₂R₁₃. In some embodiments, R⁸ is C₁-C₆ alkyl-C₁-C₆ alkoxy substituted with one or more C₃-C₁₀ cycloalkyl. In some embodiments, R⁸ is C₁-C₆ alkyl-C₁-C₆ alkoxy substituted with one or more heterocycloalkyl.

In some embodiments, R⁸ is C₁-C₆ alkyl-C₁-C₆ alkoxy wherein the alkyl is substituted with one or more halogen. In some embodiments, R⁸ is C₁-C₆ alkyl-C₁-C₆ alkoxy wherein the alkyl is substituted with one or more F. In some embodiments, R⁸ is C₁-C₆ alkyl-C₁-C₆ alkoxy wherein the alkyl is substituted with one or more Cl. In some embodiments, R⁸ is C₁-C₆ alkyl-C₁-C₆ alkoxy wherein the alkyl is substituted with one or more Br. In some embodiments, R⁸ is C₁-C₆ alkyl-C₁-C₆ alkoxy wherein the alkyl is substituted with one or more I. In some embodiments, R⁸ is C₁-C₆ alkyl-C₁-C₆ alkoxy wherein the alkyl is substituted with one or more oxo. In some embodiments, R⁸ is C₁-C₆ alkyl-C₁-C₆ alkoxy wherein the alkyl is substituted with one or more OH. In some embodiments, R⁸ is C₁-C₆ alkyl-C₁-C₆ alkoxy wherein the alkyl is substituted with one or more NH₂. In some embodiments, R⁸ is C₁-C₆ alkyl-C₁-C₆ alkoxy wherein the alkyl is substituted with one or more C₁-C₆ alkyl. In some embodiments, R⁸ is C₁-C₆ alkyl-C₁-C₆ alkoxy wherein the alkyl is substituted with one or more C₁-C₆ alkoxy. In some embodiments, R⁸ is C₁-C₆ alkyl-C₁-C₆ alkoxy wherein the alkyl is substituted with one or more NR¹²R¹³. In some embodiments, R⁸ is C₁-C₆ alkyl-C₁-C₆ alkoxy wherein the alkyl is substituted with one or more C₃-C₁₀ cycloalkyl. In some embodiments, R⁸ is C₁-C₆ alkyl-C₁-C₆ alkoxy wherein the alkyl is substituted with one or more heterocycloalkyl.

In some embodiments, R⁸ is C₁-C₆ alkyl-C₁-C₆ alkoxy wherein the alkoxy is substituted with one or more halogen. In some embodiments, R⁸ is C₁-C₆ alkyl-C₁-C₆ alkoxy wherein the alkoxy is substituted with one or more F. In some embodiments, R⁸ is C₁-C₆ alkyl-C₁-C₆ alkoxy wherein the alkoxy is substituted with one or more Cl. In some embodiments, R⁸ is C₁-C₆ alkyl-C₁-C₆ alkoxy wherein the alkoxy is substituted with one or more Br. In some embodiments, R⁸ is C₁-C₆ alkyl-C₁-C₆ alkoxy wherein the alkoxy is substituted with one or more I. In some embodiments, R⁸ is C₁-C₆ alkyl-C₁-C₆ alkoxy wherein the alkoxy is substituted with one or more oxo. In some embodiments, R⁸ is C₁-C₆ alkyl-C₁-C₆ alkoxy wherein the alkoxy is substituted with one or more OH. In some embodiments, R⁸ is C₁-C₆ alkyl-C₁-C₆ alkoxy wherein the alkoxy is substituted with one or more NH₂. In some embodiments, R⁸ is C₁-C₆ alkyl-C₁-C₆ alkoxy wherein the alkoxy is substituted with one or more C₁-C₆ alkyl. In some embodiments, R⁸ is C₁-C₆ alkyl-C₁-C₆ alkoxy wherein the alkoxy is substituted with one or more C₁-C₆ alkoxy. In some embodiments, R⁸ is C₁-C₆ alkyl-C₁-C₆ alkoxy wherein the alkoxy is substituted with one or more NR¹²R¹³. In some embodiments, R⁸ is C₁-C₆ alkyl-C₁-C₆ alkoxy wherein the alkoxy is substituted with one or more C₃-C₁₀ cycloalkyl. In some embodiments, R⁸ is C₁-C₆ alkyl-C₁-C₆ alkoxy wherein the alkoxy is substituted with one or more heterocycloalkyl.

In some embodiments, R⁸ is C₁-C₆ alkyl-NR₁₂R₁₃ substituted with one or more halogen. In some embodiments, R⁸ is C₁-C₆ alkyl-NR¹²R¹³ substituted with one or more F. In some embodiments, R⁸ is C₁-C₆ alkyl-NR₁₂R₁₃ substituted with one or more Cl. In some embodiments, R⁸ is C₁-C₆ alkyl-NR₁₂R₁₃ substituted with one or more Br. In some embodiments, R⁸ is C₁-C₆ alkyl-NR₁₂R₁₃ substituted with one or more I. In some embodiments, R⁸ is C₁-C₆ alkyl-NR¹²R¹³ substituted with one or more oxo. In some embodiments, R⁸ is Ct-C₆ alkyl-NR₁₂R₁₃ substituted with one or more OH. In some embodiments, R⁸ is C₁-C₆ alkyl-NR¹²R¹³ substituted with one or more NH₂. In some embodiments, R⁸ is C₁-C₆ alkyl-NR₁₂R₁₃ substituted with one or more C₁-C₆ alkyl. In some embodiments, R⁸ is C₁-C₆ alkyl-NR₁₂R₁₃ substituted with one or more C₁-C₆ alkoxy. In some embodiments, R⁸ is C₁-C₆ alkyl-NR₁₂R₁₃ substituted with one or more NR₁₂R₁₃. In some embodiments, R⁸ is C₁-C₆ alkyl-NR₁₂R₁₃ substituted with one or more C₃-C₁₀ cycloalkyl. In some embodiments, R⁸ is C₁-C₆ alkyl-NR₁₂R₁₃ substituted with one or more heterocycloalkyl.

In some embodiments, R⁸ is

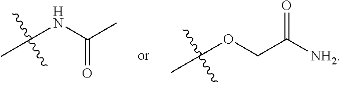

In some embodiments, R⁸ is

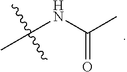 [SMILES: *NC(C)=O]

In some embodiments, R⁸ is

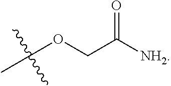 [SMILES: *OCC(N)=O]

In some embodiments, R⁶ and R⁸, together with the molecules to which they are attached and any intervening atoms, form a 5-membered heterocycle. In some embodiments, R⁶ and R⁸, together with the molecules to which they are attached and any intervening atoms, form a 6-membered heterocycle. In some embodiments, R⁶ and R⁸, together with the molecules to which they are attached and any intervening atoms, form a 7-membered heterocycle. In some embodiments, R⁶ and R⁸, together with the molecules to which they are attached and any intervening atoms, form an 8-membered heterocycle. In some embodiments, R⁶ and R⁸, together with the molecules to which they are attached and any intervening atoms, form a 9-membered heterocycle. In some embodiments, R⁶ and R⁸, together with the molecules to which they are attached and any intervening atoms, form a 10-membered heterocycle. In some embodiments, R₆ and R⁸, together with the molecules to which they are attached and any intervening atoms, form an 11-membered heterocycle. In some embodiments, R⁶ and R⁸, together with the molecules to which they are attached and any intervening atoms, form a 12-membered heterocycle. In some embodiments, R⁶ and R⁸, together with the molecules to which they are attached and any intervening atoms, form a 13-membered heterocycle. In some embodiments, R⁶ and R⁸, together with the molecules to which they are attached and any intervening atoms, form a 14-membered heterocycle.

In some embodiments, R⁶ and R⁸, together with the molecules to which they are attached and any intervening atoms, form an unsubstituted 5-membered heterocycle. In some embodiments, R⁶ and R⁸, together with the molecules to which they are attached and any intervening atoms, form an unsubstituted 6-membered heterocycle. In some embodiments, R⁶ and R⁸, together with the molecules to which they are attached and any intervening atoms, form an unsubstituted 7-membered heterocycle. In some embodiments, R⁶ and R⁸, together with the molecules to which they are attached and any intervening atoms, form an unsubstituted 8-membered heterocycle. In some embodiments, R⁶ and R⁸, together with the molecules to which they are attached and any intervening atoms, form an unsubstituted 9-membered heterocycle. In some embodiments, R⁶ and R⁸, together with the molecules to which they are attached and any intervening atoms, form an unsubstituted 10-membered heterocycle. In some embodiments, R⁶ and R⁸, together with the molecules to which they are attached and any intervening atoms, form an unsubstituted 11-membered heterocycle. In some embodiments, R⁶ and R⁸, together with the molecules to which they are attached and any intervening atoms, form an unsubstituted 12-membered heterocycle. In some embodiments, R⁶ and R⁸, together with the molecules to which they are attached and any intervening atoms, form an unsubstituted 13-membered heterocycle. In some embodiments, R⁶ and R⁸, together with the molecules to which they are attached and any intervening atoms, form an unsubstituted 14-membered heterocycle.

In some embodiments, R⁶ and R⁸, together with the molecules to which they are attached and any intervening atoms, form a 5-membered heterocycle substituted with one or more halogen. In some embodiments, R⁶ and R⁸, together with the molecules to which they are attached and any intervening atoms, form a 5-membered heterocycle substituted with one or more F. In some embodiments, R⁶ and R⁸, together with the molecules to which they are attached and any intervening atoms, form a 5-membered heterocycle substituted with one or more Cl. In some embodiments, R⁶ and R⁸, together with the molecules to which they are attached and any intervening atoms, form a 5-membered heterocycle substituted with one or more Br. In some embodiments, R⁶ and R², together with the molecules to which they are attached and any intervening atoms, form a 5-membered heterocycle substituted with one or more I. In some embodiments, R⁶ and R⁸, together with the molecules to which they are attached and any intervening atoms, form a 5-membered heterocycle substituted with one or more OH. In some embodiments, R⁶ and R⁸, together with the molecules to which they are attached and any intervening atoms, form a 5-membered heterocycle substituted with one or more NH₂. In some embodiments, R⁶ and R⁸, together with the molecules to which they are attached and any intervening atoms, form a S-membered heterocycle substituted with one or more C₁-C₆ alkyl. In some embodiments, R⁶ and R⁸, together with the molecules to which they are attached and any intervening atoms, form a 5-membered heterocycle substituted with one or more C₁-C₆ alkoxy. In some embodiments, R⁶ and R⁸, together with the molecules to which they are attached and any intervening atoms, form a 5-membered heterocycle substituted with one or more C₁-C₆ alkyl-C₁-C₆ alkoxy. In some embodiments, R⁶ and R⁸, together with the molecules to which they are attached and any intervening atoms, form a 5-membered heterocycle substituted with one or more C₁-C₆ alkyl-NHC₁-C₆ alkyl. In some embodiments, R⁶ and R⁸, together with the molecules to which they are attached and any intervening atoms, form a 5-membered heterocycle substituted with one or more NR₁₂R₁₃.

In some embodiments, R⁶ and R⁸, together with the molecules to which they are attached and any intervening atoms, form a 6-membered heterocycle substituted with one or more halogen. In some embodiments, R⁶ and R⁸, together with the molecules to which they are attached and any intervening atoms, form a 6-membered heterocycle substituted with one or more F. In some embodiments, R⁶ and R⁸, together with the molecules to which they are attached and any intervening atoms, form a 6-membered heterocycle substituted with one or more Cl. In some embodiments, R⁶ and R⁸, together with the molecules to which they are attached and any intervening atoms, form a 6-membered heterocycle substituted with one or more Br. In some embodiments, R⁶ and R⁸, together with the molecules to which they are attached and any intervening atoms, form a 6-membered heterocycle substituted with one or more I. In some embodiments, R⁶ and R⁸, together with the molecules to which they are attached and any intervening atoms, form a 6-membered heterocycle substituted with one or more OH. In some embodiments, R⁶ and R⁸, together with the molecules to which they are attached and any intervening atoms, form a 6-membered heterocycle substituted with one or more NH₂. In some embodiments, R⁶ and R⁸, together with the molecules to which they are attached and any intervening atoms, form a 6-membered heterocycle substituted with one or more C₁-C₆ alkyl. In some embodiments, R⁶ and R⁸, together with the molecules to which they are attached and any intervening atoms, form a 6-membered heterocycle substituted with one or more C₁-C₆ alkoxy. In some embodiments, R⁶ and R⁸, together with the molecules to which they are attached and any intervening atoms, form a 6-membered heterocycle substituted with one or more C₁-C₆ alkyl-C₁-C₆ alkoxy. In some embodiments, R⁶ and R⁸, together with the molecules to which they are attached and any intervening atoms, form a 6-membered heterocycle substituted with one or more C₁-C₆ alkyl-NHC₁-C₆ alkyl. In some embodiments, R⁶ and R⁸, together with the molecules to which they are attached and any intervening atoms, form a 6-membered heterocycle substituted with one or more NR₁₂R₁₃.

In some embodiments, R⁶ and R⁸, together with the molecules to which they are attached and any intervening atoms, form a 7-membered heterocycle substituted with one or more halogen. In some embodiments, R⁶ and R⁸, together with the molecules to which they are attached and any intervening atoms, form a 7-membered heterocycle substituted with one or more F. In some embodiments, R⁶ and R⁸, together with the molecules to which they are attached and any intervening atoms, form a 7-membered heterocycle substituted with one or more Cl. In some embodiments, R⁶ and R⁸, together with the molecules to which they are attached and any intervening atoms, form a 7-membered heterocycle substituted with one or more Br. In some embodiments, R⁶ and R⁸, together with the molecules to which they are attached and any intervening atoms, form a 7-membered heterocycle substituted with one or more I. In some embodiments, R⁶ and R⁸, together with the molecules to which they are attached and any intervening atoms, form a 7-membered heterocycle substituted with one or more OH. In some embodiments, R⁶ and R⁸, together with the molecules to which they are attached and any intervening atoms, form a 7-membered heterocycle substituted with one or more NH₂. In some embodiments, R⁶ and R⁸, together with the molecules to which they are attached and any intervening atoms, form a 7-membered heterocycle substituted with one or more C₁-C₆ alkyl. In some embodiments, R⁶ and R⁸, together with the molecules to which they are attached and any intervening atoms, form a 7-membered heterocycle substituted with one or more C₁-C₆ alkoxy. In some embodiments, R⁶ and R⁸, together with the molecules to which they are attached and any intervening atoms, form a 7-membered heterocycle substituted with one or more C₁-C₆ alkyl-C₁-C₆ alkoxy. In some embodiments, R⁶ and R⁸, together with the molecules to which they are attached and any intervening atoms, form a 7-membered heterocycle substituted with one or more C₁-C₆ alkyl-NHC₁-C₆ alkyl. In some embodiments, R⁶ and R⁸, together with the molecules to which they are attached and any intervening atoms, form a 7-membered heterocycle substituted with one or more NR₁₂R₁₃.

In some embodiments, R⁶ and R⁸, together with the molecules to which they are attached and any intervening atoms, form an 8-membered heterocycle substituted with one or more halogen. In some embodiments, R⁶ and R⁸, together with the molecules to which they are attached and any intervening atoms, form an 8-membered heterocycle substituted with one or more F. In some embodiments, R⁶ and R⁸, together with the molecules to which they are attached and any intervening atoms, form an 8-membered heterocycle substituted with one or more Cl. In some embodiments, R⁶ and R⁸, together with the molecules to which they are attached and any intervening atoms, form an 8-membered heterocycle substituted with one or more Br. In some embodiments, R⁶ and R⁸, together with the molecules to which they are attached and any intervening atoms, form an 8-membered heterocycle substituted with one or more I. In some embodiments, R⁶ and R⁸, together with the molecules to which they are attached and any intervening atoms, form an 8-membered heterocycle substituted with one or more OH. In some embodiments, R⁶ and R⁸, together with the molecules to which they are attached and any intervening atoms, form an 8-membered heterocycle substituted with one or more NH₂. In some embodiments, R⁶ and R⁸, together with the molecules to which they are attached and any intervening atoms, form an 8-membered heterocycle substituted with one or more C₁-C₆ alkyl. In some embodiments, R⁶ and R⁸, together with the molecules to which they are attached and any intervening atoms, form an 8-membered heterocycle substituted with one or more C₁-C₆ alkoxy. In some embodiments, R⁶ and R⁸, together with the molecules to which they are attached and any intervening atoms, form an 8-membered heterocycle substituted with one or more C₁-C₆ alkyl-C₁-C₆ alkoxy. In some embodiments, R⁶ and R⁸, together with the molecules to which they are attached and any intervening atoms, form a 8-membered heterocycle substituted with one or more C₁-C₆ alkyl-NHC₁-C₆ alkyl. In some embodiments, R⁶ and R⁸, together with the molecules to which they are attached and any intervening atoms, form a 8-membered heterocycle substituted with one or more NR¹²R¹³.

In some embodiments, R⁶ and R⁸, together with the molecules to which they are attached and any intervening atoms, form a 9-membered heterocycle substituted with one or more halogen. In some embodiments, R⁶ and R⁸, together with the molecules to which they are attached and any intervening atoms, form a 9-membered heterocycle substituted with one or more F. In some embodiments, R⁶ and R⁸, together with the molecules to which they are attached and any intervening atoms, form a 9-membered heterocycle substituted with one or more Cl. In some embodiments, R⁶ and R⁸, together with the molecules to which they are attached and any intervening atoms, form a 9-membered heterocycle substituted with one or more Br. In some embodiments, R⁶ and R⁸, together with the molecules to which they are attached and any intervening atoms, form a 9-membered heterocycle substituted with one or more I. In some embodiments, R⁶ and R⁸, together with the molecules to which they are attached and any intervening atoms, form a 9-membered heterocycle substituted with one or more OH. In some embodiments, R⁶ and R⁸, together with the molecules to which they are attached and any intervening atoms, form a 9-membered heterocycle substituted with one or more NH₂. In some embodiments, R⁶ and R⁸, together with the molecules to which they are attached and any intervening atoms, form a 9-membered heterocycle substituted with one or more C₁-C₆ alkyl. In some embodiments, R⁶ and R⁸, together with the molecules to which they are attached and any intervening atoms, form a 9-membered heterocycle substituted with one or more C₁-C₆ alkoxy. In some embodiments, R⁶ and R⁸, together with the molecules to which they are attached and any intervening atoms, form a 9-membered heterocycle substituted with one or more C₁-C₆ alkyl-C₁-C₆ alkoxy. In some embodiments, R⁶ and R⁸, together with the molecules to which they are attached and any intervening atoms, form a 9-membered heterocycle substituted with one or more C₁-C₆ alkyl-NHC₁-C₆ alkyl. In some embodiments, R⁶ and R⁸, together with the molecules to which they are attached and any intervening atoms, form a 9-membered heterocycle substituted with one or more NR¹²R¹³.

In some embodiments, R⁶ and R⁸, together with the molecules to which they are attached and any intervening atoms, form a 10-membered heterocycle substituted with one or more halogen. In some embodiments, R⁶ and R⁸, together with the molecules to which they are attached and any intervening atoms, form a 10-membered heterocycle substituted with one or more F. In some embodiments, R⁶ and R⁸, together with the molecules to which they are attached and any intervening atoms, form a 10-membered heterocycle substituted with one or more Cl. In some embodiments, R⁶ and R⁸, together with the molecules to which they are attached and any intervening atoms, form a 10-membered heterocycle substituted with one or more Br. In some embodiments, R⁶ and R⁸, together with the molecules to which they are attached and any intervening atoms, form a 10-membered heterocycle substituted with one or more I. In some embodiments, R⁶ and R⁸, together with the molecules to which they are attached and any intervening atoms, form a 10-membered heterocycle substituted with one or more OH. In some embodiments, R⁶ and R⁸, together with the molecules to which they are attached and any intervening atoms, form a 10-membered heterocycle substituted with one or more NH₂. In some embodiments, R⁶ and R⁸, together with the molecules to which they are attached and any intervening atoms, form a 10-membered heterocycle substituted with one or more C₁-C₆ alkyl. In some embodiments, R⁶ and R⁸, together with the molecules to which they are attached and any intervening atoms, form a 10-membered heterocycle substituted with one or more C₁-C₆ alkoxy. In some embodiments, R⁶ and R⁸, together with the molecules to which they are attached and any intervening atoms, form a 10-membered heterocycle substituted with one or more C₁-C₆ alkyl-C₁-C₆ alkoxy. In some embodiments, R⁶ and R⁸, together with the molecules to which they are attached and any intervening atoms, form a 10-membered heterocycle substituted with one or more C₁-C₆ alkyl-NHC₁-C₆ alkyl. In some embodiments, R⁶ and R⁸, together with the molecules to which they are attached and any intervening atoms, form a 10-membered heterocycle substituted with one or more NR¹²R¹³.

In some embodiments, R⁶ and R⁸, together with the molecules to which they are attached and any intervening atoms, form an 11-membered heterocycle substituted with one or more halogen. In some embodiments, R⁶ and R⁸, together with the molecules to which they are attached and any intervening atoms, form an 11-membered heterocycle substituted with one or more F. In some embodiments, R⁶ and R⁸, together with the molecules to which they are attached and any intervening atoms, form an 11-membered heterocycle substituted with one or more Cl. In some embodiments, R⁶ and R⁸, together with the molecules to which they are attached and any intervening atoms, form an 11-membered heterocycle substituted with one or more Br. In some embodiments, R⁶ and R⁸, together with the molecules to which they are attached and any intervening atoms, form an 11-membered heterocycle substituted with one or more I. In some embodiments, R⁶ and R⁸, together with the molecules to which they are attached and any intervening atoms, form an 11-membered heterocycle substituted with one or more OH. In some embodiments, R⁶ and R⁸, together with the molecules to which they are attached and any intervening atoms, form an 11-membered heterocycle substituted with one or more NH₂. In some embodiments, R⁶ and R⁸, together with the molecules to which they are attached and any intervening atoms, form an 11-membered heterocycle substituted with one or more C₁-C₆ alkyl. In some embodiments, R⁶ and R⁸, together with the molecules to which they are attached and any intervening atoms, form an 11-membered heterocycle substituted with one or more C₁-C₆ alkoxy. In some embodiments, R⁶ and R⁸, together with the molecules to which they are attached and any intervening atoms, form an 11-membered heterocycle substituted with one or more C₁-C₆ alkyl-C₁-C₆ alkoxy. In some embodiments, R⁶ and R⁸, together with the molecules to which they are attached and any intervening atoms, form an 11-membered heterocycle substituted with one or more C₁-C₆ alkyl-NHC₁-C₆ alkyl. In some embodiments, R⁶ and R⁸, together with the molecules to which they are attached and any intervening atoms, form an 11-membered heterocycle substituted with one or more NR¹²R¹³.

In some embodiments, R⁶ and R⁸, together with the molecules to which they are attached and any intervening atoms, form a 12-membered heterocycle substituted with one or more halogen. In some embodiments, R⁶ and R⁸, together with the molecules to which they are attached and any intervening atoms, form a 12-membered heterocycle substituted with one or more F. In some embodiments, R⁶ and R⁸, together with the molecules to which they are attached and any intervening atoms, form a 12-membered heterocycle substituted with one or more Cl. In some embodiments, R⁶ and R⁸, together with the molecules to which they are attached and any intervening atoms, form a 12-membered heterocycle substituted with one or more Br. In some embodiments, R⁶ and R⁸, together with the molecules to which they are attached and any intervening atoms, form a 12-membered heterocycle substituted with one or more I. In some embodiments, R⁶ and R⁸, together with the molecules to which they are attached and any intervening atoms, form a 12-membered heterocycle substituted with one or more OH. In some embodiments, R⁶ and R⁸, together with the molecules to which they are attached and any intervening atoms, form a 12-membered heterocycle substituted with one or more NH₂. In some embodiments, R⁶ and R⁸, together with the molecules to which they are attached and any intervening atoms, form a 12-membered heterocycle substituted with one or more C₁-C₆ alkyl. In some embodiments, R⁶ and R⁸, together with the molecules to which they are attached and any intervening atoms, form a 12-membered heterocycle substituted with one or more C₁-C₆ alkoxy. In some embodiments, R⁶ and R⁸, together with the molecules to which they are attached and any intervening atoms, form a 12-membered heterocycle substituted with one or more C₁-C₆ alkyl-C₁-C₆ alkoxy. In some embodiments, R⁶ and R⁸, together with the molecules to which they are attached and any intervening atoms, form a 12-membered heterocycle substituted with one or more C₁-C₆ alkyl-NHC₁-C₆ alkyl. In some embodiments, R⁶ and R², together with the molecules to which they are attached and any intervening atoms, form a 12-membered heterocycle substituted with one or more NR₁₂R₁₃.

In some embodiments, R⁶ and R⁸, together with the molecules to which they are attached and any intervening atoms, form a 13-membered heterocycle substituted with one or more halogen. In some embodiments, R⁶ and R⁸, together with the molecules to which they are attached and any intervening atoms, form a 13-membered heterocycle substituted with one or more F. In some embodiments, R⁶ and R⁸, together with the molecules to which they are attached and any intervening atoms, form a 13-membered heterocycle substituted with one or more Cl. In some embodiments, R⁶ and R⁸, together with the molecules to which they are attached and any intervening atoms, form a 13-membered heterocycle substituted with one or more Br. In some embodiments, R⁶ and R⁸, together with the molecules to which they are attached and any intervening atoms, form a 13-membered heterocycle substituted with one or more I. In some embodiments, R⁶ and R⁸, together with the molecules to which they are attached and any intervening atoms, form a 13-membered heterocycle substituted with one or more OH. In some embodiments, R⁶ and R⁸, together with the molecules to which they are attached and any intervening atoms, form a 13-membered heterocycle substituted with one or more NH₂. In some embodiments, R⁶ and R⁸, together with the molecules to which they are attached and any intervening atoms, form a 13-membered heterocycle substituted with one or more C₁-C₆ alkyl. In some embodiments, R⁶ and R⁸, together with the molecules to which they are attached and any intervening atoms, form a 13-membered heterocycle substituted with one or more C₁-C₆ alkoxy. In some embodiments, R⁶ and R⁸, together with the molecules to which they are attached and any intervening atoms, form a 13-membered heterocycle substituted with one or more C₁-C₆ alkyl-C₁-C₆ alkoxy. In some embodiments, R⁶ and R⁸, together with the molecules to which they are attached and any intervening atoms, form a 13-membered heterocycle substituted with one or more C₁-C₆ alkyl-NHC₁-C₆ alkyl. In some embodiments, R⁶ and R⁸, together with the molecules to which they are attached and any intervening atoms, form a 13-membered heterocycle substituted with one or more NR¹²R¹³.

In some embodiments, R⁶ and R⁸, together with the molecules to which they are attached and any intervening atoms, form a 14-membered heterocycle substituted with one or more halogen. In some embodiments, R⁶ and R⁸, together with the molecules to which they are attached and any intervening atoms, form a 14-membered heterocycle substituted with one or more F. In some embodiments, R⁶ and R⁸, together with the molecules to which they are attached and any intervening atoms, form a 14-membered heterocycle substituted with one or more Cl. In some embodiments, R⁶ and R⁸, together with the molecules to which they are attached and any intervening atoms, form a 14-membered heterocycle substituted with one or more Br. In some embodiments, R⁶ and R⁸, together with the molecules to which they are attached and any intervening atoms, form a 14-membered heterocycle substituted with one or more I. In some embodiments, R⁶ and R⁸, together with the molecules to which they are attached and any intervening atoms, form a 14-membered heterocycle substituted with one or more OH. In some embodiments, R⁶ and R⁸, together with the molecules to which they are attached and any intervening atoms, form a 14-membered heterocycle substituted with one or more NH₂. In some embodiments, R⁶ and R⁸, together with the molecules to which they are attached and any intervening atoms, form a 14-membered heterocycle substituted with one or more C₁-C₆ alkyl. In some embodiments, R⁶ and R⁸, together with the molecules to which they are attached and any intervening atoms, form a 14-membered heterocycle substituted with one or more C₁-C₆ alkoxy. In some embodiments, R⁶ and R⁸, together with the molecules to which they are attached and any intervening atoms, form a 14-membered heterocycle substituted with one or more C₁-C₆ alkyl-C₁-C₆ alkoxy. In some embodiments, R⁶ and R⁸, together with the molecules to which they are attached and any intervening atoms, form a 14-membered heterocycle substituted with one or more C₁-C₆ alkyl-NHC₁-C₆ alkyl. In some embodiments, R⁶ and R⁸, together with the molecules to which they are attached and any intervening atoms, form a 14-membered heterocycle substituted with one or more NR₁₂R₁₃.

In some embodiments, R⁷ and R⁸, together with the molecules to which they are attached and any intervening atoms, form a 5-membered heterocycle. In some embodiments, R⁷ and R², together with the molecules to which they are attached and any intervening atoms, form a 6-membered heterocycle. In some embodiments, R⁷ and R⁸, together with the molecules to which they are attached and any intervening atoms, form a 7-membered heterocycle. In some embodiments, R⁷ and R⁸, together with the molecules to which they are attached and any intervening atoms, form an 8-membered heterocycle. In some embodiments, R⁷ and R⁸, together with the molecules to which they are attached and any intervening atoms, form a 9-membered heterocycle. In some embodiments, R⁷ and R⁸, together with the molecules to which they are attached and any intervening atoms, form a 10-membered heterocycle. In some embodiments, R⁷ and R⁸, together with the molecules to which they are attached and any intervening atoms, form an 11-membered heterocycle. In some embodiments, R⁷ and R⁸, together with the molecules to which they are attached and any intervening atoms, form a 12-membered heterocycle. In some embodiments, R⁷ and R⁸, together with the molecules to which they are attached and any intervening atoms, form a 13-membered heterocycle. In some embodiments, R⁷ and R⁸, together with the molecules to which they are attached and any intervening atoms, form a 14-membered heterocycle.

In some embodiments, R⁷ and R⁸, together with the molecules to which they are attached and any intervening atoms, form an unsubstituted 5-membered heterocycle. In some embodiments, R⁷ and R⁸, together with the molecules to which they are attached and any intervening atoms, form an unsubstituted 6-membered heterocycle. In some embodiments, R⁷ and R⁸, together with the molecules to which they are attached and any intervening atoms, form an unsubstituted 7-membered heterocycle. In some embodiments, R⁷ and R⁸, together with the molecules to which they are attached and any intervening atoms, form an unsubstituted 8-membered heterocycle. In some embodiments, R⁷ and R⁸, together with the molecules to which they are attached and any intervening atoms, form an unsubstituted 9-membered heterocycle. In some embodiments, R⁷ and R⁸, together with the molecules to which they are attached and any intervening atoms, form an unsubstituted 10-membered heterocycle. In some embodiments, R¹ and R⁸, together with the molecules to which they are attached and any intervening atoms, form an unsubstituted 11-membered heterocycle. In some embodiments, R⁷ and R⁸, together with the molecules to which they are attached and any intervening atoms, form an unsubstituted 12-membered heterocycle. In some embodiments, R⁷ and R⁸, together with the molecules to which they are attached and any intervening atoms, form an unsubstituted 13-membered heterocycle. In some embodiments, R⁷ and R⁸, together with the molecules to which they are attached and any intervening atoms, form an unsubstituted 14-membered heterocycle.

In some embodiments, R⁷ and R⁸, together with the molecules to which they are attached and any intervening atoms, form a S-membered heterocycle substituted with one or more halogen. In some embodiments, R⁷ and R⁸, together with the molecules to which they are attached and any intervening atoms, form a 5-membered heterocycle substituted with one or more F. In some embodiments, R⁷ and R⁸, together with the molecules to which they are attached and any intervening atoms, form a 5-membered heterocycle substituted with one or more Cl. In some embodiments, R⁷ and R⁸, together with the molecules to which they are attached and any intervening atoms, form a 5-membered heterocycle substituted with one or more Br. In some embodiments, R⁷ and R⁸, together with the molecules to which they are attached and any intervening atoms, form a 5-membered heterocycle substituted with one or more I. In some embodiments, R⁷ and R⁸, together with the molecules to which they are attached and any intervening atoms, form a 5-membered heterocycle substituted with one or more OH. In some embodiments, R⁷ and R⁸, together with the molecules to which they are attached and any intervening atoms, form a 5-membered heterocycle substituted with one or more NH₂. In some embodiments, R⁷ and R⁸, together with the molecules to which they are attached and any intervening atoms, form a S-membered heterocycle substituted with one or more C₁-C₆ alkyl. In some embodiments, R⁷ and R⁸, together with the molecules to which they are attached and any intervening atoms, form a 5-membered heterocycle substituted with one or more C₁-C₆ alkoxy. In some embodiments, R⁷ and R⁸, together with the molecules to which they are attached and any intervening atoms, form a 5-membered heterocycle substituted with one or more C₁-C₆ alkyl-C₁-C₆ alkoxy. In some embodiments, R⁷ and R⁸, together with the molecules to which they are attached and any intervening atoms, form a 5-membered heterocycle substituted with one or more C₁-C₆ alkyl-NHC₁-C₆ alkyl. In some embodiments, R⁷ and R⁸, together with the molecules to which they are attached and any intervening atoms, form a 5-membered heterocycle substituted with one or more NR₁₂R₁₃.

In some embodiments, R⁷ and R⁸, together with the molecules to which they are attached and any intervening atoms, form a 6-membered heterocycle substituted with one or more halogen. In some embodiments, R⁷ and R⁸, together with the molecules to which they are attached and any intervening atoms, form a 6-membered heterocycle substituted with one or more F. In some embodiments, R⁷ and R⁸, together with the molecules to which they are attached and any intervening atoms, form a 6-membered heterocycle substituted with one or more Cl. In some embodiments, R⁷ and R⁸, together with the molecules to which they are attached and any intervening atoms, form a 6-membered heterocycle substituted with one or more Br. In some embodiments, R⁷ and R⁸, together with the molecules to which they are attached and any intervening atoms, form a 6-membered heterocycle substituted with one or more I. In some embodiments, R⁷ and R⁸, together with the molecules to which they are attached and any intervening atoms, form a 6-membered heterocycle substituted with one or more OH. In some embodiments, R⁷ and R⁸, together with the molecules to which they are attached and any intervening atoms, form a 6-membered heterocycle substituted with one or more NH₂. In some embodiments, R⁷ and R⁸, together with the molecules to which they are attached and any intervening atoms, form a 6-membered heterocycle substituted with one or more C₁-C₆ alkyl. In some embodiments, R⁷ and R⁸, together with the molecules to which they are attached and any intervening atoms, form a 6-membered heterocycle substituted with one or more C₁-C₆ alkoxy. In some embodiments, R⁷ and R⁸, together with the molecules to which they are attached and any intervening atoms, form a 6-membered heterocycle substituted with one or more C₁-C₆ alkyl-C₁-C₆ alkoxy. In some embodiments, R⁷ and R⁸, together with the molecules to which they are attached and any intervening atoms, form a 6-membered heterocycle substituted with one or more C₁-C₆ alkyl-NHC₁-C₆ alkyl. In some embodiments, R⁷ and R⁸, together with the molecules to which they are attached and any intervening atoms, form a 6-membered heterocycle substituted with one or more NR₁₂R₁₃.

In some embodiments, R⁷ and R⁸, together with the molecules to which they are attached and any intervening atoms, form a 7-membered heterocycle substituted with one or more halogen. In some embodiments, R⁷ and R⁸, together with the molecules to which they are attached and any intervening atoms, form a 7-membered heterocycle substituted with one or more F. In some embodiments, R⁷ and R⁸, together with the molecules to which they are attached and any intervening atoms, form a 7-membered heterocycle substituted with one or more Cl. In some embodiments, R⁷ and R⁸, together with the molecules to which they are attached and any intervening atoms, form a 7-membered heterocycle substituted with one or more Br. In some embodiments, R⁷ and R⁸, together with the molecules to which they are attached and any intervening atoms, form a 7-membered heterocycle substituted with one or more I. In some embodiments, R⁷ and R⁸, together with the molecules to which they are attached and any intervening atoms, form a 7-membered heterocycle substituted with one or more OH. In some embodiments, R⁷ and R⁸, together with the molecules to which they are attached and any intervening atoms, form a 7-membered heterocycle substituted with one or more NH₂. In some embodiments, R⁷ and R⁸, together with the molecules to which they are attached and any intervening atoms, form a 7-membered heterocycle substituted with one or more C₁-C₆ alkyl. In some embodiments, R⁷ and R⁸, together with the molecules to which they are attached and any intervening atoms, form a 7-membered heterocycle substituted with one or more C₁-C₆ alkoxy. In some embodiments, R⁷ and R⁸, together with the molecules to which they are attached and any intervening atoms, form a 7-membered heterocycle substituted with one or more C₁-C₆ alkyl-C₁-C₆ alkoxy. In some embodiments, R⁷ and R⁸, together with the molecules to which they are attached and any intervening atoms, form a 7-membered heterocycle substituted with one or more C₁-C₆ alkyl-NHC₁-C₆ alkyl. In some embodiments, R⁷ and R⁸, together with the molecules to which they are attached and any intervening atoms, form a 7-membered heterocycle substituted with one or more NR₁₂R₁₃.

In some embodiments, R⁷ and R⁸, together with the molecules to which they are attached and any intervening atoms, form an 8-membered heterocycle substituted with one or more halogen. In some embodiments, R⁷ and R⁸, together with the molecules to which they are attached and any intervening atoms, form an 8-membered heterocycle substituted with one or more F. In some embodiments, R¹ and R⁸, together with the molecules to which they are attached and any intervening atoms, form an 8-membered heterocycle substituted with one or more Cl. In some embodiments, R⁷ and R⁸, together with the molecules to which they are attached and any intervening atoms, form an 8-membered heterocycle substituted with one or more Br. In some embodiments, R⁷ and R⁸, together with the molecules to which they are attached and any intervening atoms, form an 8-membered heterocycle substituted with one or more I. In some embodiments, R⁷ and R⁸, together with the molecules to which they are attached and any intervening atoms, form an 8-membered heterocycle substituted with one or more OH. In some embodiments, R⁷ and R⁸, together with the molecules to which they are attached and any intervening atoms, form an 8-membered heterocycle substituted with one or more NH₂. In some embodiments, R⁷ and R⁸, together with the molecules to which they are attached and any intervening atoms, form an 8-membered heterocycle substituted with one or more C₁-C₆ alkyl. In some embodiments, R⁷ and R⁸, together with the molecules to which they are attached and any intervening atoms, form an 8-membered heterocycle substituted with one or more C₁-C₆ alkoxy. In some embodiments, R⁷ and R⁸, together with the molecules to which they are attached and any intervening atoms, form an 8-membered heterocycle substituted with one or more C₁-C₆ alkyl-C₁-C₆ alkoxy. In some embodiments, R⁷ and R⁸, together with the molecules to which they are attached and any intervening atoms, form an 8-membered heterocycle substituted with one or more C₁-C₆ alkyl-NHC₁-C₆ alkyl. In some embodiments, R⁷ and R⁸, together with the molecules to which they are attached and any intervening atoms, form an 8-membered heterocycle substituted with one or more NR₁₂R₁₃.

In some embodiments, R⁷ and R⁸, together with the molecules to which they are attached and any intervening atoms, form a 9-membered heterocycle substituted with one or more halogen. In some embodiments, R⁷ and R⁸, together with the molecules to which they are attached and any intervening atoms, form a 9-membered heterocycle substituted with one or more F. In some embodiments, R⁷ and R⁸, together with the molecules to which they are attached and any intervening atoms, form a 9-membered heterocycle substituted with one or more Cl. In some embodiments, R⁷ and R⁸, together with the molecules to which they are attached and any intervening atoms, form a 9-membered heterocycle substituted with one or more Br. In some embodiments, R⁷ and R⁸, together with the molecules to which they are attached and any intervening atoms, form a 9-membered heterocycle substituted with one or more I. In some embodiments, R⁷ and R⁸, together with the molecules to which they are attached and any intervening atoms, form a 9-membered heterocycle substituted with one or more OH. In some embodiments, R⁷ and R⁸, together with the molecules to which they are attached and any intervening atoms, form a 9-membered heterocycle substituted with one or more NH₂. In some embodiments, R⁷ and R⁸, together with the molecules to which they are attached and any intervening atoms, form a 9-membered heterocycle substituted with one or more C₁-C₆ alkyl. In some embodiments, R⁷ and R⁸, together with the molecules to which they are attached and any intervening atoms, form a 9-membered heterocycle substituted with one or more C₁-C₆ alkoxy. In some embodiments, R⁷ and R⁸, together with the molecules to which they are attached and any intervening atoms, form a 9-membered heterocycle substituted with one or more C₁-C₆ alkyl-C₁-C₆ alkoxy. In some embodiments, R⁷ and R⁸, together with the molecules to which they are attached and any intervening atoms, form a 9-membered heterocycle substituted with one or more C₁-C₆ alkyl-NHC₁-C₆ alkyl. In some embodiments, R⁷ and R⁸, together with the molecules to which they are attached and any intervening atoms, form a 9-membered heterocycle substituted with one or more NR₁₂R₁₃.

In some embodiments, R⁷ and R⁸, together with the molecules to which they are attached and any intervening atoms, form a 10-membered heterocycle substituted with one or more halogen. In some embodiments, R⁷ and R⁸, together with the molecules to which they are attached and any intervening atoms, form a 10-membered heterocycle substituted with one or more F. In some embodiments, R⁷ and R⁸, together with the molecules to which they are attached and any intervening atoms, form a 10-membered heterocycle substituted with one or more Cl. In some embodiments, R⁷ and R⁸, together with the molecules to which they are attached and any intervening atoms, form a 10-membered heterocycle substituted with one or more Br. In some embodiments, R⁷ and R⁸, together with the molecules to which they are attached and any intervening atoms, form a 10-membered heterocycle substituted with one or more I. In some embodiments, R⁷ and R⁸, together with the molecules to which they are attached and any intervening atoms, form a 10-membered heterocycle substituted with one or more OH. In some embodiments, R⁷ and R⁸, together with the molecules to which they are attached and any intervening atoms, form a 10-membered heterocycle substituted with one or more NH₂. In some embodiments, R⁷ and R⁸, together with the molecules to which they are attached and any intervening atoms, form a 10-membered heterocycle substituted with one or more C₁-C₆ alkyl. In some embodiments, R⁷ and R⁸, together with the molecules to which they are attached and any intervening atoms, form a 10-membered heterocycle substituted with one or more C₁-C₆ alkoxy. In some embodiments, R⁷ and R⁸, together with the molecules to which they are attached and any intervening atoms, form a 10-membered heterocycle substituted with one or more C₁-C₆ alkyl-C₁-C₆ alkoxy. In some embodiments, R⁷ and R⁸, together with the molecules to which they are attached and any intervening atoms, form a 10-membered heterocycle substituted with one or more C₁-C₆ alkyl-NHC₁-C₆ alkyl. In some embodiments, R⁷ and R⁸, together with the molecules to which they are attached and any intervening atoms, form a 10-membered heterocycle substituted with one or more NR₁₂R₁₃.

In some embodiments, R⁷ and R⁸, together with the molecules to which they are attached and any intervening atoms, form an 11-membered heterocycle substituted with one or more halogen. In some embodiments, R⁷ and R⁸, together with the molecules to which they are attached and any intervening atoms, form an 11-membered heterocycle substituted with one or more F. In some embodiments, R⁷ and R⁸, together with the molecules to which they are attached and any intervening atoms, form an 11-membered heterocycle substituted with one or more Cl. In some embodiments, R⁷ and R⁸, together with the molecules to which they are attached and any intervening atoms, form an 11-membered heterocycle substituted with one or more Br. In some embodiments, R⁷ and R⁸, together with the molecules to which they are attached and any intervening atoms, form an 11-membered heterocycle substituted with one or more I. In some embodiments, R⁷ and R⁸, together with the molecules to which they are attached and any intervening atoms, form an 11-membered heterocycle substituted with one or more OH. In some embodiments, R⁷ and R⁸, together with the molecules to which they are attached and any intervening atoms, form an 11-membered heterocycle substituted with one or more NH₂. In some embodiments, R⁷ and R⁸, together with the molecules to which they are attached and any intervening atoms, form an 11-membered heterocycle substituted with one or more C₁-C₆ alkyl. In some embodiments, R⁷ and R⁸, together with the molecules to which they are attached and any intervening atoms, form a 11-membered heterocycle substituted with one or more C₁-C₆ alkoxy. In some embodiments, R⁷ and R⁸, together with the molecules to which they are attached and any intervening atoms, form an 11-membered heterocycle substituted with one or more C₁-C₆ alkyl-C₁-C₆ alkoxy. In some embodiments, R⁷ and R⁸, together with the molecules to which they are attached and any intervening atoms, form an 11-membered heterocycle substituted with one or more C₁-C₆ alkyl-NHC₁-C₆ alkyl. In some embodiments, R⁷ and R⁸, together with the molecules to which they are attached and any intervening atoms, form an 11-membered heterocycle substituted with one or more NR₁₂R₁₃.

In some embodiments, R⁷ and R⁸, together with the molecules to which they are attached and any intervening atoms, form a 12-membered heterocycle substituted with one or more halogen. In some embodiments, R⁷ and R⁸, together with the molecules to which they are attached and any intervening atoms, form a 12-membered heterocycle substituted with one or more F. In some embodiments, R⁷ and R⁸, together with the molecules to which they are attached and any intervening atoms, form a 12-membered heterocycle substituted with one or more Cl. In some embodiments, R⁷ and R⁸, together with the molecules to which they are attached and any intervening atoms, form a 12-membered heterocycle substituted with one or more Br. In some embodiments, R⁷ and R⁸, together with the molecules to which they are attached and any intervening atoms, form a 12-membered heterocycle substituted with one or more I. In some embodiments, R⁷ and R⁸, together with the molecules to which they are attached and any intervening atoms, form a 12-membered heterocycle substituted with one or more OH. In some embodiments, R⁷ and R⁸, together with the molecules to which they are attached and any intervening atoms, form a 12-membered heterocycle substituted with one or more NH₂. In some embodiments, R⁷ and R⁸, together with the molecules to which they are attached and any intervening atoms, form a 12-membered heterocycle substituted with one or more C₁-C₆ alkyl. In some embodiments, R⁷ and R⁸, together with the molecules to which they are attached and any intervening atoms, form a 12-membered heterocycle substituted with one or more C₁-C₆ alkoxy. In some embodiments, R⁷ and R⁸, together with the molecules to which they are attached and any intervening atoms, form a 12-membered heterocycle substituted with one or more C₁-C₆ alkyl-C₁-C₆ alkoxy. In some embodiments, R⁷ and R⁸, together with the molecules to which they are attached and any intervening atoms, form a 12-membered heterocycle substituted with one or more C₁-C₆ alkyl-NHC₁-C₆ alkyl. In some embodiments, R⁷ and R², together with the molecules to which they are attached and any intervening atoms, form a 12-membered heterocycle substituted with one or more NR₁₂R₁₃.

In some embodiments, R¹ and R⁸, together with the molecules to which they are attached and any intervening atoms, form a 13-membered heterocycle substituted with one or more halogen. In some embodiments, R⁷ and R⁸, together with the molecules to which they are attached and any intervening atoms, form a 13-membered heterocycle substituted with one or more F. In some embodiments, R⁷ and R⁸, together with the molecules to which they are attached and any intervening atoms, form a 13-membered heterocycle substituted with one or more Cl. In some embodiments, R⁷ and R⁸, together with the molecules to which they are attached and any intervening atoms, form a 13-membered heterocycle substituted with one or more Br. In some embodiments, R⁷ and R⁸, together with the molecules to which they are attached and any intervening atoms, form a 13-membered heterocycle substituted with one or more I. In some embodiments, R⁷ and R⁸, together with the molecules to which they are attached and any intervening atoms, form a 13-membered heterocycle substituted with one or more OH. In some embodiments, R⁷ and R⁸, together with the molecules to which they are attached and any intervening atoms, form a 13-membered heterocycle substituted with one or more NH₂. In some embodiments, R⁷ and R⁸, together with the molecules to which they are attached and any intervening atoms, form a 13-membered heterocycle substituted with one or more C₁-C₆ alkyl. In some embodiments, R⁷ and R⁸, together with the molecules to which they are attached and any intervening atoms, form a 13-membered heterocycle substituted with one or more C₁-C₆ alkoxy. In some embodiments, R⁷ and R⁸, together with the molecules to which they are attached and any intervening atoms, form a 13-membered heterocycle substituted with one or more C₁-C₆ alkyl-C₁-C₆ alkoxy. In some embodiments, R⁷ and R⁸, together with the molecules to which they are attached and any intervening atoms, form a 13-membered heterocycle substituted with one or more C₁-C₆ alkyl-NHC₁-C₆ alkyl. In some embodiments, R⁷ and R⁸, together with the molecules to which they are attached and any intervening atoms, form a 13-membered heterocycle substituted with one or more NR₁₂R₁₃.

In some embodiments, R⁷ and R⁸, together with the molecules to which they are attached and any intervening atoms, form a 14-membered heterocycle substituted with one or more halogen. In some embodiments, R⁷ and R⁸, together with the molecules to which they are attached and any intervening atoms, form a 14-membered heterocycle substituted with one or more F. In some embodiments, R⁷ and R⁸, together with the molecules to which they are attached and any intervening atoms, form a 14-membered heterocycle substituted with one or more Cl. In some embodiments, R⁷ and R⁸, together with the molecules to which they are attached and any intervening atoms, form a 14-membered heterocycle substituted with one or more Br. In some embodiments, R⁷ and R⁸, together with the molecules to which they are attached and any intervening atoms, form a 14-membered heterocycle substituted with one or more I. In some embodiments, R⁷ and R⁸, together with the molecules to which they are attached and any intervening atoms, form a 14-membered heterocycle substituted with one or more OH. In some embodiments, R⁶ and R⁸, together with the molecules to which they are attached and any intervening atoms, form a 14-membered heterocycle substituted with one or more NH₂. In some embodiments, R⁷ and R⁸, together with the molecules to which they are attached and any intervening atoms, form a 14-membered heterocycle substituted with one or more C₁-C₆ alkyl. In some embodiments, R⁷ and R⁸, together with the molecules to which they are attached and any intervening atoms, form a 14-membered heterocycle substituted with one or more C₁-C₆ alkoxy. In some embodiments, R⁷ and R⁸, together with the molecules to which they are attached and any intervening atoms, form a 14-membered heterocycle substituted with one or more C₁-C₆ alkyl-C₁-C₆ alkoxy. In some embodiments, R⁷ and R⁸, together with the molecules to which they are attached and any intervening atoms, form a 14-membered heterocycle substituted with one or more C₁-C₆ alkyl-NHC₁-C₆ alkyl. In some embodiments, R⁷ and R⁸, together with the molecules to which they are attached and any intervening atoms, form a 14-membered heterocycle substituted with one or more NR₁₂R₁₃.

In some embodiments, R¹² is H. In some embodiments, R¹² is C₁-C₆ alkyl, C₂-C₆ alkenyl, C₂-C₆ alkynyl or C₁-C₆ alkoxy. In some embodiments, R¹² is C₃-C₁₀ cycloalkyl, heterocycle, aryl, or heteroaryl.

In some embodiments, R¹² is C₁-C₆ alkyl.

In some embodiments, R¹² is methyl. In some embodiments, R¹² is ethyl. In some embodiments, R¹² is propyl. In some embodiments, R¹² is n-propyl. In some embodiments, R¹² is isopropyl. In some embodiments, R¹² is butyl. In some embodiments, R¹² is n-butyl. In some embodiments, R¹² is isobutyl. In some embodiments, R¹² is sec-butyl. In some embodiments, R¹² is tert-butyl. In some embodiments, R¹² is pentyl. In some embodiments, R¹² is hexyl.

In some embodiments, R¹² is C₁-C₆ alkoxy.

In some embodiments, R¹² is methoxy. In some embodiments, R¹² is ethoxy. In some embodiments, R¹² is propoxy. In some embodiments, R¹² is n-propoxy. In some embodiments, R¹² is isopropoxy. In some embodiments, R¹² is butoxy. In some embodiments, R¹² is n-butoxy. In some embodiments, R¹² is isobutoxy. In some embodiments, R¹² is sec-butoxy. In some embodiments, R¹² is tert-butoxy. In some embodiments, R¹² is pentoxy. In some embodiments, R¹² is hexoxy.

In some embodiments, R¹² is C₃-C₁₀ cycloalkyl.

In some embodiments, R¹² is cyclopropyl. In some embodiments, R¹² is cyclobutyl. In some embodiments, R¹² is cyclopentyl. In some embodiments, R¹² is cyclohexyl. In some embodiments, R¹² is cycloheptyl. In some embodiments, R¹² is cyclooctyl. In some embodiments, R¹² is cyclononyl. In some embodiments, R¹² is cyclodecyl.

In some embodiments, R¹² is C₂-C₆ alkenyl.

In some embodiments, R¹² is C₂ alkenyl. In some embodiments, R¹² is C₃ alkenyl. In some embodiments, R¹² is C₄ alkenyl. In some embodiments, R¹² is C₅ alkenyl. In some embodiments, R¹² is C₁₆ alkenyl.

In some embodiments, R¹² is C₂-C₆ alkynyl.

In some embodiments, R¹² is C₂ alkynyl. In some embodiments, R¹² is C₃ alkynyl. In some embodiments, R¹² is C₄ alkynyl. In some embodiments, R¹² is C₅ alkynyl. In some embodiments, R¹² is C₆ alkynyl.

In some embodiments, R¹² is heterocyclyl.

In some embodiments R¹² is 3- to 8-membered heterocyclyl. In some embodiments R¹² is 3- to 10-membered heterocyclyl.

In some embodiments, R¹² is a monocyclic heterocyclyl. In some embodiments, R¹² is a polycyclic heterocyclyl.

In some embodiments, R¹² is 3-membered heterocyclyl. In some embodiments, R¹² is 4-membered heterocyclyl. In some embodiments, R¹² is 5-membered heterocyclyl. In some embodiments, R¹² is 6-membered heterocyclyl. In some embodiments, R¹² is 7-membered heterocyclyl. In some embodiments, R¹² is 8-membered heterocyclyl. In some embodiments, R¹² is 9-membered heterocyclyl. In some embodiments, R¹² is 10-membered heterocyclyl.

In some embodiments, R¹² is aryl. In some embodiments, R¹² is C₆ aryl (e.g., phenyl).

In some embodiments, R¹² is heteroaryl.

In some embodiments R¹² is heteroaryl. In some embodiments, R¹² is 5- to 6-membered heteroaryl. In some embodiments, R¹² is 5- to 10-membered heteroaryl.

In some embodiments, R¹² is heteroaryl comprising one, two, or three heteroatoms.

In some embodiments, R¹² is heteroaryl comprising one, two, or three heteroatoms selected from N, O, and S.

In some embodiments, R¹² is heteroaryl comprising one, two, or three heteroatoms selected from N and O.

In some embodiments, R¹² is 5-membered heteroaryl comprising 1-4 heteroatoms selected from O, N, and S. In some embodiments, R¹² is 6-membered heteroaryl comprising 1-4 heteroatoms selected from O, N, and S. In some embodiments, R¹² is 7-membered heteroaryl comprising 1-4 heteroatoms selected from O, N, and S. In some embodiments, R¹² is 8-membered heteroaryl comprising 1-4 heteroatoms selected from O, N, and S. In some embodiments, R¹² is 9-membered heteroaryl comprising 1-4 heteroatoms selected from O, N, and S. In some embodiments, R¹² is 10-membered heteroaryl comprising 1-4 heteroatoms selected from O, N, and S.

In some embodiments, R¹³ is H. In some embodiments, R¹³ is C₁-C₆ alkyl, C₂-C₆ alkenyl, C₂-C₆ alkynyl or C₁-C₆ alkoxy. In some embodiments, R¹³ is C₃-C₁₀ cycloalkyl, heterocyclyl, aryl, or heteroaryl.

In some embodiments, R¹³ is C₁-C₆ alkyl.

In some embodiments, R¹³ is methyl. In some embodiments, R¹³ is ethyl. In some embodiments, R¹³ is propyl. In some embodiments, R¹³ is n-propyl. In some embodiments, R¹³ is isopropyl. In some embodiments, R¹³ is butyl. In some embodiments, R¹³ is n-butyl. In some embodiments, R¹³ is isobutyl. In some embodiments, R¹³ is sec-butyl. In some embodiments, R¹³ is tert-butyl. In some embodiments, R¹³ is pentyl. In some embodiments, R¹³ is hexyl.

In some embodiments, R¹³ is C₁-C₆ alkoxy.

In some embodiments, R¹³ is methoxy. In some embodiments, R¹³ is ethoxy. In some embodiments, R¹³ is propoxy. In some embodiments, R¹³ is n-propoxy. In some embodiments, R¹³ is isopropoxy. In some embodiments, R¹³ is butoxy. In some embodiments, R¹³ is n-butoxy. In some embodiments, R¹³ is isobutoxy. In some embodiments, R¹³ is sec-butoxy. In some embodiments, R¹³ is tert-butoxy. In some embodiments, R¹³ is pentoxy. In some embodiments, R¹³ is hexoxy.

In some embodiments, R¹³ is C₃-C₁₀ cycloalkyl.

In some embodiments, R¹³ is cyclopropyl. In some embodiments, R¹³ is cyclobutyl. In some embodiments, R¹³ is cyclopentyl. In some embodiments, R¹³ is cyclohexyl. In some embodiments, R¹³ is cycloheptyl. In some embodiments, R¹³ is cyclooctyl. In some embodiments, R¹³ is cyclononyl. In some embodiments, R¹³ is cyclodecyl.

In some embodiments, R¹³ is C₂-C₆ alkenyl.

In some embodiments, R¹³ is C₂ alkenyl. In some embodiments, R¹³ is C₃ alkenyl. In some embodiments, R¹³ is C₄ alkenyl. In some embodiments, R¹³ is C₅ alkenyl. In some embodiments, R¹³ is C₁₆ alkenyl.

In some embodiments, R¹³ is C₂-C₆ alkynyl.

In some embodiments, R¹³ is C₂ alkynyl. In some embodiments, R¹³ is C₃ alkynyl. In some embodiments, R¹³ is C₄ alkynyl. In some embodiments, R¹³ is C₅ alkynyl. In some embodiments, R¹³ is C₆ alkynyl.

In some embodiments, R¹³ is heterocyclyl.

In some embodiments R¹³ is 3- to 8-membered heterocyclyl. In some embodiments R¹³ is 3- to 10-membered heterocyclyl.

In some embodiments, R¹³ is a monocyclic heterocyclyl. In some embodiments, R¹³ is a polycyclic heterocyclyl.

In some embodiments, R¹³ is 3-membered heterocyclyl. In some embodiments, R¹³ is 4-membered heterocyclyl. In some embodiments, R¹³ is 5-membered heterocyelyl. In some embodiments, R¹³ is 6-membered heterocyclyl. In some embodiments, R¹³ is 7-membered heterocyclyl. In some embodiments, R¹³ is 8-membered heterocyclyl. In some embodiments, R¹³ is 9-membered heterocyclyl. In some embodiments, R¹³ is 10-membered heterocyclyl.

In some embodiments, R¹³ is aryl. In some embodiments, R¹³ is C₆ aryl (e.g., phenyl).

In some embodiments, R¹³ is heteroaryl.

In some embodiments R¹³ is heteroaryl. In some embodiments, R¹³ is 5- to 6-membered heteroaryl. In some embodiments, R¹³ is 5- to 10-membered heteroaryl.

In some embodiments, R¹³ is heteroaryl comprising one, two, or three heteroatoms.

In some embodiments, R¹³ is heteroaryl comprising one, two, or three heteroatoms selected from N, O, and S.

In some embodiments, R¹³ is heteroaryl comprising one, two, or three heteroatoms selected from N and O.

In some embodiments, R¹³ is 5-membered heteroaryl comprising 1-4 heteroatoms selected from O, N, and S. In some embodiments, R¹³ is 6-membered heteroaryl comprising 1-4 heteroatoms selected from O), N, and S. In some embodiments, R¹³ is 7-membered heteroaryl comprising 1-4 heteroatoms selected from O, N, and S. In some embodiments, R¹³ is 8-membered heteroaryl comprising 1-4 heteroatoms selected from O, N, and S. In some embodiments, R¹³ is 9-membered heteroaryl comprising 1-4 heteroatoms selected from O, N, and S. In some embodiments, R¹³ is 10-membered heteroaryl comprising 1-4 heteroatoms selected from O, N, and S.

In some embodiments, n is 0. In some embodiments, n is 1 or 2. In some embodiments, n is 1. In some embodiments, n is 2.

In some embodiments, p is 0. In some embodiments, p is 1 or 2. In some embodiments, p is 1. In some embodiments, p is 2.

In some embodiments, q is 0. In some embodiments, q is 1 or 2. In some embodiments, q is 1. In some embodiments, q is 2.

In some embodiments, s is 0. In some embodiments, s is 1 or 2. In some embodiments, s is 1. In some embodiments, s is 2.

In some embodiments, the compound is of Formula (I-A), (I-B), (I-D), (I-E), (I-F), or (I-G):

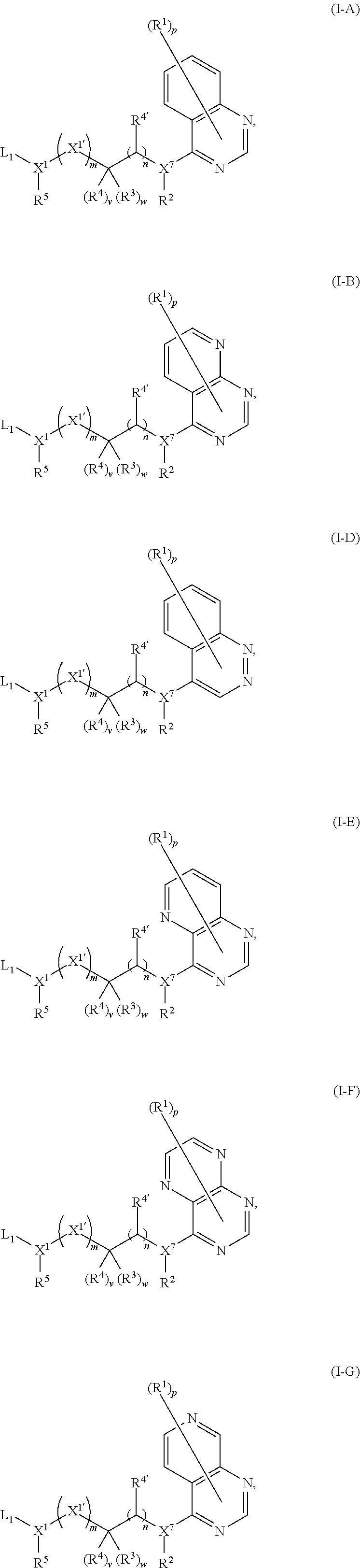

or a pharmaceutically acceptable salt, stereoisomer, solvate, prodrug, isotopic derivative, or

In some embodiments, the compound is of Formula (I-A-I), (I-A-II), (I-A-III), (I-A-IV), (I-A-V), or (I-A-VI):

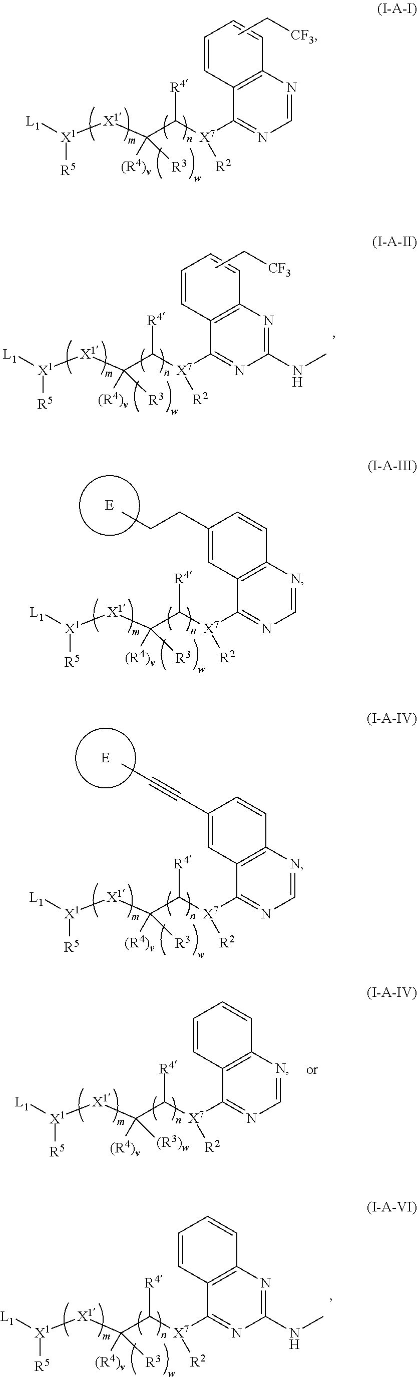

or a pharmaceutically acceptable salt, stereoisomer, solvate, prodrug, isotopic derivative, or tautomer thereof, wherein Ring E is aryl or heteroaryl, optionally substituted with one or more substituents independently selected from halogen, OH, NH₂, C₁-C₆ alkyl, C₁-C₆ alkoxy, and NR¹²R¹³, and wherein all other variables are as defined herein.

In some embodiments, Ring E is aryl. In some embodiments, Ring E is heteroaryl. In some embodiments, Ring E is unsubstituted aryl. In some embodiments, Ring E is unsubstituted heteroaryl.

In some embodiments, Ring E is C₆ aryl (e.g., phenyl).

In some embodiments Ring E is heteroaryl. In some embodiments, Ring E is 5- to 6-membered heteroaryl. In some embodiments, Ring E is 5- to 10-membered heteroaryl.

In some embodiments, Ring E is heteroaryl comprising one, two, or three heteroatoms.

In some embodiments, Ring E is heteroaryl comprising one, two, or three heteroatoms selected from N, O, and S.

In some embodiments, Ring E is heteroaryl comprising one, two, or three heteroatoms selected from N and O.

In some embodiments, Ring E is 5-membered heteroaryl comprising 1-4 heteroatoms selected from O, N, and S. In some embodiments, Ring E is 6-membered heteroaryl comprising 1-4 heteroatoms selected from O, N, and S. In some embodiments, Ring E is 7-membered heteroaryl comprising 1-4 heteroatoms selected from O, N, and S. In some embodiments, Ring E is 8-membered heteroaryl comprising 1-4 heteroatoms selected from O, N, and S. In some embodiments, Ring E is 9-membered heteroaryl comprising 1-4 heteroatoms selected from O, N, and S. In some embodiments, Ring E is 10-membered heteroaryl comprising 1-4 heteroatoms selected from O, N, and S.

In some embodiments, Ring E is heteroaryl comprising one heteroatom selected from N and O. In some embodiments, Ring E is heteroaryl comprising two heteroatoms selected from N and O. In some embodiments, Ring E is heteroaryl comprising three heteroatoms selected from N and O.

In some embodiments, Ring E is monocyclic heteroaryl. In some embodiments, Ring E is bicyclic heteroaryl.

In some embodiments, Ring E is:

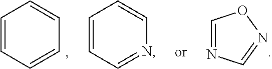

In some embodiments, Ring E is

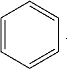

In some embodiments, Ring E is

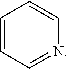

In some embodiments, Ring E is

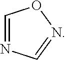

In some embodiments, the compound is of Formula (I-A-I). In some embodiments, the compound is of Formula (I-A-II). In some embodiments, the compound is of Formula (I-A-III). In some embodiments, the compound is of Formula (I-A-IV). In some embodiments, the compound is of Formula (I-A-V). In some embodiments, the compound is of Formula (I-A-VI).

In some embodiments the compound is of Formula (I-1):

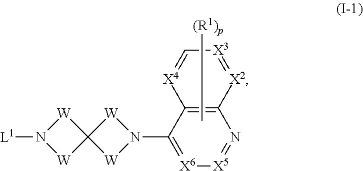

or a pharmaceutically acceptable salt, stereoisomer, solvate, prodrug, isotopic derivative, or tautomer thereof, wherein W is independently, at each occurrence —CH₂—, —(CH₂)₂—, or —(CH₂)₃—.

In various embodiments, inhibitors of menin-MLL interaction comprise compounds having a structure represented by Formula (I-1-A):

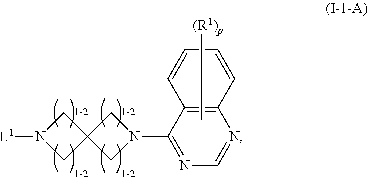

or a pharmaceutically acceptable salt, stereoisomer, solvate, prodrug, isotopic derivative, or tautomer thereof.

In various embodiments, compounds according to Formula (I-1-A) are 4-(3,9-diazaspiro[5.5]undecan-3-yl)-substituted quinazolines. In specific embodiments of these quinazolines, L¹ is H or a sulfonamide spaced apart from the 3,9-diazaspiro[5.5]undecane moiety by a tether that includes an aryl or carbocyclic ring as a spacer. In these and other embodiments, p is 2 with one R¹ being a tethered trifluoromethyl group and the other R¹ being an amino or substituted amino group. In other examples, p=0 and R¹ is thus null.

In various embodiments, compounds according to Formula (I-1-A) are 4-(2,7-diazaspiro[3.5]nonan-2-yl)-substituted quinazolines. In specific embodiments of these quinazolines, L¹ is H. In these and other embodiments, p is 1 and R¹ is a tethered aryl group, such as a phenyl or pyridinyl group tethered to the quinazoline core structure, such as at the 6-position, by a 1-4 carbon length chain, which is saturated or that contains an alkene or alkyne functionality within the carbon chain.

In some embodiments the compound is of Formula (I-1-A-1), (I-1-A-2), (I-1-A-3), or (I-A-VII):

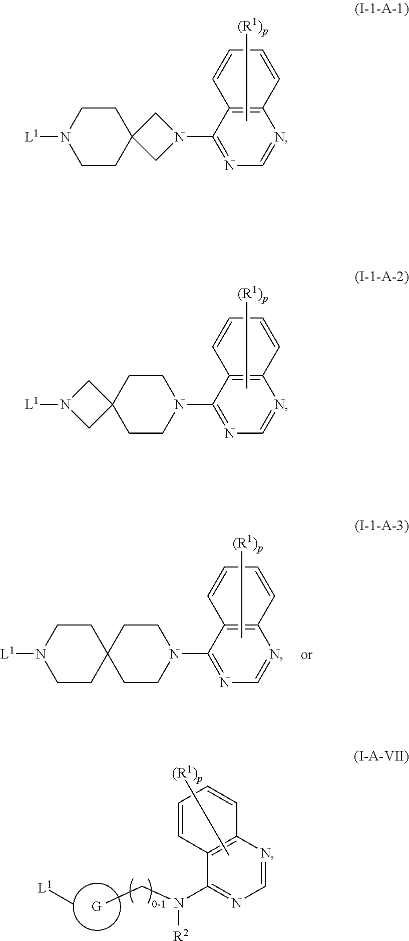

or a pharmaceutically acceptable salt, stereoisomer, solvate, prodrug, isotopic derivative, or tautomer thereof, wherein Ring G is a 3-8 membered heterocycle, optionally substituted with one or more substituents independently selected from halogen, OH, NH₂, C₁-C₆ alkyl, C₁-C₆ alkoxy, NR¹²R¹³ and wherein all other variables are as defined herein.

In some embodiments, Ring G is 5- to 8-membered heterocycle. In some embodiments, Ring G is 5- to 6-membered heterocycle.

In some embodiments, Ring G is 5- to 8-membered heterocycle comprising one, two, or three heteroatoms.

In some embodiments, Ring G is 5- to 8-membered heterocycle comprising one, two, or three heteroatoms selected from N, O, and S.

In some embodiments, Ring G is 5- to 8-membered heterocycle comprising one, two, or three heteroatoms selected from N and O.

In some embodiments, Ring G is 5-membered heterocycle comprising 1-4 heteroatoms selected from O, N, and S. In some embodiments, Ring G is 6-membered heterocycle comprising 1-4 heteroatoms selected from O, N, and S. In some embodiments, Ring G is 7-membered heterocycle comprising 1-4 heteroatoms selected from O, N, and S. In some embodiments, Ring G is 8-membered heterocycle comprising 1-4 heteroatoms selected from O, N, and S.

In some embodiments, Ring G is 5- to 8-membered heterocycle comprising one heteroatom selected from N and O. In some embodiments, Ring G is 5- to 8-membered heterocycle comprising two heteroatoms selected from N and O. In some embodiments, Ring G is 5- to 8-membered heterocycle comprising three heteroatoms selected from N and O.

In some embodiments the compound is of Formula (I-1-A-1). In some embodiments the compound is of Formula (I-1-A-2). In some embodiments the compound is of Formula (I-1-A-3). In some embodiments the compound is of Formula (I-A-VII).

In some embodiments the compound is of Formula (I-1-A-1):

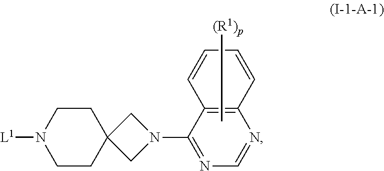

or a pharmaceutically acceptable salt, stereoisomer, solvate, prodrug, isotopic derivative, or tautomer thereof.

In some embodiments the compound is of Formula (I-1-A-1-1):

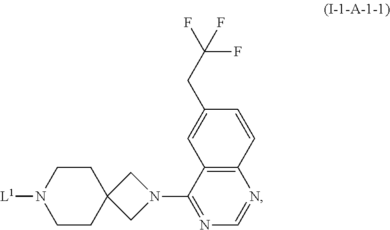

or a pharmaceutically acceptable salt, stereoisomer, solvate, prodrug, isotopic derivative, or tautomer thereof.

In some embodiments the compound is of Formula (I-1-A-1-2):

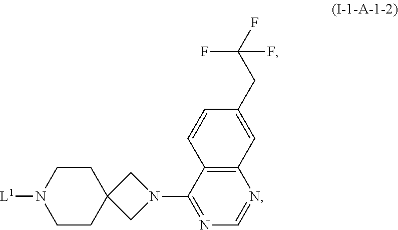

or a pharmaceutically acceptable salt, stereoisomer, solvate, prodrug, isotopic derivative, or tautomer thereof.

In some embodiments the compound is of Formula (I-1-A-1-3):

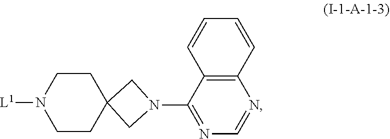

or a pharmaceutically acceptable salt, stereoisomer, solvate, prodrug, isotopic derivative, or

In some embodiments the compound is of Formula (I-1-A-1-4):

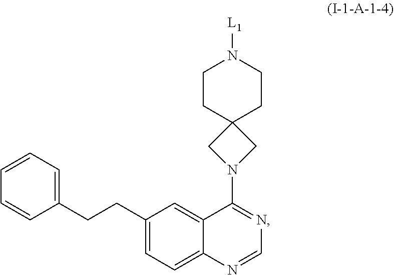

or a pharmaceutically acceptable salt, stereoisomer, solvate, prodrug, isotopic derivative, or tautomer thereof.

In some embodiments the compound is of Formula (I-1-A-1-5):

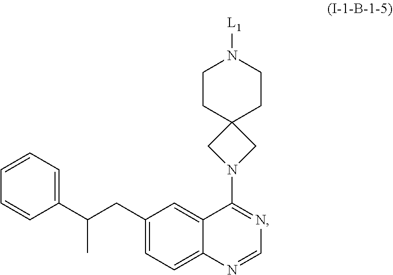

or a pharmaceutically acceptable salt, stereoisomer, solvate, prodrug, isotopic derivative, or tautomer thereof.

In some embodiments the compound is of Formula (I-1-A-1-6):

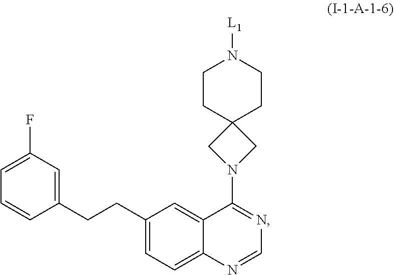

or a pharmaceutically acceptable salt, stereoisomer, solvate, prodrug, isotopic derivative, or tautomer thereof.
In some embodiments the compound is of Formula (I-1-A-1-7):

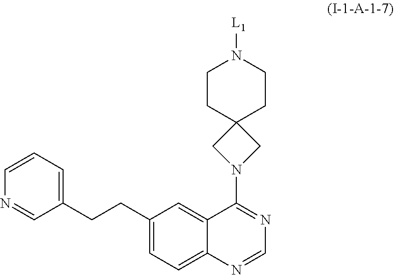

or a pharmaceutically acceptable salt, stereoisomer, solvate, prodrug, isotopic derivative, or tautomer thereof.

In some embodiments the compound is of Formula (I-1-A-1-8):

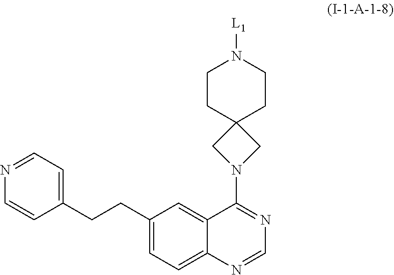

or a pharmaceutically acceptable salt, stereoisomer, solvate, prodrug, isotopic derivative, or tautomer thereof.

In some embodiments the compound is of Formula (I-1-A-1-9):

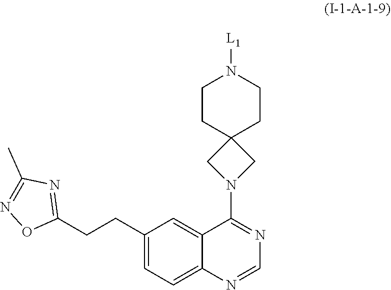

or a pharmaceutically acceptable salt, stereoisomer, solvate, prodrug, isotopic derivative, or tautomer thereof.

In some embodiments the compound is of Formula (I-1-A-1-10):

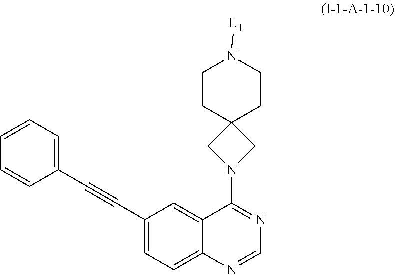

or a pharmaceutically acceptable salt, stereoisomer, solvate, prodrug, isotopic derivative, or tautomer thereof.

In some embodiments the compound is of Formula (I-1-A-1-11):

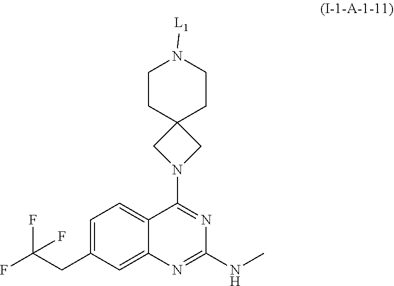

or a pharmaceutically acceptable salt, stereoisomer, solvate, prodrug, isotopic derivative, or tautomer thereof.

In some embodiments the compound is of Formula (I-1-A-1-12):

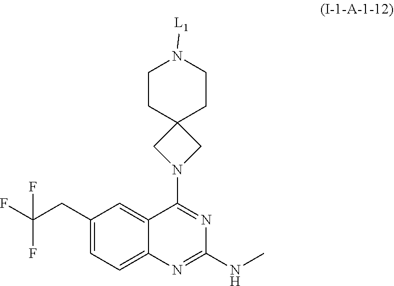

or a pharmaceutically acceptable salt, stereoisomer, solvate, prodrug, isotopic derivative, or tautomer thereof.

In some embodiments the compound is of Formula (I-1-A-1-13):

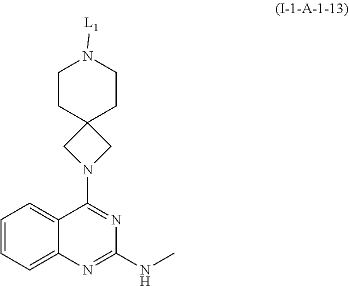

or a pharmaceutically acceptable salt, stereoisomer, solvate, prodrug, isotopic derivative, or tautomer thereof.

In some embodiments the compound is of Formula (I-1-A-1-14):

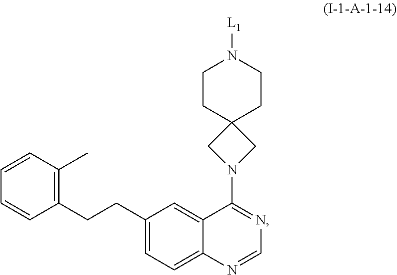

or a pharmaceutically acceptable salt, stereoisomer, solvate, prodrug, isotopic derivative, or tautomer thereof.

In some embodiments the compound is of Formula (I-1-A-1-15):

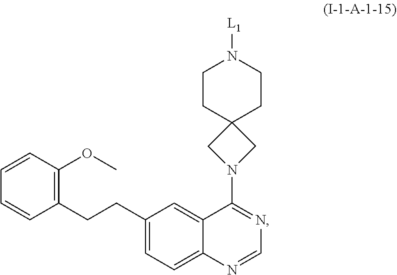

or a pharmaceutically acceptable salt, stereoisomer, solvate, prodrug, isotopic derivative, or tautomer thereof.

In some embodiments the compound is of Formula (I-1-A-1-16):

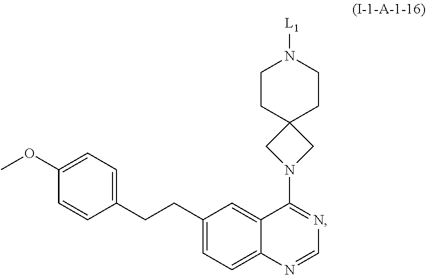

or a pharmaceutically acceptable salt, stereoisomer, solvate, prodrug, isotopic derivative, or tautomer thereof.

In some embodiments the compound is of Formula (I-1-A-1-17):

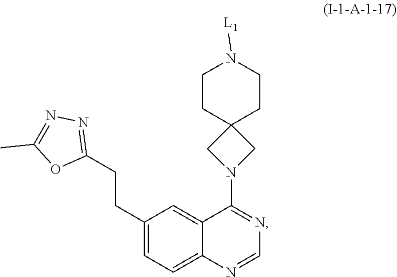

or a pharmaceutically acceptable salt, stereoisomer, solvate, prodrug, isotopic derivative, or tautomer thereof.

In some embodiments the compound is of Formula (I-1-B-1):

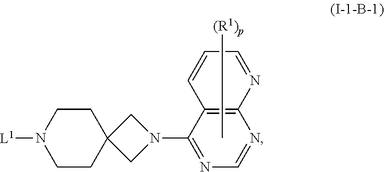

or a pharmaceutically acceptable salt, stereoisomer, solvate, prodrug, isotopic derivative, or tautomer thereof.

In some embodiments, the compound is of Formula (I-1-B-1-1):

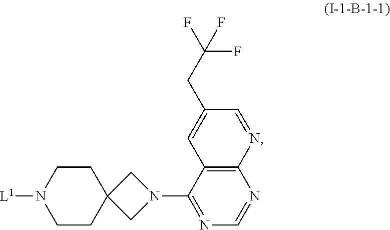

or a pharmaceutically acceptable salt, stereoisomer, solvate, prodrug, isotopic derivative, or tautomer thereof.

In some embodiments, the compound is of Formula (I-1-B-1-2):

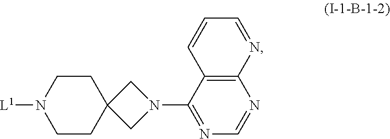

or a pharmaceutically acceptable salt, stereoisomer, solvate, prodrug, isotopic derivative, or tautomer thereof.

In some embodiments, the compound is of Formula (I-1-B-1-3):

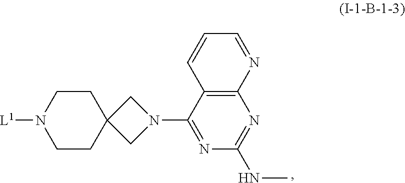

or a pharmaceutically acceptable salt, stereoisomer, solvate, prodrug, isotopic derivative, or tautomer thereof.

In some embodiments, the compound is of Formula (I-1-D-1):

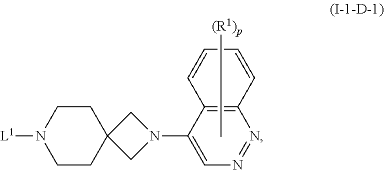

or a pharmaceutically acceptable salt, stereoisomer, solvate, prodrug, isotopic derivative, or tautomer thereof.

In some embodiments, the compound is of Formula (I-1-D-2):

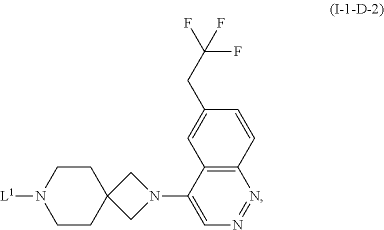

or a pharmaceutically acceptable salt, stereoisomer, solvate, prodrug, isotopic derivative, or tautomer thereof.

In some embodiments, the compound is of Formula (I-1-E-1):

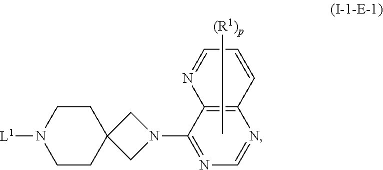

or a pharmaceutically acceptable salt, stereoisomer, solvate, prodrug, isotopic derivative, or tautomer thereof.

In some embodiments, the compound is of Formula (I-1-E-1-1):

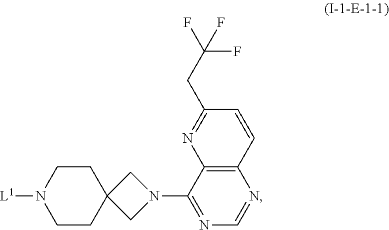

or a pharmaceutically acceptable salt, stereoisomer, solvate, prodrug, isotopic derivative, or tautomer thereof.

In some embodiments, the compound is of Formula (I-1-F-1):

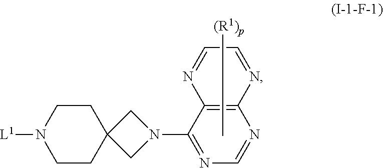

or a pharmaceutically acceptable salt, stereoisomer, solvate, prodrug, isotopic derivative, or tautomer thereof.

In some embodiments, the compound is of Formula (I-1-F-1-1):

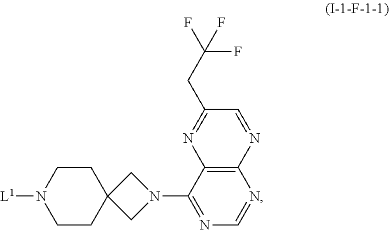

or a pharmaceutically acceptable salt, stereoisomer, solvate, prodrug, isotopic derivative, or tautomer thereof.

In some embodiments, the compound is of Formula (I-1-G-1):

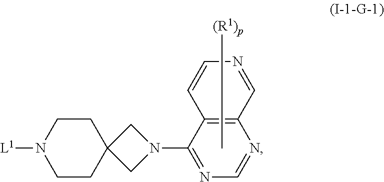

or a pharmaceutically acceptable salt, stereoisomer, solvate, prodrug, isotopic derivative, or tautomer thereof.

In some embodiments, the compound is of Formula (I-1-G-1-1):

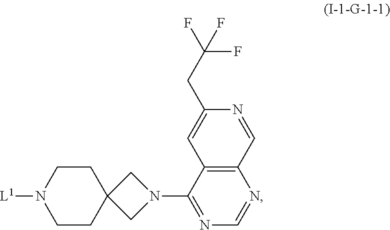

or a pharmaceutically acceptable salt, stereoisomer, solvate, prodrug, isotopic derivative, or tautomer thereof.

In some embodiments, the compound is of Formula (I-1-A-2):

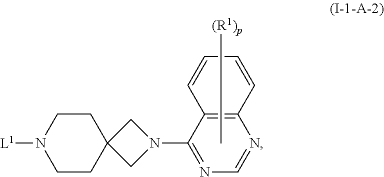

or a pharmaceutically acceptable salt, stereoisomer, solvate, prodrug, isotopic derivative, or tautomer thereof.

In some embodiments, the compound is of Formula (I-1-A-2-1):

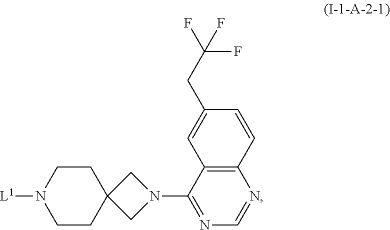

or a pharmaceutically acceptable salt, stereoisomer, solvate, prodrug, isotopic derivative, or tautomer thereof.

In some embodiments, the compound is of Formula (I-1-A-2-2):

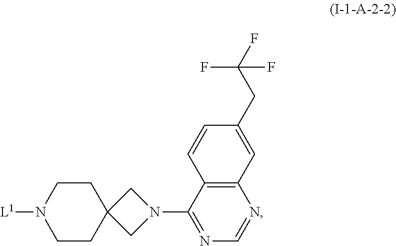

or a pharmaceutically acceptable salt, stereoisomer, solvate, prodrug, isotopic derivative, or tautomer thereof.

In some embodiments, the compound is of Formula (I-1-A-2-3):

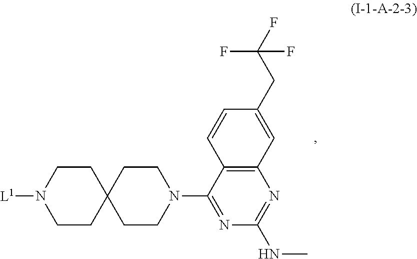

or a pharmaceutically acceptable salt, stereoisomer, solvate, prodrug, isotopic derivative, or tautomer thereof.

In some embodiments, the compound is of Formula (I-1-A-2-4):

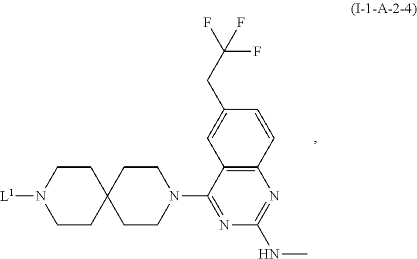

or a pharmaceutically acceptable salt, stereoisomer, solvate, prodrug, isotopic derivative, or tautomer thereof.

In some embodiments, the compound is of Formula (I-1-B-2):

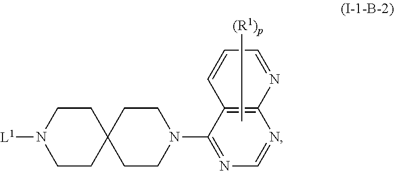

or a pharmaceutically acceptable salt, stereoisomer, solvate, prodrug, isotopic derivative, or tautomer thereof.

In some embodiments, the compound is of Formula (I-1-D-2):

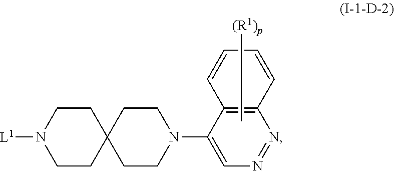

or a pharmaceutically acceptable salt, stereoisomer, solvate, prodrug, isotopic derivative, or tautomer thereof.

In some embodiments, the compound is of Formula (I-1-D-2-1):

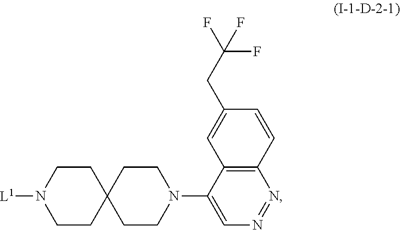

or a pharmaceutically acceptable salt, stereoisomer, solvate, prodrug, isotopic derivative, or tautomer thereof.

In some embodiments, the compound is of Formula (I-1-E-2):

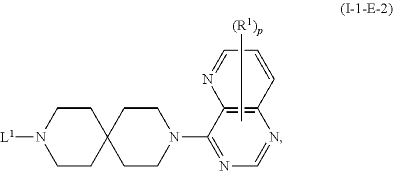

or a pharmaceutically acceptable salt, stereoisomer, solvate, prodrug, isotopic derivative, or tautomer thereof.

In some embodiments, the compound is of Formula (I-1-E-2-1):

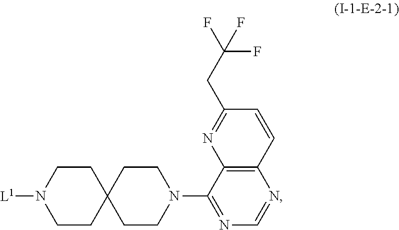

or a pharmaceutically acceptable salt, stereoisomer, solvate, prodrug, isotopic derivative, or tautomer thereof.

In some embodiments, the compound is of Formula (I-1-F-2):

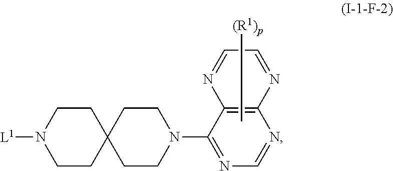

or a pharmaceutically acceptable salt, stereoisomer, solvate, prodrug, isotopic derivative, or tautomer thereof.

In some embodiments, the compound is of Formula (I-1-F-2-1):

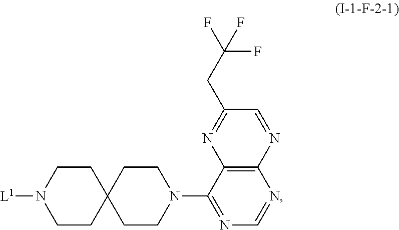

or a pharmaceutically acceptable salt, stereoisomer, solvate, prodrug, isotopic derivative, or tautomer thereof.

In some embodiments, the compound is of Formula (I-1-G-2):

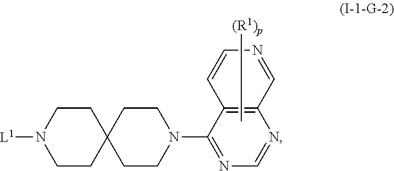

or a pharmaceutically acceptable salt, stereoisomer, solvate, prodrug, isotopic derivative, or tautomer thereof.

In some embodiments, the compound is of Formula (I-1-G-2-1):

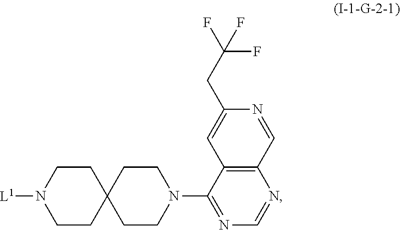

or a pharmaceutically acceptable salt, stereoisomer, solvate, prodrug, isotopic derivative, or tautomer thereof.

In some embodiments, the compound is of Formula (I-1-A-3):

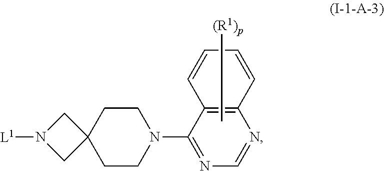

or a pharmaceutically acceptable salt, stereoisomer, solvate, prodrug, isotopic derivative, or tautomer thereof.

In some embodiments, the compound is of Formula (I-1-A-3-1):

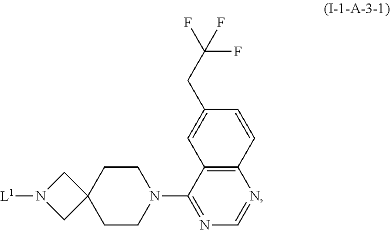

or a pharmaceutically acceptable salt, stereoisomer, solvate, prodrug, isotopic derivative, or tautomer thereof.

In some embodiments, the compound is of Formula (I-1-A-3-2):

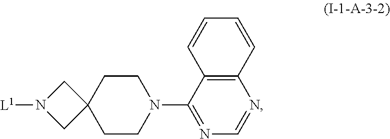

or a pharmaceutically acceptable salt, stereoisomer, solvate, prodrug, isotopic derivative, or tautomer thereof.

In some embodiments, the compound is of Formula (I-1-A-4):

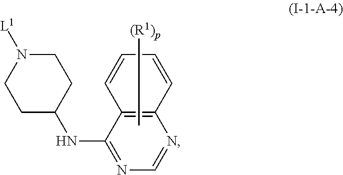

or a pharmaceutically acceptable salt, stereoisomer, solvate, prodrug, isotopic derivative, or tautomer thereof.

In some embodiments, the compound is of Formula (I-1-A-4-1):

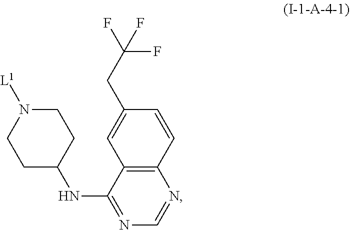

or a pharmaceutically acceptable salt, stereoisomer, solvate, prodrug, isotopic derivative, or tautomer thereof.

In some embodiments, the compound is of Formula (I-1-A-4-2):

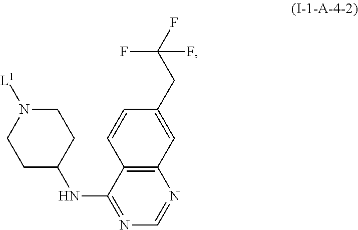

or a pharmaceutically acceptable salt, stereoisomer, solvate, prodrug, isotopic derivative, or tautomer thereof.

In some embodiments, the compound is of Formula (I-1-A-4-3):

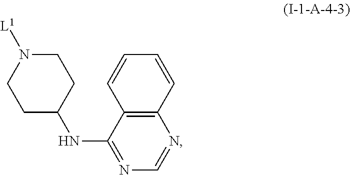

or a pharmaceutically acceptable salt, stereoisomer, solvate, prodrug, isotopic derivative, or tautomer thereof.

In some embodiments, the compound is of Formula (I-1-A-5):

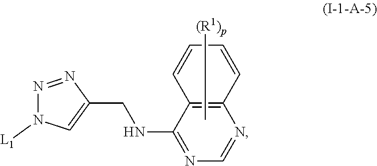

or a pharmaceutically acceptable salt, stereoisomer, solvate, prodrug, isotopic derivative, or tautomer thereof.

In some embodiments, the compound is of Formula (I-1-A-5-1):

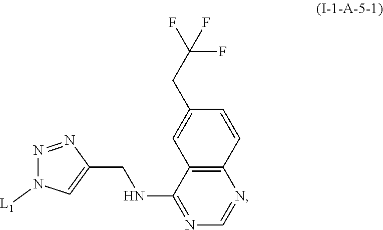

or a pharmaceutically acceptable salt, stereoisomer, solvate, prodrug, isotopic derivative, or tautomer thereof.

In some embodiments, the compound is of Formula (I-1-A-6):

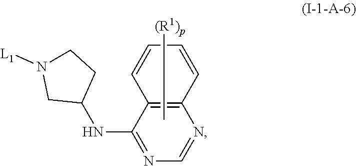

or a pharmaceutically acceptable salt, stereoisomer, solvate, prodrug, isotopic derivative, or tautomer thereof.

In some embodiments, the compound is of Formula (I-1-A-6-1):

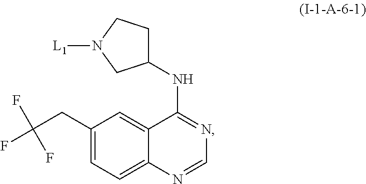

or a pharmaceutically acceptable salt, stereoisomer, solvate, prodrug, isotopic derivative, or tautomer thereof.

In some embodiments, the compound is of Formula (I-2-1), (I-2-II), (I-2-III), or (I-2-IV):

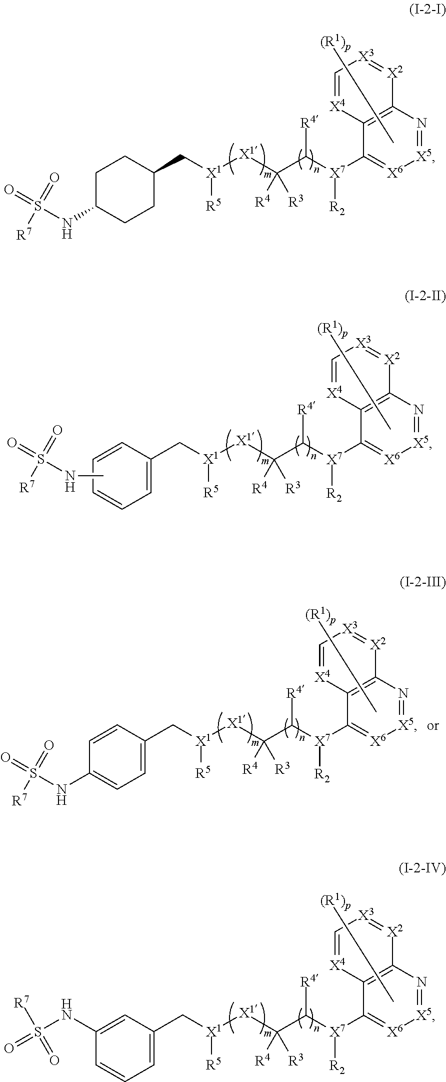

or a pharmaceutically acceptable salt, stereoisomer, solvate, prodrug, isotopic derivative, or tautomer thereof wherein all variables are as defined herein.

In some embodiments, the compound is of Formula (I-2-1). In some embodiments, the compound is of Formula (I-2-II). In some embodiments, the compound is of Formula (I-2-III). In some embodiments, the compound is of Formula (I-2-IV).

In some embodiments, the compound is of Formula (I-2-A-I), (I-2-A-II), (I-2-A-III), or (I-2-A-IV):

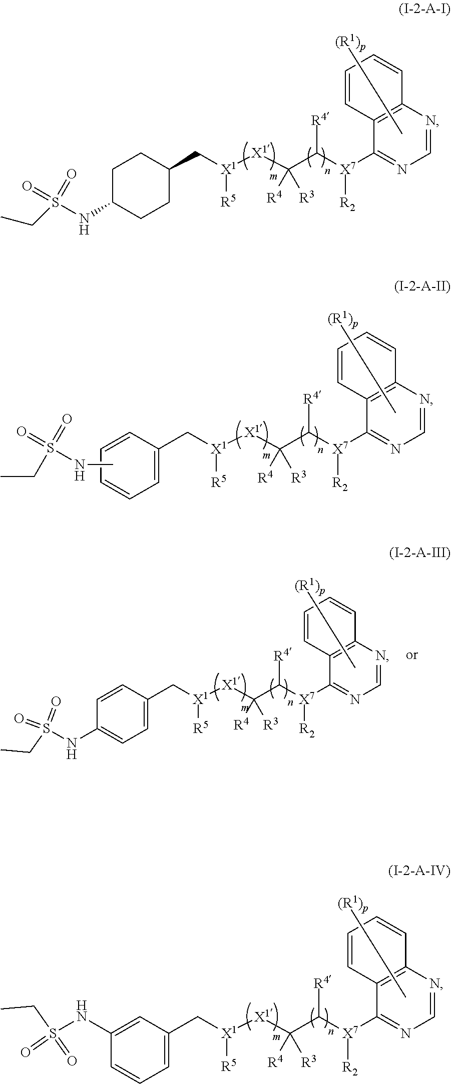

or a pharmaceutically acceptable salt, stereoisomer, solvate, prodrug, isotopic derivative, or tautomer thereof wherein all variables are as defined herein.

In some embodiments, the compound is of Formula (I-2-B—I), (I-2-B-II), (I-2-B—III), or (I-2-B—IV):

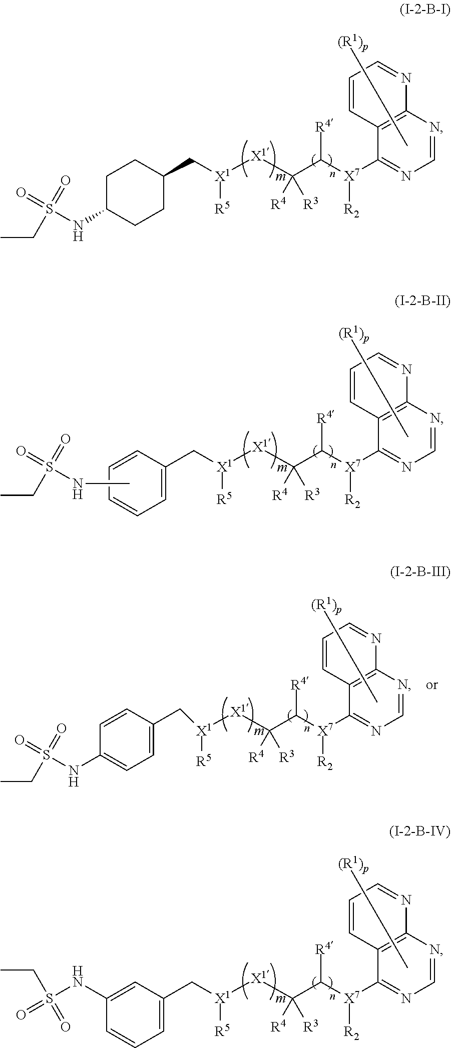

or a pharmaceutically acceptable salt, stereoisomer, solvate, prodrug, isotopic derivative, or tautomer thereof wherein all variables are as defined herein.

In some embodiments, the compound is selected from the compounds described in Table 1 and pharmaceutically acceptable salts, stereoisomers, solvates, prodrugs, isotopic derivatives, or tautomers thereof.

In some embodiments, the compound is selected from the compounds described in Table 1 and prodrugs and pharmaceutically acceptable salts thereof.

In some embodiments, the compound is selected from the compounds described in Table 1 and pharmaceutically acceptable salts thereof.

In some embodiments, the compound is selected from the prodrugs of the compounds described in Table 1 and pharmaceutically acceptable salts thereof.

In some embodiments, compounds in accordance with the present disclosure are selected from the compounds set forth in Table 1.

TABLE 1
Certain examples of the compound of Formula I

| # | IUPAC Name |
|---|---|
| 1 | N-[4-[[3-[6-(2,2,2-trifluoroethyl)quinazolin-4-y1]-3,9-diazaspiro[5.5]undecan-9- |
| | yl]methyl]phenyl]ethanesulfonamide |
| 2 | N-[4-[3-[7-(2,2,2-trifluoroethyl)quinazolin-4-yl]-3,9-diazaspiro[5.5]undecan-9- |
| | yl]methyl]phenyl]ethanesulfonamide |
| 3 | N-((1R,4R)-4-((9-(6-(2,2,2-trifluoroethyl)quinazolin-4-yl)-3,9-diazaspiro[5.5]undecan-3- |
| | yl)methyl)cyclohexyl)ethanesulfonamide |
| 4 | N-((1R,4R)-4-((9-(7-(2,2,2-trifluoroethyl)quinazolin-4-yl)-3,9-diazaspiro[5.5]undecan-3- |
| | yl)methyl)cyclohexyl)ethanesulfonamide |
| 5 | N-((1R,4R)-4-((2-(6-(2,2,2-trifluoroethyl)quinazolin-4-yl)-2,7-diazaspiro[3.5]nonan-7- |
| | yl)methyl)cyclohexyl)ethanesulfonamide |
| 6 | N-((1R,4R)-4-((2-(7-(2,2,2-trifluoroethyl)quinazolin-4-yl)-2,7-diazaspiro[3.5]nonan-7- |
| | yl)methyl)cyclohexyl)ethanesulfonamide |
| 7 | N-(4-((2-(6-(2,2,2-trifluoroethyl)quinazolin-4-yl)-2,7-diazaspiro[3.5]nonan-7- |
| | yl)methyl)phenyl)ethanesulfonamide |
| 8 | N-(4-((2-(7-(2,2,2-trifluoroethyl)quinazolin-4-yl)-2,7-diazaspiro[3.5]nonan-7- |
| | yl)methyl)phenyl)ethanesulfonamide |
| 9 | N-(4-((9-(2-(methylamino)-6-(2,2,2-trifluoroethyl)quinazolin-4-yl)-3,9-diazaspiro[5.5]undecan-3- |
| | yl)methyl)phenyl)ethanesulfonamide |
| 10 | N-(4-((9-(2-(methylamino)-7-(2,2,2-trifluoroethyl)quinazolin-4-yl)-3,9-diazaspiro[5.5]undecan-3- |
| | yl)methyl)phenyl)ethanesulfonamide |
| 11 | N-((1R,4R)-4-((9-(2-(methylamino)-6-(2,2,2-trifluoroethyl)quinazolin-4-yl)-3,9- |
| | diazaspiro[5.5]undecan-3-yl)methyl)cyclohexyl)ethanesulfonamide |
| 12 | N-((1R,4R)-4-((9-(2-(methylamino)-7-(2,2,2-trifluoroethyl)quinazolin-4-yl)-3,9- |
| | diazaspiro[5.5]undecan-3-yl)methyl)cyclohexyl)ethanesulfonamide |
| 13 | N-((1R,4R)-4-((2-(2-(methylamino)-6-(2,2,2-trifluoroethyl)quinazolin-4-yl)-2,7- |
| | diazaspiro[3.5]nonan-7-yl)methyl)cyclohexyl)ethanesulfonamide |
| 14 | N-((1R,4R)-4-((2-(2-(methylamino)-7-(2,2,2-trifluoroethyl)quinazolin-4-y1)-2,7- |
| | diazaspiro[3.5]nonan-7-yl)methyl)cyclohexyl)ethanesulfonamide |
| 15 | N-(4-((2-(2-(methylamino)-6-(2,2,2-trifluoroethyl)quinazolin-4-yl)-2,7-diazaspiro[3.5]nonan-7- |
| | yl)methyl)phenyl)ethanesulfonamide |
| 16 | N-(4-((2-(2-(methylamino)-7-(2,2,2-trifluoroethyl)quinazolin-4-yl)-2,7-diazaspiro[3.5]nonan-7- |
| | yl)methyl)phenyl)ethanesulfonamide |
| 21 | N-(4-((2-(quinazolin-4-yl)-2,7-diazaspiro[3.5]nonan-7-yl)methyl)phenyl)ethanesulfonamide |
| 22 | N-((1R,4R)-4-((2-(quinazolin-4-yl)-2,7-diazaspiro[3.5]nonan-7- |
| | yl)methyl)cyclohexyl)ethanesulfonamide |
| 23 | N-(3-((2-(quinazolin-4-yl)-2,7-diazaspiro[3.5]nonan-7-yl)methyl)phenyl)ethanesulfonamide |
| 24 | N-[4-[(2-pyrido[2,3-d]pyrimidin-4-yl-2,7-diazaspiro[3.5]nonan-7- |
| | yl)methyl]phenyl]ethanesulfonamide |
| 25 | N-[(1R,4R)-4-[(2-pyrido[2,3-d]pyrimidin-4-yl-2,7-diazaspiro[3.5]nonan-7- |
| | yl)methyl]cyclohexyl]ethanesulfonamide |
| 26 | N-[3-[(2-pyrido[2,3-d]pyrimidin-4-yl-2,7-diazaspiro[3.5]nonan-7- |
| | yl)methyl]phenyl]ethanesulfonamide |
| 27 | N-(4-((2-(2-(methylamino)quinazolin-4-yl)-2,7-diazaspiro[3.5]nonan-7- |
| | yl)methyl)phenyl)ethanesulfonamide |
| 28 | N-((1R,4R)-4-((2-(2-(methylamino)quinazolin-4-yl)-2,7-diazaspiro[3.5]nonan-7- |
| | yl)methyl)cyclohexyl)ethanesulfonamide |
| 29 | N-(3-((2-(2-(methylamino)quinazolin-4-yl)-2,7-diazaspiro[3.5]nonan-7- |
| | yl)methyl)phenyl)ethanesulfonamide |
| 30 | N-[3-[[2-[2-(methylamino)pyrido[2,3-d]pyrimidin-4-yl]-2,7-diazaspiro[3.5]nonan-7- |
| | yl]methyl]phenyl]ethanesulfonamide |
| 31 | N-[4-[2-[2-(methylamino)pyrido[2,3-d]pyrimidin-4-yl]-2,7-diazaspiro[3.5]nonan-7- |
| | yl]methyl]phenyl]ethanesulfonamide |
| 32 | N-(1R,4R)[4-[2-[2-(methylamino)pyrido[2,3-d]pyrimidin-4-yl]-2,7-diazaspiro[3.5]nonan-7- |
| | yl]methyl]cyclohexyl]ethanesulfonamide |
| 33 | N-(4-((4-((7-(2,2,2-trifluoroethyl)quinazolin-4-yl)amino)piperidin-1- |
| | yl)methyl)phenyl)ethanesulfonamide |
| 34 | N-((1R,4R)-4-((4-((7-(2,2,2-trifluoroethyl)quinazolin-4-yl)amino)piperidin-1- |
| | yl)methyl)cyclohexyl)ethanesulfonamide |
| 35 | N-(3-((4-((7-(2,2,2-trifluoroethyl)quinazolin-4-yl)amino)piperidin-1- |
| | yl)methyl)phenyl)ethanesulfonamide |
| 36 | N-(4-((4-((6-(2,2,2-trifluoroethyl)quinazolin-4-yl)amino)piperidin-1- |
| | yl)methyl)phenyl)ethanesulfonamide |
| 37 | N-((1R,4R)-4-((4-((6-(2,2,2-trifluoroethyl)quinazolin-4-yl)amino)piperidin-1- |
| | yl)methyl)cyclohexyl)ethanesulfonamide |
| 38 | N-(3-((4-((6-(2,2,2-trifluoroethyl)quinazolin-4-yl)amino)piperidin-1- |
| | yl)methyl)phenyl)ethanesulfonamide |
| 39 | N-(4-((4-(quinazolin-4-ylamino)piperidin-1-yl)methyl)phenyl)ethanesulfonamide |
| 40 | N-(3-((4-(quinazolin-4-ylamino)piperidin-1-yl)methyl)phenyl)ethanesulfonamide |
| 41 | N-(4-((7-(quinazolin-4-yl)-2,7-diazaspiro[3.5]nonan-2-yl)methyl)phenyl)ethanesulfonamide |
| 42 | N-(3-((7-(quinazolin-4-yl)-2,7-diazaspiro[3.5]nonan-2-yl)methyl)phenyl)ethanesulfonamide |
| 43 | N-(4-((7-(6-(2,2,2-trifluoroethyl)quinazolin-4-yl)-2,7-diazaspiro[3.5]nonan-2- |
| | yl)methyl)phenyl)ethanesulfonamide |
| 44 | N-(3-((7-(6-(2,2,2-trifluoroethyl)quinazolin-4-yl)-2,7-diazaspiro[3.5]nonan-2- |
| | yl)methyl)phenyl)ethanesulfonamide |
| 45 | N-ethyl-4-((7-(6-(2,2,2-trifluoroethyl)quinazolin-4-yl)-2,7-diazaspiro[3.5]nonan-2- |
| | yl)methyl)benzenesulfonamide |
| 46 | N-ethyl-4-((2-(6-(2,2,2-trifluoroethyl)quinazolin-4-yl)-2,7-diazaspiro[3.5]nonan-7- |
| | yl)methyl)benzenesulfonamide |
| 48 | N-((1R,4R)-4-((2-(6-(2-(pyridin-4-yl)ethyl)quinazolin-4-yl)-2,7-diazaspiro[3.5]nonan-7- |
| | yl)methyl)cyclohexyl)ethanesulfonamide |
| 49 | N-(4-((2-(6-(2-(pyridin-4-yl)ethyl)quinazolin-4-yl)-2,7-diazaspiro[3.5]nonan-7- |
| | yl)methyl)phenyl)ethanesulfonamide |
| 51 | N-((1R,4R)-4-((2-(6-phenethylquinazolin-4-yl)-2,7-diazaspiro[3.5]nonan-7- |
| | yl)methyl)cyclohexyl)ethanesulfonamide |
| 52 | N-(4-((2-(6-phenethylquinazolin-4-yl)-2,7-diazaspiro[3.5]nonan-7- |
| | yl)methyl)phenyl)ethanesulfonamide |
| 53 | N-((1R,4R)-4-((2-(6-(phenylethynyl)quinazolin-4-yl)-2,7-diazaspiro[3.5]nonan-7- |
| | yl)methyl)cyclohexyl)ethanesulfonamide |
| 54 | N-(3-((2-(6-(phenylethynyl)quinazolin-4-yl)-2,7-diazaspiro[3.5]nonan-7- |
| | yl)methyl)phenyl)ethanesulfonamide |
| 55 | N-(4-((2-(6-(phenylethynyl)quinazolin-4-yl)-2,7-diazaspiro[3.5]nonan-7- |
| | yl)methyl)phenyl)ethanesulfonamide |
| 56 | N-((1R,4R)-4-((2-(6-(2-(pyridin-3-yl)ethyl)quinazolin-4-yl)-2,7-diazaspiro[3.5]nonan-7- |
| | yl)methyl)cyclohexyl)ethanesulfonamide |
| 57 | N-((1R,4R)-4-((2-(6-(2-methylphenethyl)quinazolin-4-yl)-2,7-diazaspiro[3.5]nonan-7- |
| | yl)methyl)cyclohexyl)ethanesulfonamide |
| 58 | N-((1R,4R)-4-((2-(6-(2-methoxyphenethyl)quinazolin-4-yl)-2,7-diazaspiro[3.5]nonan-7- |
| | yl)methyl)cyclohexyl)ethanesulfonamide |
| 59 | N-((1R,4R)-4-((2-(6-((RS)-2-phenylpropyl)quinazolin-4-yl)-2,7-diazaspiro[3.5]nonan-7- |
| | yl)methyl)cyclohexyl)ethanesulfonamide |
| 60 | N-((1R,4R)-4-((2-(6-(4-fluorophenethyl)quinazolin-4-yl)-2,7-diazaspiro[3.5]nonan-7- |
| | yl)methyl)cyclohexyl)ethanesulfonamide |
| 61 | N-((1R,4R)-4-((2-(6-(3-fluorophenethyl)quinazolin-4-yl)-2,7-diazaspiro[3.5]nonan-7- |
| | yl)methyl)cyclohexyl)ethanesulfonamide |
| 62 | N-((1R,4R)-4-((2-(6-(2-(5-methyl-1,3,4-oxadiazol-2-yl)ethyl)quinazolin-4-yl)-2,7- |
| | diazaspiro[3.5]nonan-7-yl)methyl)cyclohexyl)ethanesulfonamide |
| 63 | N-((1R,4R)-4-((2-(6-(2-(3-methyl-1,2,4-oxadiazol-5-yl)ethyl)quinazolin-4-yl)-2,7- |
| | diazaspiro[3.5]nonan-7-yl)methyl)cyclohexyl)ethanesulfonamide |
| 64 | N-((1R,4R)-4-((2-(6-(4-methoxyphenethyl)quinazolin-4-yl)-2,7-diazaspiro[3.5]nonan-7- |
| | yl)methyl)cyclohexyl)ethanesulfonamide |
| 65 | N-(1R,4R)-[4-[[2-[6-(2,2,2-trifluoroethyl)pyrido[2,3-d]pyrimidin-4-yl]-2,7-diazaspiro[3.5]nonan-7- |
| | yl]methyl]cyclohexyl]ethanesulfonamide |
| 66 | N-(1R,4R)-[4-[2-[2-(methylamino)-6-(2,2,2-trifluoroethyl)pyrido[2,3-d]pyrimidin-4-yl]-2,7- |
| | diazaspiro[3.5]nonan-7-yl]methyl]cyclohexyl]ethanesulfonamide |
| 69 | N-((1R,4R)-4-((4-(((6-(2,2,2-trifluoroethyl)quinazolin-4-yl)amino)methyl)-1H-1,2,3-triazol-1- |
| | yl)methyl)cyclohexyl)ethanesulfonamide |
| 70 | rac-(R)-N-(2-(2-(4-(methylsulfonyl)piperazin-1-yl)propoxy)-4-((2-(6-(2,2,2- |
| | trifluoroethyl)quinazolin-4-yl)-2,7-diazaspiro[3.5]nonan-7-yl)methyl)phenyl)ethanesulfonamide |
| 71 | N-((1R,4R)-4-(((RS)-3-((6-(2,2,2-trifluoroethyl)quinazolin-4-yl)amino)pyrrolidin-1- |
| | yl)methyl)cyclohexyl)ethanesulfonamide |
| 72 | N-(2-(ethylsulfonamido)-5-((2-(6-(2,2,2-trifluoroethyl)quinazolin-4-yl)-2,7-diazaspiro[3.5]nonan-7- |
| | yl)methyl)phenyl)acetamide |
| 73 | 2-(2-(ethylsulfonamido)-5-((2-(6-(2,2,2-trifluoroethyl)quinazolin-4-yl)-2,7-diazaspiro[3.5]nonan-7- |
| | yl)methyl)phenoxy)acetamide |
| 74 | N-(3-((2-(6-(2,2,2-trifluoroethyl)quinazolin-4-yl)-2,7-diazaspiro[3.5]nonan-7- |
| | yl)methyl)bicyclo[1.1.1]pentan-1-yl)ethanesulfonamide |
| 75 | 7-((2-(6-(2,2,2-trifluoroethyl)quinazolin-4-yl)-2,7-diazaspiro[3.5]nonan-7-yl)methyl)-2H- |
| | benzo[b][1,4]oxazin-3(4H)-one |
| 77 | rac-(R)-N-(8-fluoro-6-((2-(6-(2,2,2-trifluoroethyl)quinazolin-4-yl)-2,7-diazaspiro[3.5]nonan-7- |
| | yl)methyl)-1,2,3,4-tetrahydronaphthalen-2-yl)ethanesulfonamide |
| 78 | N-((1R,4R)-4-((methyl(4-((2-(6-(2,2,2-trifluoroethyl)quinazolin-4-yl)-2,7-diazaspiro[3.5]nonan-7- |
| | yl)methyl)phenyl)amino)methyl)cyclohexyl)ethanesulfonamide |
| 79 | N-((1R,4R)-4-(((4-((2-(6-(2,2,2-trifluoroethyl)quinazolin-4-yl)-2,7-diazaspiro[3.5]nonan-7- |
| | yl)methyl)phenyl)amino)methyl)cyclohexyl)ethanesulfonamide |
| 80 | 4-[7-[[4-(sulfamoylamino)phenyl]methyl]-2,7-diazaspiro[3.5]nonan-2-yl]-6-(2,2,2- |
| | trifluoroethyl)quinazoline |
| 81 | 7-((2-(6-(2,2,2-trifluoroethyl)quinazolin-4-yl)-2,7-diazaspiro[3.5]nonan-7-yl)methyl)-3,4-dihydro- |
| | 1H-benzo[f][1,4,5]oxathiazepine 2,2-dioxide |
| 82 | 6-((2-(6-(2,2,2-trifluoroethyl)quinazolin-4-yl)-2,7-diazaspiro[3.5]nonan-7-yl)methyl)-1H,3H- |
| | benzo[e][1,3,4]oxathiazine 2,2-dioxide |
| 83 | 4-(7-((1H-indol-4-yl)methyl)-2,7-diazaspiro[3.5]nonan-2-yl)-6-(2,2,2-trifluoroethyl)quinazoline |
| 84 | rac-(R)-7-hydroxy-5-methyl-11-((2-(6-(2,2,2-trifluoroethyl)quinazolin-4-yl)-2,7- |
| | diazaspiro[3.5]nonan-7-yl)methyl)-3,4,5,6,7,8-hexahydro-1H- |
| | benzo[b][1]oxa[5]thia[4,8]diazacycloundecine 2,2-dioxide |
| 85 | rac-(R)-7-hydroxy-5-(2-methoxyethyl)-11-((2-(6-(2,2,2-trifluoroethyl)quinazolin-4-yl)-2,7- |
| | diazaspiro[3.5]nonan-7-yl)methyl)-3,4,5,6,7,8-hexahydro-1H- |
| | benzo[b][1]oxa[5]thia[4,8]diazacycloundecine 2,2-dioxide |
| 86 | rac-(R)-7-hydroxy-5-isopentyl-11-((2-(6-(2,2,2-trifluoroethyl)quinazolin-4-yl)-2,7- |
| | diazaspiro[3.5]nonan-7-yl)methyl)-3,4,5,6,7,8-hexahydro-1H- |
| | benzo[b][1]oxa[5]thia[4,8]diazacycloundecine 2,2-dioxide |
| 87 | 5-(2-methoxyethyl)-7-((2-(6-(2,2,2-trifluoroethyl)quinazolin-4-yl)-2,7-diazaspiro[3.5]nonan-7- |
| | yl)methyl)-1,3,4,5-tetrahydrobenzo[c][1,2,5]thiadiazepine 2,2-dioxide |
| 88 | 5-isopentyl-7-((2-(6-(2,2,2-trifluoroethyl)quinazolin-4-yl)-2,7-diazaspiro[3.5]nonan-7-yl)methyl)- |
| | 1,3,4,5-tetrahydrobenzo[c][1,2,5]thiadiazepine 2,2-dioxide |
| 89 | 4-(7-((1H-indol-6-yl)methyl)-2,7-diazaspiro[3.5]nonan-2-yl)-6-(2,2,2-trifluoroethyl)quinazoline |
| 90 | 4-(7-((1H-indol-5-yl)methyl)-2,7-diazaspiro[3.5]nonan-2-yl)-6-(2,2,2-trifluoroethyl)quinazoline |
| 91 | 2-amino-N-(2-morpholinoethyl)-5-((2-(6-(2,2,2-trifluoroethyl)quinazolin-4-yl)-2,7- |
| | diazaspiro[3.5]nonan-7-yl)methyl)benzamide |
| 92 | 2-(ethylsulfonamido)-N-(2-morpholinoethyl)-5-((2-(6-(2,2,2-trifluoroethyl)quinazolin-4-yl)-2,7- |
| | diazaspiro[3.5]nonan-7-yl)methyl)benzamide |
| 93 | 4-(7-(isoindolin-5-ylmethyl)-2,7-diazaspiro[3.5]nonan-2-yl)-6-(2,2,2-trifluoroethyl)quinazoline |
| 94 | 4-(7-((2-(ethylsulfonyl)isoindolin-5-yl)methyl)-2,7-diazaspiro[3.5]nonan-2-yl)-6-(2,2,2- |
| | trifluoroethyl)quinazoline |
| 100 | N-((1R,4R)-4-((ethyl(4-((2-(6-(2,2,2-trifluoroethyl)quinazolin-4-yl)-2,7-diazaspiro[3.5]nonan-7- |
| | yl)methyl)phenyl)amino)methyl)cyclohexyl)ethanesulfonamide |
| 101 | 2-((2-(4-(methylsulfonyl)piperazin-1-yl)ethyl)amino)-5-((2-(6-(2,2,2-trifluoroethyl)quinazolin-4- |
| | yl)-2,7-diazaspiro[3.5]nonan-7-yl)methyl)benzonitrile |
| 102 | 2-fluoro-5-((2-(6-(2,2,2-trifluoroethyl)quinazolin-4-yl)-2,7-diazaspiro[3.5]nonan-7- |
| | yl)methyl)benzonitrile |
| 110 | 4-(7-((2-(ethylsulfonyl)-1,2,3,4-tetrahydroisoquinolin-6-yl)methyl)-2,7-diazaspiro[3.5]nonan-2-yl)- |
| | 6-(2,2,2-trifluoroethyl)quinazoline |
| 111 | 4-(7-((2-(ethylsulfonyl)-1,2,3,4-tetrahydroisoquinolin-7-yl)methyl)-2,7-diazaspiro[3.5]nonan-2-y1)- |
| | 6-(2,2,2-trifluoroethyl)quinazoline |
| 138 | 2-[2-ethylsulfonyl-6-[[2-[6-(2,2,2-trifluoroethyl)quinazolin-4-yl]-2,7-diazaspiro[3.5]nonan-7- |
| | yl]methyl]-3,4-dihydro-1H-isoquinolin-3-yl]ethanol |

In some embodiments, the compound is a pharmaceutically acceptable salt of any one of the compounds described in Table 1.

In some embodiments, the compound is a lithium salt, sodium salt, potassium salt, calcium salt, or magnesium salt of any one of the compounds described in Table 1.

In some embodiments, the compound is a sodium salt or potassium salt of any one of the compounds described in Table 1.

In some embodiments, the compound is a sodium salt of any one of the compounds described in Table 1.

In some embodiments, the compound is a potassium salt of any one of the compounds described in Table 1.

In some aspects, the present disclosure provides a compound being an isotopic derivative (e.g., isotopically labeled compound) of any one of the compounds of the Formulae disclosed herein.

In some embodiments, the compound is an isotopic derivative of any one of the compounds described in Table 1 and prodrugs and pharmaceutically acceptable salts thereof.

In some embodiments, the compound is an isotopic derivative of any one of the compounds described in Table 1 and pharmaceutically acceptable salts thereof.

In some embodiments, the compound is an isotopic derivative of any one of prodrugs of the compounds described in Table 1 and pharmaceutically acceptable salts thereof.

In some embodiments, the compound is an isotopic derivative of any one of the compounds described in Table 1.

It is understood that the isotopic derivative can be prepared using any of a variety of art-recognized techniques. For example, the isotopic derivative can generally be prepared by carrying out the procedures disclosed in the Schemes and/or in the Examples described herein, by substituting an isotopically labeled reagent for a non-isotopically labeled reagent.

In some embodiments, the isotopic derivative is a deuterium labeled compound.

In some embodiments, the isotopic derivative is a deuterium labeled compound of any one of the compounds of the Formulae disclosed herein.

The term “isotopic derivative”, as used herein, refers to a derivative of a compound in which one or more atoms are isotopically enriched or labelled. For example, an isotopic derivative of a compound of Formula (I) is isotopically enriched with regard to, or labelled with, one or more isotopes as compared to the corresponding compound of Formula (I). In some embodiments, the isotopic derivative is enriched with regard to, or labelled with, one or more atoms selected from ²H, ¹³C, ¹⁴C, ¹⁵N, ¹⁸O, ²⁹Si, ³¹P, and ³⁴S. In some embodiments, the isotopic derivative is a deuterium labeled compound (i.e., being enriched with 2H with regard to one or more atoms thereof).

In some embodiments, the compound is a deuterium labeled compound of any one of the compounds described in Table 1 and prodrugs and pharmaceutically acceptable salts thereof.

In some embodiments, the compound is a deuterium labeled compound of any one of the compounds described in Table 1 and pharmaceutically acceptable salts thereof.

In some embodiments, the compound is a deuterium labeled compound of any one of the prodrugs of the compounds described in Table 1 and pharmaceutically acceptable salts thereof.

In some embodiments, the compound is a deuterium labeled compound of any one of the compounds described in Table 1.

It is understood that the deuterium labeled compound comprises a deuterium atom having an abundance of deuterium that is substantially greater than the natural abundance of deuterium, which is 0.015%.

In some embodiments, the deuterium labeled compound has a deuterium enrichment factor for each deuterium atom of at least 3500 (52.5% deuterium incorporation at each deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). As used herein, the term “deuterium enrichment factor” means the ratio between the deuterium abundance and the natural abundance of a deuterium.

It is understood that the deuterium labeled compound can be prepared using any of a variety of art-recognized techniques. For example, the deuterium labeled compound can generally be prepared by carrying out the procedures disclosed in the Schemes and/or in the Examples described herein, by substituting a deuterium labeled reagent for a non-deuterium labeled reagent.

A compound of the disclosure or a pharmaceutically acceptable salt or solvate thereof that contains the aforementioned deuterium atom(s) is within the scope of the disclosure. Further, substitution with deuterium (i.e., ²H) may afford certain therapeutic advantages resulting from greater metabolic stability, e.g., increased in vivo half-life or reduced dosage requirements.

In some embodiments, the compound is a ¹⁸F labeled compound.

In some embodiments, the compound is a ¹²³I labeled compound, a ¹²⁴I labeled compound, a ¹²⁵I labeled compound, a ¹²⁹I labeled compound, a ¹³¹I labeled compound, a ¹³⁵I labeled compound, or any combination thereof.

In some embodiments, the compound is a ³³S labeled compound, a ³⁴S labeled compound, a ³⁵S labeled compound, a ³⁶S labeled compound, or any combination thereof.

It is understood that the ¹⁸F, ¹²³I, ¹²⁴I, ¹²⁵ I, ¹²⁹I, ¹³¹I, ¹³⁵I, ³S, ³⁴S, ³⁵S, and/or ³⁶S labeled compound, can be prepared using any of a variety of art-recognized techniques. For example, the deuterium labeled compound can generally be prepared by carrying out the procedures disclosed in the Schemes and/or in the Examples described herein, by substituting a ¹⁸F, ¹²³I, ¹²⁴I, ¹²⁵I, ¹²⁹I, ¹³¹I, ¹³⁵I, ³S, ³⁴S, ³⁵S, and/or ³⁶S labeled reagent for a non-isotope labeled reagent.

A compound of the disclosure or a pharmaceutically acceptable salt or solvate thereof that contains one or more of the aforementioned ¹⁸F, ¹²³I, ¹²⁴I, ¹²⁵I, ¹²⁹I, ¹³¹I, ¹³⁵I, ³S, ³⁴S, ³⁵S, and ³⁶S atom(s) is within the scope of the disclosure. Further, substitution with isotope (e.g., ¹⁸F, ¹²³I, ¹²⁴I, ¹²⁵I, ¹²⁹I, ¹³¹I, ¹³⁵I, ³S, ³⁴S, ³⁵S, and/or ³⁶S) may afford certain therapeutic advantages resulting from greater metabolic stability, e.g., increased in vivo half-life or reduced dosage requirements.

For the avoidance of doubt, it is to be understood that, where in this specification a group is qualified by “described herein”, the said group encompasses the first occurring and broadest definition as well as each and all of the particular definitions for that group.

The various functional groups and substituents making up the compounds of the Formula (I) are typically chosen such that the molecular weight of the compound does not exceed 1000 Daltons. More usually, the molecular weight of the compound will be less than 900, for example less than 800, or less than 750, or less than 700, or less than 650 Daltons. More conveniently, the molecular weight is less than 600 and, for example, is 550 Daltons or less.

A suitable pharmaceutically acceptable salt of a compound of the disclosure is, for example, an acid-addition salt of a compound of the disclosure, which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the disclosure which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, diethylamine, trimethylamine, piperidine, morpholine or tris-(2-hydroxyethyl) amine.

It will be understood that the compounds of any one of the Formulae disclosed herein and any pharmaceutically acceptable salts thereof, comprise stereoisomers, mixtures of stereoisomers, polymorphs of all isomeric forms of said compounds.

As used herein, the term “isomerism” means compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers” or sometimes optical isomers. A mixture containing equal amounts of individual enantiomeric forms of opposite chirality is termed a “racemic mixture.”

As used herein, the term “chiral center” refers to a carbon atom bonded to four nonidentical substituents.

As used herein, the term “chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

As used herein, the term “geometric isomer” means the diastereomers that owe their existence to hindered rotation about double bonds or a cycloalkyl linker (e.g., 1,3-cyclobutyl). These configurations are differentiated in their names by the prefixes cis and trans, or Z and E, which indicate that the groups are on the same or opposite side of the double bond in the molecule according to the Cahn-Ingold-Prelog rules.

It is to be understood that the compounds of the present disclosure may be depicted as different chiral isomers or geometric isomers. It is also to be understood that when compounds have chiral isomeric or geometric isomeric forms, all isomeric forms are intended to be included in the scope of the present disclosure, and the naming of the compounds does not exclude any isomeric forms, it being understood that not all isomers may have the same level of activity.

It is to be understood that the structures and other compounds discussed in this disclosure include all atropic isomers thereof. It is also to be understood that not all atropic isomers may have the same level of activity.

As used herein, the term “atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, the term “tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerisation is possible, a chemical equilibrium of the tautomers will be reached. The exact ratio of the tautomers depends on several factors, including temperature, solvent, and pH. The concept of tautomers that are interconvertible by tautomerisations is called tautomerism. Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric centre, for example, it is bonded to four different groups, a pair of enantiomers is possible. An enantiomer can be characterised by the absolute configuration of its asymmetric centre and is described by the R- and S-sequencing rules of Cahn and Prelog, or by the manner in which the molecule rotates the plane of polarised light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this disclosure may possess one or more asymmetric centres; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the disclosure may have geometric isomeric centres (E- and Z-isomers). It is to be understood that the present disclosure encompasses all optical, diastereoisomers and geometric isomers and mixtures thereof that possess inflammasome inhibitory activity.

The present disclosure also encompasses compounds of the disclosure as defined herein which comprise one or more isotopic substitutions.

It is to be understood that the compounds of any Formula described herein include the compounds themselves, as well as their salts, and their solvates, if applicable. A salt, for example, can be formed between an anion and a positively charged group (e.g., amino) on a substituted compound disclosed herein. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted compound disclosed herein. Suitable cations include sodium ion, potassium ion, magnesium ion, calcium ion, and an ammonium cation such as tetramethylammonium ion or diethylamine ion. The substituted compounds disclosed herein also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonamides, tetrazoles, sulfonates and phosphonates. See, e.g., Patani and LaVoie, Chem. Rev. 96, 3147-3176, 1996.

It is also to be understood that certain compounds of any one of the Formulae disclosed herein may exist in solvated as well as unsolvated forms such as, for example, hydrated forms. A suitable pharmaceutically acceptable solvate is, for example, a hydrate such as hemihydrate, a monohydrate, a di-hydrate or a tri-hydrate. It is to be understood that the disclosure encompasses all such solvated forms that possess inflammasome inhibitory activity.

It is also to be understood that certain compounds of any one of the Formulae disclosed herein may exhibit polymorphism, and that the disclosure encompasses all such forms, or mixtures thereof, which possess inflammasome inhibitory activity. It is generally known that crystalline materials may be analysed using conventional techniques such as X-Ray Powder Diffraction analysis, Differential Scanning calorimetry, Thermal Gravimetric Analysis, Diffuse Reflectance Infrared Fourier Transform (DRIFT) spectroscopy, Near Infrared (NIR) spectroscopy, solution and/or solid state nuclear magnetic resonance spectroscopy. The water content of such crystalline materials may be determined by Karl Fischer analysis.

Compounds of any one of the Formulae disclosed herein may exist in a number of different tautomeric forms and references to compounds of Formula (I) or (II) include all such forms. For the avoidance of doubt, where a compound can exist in one of several tautomeric forms, and only one is specifically described or shown, all others are nevertheless embraced by Formula (I) or (II). Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

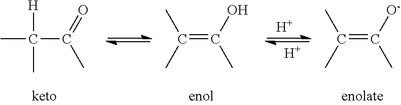

Compounds of any one of the Formulae disclosed herein containing an amine function may also form N-oxides. A reference herein to a compound of Formula (I) or (II) that contains an amine function also includes the N-oxide. Where a compound contains several amine functions, one or more than one nitrogen atom may be oxidised to form an N-oxide. Particular examples of N-oxides are the N-oxides of a tertiary amine or a nitrogen atom of a nitrogen-containing heterocycle. N-oxides can be formed by treatment of the corresponding amine with an oxidising agent such as hydrogen peroxide or a peracid (e.g., a peroxycarboxylic acid), see for example Advanced Organic Chemistry, by Jerry March 4th Edition, Wiley-Interscience, pages. More particularly, N-oxides can be made by the procedure of L. W. Deady (Syn. Comm. 1977, 7, 509-514) in which the amine compound is reacted with meta-chloroperoxybenzoic acid (mCPBA), for example, in an inert solvent such as dichloromethane.

The compounds of any one of the Formulae disclosed herein may be administered in the form of a prodrug which is broken down in the human or animal body to release a compound of the disclosure. A prodrug may be used to alter the physical properties and/or the pharmacokinetic properties of a compound of the disclosure. A prodrug can be formed when the compound of the disclosure contains a suitable group or substituent to which a property-modifying group can be attached. Examples of prodrugs include derivatives containing in vivo cleavable alkyl or acyl substituents at the ester or amide group in any one of the Formulae disclosed herein.

Accordingly, the present disclosure includes those compounds of any one of the Formulae disclosed herein as defined hereinbefore when made available by organic synthesis and when made available within the human or animal body by way of cleavage of a prodrug thereof. Accordingly, the present disclosure includes those compounds of any one of the Formulae disclosed herein that are produced by organic synthetic means and also such compounds that are produced in the human or animal body by way of metabolism of a precursor compound, that is a compound of any one of the Formulae disclosed herein may be a synthetically produced compound or a metabolically produced compound.

A suitable pharmaceutically acceptable prodrug of a compound of any one of the Formulae disclosed herein is one that is based on reasonable medical judgment as being suitable for administration to the human or animal body without undesirable pharmacological activities and without undue toxicity. Various forms of prodrug have been described, for example in the following documents: a) Methods in Enzymology, Vol. 42, p. 309-396, edited by K. Widder, et al. (Academic Press, 1985); b) Design of Pro-drugs, edited by H. Bundgaard, (Elsevier, 1985); c) A Textbook of Drug Design and Development, edited by Krogsgaard-Larsen and H. Bundgaard, Chapter 5 “Design and Application of Pro-drugs”, by H. Bundgaard p. 113-191 (1991); d) H. Bundgaard, Advanced Drug Delivery Reviews, 8, 1-38 (1992); e) H. Bundgaard, et al., Journal of Pharmaceutical Sciences, 77, 285 (1988); f) N. Kakeya, et al., Chem. Pharm. Bull., 32, 692 (1984); g) T. Higuchi and V. Stella, “Pro-Drugs as Novel Delivery Systems”, A.C.S. Symposium Series, Volume 14; and h) E. Roche (editor), “Bioreversible Carriers in Drug Design”, Pergamon Press, 1987.

A suitable pharmaceutically acceptable prodrug of a compound of any one of the Formulae disclosed herein that possesses a hydroxy group is, for example, an in vivo cleavable ester or ether thereof. An in vivo cleavable ester or ether of a compound of any one of the Formulae disclosed herein containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C₁-C₁₀ alkanoyl groups such as acetyl, benzoyl, phenylacetyl and substituted benzoyl and phenylacetyl groups, C₁-C₁₀ alkoxycarbonyl groups such as ethoxycarbonyl, N,N—(C₁-C₆ alkyl)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl. Suitable pharmaceutically acceptable ether forming groups for a hydroxy group include a-acyloxyalkyl groups such as acetoxymethyl and pivaloyloxymethyl groups.

A suitable pharmaceutically acceptable prodrug of a compound of any one of the Formulae disclosed herein that possesses a carboxy group is, for example, an in vivo cleavable amide thereof, for example an amide formed with an amine such as ammonia, a C₁₋₄alkylamine such as methylamine, a (C₁-C₄ alkyl)₂amine such as dimethylamine, N-ethyl-N-methylamine or diethylamine, a C₁-C₄ alkoxy-C₂-C₄ alkylamine such as 2-methoxyethylamine, a phenyl-C₁-C₄ alkylamine such as benzylamine and amino acids such as glycine or an ester thereof.

A suitable pharmaceutically acceptable prodrug of a compound of any one of the Formulae disclosed herein that possesses an amino group is, for example, an in vivo cleavable amide derivative thereof. Suitable pharmaceutically acceptable amides from an amino group include, for example an amide formed with C₁-C₁₀ alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁-C₄ alkyl) piperazin-1-ylmethyl.

The in vivo effects of a compound of any one of the Formulae disclosed herein may be exerted in part by one or more metabolites that are formed within the human or animal body after administration of a compound of any one of the Formulae disclosed herein. As stated hereinbefore, the in vivo effects of a compound of any one of the Formulae disclosed herein may also be exerted by way of metabolism of a precursor compound (a prodrug).

### Method of Synthesizing the Compounds

The compounds of the present invention may be made by a variety of methods, including standard chemistry. Suitable synthetic routes are depicted in the Schemes given below.

The compounds of Formula (I) may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of those skilled in the art will recognize if a stereocenter exists in the compounds of Formula (I). Accordingly, the present invention includes both possible stereoisomers (unless specified in the synthesis) and includes not only racemic compounds but the individual enantiomers and/or diastercomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

The compounds described herein may be made from commercially available starting materials or synthesized using known organic, inorganic, and/or enzymatic processes.

### Preparation of Compounds

The compounds of the present invention can be prepared in a number of ways well known to those skilled in the art of organic synthesis. By way of example, compounds of the present invention can be synthesized using the methods described below, together with synthetic methods known in the art of synthetic organic chemistry, or variations thereon as appreciated by those skilled in the art. Suitable methods include but are not limited to those methods described below. Compounds of the present invention can be synthesized by following the steps outlined in General Procedures A or B which comprise different sequences of assembling intermediates or compounds. Starting materials are either commercially available or made by known procedures in the reported literature or as illustrated below.

### General Procedure A

In general, the compound of the Formula (I) can be prepared using reaction of reductive amination of carbonyl compound (A) with amine (B) in the presence of appropriate reducing agent ([H]):

$$A - C ( O ) H + B - {NH}_{2} + [ H ] \to Compound of Formula ( I )$$

In more specific aspects, the compounds according to Formula (I) can be obtained by using the scheme presented below:

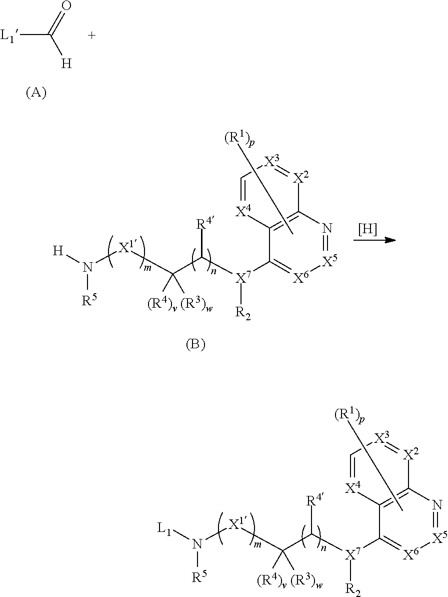

As a specific non-limiting example of this procedure can be presented by the reaction of preparation of the compound 7 (Example 7):

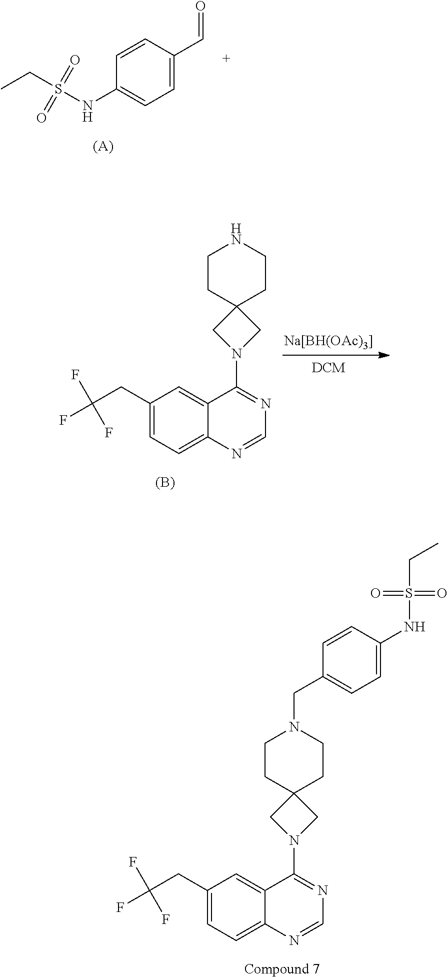

Reagents (A) and (B) may be commercially available compounds itself or products of synthesis from commercially available reagents. For compounds (A) and (B) preparation may be used one step or multistep synthetic procedures, including but not limited procedures described herein in preparative part.

### General Procedure B

In general, the compound of the Formula (1) can be prepared using reaction substitution of halogen in halogen derivative (A) by amine (B) in the presence of base (Base):

$$A - Hal + B - {NH}_{2} + Base \to Compound of Formula ( I ) + Base \text{:} HCl$$

In more specific aspects, compounds of Formula (I) can be obtained according to the scheme presented below:

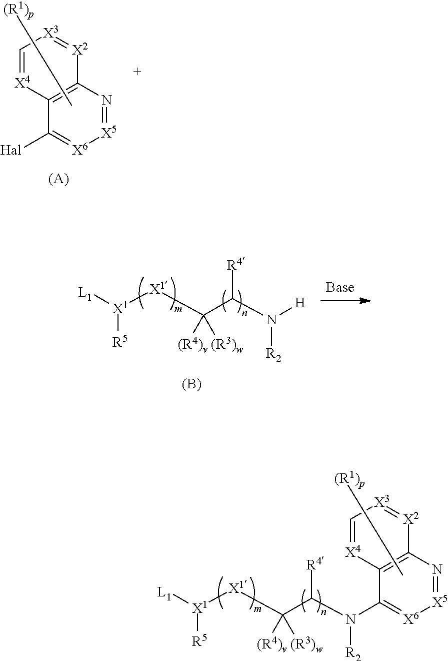

As a specific non-limiting example of this procedure can be presented by the reaction of preparation of the compound 75 (Example 16):

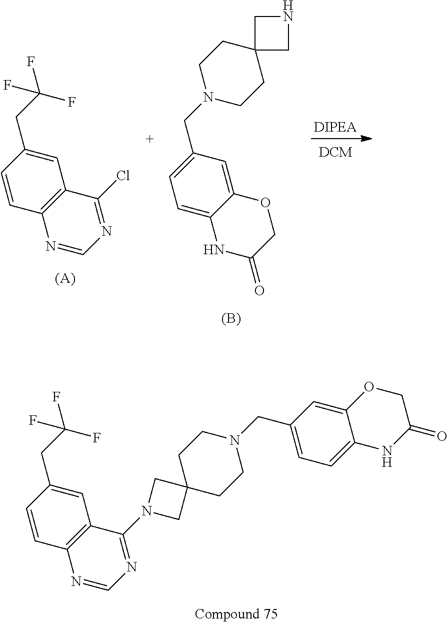

Reagents (A) and (B) may be commercially available compounds itself or products of synthesis from commercially available reagents. For compounds (A) and (B) preparation may be used one step or multistep synthetic procedures, including but not limited procedures described herein in preparative part.

It should be evident for specialists in this field that any of compounds of Formula (I) obtained according to the procedures A or B described above may be a subject for further transformation and modification that will led to obtain other compound of Formula (I).

### Biological Assays

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described below, to determine whether they have a predicted activity, binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

Various in vitro or in vivo biological assays may be suitable for detecting the effect of the compounds of the present disclosure. These in vitro or in vivo biological assays can include, but are not limited to, enzymatic activity assays, electrophoretic mobility shift assays, reporter gene assays, in vitro cell viability assays, and the assays described herein.

### Pharmaceutical Compositions

In some aspects, the present disclosure provides a pharmaceutical composition comprising a compound of the present disclosure as an active ingredient. In some embodiments, the present disclosure provides a pharmaceutical composition comprising at least one compound of each of the formulae described herein, or a pharmaceutically acceptable salt or solvate thereof, and one or more pharmaceutically acceptable carriers or excipients. In some embodiments, the present disclosure provides a pharmaceutical composition comprising at least one compound selected from Table 1.

As used herein, the term “composition” is intended to encompass a product comprising the specified ingredients in the specified amounts, as well as any product which results, directly or indirectly, from combination of the specified ingredients in the specified amounts.

The compounds of present disclosure can be formulated for oral administration in forms such as tablets, capsules (each of which includes sustained release or timed-release formulations), pills, powders, granules, elixirs, tinctures, suspensions, syrups and emulsions. The compounds of present disclosure on can also be formulated for intravenous (bolus or in-fusion), intraperitoneal, topical, subcutaneous, intramuscular or transdermal (e.g., patch) administration, all using forms well known to those of ordinary skill in the pharmaceutical arts.

The formulation of the present disclosure may be in the form of an aqueous solution comprising an aqueous vehicle. The aqueous vehicle component may comprise water and at least one pharmaceutically acceptable excipient. Suitable acceptable excipients include those selected from the group consisting of a solubility enhancing agent, chelating agent, preservative, tonicity agent, viscosity/suspending agent, buffer, and pH modifying agent, and a mixture thereof.

Any suitable solubility enhancing agent can be used. Examples of a solubility enhancing agent include cyclodextrin, such as those selected from the group consisting of hydroxypropyl-β-cyclodextrin, methyl-β-cyclodextrin, randomly methylated-β-cyclodextrin, ethylated-β-cyclodextrin, triacetyl-β-cyclodextrin, peracetylated-β-cyclodextrin, carboxymethyl-β-cyclodextrin, hydroxyethyl-β-cyclodextrin, 2-hydroxy-3-(trimethylammonio) propyl-β-cyclodextrin, glucosyl-β-cyclodextrin, sulfated β-cyclodextrin (S-β-CD), maltosyl-β-cyclodextrin, β-cyclodextrin sulfobutyl ether, branched-β-cyclodextrin, hydroxypropyl-γ-cyclodextrin, randomly methylated-γ-cyclodextrin, and trimethyl-γ-cyclodextrin, and mixtures thereof.

Any suitable chelating agent can be used. Examples of a suitable chelating agent include those selected from the group consisting of ethylenediaminetetraacetic acid and metal salts thereof, disodium edetate, trisodium edetate, and tetrasodium edetate, and mixtures thereof.

Any suitable preservative can be used. Examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, phenylmercury acetate, phenylmercury neodecanoate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

In some embodiments, examples of a preservative include those selected from the group consisting of quaternary ammonium salts such as benzalkonium halides (preferably benzalkonium chloride), chlorhexidine gluconate, benzethonium chloride, cetyl pyridinium chloride, benzyl bromide, phenylmercury nitrate, merthiolate, methylparaben, propylparaben, sorbic acid, potassium sorbate, sodium benzoate, sodium propionate, ethyl p-hydroxybenzoate, propylaminopropyl biguanide, and butyl-p-hydroxybenzoate, and sorbic acid, and mixtures thereof.

The aqueous vehicle may also include a tonicity agent to adjust the tonicity (osmotic pressure). The tonicity agent can be selected from the group consisting of a glycol (such as propylene glycol, diethylene glycol, triethylene glycol), glycerol, dextrose, glycerin, mannitol, potassium chloride, and sodium chloride, and a mixture thereof. In some embodiments, the tonicity agent is selected from the group consisting of a glycol (such as propylene glycol, triethylene glycol), glycerol, dextrose, glycerin, mannitol, potassium chloride, and sodium chloride, and a mixture thereof.

The aqueous vehicle may also contain a viscosity/suspending agent. Suitable viscosity/suspending agents include those selected from the group consisting of cellulose derivatives, such as methyl cellulose, ethyl cellulose, hydroxyethylcellulose, polyethylene glycols (such as polyethylene glycol 300, polyethylene glycol 400), carboxymethyl cellulose, hydroxypropylmethyl cellulose, and cross-linked acrylic acid polymers (carbomers), such as polymers of acrylic acid cross-linked with polyalkenyl ethers or divinyl glycol (Carbopols-such as Carbopol 934, Carbopol 934P, Carbopol 971, Carbopol 974 and Carbopol 974P), and a mixture thereof.

In order to adjust the formulation to an acceptable pH (typically a pH range of about 5.0 to about 9.0, more preferably about 5.5 to about 8.5, particularly about 6.0 to about 8.5, about 7.0 to about 8.5, about 7.2 to about 7.7, about 7.1 to about 7.9, or about 7.5 to about 8.0), the formulation may contain a pH modifying agent. The pH modifying agent is typically a mineral acid or metal hydroxide base, selected from the group of potassium hydroxide, sodium hydroxide, and hydrochloric acid, and mixtures thereof, and preferably sodium hydroxide and/or hydrochloric acid. These acidic and/or basic pH modifying agents are added to adjust the formulation to the target acceptable pH range. Hence it may not be necessary to use both acid and base-depending on the formulation, the addition of one of the acid or base may be sufficient to bring the mixture to the desired pH range.

The aqueous vehicle may also contain a buffering agent to stabilize the pH. When used, the buffer is selected from the group consisting of a phosphate buffer (such as sodium dihydrogen phosphate and disodium hydrogen phosphate), a borate buffer (such as boric acid, or salts thereof including disodium tetraborate), a citrate buffer (such as citric acid, or salts thereof including sodium citrate), and e-aminocaproic acid, and mixtures thereof.

The formulation may further comprise a wetting agent. Suitable classes of wetting agents include those selected from the group consisting of polyoxypropylene-polyoxyethylene block copolymers (poloxamers), polyethoxylated ethers of castor oils, polyoxyethylenated sorbitan esters (polysorbates), polymers of oxyethylated octyl phenol (Tyloxapol), polyoxyl 40 stearate, fatty acid glycol esters, fatty acid glyceryl esters, sucrose fatty esters, and polyoxyethylene fatty esters, and mixtures thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

According to a further aspect of the disclosure there is provided a pharmaceutical composition which comprises a compound of the disclosure as defined hereinbefore, or a pharmaceutically acceptable salt, hydrate or solvate thereof, in association with a pharmaceutically acceptable diluent or carrier.

In some embodiments, a pharmaceutical composition described herein may further comprise one or more additional pharmaceutically active agents.

The compositions of the disclosure may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the disclosure may be obtained by conventional procedures using conventional pharmaceutical excipients, well known in the art. Thus, compositions intended for oral use may contain, for example, one or more coloring, sweetening, flavoring and/or preservative agents.

A therapeutically effective amount of a compound of the present disclosure for use in therapy is an amount sufficient to treat or prevent a MLL related condition referred to herein, slow its progression and/or reduce the symptoms associated with the condition.

A therapeutically effective amount of a compound of the present disclosure for use in therapy is an amount sufficient to treat an MLL related condition referred to herein, slow its progression and/or reduce the symptoms associated with the condition.

The size of the dose for therapeutic or prophylactic purposes of a compound of Formula (I) will naturally vary according to the nature and severity of the conditions, the age and sex of the animal or subject and the route of administration, according to well-known principles of medicine.

### Methods of Use

In some aspects, the present disclosure provides a method of inhibiting the interaction of menin with MLL (e.g., in vitro or in vivo), comprising contacting a cell with a therapeutically effective amount of a compound of the present disclosure or a pharmaceutically acceptable salt thereof.

In some aspects, the present disclosure provides a method of treating or preventing a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a disease or disorder disclosed herein in a subject in need thereof, comprising administering to the subject a therapeutically effective amount of a compound of the present disclosure or a pharmaceutically acceptable salt thereof, or a pharmaceutical composition of the present disclosure.

In some embodiments, the disease or disorder is associated with MLL. In some embodiments, the disease or disorder is a disease or disorder in which menin-MLL binding is implicated.

The compounds of the invention are inhibitors of the interaction of menin with MLL and MLL fusion proteins. In some embodiments, the present invention is directed to a method of inhibiting the interaction between menin and MLL or an MLL fusion protein by contacting menin and MLL or the MLL fusion protein with a compound of the invention. The contacting can be carried out in vitro or in vivo. In some embodiments, the compounds of the invention can bind to menin, thereby interfering with the binding of MLL to menin. In some embodiments, the present invention provides a method of inhibiting the activity of menin by contacting menin with a compound of the invention in the presence of MLL or an MLL fusion protein. In further embodiments, the present invention provides a method of inhibiting the binding of MLL or an MLL fusion protein to menin, comprising contacting menin with a compound of the invention in the presence of the MLL or MLL fusion protein.

The compounds of the invention are also useful in treating diseases associated with the menin-MLL interaction or menin-MLL fusion protein interaction. For example, diseases and conditions treatable according to the methods of the invention include cancer, such as leukemia, and other diseases or disorders mediated by the menin-MLL interaction or menin-MLL fusion protein interaction such as diabetes.

In some embodiments, the disease or disorder is selected from the group consisting of a leukemia, hematologic malignancy, solid tumor cancer, prostate cancer, breast cancer, liver cancer, brain tumor, and diabetes. In some embodiments, the leukemia is selected from the group consisting of AML, ALL, Mixed Lineage Leukemia, and a leukemia with Partial Tandem Duplications of MLL.

In some embodiments, the disease or disorder is a cancer.

In some embodiments, the cancer is selected from hematological cancer (e.g., leukemia and lymphoma), bladder cancer, brain cancer (e.g., glioma), diffuse intrinsic pontine glioma (DIPG)), breast cancer (e.g., triple-negative breast cancer), colorectal cancer, cervical cancer, gastrointestinal cancer (e.g., colorectal carcinoma, gastric cancer), genitourinary cancer, head and neck cancer, liver cancer, lung cancer, melanoma, ovarian cancer, pancreatic cancer, prostate cancer, renal cancer (e.g., renal cell carcinoma), skin cancer, thyroid cancer (e.g., papillary thyroid carcinoma), testicular cancer, sarcoma (e.g., Ewing's sarcoma), and AIDS-related cancers. In some embodiments, cancer is selected from cardiac cancers, such as for example, sarcoma (e.g., angiosarcoma, fibrosarcoma, rhabdomyosarcoma, and liposarcoma), myxoma, rhabdomyoma, fibroma, lipoma and teratoma; lung cancers, including, for example, bronchogenic carcinoma (e.g., squamous cell, undifferentiated small cell, undifferentiated large cell, and adenocarcinoma), alveolar and bronchiolar carcinoma, bronchial adenoma, sarcoma, lymphoma, chondromatous hamartoma, mesothelioma, non-small cell lung cancer, small cell lung cancer, bronchial adenomas/carcinoids, and pleuropulmonary blastoma; gastrointestinal cancer, including, for example, cancers of the esophagus (e.g., squamous cell carcinoma, adenocarcinoma, leiomyosarcoma, and lymphoma), cancers of the stomach (e.g., carcinoma, lymphoma, and leiomyosarcoma), cancers of the pancreas (e.g., ductal adenocarcinoma, insulinoma, glucagonoma, gastrinoma, carcinoid tumors, and vipoma), cancers of the small bowel (e.g., adenocarcinoma, lymphoma, carcinoid tumors, Kaposi's sarcoma, leiomyoma, hemangioma, lipoma, neurofibroma, and fibroma), cancers of the large bowel or colon, (e.g., adenocarcinoma, tubular adenoma, villous adenoma, hamartoma, and leiomyoma), and other cancers of the digestive tract (e.g., anal cancer, anorectal cancer, appendix cancer, cancer of the anal canal, cancer of the tongue, gallbladder cancer, gastrointestinal stromal tumor (GIST), colon cancer, colorectal cancer, extrahepatic bile duct cancer, intrahepatic bile duct cancer, rectal cancer, and small intestine cancer); genitourinary tract cancers, including, for example, cancers of the kidney (e.g., adenocarcinoma, Wilm's tumor (nephroblastoma), lymphoma, and leukemia), cancers of the bladder and urethra (e.g., squamous cell carcinoma, transitional cell carcinoma, and adenocarcinoma), cancers of the prostate (e.g., adenocarcinoma and sarcoma), cancers of the testis, (e.g., seminoma, teratoma, embryonal carcinoma, teratocarcinoma, choriocarcinoma, sarcoma, interstitial cell carcinoma, fibroma, fibroadenoma, adenomatoid tumors, and lipoma), as well as transitional cell cancer, transitional cell cancer of the renal pelvis and ureter and other urinary organs, urethral cancer, and urinary bladder cancer; liver cancers, including, for example, hepatoma (e.g., hepatocellular carcinoma), cholangiocarcinoma, hepatoblastoma, angiosarcoma, hepatocellular adenoma, and hemangioma; bone cancers, including, for example, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochrondroma (osteocartilaginous exostoses), benign chondroma, chondroblastoma, chondromyxofibroma, osteoid osteoma and giant cell tumors; nervous system cancers, including, for example, cancers of the skull (e.g., osteoma, hemangioma, granuloma, xanthoma, and osteitis deformans); cancers of the meninges (e.g., meningioma, meningiosarcoma, and gliomatosis); cancers of the brain (e.g., astrocytoma, medulloblastoma, glioma, ependymoma, germinoma (pinealoma), glioblastoma multiforme, oligodendroglioma, schwannoma, retinoblastoma, and congenital tumors); cancers of the spinal cord (e.g., neurofibroma, meningioma, glioma, and sarcoma), and other nervous system cancers (e.g., brain stem glioma, diffuse intrinsic pontine glioma (DIPG), brain tumor, central nervous system cancer, cerebellar astrocytoma, cerebral astrocytoma/malignant glioma, childhood cerebellar astrocytoma, childhood cerebral astrocytoma, primary central nervous system lymphoma, visual pathway and hypothalamic glioma, nervous system lymphoma, supratentorial primitive neuroectodeimal tumors, pincoblastoma and supratentorial primitive neuroectodermal tumors); gynecological cancers, including, for example, cancers of the uterus (e.g., endometrial carcinoma), cancers of the cervix (e.g., cervical carcinoma, and pre tumor cervical dysplasia), cancers of the ovaries (e.g., ovarian carcinoma, including serous cystadenocarcinoma, mucinous cystadenocarcinoma, unclassified carcinoma, granulosa thecal cell tumors, Sertoli Leydig cell tumors, dysgerminoma, and malignant teratoma), cancers of the vulva (e.g., squamous cell carcinoma, intraepithelial carcinoma, adenocarcinoma, fibrosarcoma, and melanoma), cancers of the vagina (e.g., clear cell carcinoma, squamous cell carcinoma, botryoid sarcoma, and embryonal rhabdomyosarcoma), and cancers of the fallopian tubes (e.g., carcinoma); other reproductive tract cancers, including, for example, endometrial cancer, endometrial uterine cancer, germ cell tumor, gestational trophoblastic tumor, gestational trophoblastic tumor glioma, ovarian epithelial cancer, ovarian germ cell tumor, ovarian low malignant potential tumor, penile cancer, vaginal cancer, vulvar cancer, extracranial germ cell tumor, extragonadal germ cell tumor, uterine cancer, uterine corpus cancer, uterine sarcoma; lymphatic and hematologic cancers, including, for example, cancers of the blood (e.g., acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, and myelodysplastic syndrome, Hodgkin's lymphoma, non-Hodgkin's lymphoma (malignant lymphoma) and Waldenstrom's macroglobulinemia), and other lymphatic or hematologic cancers including, for example, childhood leukemia, myeloproliferative disorders (e.g., primary myelofibrosis), plasma cell neoplasm/multiple myeloma, myelodysplasia, myelodysplastic syndrome, cutaneous T-cell lymphoma, lymphoid neoplasm, AIDS-related lymphoma, thymoma, thymoma and thymic carcinoma, mycosis fungoides, and Sezary Syndrome; skin cancers, including, for example, malignant melanoma, basal cell carcinoma, squamous cell carcinoma, Kaposi's sarcoma, moles dysplastic nevi, lipoma, angioma, dermatofibroma, keloids, psoriasis, merkel cell carcinoma, merkel cell skin carcinoma, melanoma, and carcinoid tumor; adrenal gland cancers, including, for example, neuroblastoma; other cancers associated with the endocrine system including, for example, adrenocortical carcinoma, multiple endocrine neoplasia (e.g., multiple endocrine neoplasia type I), multiple endocrine neoplasia syndrome, parathyroid cancer, pituitary tumor, pheochromocytoma, islet cell pancreatic cancer, and islet cell tumors); connective tissue cancer (e.g., bone cancer, bone and joint cancer, osteosarcoma and malignant fibrous histiocytoma); cancer associated with the head, neck, and mouth (e.g., head and neck cancer, paranasal sinus and nasal cavity cancer, metastatic squamous neck cancer, mouth cancer, throat cancer, esophageal cancer, laryngeal cancer, pharyngeal cancer, hypopharyngeal cancer, lip and oral cavity cancer, nasopharyngeal cancer, oral cancer, oropharyngeal cancer, and salivary gland cancer); and cancer associated with the eye (e.g., ocular cancer, intraocular melanoma). In some embodiments, the cancer is Ewing's sarcoma.

In some embodiments, the cancer is a hematological cancer such as leukemia or lymphoma. Example leukemia and lymphomas treatable by the compounds of the invention include mixed lineage leukemia (MLL), MLL-related leukemia, MLL-associated leukemia, MLL-positive leukemia, MLL-induced leukemia, rearranged mixed lineage leukemia (MLL-r), leukemia associated with a MLL rearrangement or a rearrangement of the LJ gene, acute leukemia, chronic leukemia, indolent leukemia, lymphoblastic leukemia, lymphocytic leukemia, myeloid leukemia, myelogenous leukemia, childhood leukemia, acute lymphocytic leukemia (ALL) (also referred to as acute lymphoblastic leukemia or acute lymphoid leukemia), acute myeloid leukemia (AML) (also referred to as acute myelogenous leukemia or acute myeloblastic leukemia), acute granulocytic leukemia, acute nonlymphocytic leukemia, chronic lymphocytic leukemia (CLL) (also referred to as chronic lymphoblastic leukemia), chronic myelogenous leukemia (CML) (also referred to as chronic myeloid leukemia), therapy related leukemia, myelodysplastic syndrome (MDS), myeloproliferative disease (MPD) (such as primary myelofibrosis (PMF)), myeloproliferative neoplasia (MPN), plasma cell neoplasm, multiple myeloma, myelodysplasia, cutaneous T-cell lymphoma, lymphoid neoplasm, AIDS-related lymphoma, thymoma, thymic carcinoma, mycosis fungoides, Alibert-Bazin syndrome, granuloma fungoides, Sezary Syndrome, hairy cell leukemia, T-cell prolymphocytic leukemia (T-PLL), large granular lymphocytic leukemia, meningeal leukemia, leukemic leptomeningitis, leukemic meningitis, multiple myeloma, Hodgkin's lymphoma, non-Hodgkin's lymphoma (malignant lymphoma), and Waldenstrom's macroglobulinemia.

In some embodiments, diseases and conditions treatable with compounds of the invention include insulin resistance, pre-diabetes, diabetes (e.g., Type 2 diabetes or Type 1 diabetes), and risk of diabetes. In some embodiments, diseases and conditions treatable with compounds of the invention include hyperglycemia. In some embodiments, the hyperglycemia is associated with diabetes, such as Type 2 diabetes. In some embodiments, compounds of the invention are used to treat loss of response to other anti-diabetic agents and/or reduced beta cell function in a patient or subject. In some embodiments, compounds of the invention are used to restore response to other anti-diabetic agents and/or to restore beta cell function and/or to reduce the need for insulin in a patient or subject. In some embodiments, compounds of the invention are used to reduce insulin resistance, reduce the risk of diabetes, or reduce increases in blood glucose caused by a statin in a subject taking a statin. In some embodiments, compounds of the invention are used to treat diabetes in a subject taking a statin or to prevent diabetes in a subject taking a statin. Methods of the invention include decreasing, reducing, inhibiting, suppressing, limiting or controlling in the subject elevated blood glucose levels. In further aspects, methods of the invention include increasing, stimulating, enhancing, promoting, inducing or activating in the subject insulin sensitivity. Statins include, but are not limited to atorvastatin, cerivastatin, fluvastatin, lovastatin, mevastatin, pitavastatin, pravastatin, rousuvastatin and simvastatin.

In some aspects, the present disclosure provides a method of treating or preventing a cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a method of treating a cancer in a subject in need thereof, comprising administering to the subject a therapeutically effective amount of a compound of the present disclosure or a pharmaceutically acceptable salt thereof, or a pharmaceutical composition of the present disclosure.

In some aspects, the present disclosure provides a compound of the present disclosure or a pharmaceutically acceptable salt thereof for use in inhibiting the interaction of menin with MLL (e.g., in vitro or in vivo).

In some aspects, the present disclosure provides a compound of the present disclosure or a pharmaceutically acceptable salt thereof for use in treating or preventing a disease or disorder disclosed herein.

In some aspects, the present disclosure provides a compound of the present disclosure or a pharmaceutically acceptable salt thereof for use in treating a disease or disorder disclosed herein.

In some aspects, the present disclosure provides a compound of the present disclosure or a pharmaceutically acceptable salt thereof for use in treating or preventing a cancer in a subject in need thereof.

In some aspects, the present disclosure provides a compound of the present disclosure or a pharmaceutically acceptable salt thereof for use in treating a cancer in a subject in need thereof.

In some aspects, the present disclosure provides use of a compound of the present disclosure or a pharmaceutically acceptable salt thereof in the manufacture of a medicament for inhibiting the interaction of menin with MLL (e.g., in vitro or in vivo).

In some aspects, the present disclosure provides use of a compound of the present disclosure or a pharmaceutically acceptable salt thereof in the manufacture of a medicament for treating or preventing a disease or disorder disclosed herein.

In some aspects, the present disclosure provides use of a compound of the present disclosure or a pharmaceutically acceptable salt thereof in the manufacture of a medicament for treating a disease or disorder disclosed herein.

In some aspects, the present disclosure provides use of a compound of the present disclosure or a pharmaceutically acceptable salt thereof in the manufacture of a medicament for treating or preventing a cancer in a subject in need thereof.

In some aspects, the present disclosure provides use of a compound of the present disclosure or a pharmaceutically acceptable salt thereof in the manufacture of a medicament for treating a cancer in a subject in need thereof.

The present disclosure provides compounds that function as inhibitors of the interaction of menin with MLL (e.g., in vitro or in vivo). The present disclosure therefore provides a method of inhibiting the interaction of menin with MLL in vitro or in vivo, said method comprising contacting a cell with a therapeutically effective amount of a compound, or a pharmaceutically acceptable salt thereof, as defined herein.

In some embodiments, the menin/MLL interaction inhibitor is a compound of the present disclosure.

Effectiveness of compounds of the disclosure can be determined by industry-accepted assays/disease models according to standard practices of elucidating the same as described in the art and are found in the current general knowledge.

The present disclosure also provides a method of treating a disease or disorder in which interaction of menin with MLL is implicated in a subject in need of such treatment, said method comprising administering to said subject a therapeutically effective amount of a compound, or a pharmaceutically acceptable salt thereof, or a pharmaceutical composition as defined herein.

In some embodiments, the subject is a mammal. In some embodiments, the subject is a human.

### Routes of Administration

The compounds of the disclosure or pharmaceutical compositions comprising these compounds may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

Abbreviations used in the following examples and elsewhere herein are:
AcOH acetic acid
aq. aqueous
Boc₂O di-tert-butyl dicarbonate
br. broad
BSA bovine serum albumin
d duplet
DCM dichloromethane
DIPEA N,N-diisopropylethylamine
DMAA 1,3-dimethylamylamine
DMAP 4-(dimethylamino) pyridine
DMEM Dulbecco's modified Eagle's medium
DMF N,N-dimethyl formamide
DMSO dimethyl sulfoxide
DTT dithiothreitol
FBS fetal bovine serum
h hour(s)
HATU (1-[bis(dimethylamino)methylene]-1H-1,2,3-triazolo[4,5-b]pyridinium 3-oxide hexafluorophosphate
HPLC high pressure (or performance) liquid chromatography
LAH lithium aluminum hydride
LCMS liquid chromatography mass spectrometry
m multiplet
M molar
MHz megahertz
min minutes
Ms Mesyl
NBS N-bromosuccinimide
NMR nuclear magnetic resonance
Pd₂(dba)₃ tris(dibenzylideneacetone)dipalladium(0)
Pd(dppf)Cl₂ [1,1′-bis(diphenylphosphino)ferrocene]dichloropalladium(II)
q quadruplet
rt room temperature
S singlet
sat. saturated
STAB sodium triacetoxyborohydride
t temperature, triplet
T3P Propylphosphonic anhydride
TEA Triethyl amine
TFA trifluoroacetic acid
THF tetrahydrofuran
TLC thin layer chromatography
TRIS 2-amino-2-(hydroxymethyl) propane-1,3-diol
TsOH toluenesulfonic acid (e.g., p-TsOH)
Xantphos 4,5-Bis(diphenylphosphino)-9,9-dimethylxanthene

### EXAMPLES

### General Synthetical Procedures and Examples of the Compound's Preparation.

### Synthesis of Building Blocks

### Synthesis of 4-Chloro-6-(2,2,2-trifluoroethyl)quinazoline (P7)

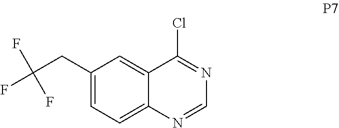 [SMILES: FC(F)(F)Cc1ccc2ncnc(Cl)c2c1]

### Preparation 1. Methyl 5-bromo-2-[(tert-butoxycarbonyl)amino]benzoate (P1)

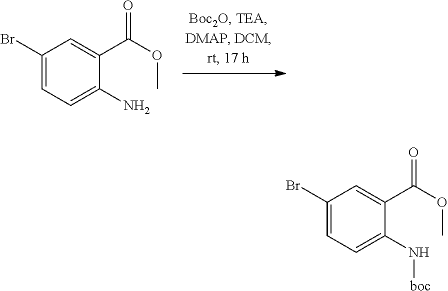

To solution of methyl 2-amino-5-bromobenzoate (25 g, 0.109 mol) in DCM (250 ml) was added Boc₂O (26.5 g, 0.122 mol), triethylamine (62 g, 0.61 mol) and DMAP (4 g, 0.033 mol). The solution was stirred at ambient temperature for 17 h. Then 250 ml of water was added, and potassium hydrosulfate was added in small portions with stirring until pH reached 3. Water phase was washed with dichloromethane (200 ml), organic solution was concentrated, and residue was purified using silica gel column chromatography (30% dichloromethane in hexane). 5-Bromo-2-[(tert-butoxycarbonyl)amino]benzoate (P1) was isolated as white solid (14.3 g, 40%). ¹H NMR (400 MHZ, DMSO-d₆), δ: 10.02 (s, 1H), 8.10 (d, J=9.0 Hz, 1H), 7.98 (d, J=2.4 Hz, 1H), 7.76 (d, J=9.0, 2.4 Hz, 1H), 3.84 (s, 3H), 1.43 (s, 9H).

### Preparation 2. Methyl 2-[(tert-butoxycarbonyl)amino]-5-(4,4,5,5-tetramethyl-1,3,2-dioxaborolan-2-yl)benzoate (P2)

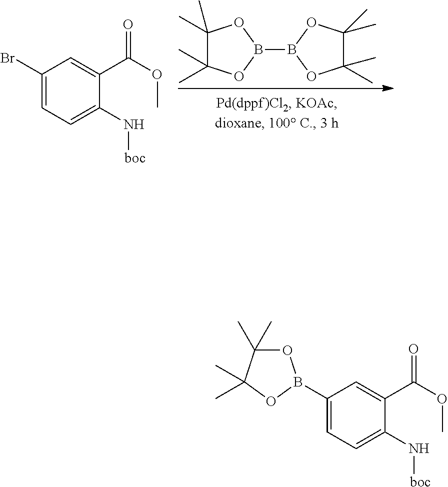

5-Bromo-2-[(tert-butoxycarbonyl)amino]benzoate (P1, g, 0.043 mol) and bis(pinacolato)diboron (22 g, 0.087 mol) were dissolved in dioxane (120 ml). Potassium acetate (12.6 g, 0.129 mol) was added to the solution. The mixture was stirred for 30 min at 70° C. under argon atmosphere. Then Pd(dppf)Cl₂ (3.2 g) was added, and reaction mixture was stirred at 100° C. for additional 3 h (TLC control). The mixture was cooled down to rt and filtered through a pad of celite. Filtrate was then concentrated, and residue was purified by silica gel column chromatography (dichloromethane) to give the product (P2) as a white solid (16 g, 99% yield). ¹H NMR (400 MHz, DMSO-d₆), δ: 10.31 (s, 1H), 8.26 (d, J=8.8 Hz, 2H), 7.83 (d, J=8.4 Hz, 1H), 3.87 (s, 3H), 1.48 (s, 9H), 1.29 (s, 12H).

### Preparation 3. Methyl 2-[(tert-butoxycarbonyl)amino]-5-(2,2,2-trifluoroethyl)benzoate (P3)

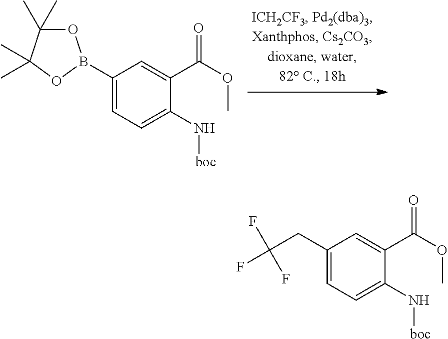

Methyl 2-[(tert-butoxycarbonyl)amino]-5-(4,4,5,5-tetramethyl-1,3,2-dioxaborolan-2-yl)benzoate (P2, 11.2 g, 30 mmol) was dissolved in dioxane (100 ml), cesium carbonate (32.6 g, 118 mmol) in water (8 ml) was added. The mixture was stirred 30 min at 70° C. under argon atmosphere. Then 1,1,1-trifluoro-2-iodo-ethane (18.6 g, 89 mmol), Pd₂(dba)₃ (2.6 g), and Xantphos (2.6 g) were added. The reaction mixture was stirred at 82° C. for 18 h, then the mixture was cooled down and filtered through a pad of celite. The filtrate was concentrated. The residue was purified by silica gel column chromatography (dichloromethane-hexane-1:1) to give the product (P3) as a white solid (5.85 g, 59% yield). ¹H NMR (400 MHZ, DMSO-d₆), δ: 10.10 (s, 1H), 8.20 (t, J=20.8 Hz, 1H), 7.92 (s, 1H), 7.50 (d, J=23.8 Hz, 1H), 3.86 (s, 3H), 3.74-3.48 (m, 2H), 1.48 (s, 9H).

### Preparation 4. Methyl 2-amino-5-(2,2,2-trifluoroethyl)benzoate (P4)

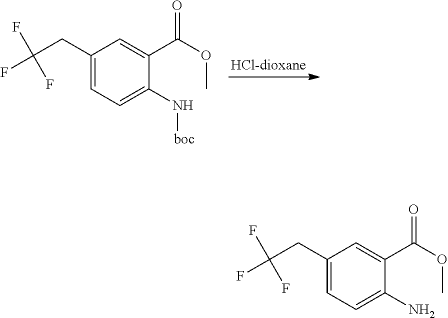

3M Solution of HCl in dioxane was added to methyl 2-[(tert-butoxycarbonyl)amino]-5-(2,2,2-trifluoroethyl)benzoate (P3, 5.85 g, 17.6 mmol). Reaction mixture was stirred at rt for 2 h (NMR control) and concentrated to dryness to give the product (P4, 4.6 g, 100%) as hydrochloride. ¹H NMR (400 MHZ, DMSO-d₆), δ: 7.62-7.70 (m, 1H), 7.19-7.27 (m, 1H), 6.86-6.84 (m, 1H), 3.79 (s, 3H), 4.03-3.58 (m, 2H).

### Preparation 5. 2-Amino-5-(2,2,2-trifluoroethyl)benzoic acid (P5)

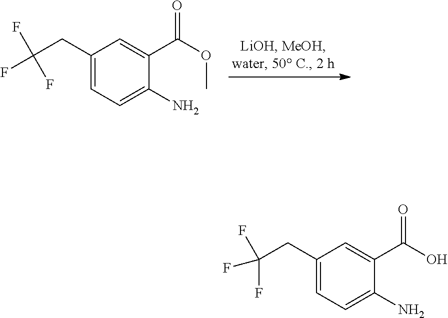 [SMILES: COC(=O)c1cc(CC(F)(F)F)ccc1N ; Nc1ccc(CC(F)(F)F)cc1C(=O)O]

Lithium hydroxide (2 g, 83 mmol) in water (35 ml) was added to the solution of methyl 2-amino-5-(2,2,2-trifluoroethyl)benzoate (P4, 4.6 g, 17.6 mmol) in methanol (100 ml). The reaction mixture was stirred at 50° C. for 2 h. Then reaction mixture was cooled, concentrated to dryness, and water (50 ml) was added. Concentrated HCl was added to the obtained solution with stirring and cooling with cold water up to pH=3. Obtained precipitate was filtered off, washed with water (15 ml) and dried to give the product P5 as a white solid (3 g, 78%). ¹H NMR (400 MHz, DMSO-d₆), δ: 7.62-7,70 (s, 1H), 7.23 (d, J=8.3 Hz, 1H), 6.81 (d, J=8.1 Hz, 1H), 3.55-3.33 (m, 2H).

### Preparation 6. 6-(2,2,2-Trifluoroethyl)quinazolin-4(3H)-one (P6)

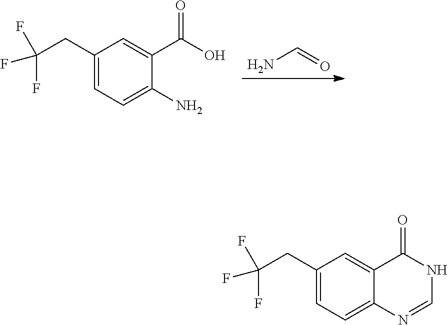

2-Amino-5-(2,2,2-trifluoroethyl)benzoic acid (P5, 3 g, 13.7 mmol) and formamide (2.2 g, 49 mmol) were stirred in vial at 155° C. for 1.5 h, and then at 165° C. for 40 min. The reaction mixture was cooled, saturated solution of sodium bicarbonate (50 ml) was added, and the mixture stirred at rt for 30 min. Formed precipitate was filtered off, washed with water (55 ml) and dried to give the product P6 as a white solid (2.52 g, 85%). ¹H NMR (400 MHZ, DMSO-d₆), δ: 12.30 (s, 1H), 8.4-8,16 (m, 1H), 8,07-8,16 (m, 1H), 7.78 (d, J=8.6 Hz, 1H), 7.68 (d, J=7.8 Hz, 1H), 3.79-3.90 (m, 2H).

### Preparation 7. 4-Chloro-6-(2,2,2-trifluoroethyl)quinazoline (P7)

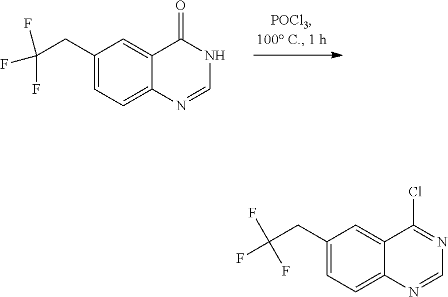 [SMILES: O=c1[nH]cnc2ccc(CC(F)(F)F)cc12 ; FC(F)(F)Cc1ccc2ncnc(Cl)c2c1]

POCl₃ (35 ml) was added to 6-(2,2,2-trifluoroethyl)quinazolin-4(3H)-one (P6, 2.52 g, 11 mmol), the reaction mixture was stirred and refluxed for 1 h. The solution was cooled down and poured to ice-cold water solution of sodium bicarbonate. The mixture was stirred for 30 min, then extracted with dichloromethane (2×50 ml). After evaporation of dichloromethane, residue was purified by silica gel column chromatography (dichloromethane-ethyl acetate-10:1) to give the product (P7, 1.9 g 70%) as a brown solid. ¹H NMR (400 MHZ, CDCl₃), δ: 9.12-9.07 (m, 1H), 8.25-8.22 (m, 1H), 8.12 (d, J=8.3 Hz, 1H), 7.93 (d, J=8.1, Hz, 1H), 3.70-3.60 (m, 2H).

### Synthesis of 4-(2,7-diazaspiro[3.5]non-2-yl)-6-(2,2,2-trifluoroethyl)quinazoline (P9)

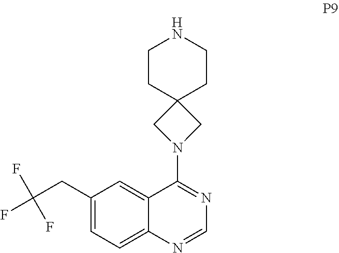 [SMILES: FC(F)(F)Cc1ccc2ncnc(N3CC4(CCNCC4)C3)c2c1]

### Preparation 8. tert-Butyl 2-[6-(2,2,2-trifluoroethyl)quinazolin-4-yl]-2,7-diazaspiro[3.5]nonane-7-carboxylate (P8)

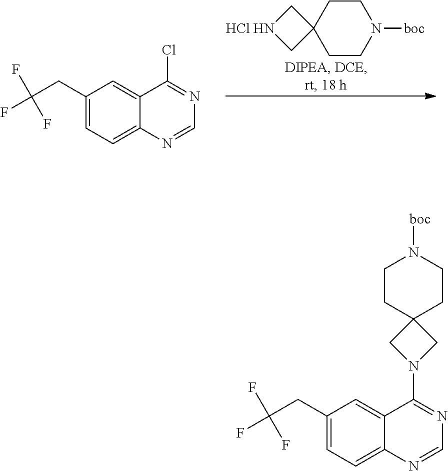

DIPEA (7/9 g, 61 mmol) and tert-butyl 2,7-diazaspiro[3.5]nonane-7-carboxylate hydrochloride (3.2 g, 12.2 mmol) were added to a solution of 4-chloro-6-(2,2,2-trifluoroethyl)quinazoline (P7, 3 g, 12.2 mmol) in dichloroethane (60 ml). The reaction solution was stirred at rt for 18 h (LCMS control). Then product was purified by column chromatography on silica (ethyl acetate-methanol 10:1) to give P8 as a white solid (5.1 g, 96%). ¹H NMR (400 MHZ, DMSO-d₆) δ 8.47-8.43 (m, 1H), 7.99-7.95 (m, 1H), 7.68 (m, 2H), 4.65-3.96 (m, 4H), 3.96-3.75 (m, 2H), 3.48-3.40 (m, 4H), 2.63-2.48 (m, 4H), 1.86-1.63 (m, 4H), 1.41 (s, 9H).

### Preparation 9. 4-(2,7-diazaspiro[3.5]non-2-yl)-6-(2,2,2-trifluoroethyl)quinazoline (P9)

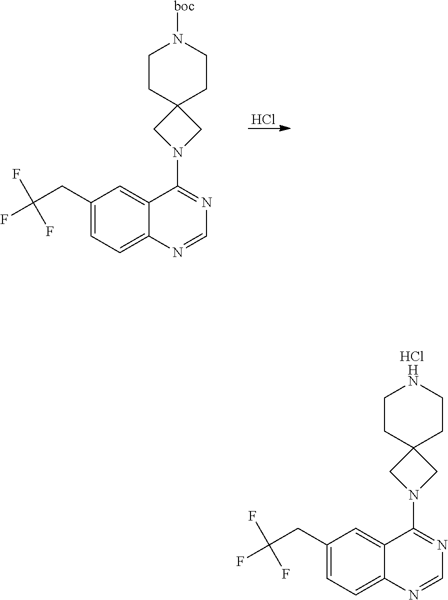

To tert-butyl 2-[6-(2,2,2-trifluoroethyl)quinazolin-4-yl]-2,7-diazaspiro[3.5]nonane-7-carboxylate (P8, 5.1 g, 11.7 mmol) was added dioxane-HCl solution (3 M, 120 ml). The reaction mixture was stirred at rt for 3 h (LCMS control), then evaporated under vacuum to dryness to give 4-(2,7-diazaspiro[3.5]non-2-yl)-6-(2,2,2-trifluoroethyl)quinazoline hydrochloride (P9, 4.4 g, 100%) as a white solid. ¹H NMR (400 MHZ, DMSO-d₆), δ: 9.44 (s, 1H), 9.29 (s, 1H), 8.85-8.80 (m, 1H), 8.14-8.08 (m, 1H), 8.06-7.98 (m, 2H), 4.90-4.76 (m, 2H), 4.38-4.24 (m, 2H), 4.03-3.89 (m, 2H), 3.18-2.97 (m, 4H), 2.20-2.02 (m, 4H).

### Synthesis of [4-({2-[6-(2,2,2-Trifluoroethyl)quinazolin-4-yl]-2,7-diazaspiro[3.5]non-7-yl}methyl)phenyl]amine (P11)

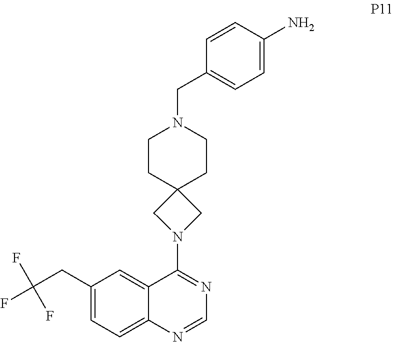 [SMILES: Nc1ccc(CN2CCC3(CC2)CN(c2ncnc4ccc(CC(F)(F)F)cc24)C3)cc1]

### Preparation 10. 4-[7-(4—Nitrobenzyl)-2,7-diazaspiro[3.5]non-2-yl]-6-(2,2,2-trifluoroethyl)quinazoline (P10)

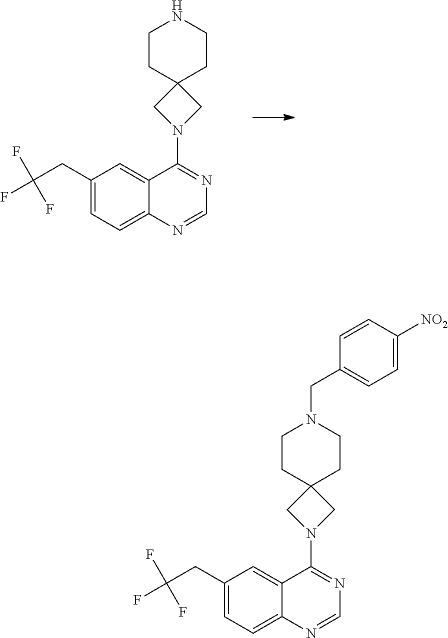 [SMILES: FC(F)(F)Cc1ccc2ncnc(N3CC4(CCNCC4)C3)c2c1 ; O=[N+]([O-])c1ccc(CN2CCC3(CC2)CN(c2ncnc4ccc(CC(F)(F)F)cc24)C3)cc1]

A mixture of 4-(2,7-diazaspiro[3.5]non-2-yl)-6-(2,2,2-trifluoroethyl)quinazoline (P9, 194 mg, 0.55 mmol), 4-nitrobenzaldehyde (165 mg, 0.8 mmol) and sodium triacethoxyborohydride (170 mg, 0.8 mmol) in dichloromethane (2 ml) was stirred at rt for 5 h (LCMS control). After reaction was completed a water solution of sodium bicarbonate was added and the mixture was stirred for additional 30 min. Then the mixture was extracted with DCM (20 ml). Solvent was evaporated and residue was purified by silica column chromatography (ethyl acetate-methanol 95:5) to yield 4-[7-(4-nitrobenzyl)-2,7-diazaspiro[3.5]non-2-yl]-6-(2,2,2-trifluoroethyl)quinazoline (P10, 170 mg, 65%).

### Preparation 11. [4-({2-[6-(2,2,2-Trifluoroethyl)quinazolin-4-yl]-2,7-diazaspiro[3.5]non-7-yl}methyl)phenyl]amine (P11)

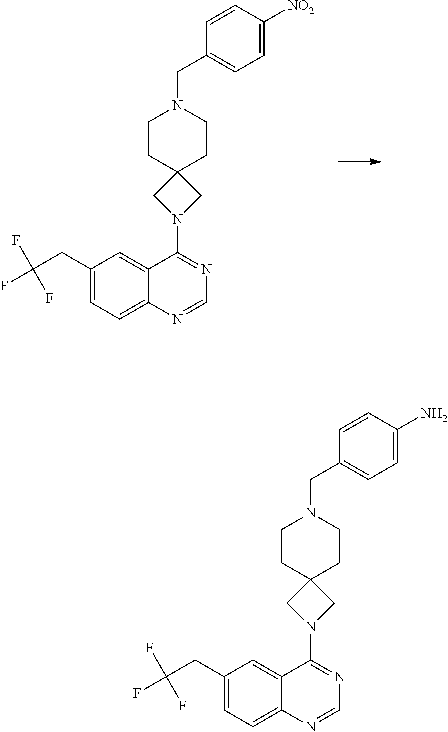 [SMILES: O=[N+]([O-])c1ccc(CN2CCC3(CC2)CN(c2ncnc4ccc(CC(F)(F)F)cc24)C3)cc1 ; Nc1ccc(CN2CCC3(CC2)CN(c2ncnc4ccc(CC(F)(F)F)cc24)C3)cc1]

To the solution of 4-[7-(4-nitrobenzyl)-2,7-diazaspiro[3.5]non-2-yl]-6-(2,2,2-trifluoroethyl)quinazoline (P10, 170 mg, 0.36 mmol) in THF (3 ml) were added acetic acid (220 mg, 3.6 mmol), and Zn dust (400 mg, 7.2 mmol). The mixture was stirred at rt for 18 h (LCMS control). After reaction completed the mixture was filtered from Zn, concentrated, washed with water solution of sodium bicarbonate, and extracted with DCM (2×20 ml). The organic extract was evaporated to dryness to yield the product P11 (159 mg, 100%). LCMS (ESI) [MH]⁺: 442.

### Synthesis of 4-[7-(1,2,3,4-tetrahydroisoquinolin-6-ylmethyl)-2,7-diazaspiro[3.5]non-2-yl]-6-(2,2,2-trifluoroethyl)quinazoline (P36)

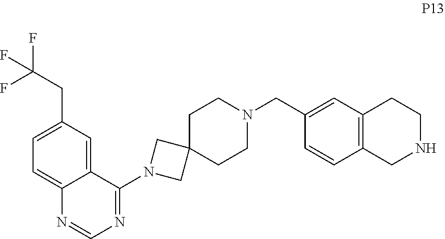 [SMILES: FC(F)(F)Cc1ccc2ncnc(N3CC4(CCN(Cc5ccc6c(c5)CCNC6)CC4)C3)c2c1]

### Preparation 12. 6-(2,2,2-trifluoroethyl)-4-{7-[(2-trityl-1,2,3,4-tetrahydroisoquinolin-6-yl)methyl]-2,7-diazaspiro[3.5]non-2-yl}quinazoline (P12)

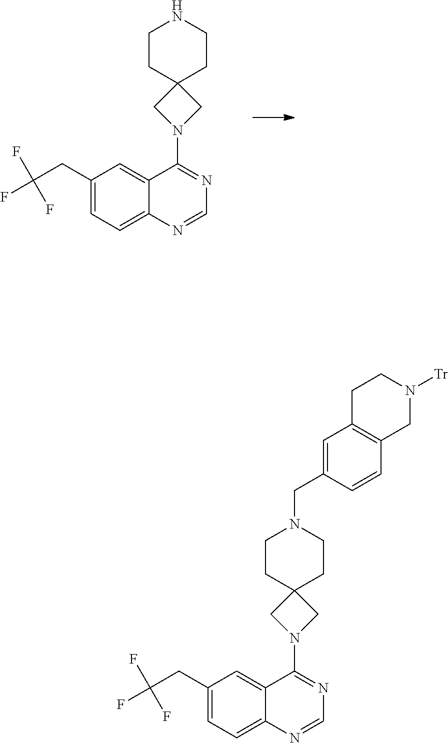

Sodium triacethoxyborohydride (227 mg, 1.07 mmol) was added to the mixture of 4-(2,7-diazaspiro[3.5]non-2-yl)-6-(2,2,2-trifluoroethyl)quinazoline (P9, 60 mg, 0.16 mmol), 2-trityl-1,2,3,4-tetrahydroisoquinoline-6-carbaldehyde P87 (97 mg, 0.24 mmol) in DCM (5 ml) and reaction mixture was stirred at rt for 24 h (LCMS control). Then water solution of sodium bicarbonate was added under vigorous stirring. The mixture was stirred for 30 min, phases were separated, water phase was extracted with DCM (100 ml). Combined DCM solution was concentrated, and residue was purified by column chromatography on silica with ethylacetate-methanol (10:2) to yield the product (P12, 47 mg, 39%). LCMS [MH]⁺: 725.

### Preparation 13. 4-[7-(1,2,3,4-tetrahydroisoquinolin-6-ylmethyl)-2,7-diazaspiro[3.5]non-2-yl]-6-(2,2,2-trifluoroethyl)quinazoline (P13)

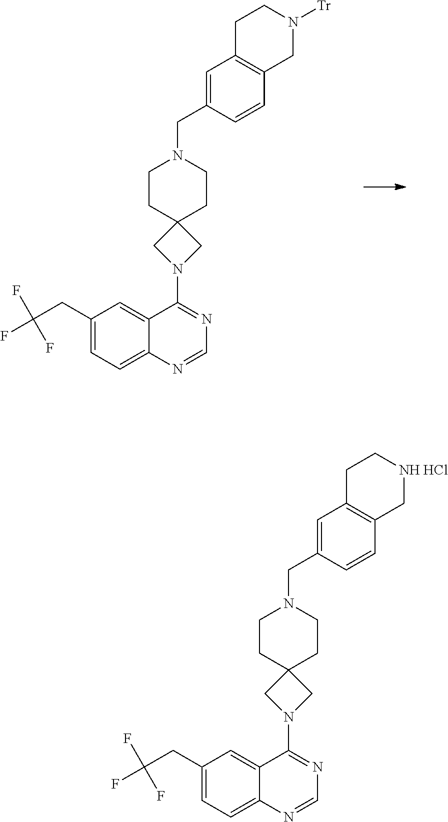

6-(2,2,2-Trifluoroethyl)-4-{7-[(2-trityl-1,2,3,4-tetrahydroisoquinolin-6-yl)methyl]-2,7-diazaspiro[3.5]non-2-yl}quinazoline (P12, 47 mg, 0.62 mmol) was added to HCl in dioxane solution (3M, 1 ml). The reaction mixture was stirred at rt for 3 h (LCMS control), then evaporated under reduce pressure to dryness, and washed with hexane to yield the product P13 (31 mg, 97%). LCMS(ESI) [MH]⁺: 482.

### Synthesis of 4-[7-(2,3-dihydro-1H-isoindol-5-ylmethyl)-2,7-diazaspiro[3.5]non-2-yl]-6-(2,2,2-trifluoroethyl)quinazoline (P15)

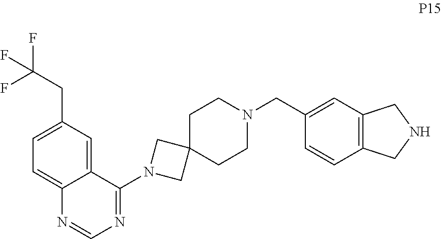 [SMILES: FC(F)(F)Cc1ccc2ncnc(N3CC4(CCN(Cc5ccc6c(c5)CNC6)CC4)C3)c2c1]

### Preparation 14. tert-Butyl 5-({2-[6-(2,2,2-trifluoroethyl)quinazolin-4-yl]-2,7-diazaspiro[3.5]non-7-yl}methyl)-1,3-dihydro-2H-isoindole-2-carboxylate (P14)

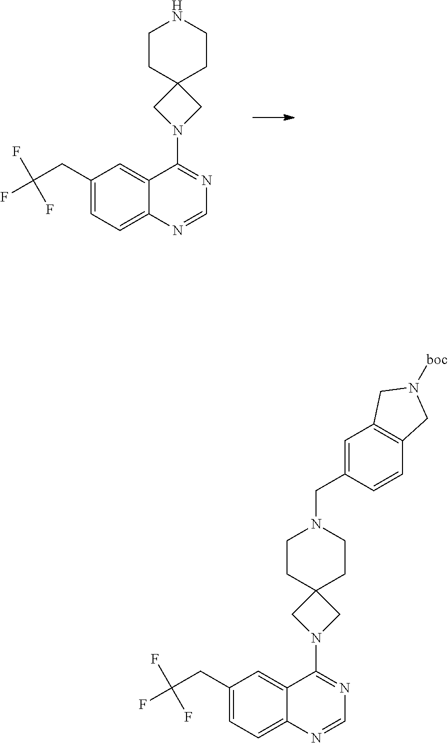

To the mixture of tert-butyl 5-formyl-1,3-dihydro-2H-isoindole-2-carboxylate (P88, 110 mg, 0.44 mmol) and 4-(2,7-diazaspiro[3.5]non-2-yl)-6-(2,2,2-trifluoroethyl)quinazoline (P9, 166 mg, 0.44 mmol) in DCM (5 ml) was added sodium triacethoxyborohydride (227 mg, 1.07 mmol). The mixture was stirred at rt for 24 h (LCMS control). Then water solution of sodium bicarbonate was added under vigorous stirring. The mixture was stirred for 30 min, phases were separated, water phase was extracted with DCM (100 ml). Combined DCM solution was concentrated, and residue was purified by column chromatography on silica with ethylacetate-methanol (10:2) to yield the product P14 (30.8 mg, 12%). LCMS(ESI) [MH]⁺: 568.

### Preparation 15. 4-[7-(2,3-dihydro-1H-isoindol-5-ylmethyl)-2,7-diazaspiro[3.5]non-2-yl]-6-(2,2,2-trifluoroethyl)quinazoline (P15)

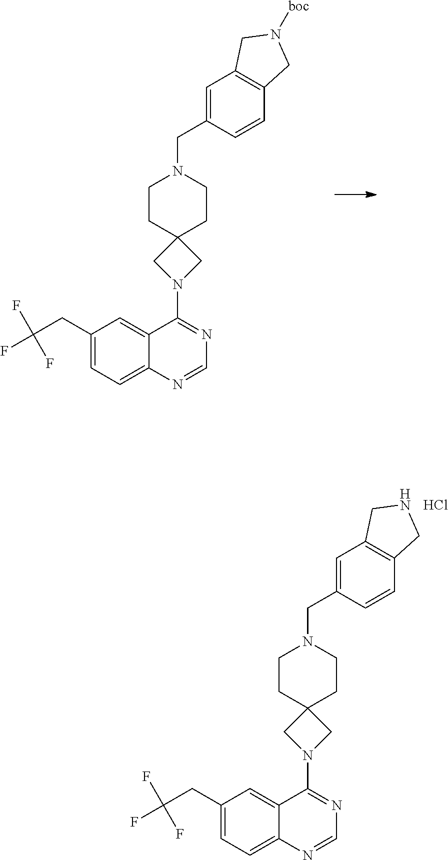

To the HCl/dioxane solution (3M, 1 ml) portionwise was added tert-butyl 5-({2-[6-(2,2,2-trifluoroethyl)quinazolin-4-yl]-2,7-diazaspiro[3.5]non-7-yl}methyl)-1,3-dihydro-2H-isoindole-2-carboxylate (P14, 30.8 mg, 0.054 mmol). The reaction mixture was stirred at rt for 3 h (LCMS control), then evaporated under vacuum to dryness to yield the product (P15, 28 mg, 100%). LCMS(ESI) [MH]⁺: 468.

### Synthesis of 4-(2,7-diazaspiro[3.5]non-7-yl)-6-(2,2,2-trifluoroethyl)quinazoline (P28)

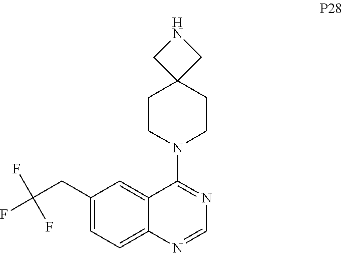 [SMILES: FC(F)(F)Cc1ccc2ncnc(N3CCC4(CC3)CNC4)c2c1]

### Preparation 27. tert-Butyl 7-[6-(2,2,2-trifluoroethyl)quinazolin-4-yl]-2,7-diazaspiro[3.5]nonane-2-carboxylate (P27)

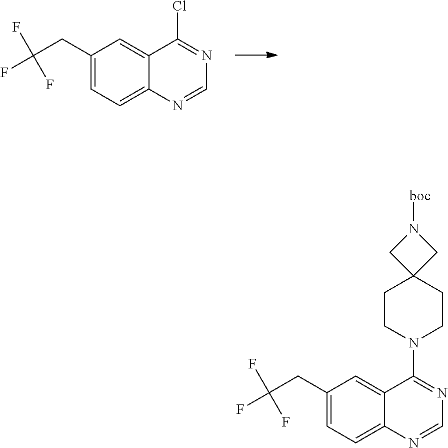

To a solution of 4-chloro-6-(2,2,2-trifluoroethyl)quinazoline (P7, 3 g, 12.2 mmol) in DCM (60 ml) was added DIPEA (7.9 g, 61 mmol) and tert-butyl 2,7-diazaspiro[3.5]nonane-2-carboxylate hydrochloride (3.2 g, 12.2 mmol). The reaction mixture was stirred at rt for 18 h (LCMS control). After completing of the reaction, the solvent was evaporated, and residue was purified by column chromatography on silica with ethylacetate-methanol (10:1) to yield 5.1 g of the product P27 as a white solid. LCMS(ESI) [MH]⁺: 437.

### Preparation 28. 4-(2,7-diazaspiro[3.5]non-7-yl)-6-(2,2,2-trifluoroethyl)quinazoline (P28)

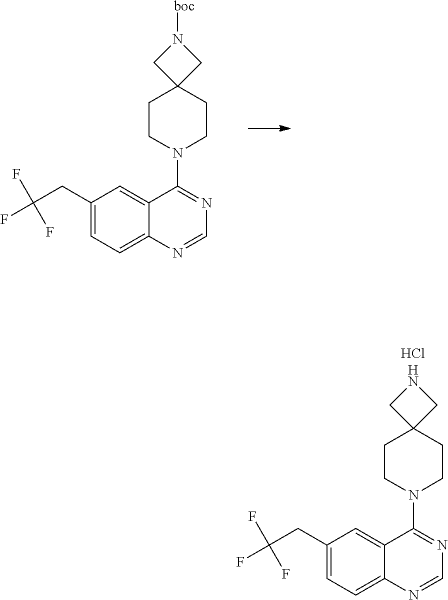

tert-Butyl 7-[6-(2,2,2-trifluoroethyl)quinazolin-4-yl]-2,7-diazaspiro[3.5]nonane-2-carboxylate (P27, 5.1 g, 11.7 mmol) was added to dioxane-HCl solution (3M, 120 ml). The reaction mixture was stirred at rt for 3 h (LCMS control), then evaporated under reduce pressure to dryness to yield 4-(2,7-diazaspiro[3.5]non-7-yl)-6-(2,2,2-trifluoroethyl)quinazoline hydrochloride (P28, 4.4 g, 100%) as a white solid. LCMS(ESI) [MH]⁺: 337.

### Synthesis of 3-[6-(2,2,2-trifluoroethyl)quinazolin-4-yl]-3,9-diazaspiro[5.5]undecane (P30)

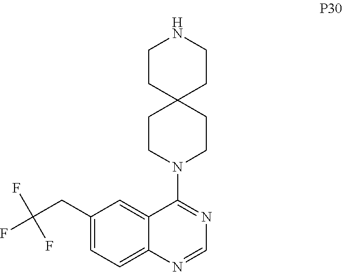 [SMILES: FC(F)(F)Cc1ccc2ncnc(N3CCC4(CCNCC4)CC3)c2c1]

### Preparation 29. tert-Butyl 9-[6-(2,2,2-trifluoroethyl)quinazolin-4-yl]-3,9-diazaspiro[5.5]undecane-3-carboxylate (P29)

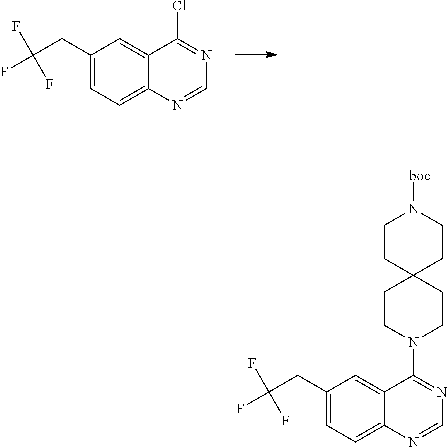

To a solution of 4-chloro-6-(2,2,2-trifluoroethyl)quinazoline (P7, 3 g, 12.2 mmol) in dichloroethane (60 ml) were added DIPEA (7.9 g, 61 mmol), and tert-butyl 3,9-diazaspiro[5.5]undecane-3-carboxylate (3.5 g, 12.2 mmol). The reaction mixture was stirred at rt for 18 h (LCMS control). The residue after solvent evaporation was purified by column chromatography on silica with ethylacetate-methanol (10:1) to yield the product P29 as a white solid (5.1 g, 92%). LCMS(ESI) [MH]⁺: 465.

### Preparation 30. 3-[6-(2,2,2-trifluoroethyl)quinazolin-4-yl]-3,9-diazaspiro[5.5]undecane (P30)

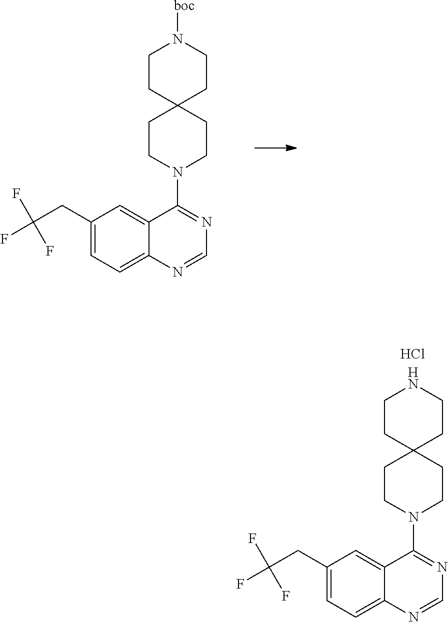

tert-Butyl 9-[6-(2,2,2-trifluoroethyl)quinazolin-4-yl]-3,9-diazaspiro[5.5]undecane-3-carboxylate (P29, 5.1 g, 11.7 mmol) was added to HCl/dioxane solution (3M, 120 ml). The reaction mixture was stirred at rt for 3 h (LCMS control), then solvent was evaporated under reduce pressure to dryness to yield 3-[6-(2,2,2-trifluoroethyl)quinazolin-4-yl]-3,9-diazaspiro[5.5]undecane (P30, 4.4 g, 100%) as a white solid. LCMS(ESI) [MH]⁺: 364.

### Synthesis of 4-(2,7-diazaspiro[3.5]non-2-yl)-6-(2,2,2-trifluoroethyl) pyrido[2,3-d]pyrimidine (P39)

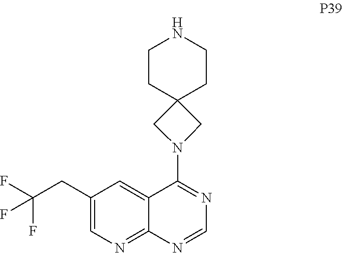 [SMILES: FC(F)(F)Cc1cnc2ncnc(N3CC4(CCNCC4)C3)c2c1]

### Preparation 31. Methyl 2-amino-5-bromonicotinate (P31)

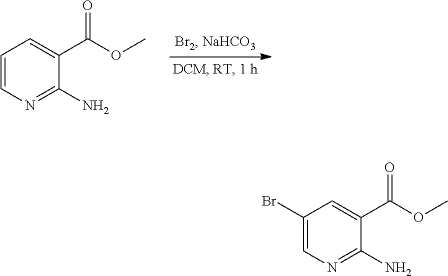

To a stirred solution of methyl 2-aminonicotinate (2 g, 13.15 mmol) and sodium bicarbonate (2.2 g, 26.31 mmol) in DCM (30 ml) was added a solution of bromine (1.01 ml, 39 mmol) in DCM (20 ml) dropwise at 0° C. The reaction mixture was stirred at room temperature for 1 h. The reaction mixture was quenched with sodium bisulfate solution (50 ml) and extracted with DCM (2×40 ml). The combined organic layers are washed with brine (40 ml), dried over sodium sulfate, filtered, and evaporated under reduced pressure to give the title compound (P31) as a yellow solid (2.76 g, 91%). ¹H NMR (400 MHZ, DMSO-d₆), δ: 8.29 (d, J=2.5 Hz, 1H), 8.12 (d, J=2.5 Hz, 1H), 7.32 (s, 2H), 3.82 (s, 3H).

### Preparation 32. 2-Amino-5-bromonicotinic acid (P32)

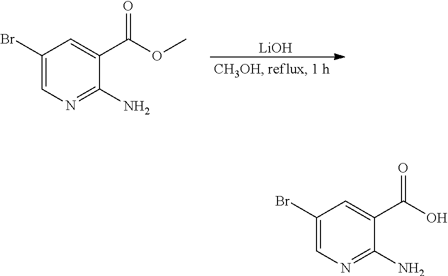

To a solution of P31 (2.76 g, 11.95 mmol) in DCM (100 ml) was added a solution LiOH (1.72 g, 71.7 mmol) in water, the reaction mixture was refluxed for 1 hour, then evaporated under reduced pressure and dissolved in water, pH was adjusted to ˜3 with HCl. The residue was filtered and washed with water to give the title compound (P32) as a white solid (2.05 g, 79%). ¹H NMR (400 MHZ, DMSO-d₆), δ: 13.30 (s, 1H), 8.25 (d, J=2.5 Hz, 1H), 8.09 (d, J=2.5 Hz, 1H), 7.38 (s, 1H).

### Preparation 33. 6-Bromopyrido[2,3-d]pyrimidin-4 (3H)-one (P33)

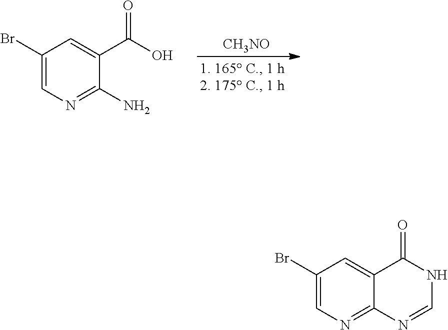

The solution of P32 (2 g, 9.22 mmol) in formamide (1.66 g, 36.88 mmol) was refluxed at 165° C. for 1 h and then at 175° C. at 1 h. The residue was washed with water and added to a solution of sodium bicarbonate (30 ml) and stirred to shredding the residue. Then the precipitate was filtered and washed with water to give the title compound (P33) as a white solid (2.03 g, 97%). ¹H NMR (400 MHZ, DMSO-d₆), δ: 12.69 (s, 1H), 9.03 (d, J=2.6 Hz, 1H), 8.60 (d, J=2.6 Hz, 1H), 8.35 (s, 1H).

### Preparation 34. tert-Butyl-2-(6-bromopyrido[2,3-d]pyrimidin-4-yl)-2,7-diazaspiro[3.5]nonane-7-carboxylate (P34)

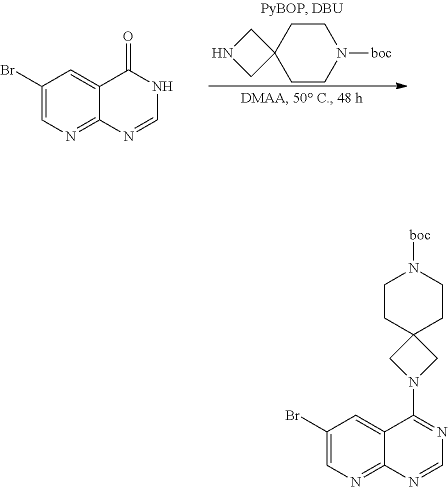

The P33 (144 mg, 0.64 mmol), amine (205 mg, 0.78 mmol), PyBOP(416 mg, 1.11 mmol) and DBU (474 mg, 3.12 mmol) were dissolved in DMAA (2 ml). The reaction mixture was stirred at 60° C. for 48 h. Then the reaction mixture was cooled to rt, extracted with EtOAc (2×10 ml), washed with brine (40 ml), dried over sodium sulfate, filtered, concentrated, and purified by silica gel column chromatography (gradient from EtOAc to 1% MeOH in EtOAc) to give the title compound (P34) as a white solid (150 mg, 44%). ¹H NMR (400 MHZ, DMSO-d₆), δ: 9.03 (d, J=2.4 Hz, 1H), 8.59 (s, 1H), 8.46 (d, J=2.5 Hz, 1H), 3.35 (br. s, 2H), 3.29 (br. s, 2H), 2.51 (br. s, 2H), 2.49 (br. s, 2H), 1.74 (s, 4H), 1.40 (s, 9H).

### Preparation 35. tert-Butyl-2-(6-vinylpyrido[2,3-d]pyrimidin-4-yl)-2,7-diazaspiro[3.5]nonane-7-carboxylate (P35)

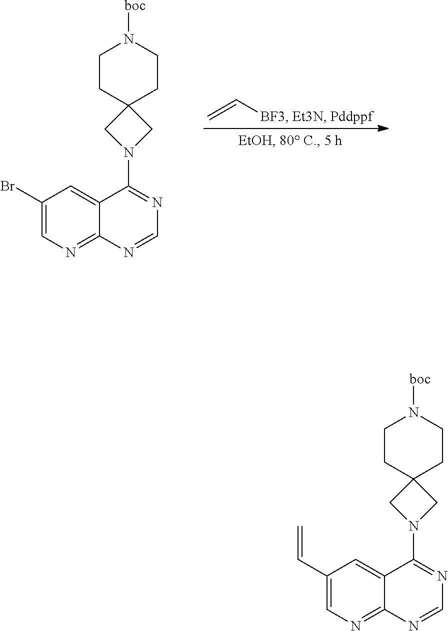

To a solution of P34 (150 mg, 0.35 mmol) in EtOH (2 ml) DIPEA (87 mg, 0.75 mmol) and vinyl-BF3 (92 mg, 0.70 mmol) were added. Then the mixture was filled with argon and Pddppf (30 mg) was added. The reaction mixture was stirred at 80° C. for 5 h. Then the reaction mixture was cooled to room temperature, extracted with EtOAc and water, dried over sodium sulfate, filtered, concentrated, and purified by silica gel column chromatography (gradient from EtOAc to 1% MeOH in EtOAc) to give the title compound (P35) as a white solid (130 mg, 99%). LCMS(ESI) [MH]⁺: 382.

### Preparation 36. tert-Butyl-2-(6-formylpyrido[2,3-d]pyrimidin-4-yl)-2,7-diazaspiro[3.5]nonane-7-carboxylate (P36)

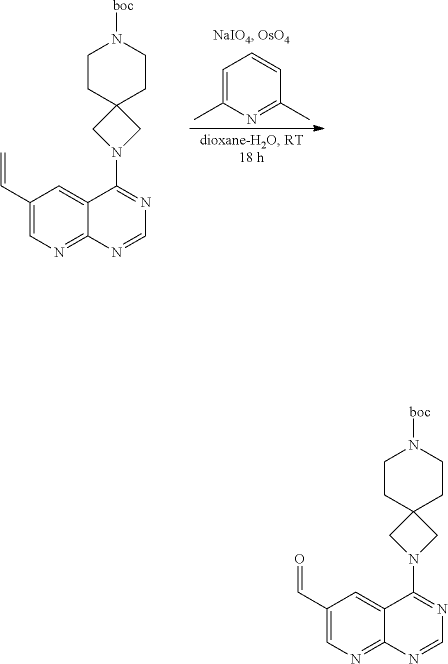

To the solution of P35 (130 mg, 0.3 mmol), 2,6-lutidine (58 mg, 0.48 mmol) and NaIO₄ (241 mg, 0.99 mmol) in mixture of dioxane (6 ml) and water (2 ml) that filled by argon was added the solution of OsO₄ (17 mg, 0.06 mmol) in t-BuOH (1 ml). The reaction mixture was stirred at room temperature for 20 h. The reaction mixture was extracted with DCM (2×10 ml) and purified by silica gel column chromatography (gradient from EtOAc to 1% MeOH in EtOAc) to give the title compound (P36) as a white solid (110 mg, 84%). ¹H NMR (400 MHZ, CDCl₃), δ: 10.19 (s, 1H), 9.46 (d, J=2.2 Hz, 1H), 8.83 (s, 1H), 8.65 (d, J=2.2 Hz, 1H), 4.36 (br. s, 2H), 3.17 (d, J=3.4 Hz, 2H), 1.89 (t, J=5.4 Hz, 4H), 1.72 (s, 2H), 1.62 (s, 2H), 1.49 (s, 9H).

### Preparation 37. tert-Butyl-2-{6-[(E)-hydrazonomethyl]pyrido[2,3-d]pyrimidin-4-yl}-2,7-diazaspiro[3.5]nonane-7-carboxylate (P37)

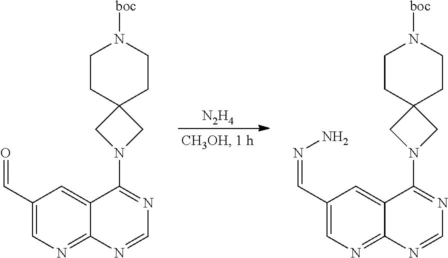

To a stirred solution of P36 (100 mg, 0.26 mmol) in MeOH (1 ml) was added dropwise aqua solution of hydrazine hydrate (39 mg, 0.72 mmol). The reaction mixture was stirred at rt for 1 h. The reaction mixture was evaporated under reduced pressure to give the title compound (P37) as white solid (87 mg, 84%). LCMS(ESI) [MH]⁺: 398.

### Preparation 38. tert-Butyl-2-[6-(2,2,2-trifluoroethyl) pyrido[2,3-d]pyrimidin-4-yl]-2,7-diazaspiro[3.5]nonane-7-carboxylate (P38)

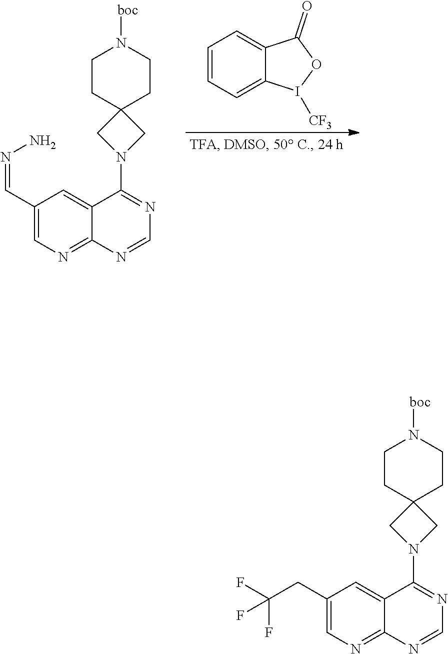

A solution of (P37) (87 mg, 0.22 mmol), 3-(trifluoromethyl)-2-benzofuran-1(3H)-one (97 mg, 0.31 mmol) and TFA (25 mg, 0.22 mmol) in DMSO (2 ml) was filled with argon. The reaction mixture was stirred at 50° C. for 24 h. The reaction mixture was extracted with EtOAc, combined organic layers were evaporated under reduced pressure and purified by silica gel column chromatography (gradient from EtOAc to 50% MeOH in EtOAc) to give the title compound P38 as white solid (39 mg, 41%). ¹H NMR (400 MHZ, CDCl₃), δ: 8.95 (s, 1H), 8.78 (s, 1H), 8.12 (s, 1H), 4.29 (s, 4H), 3.55 (d, J=10.4 Hz, 2H), 3.47 (s, 4H), 1.94-1.84 (m, 4H), 1.48 (s, 9H).

### Preparation 39. 4-(2,7-diazaspiro[3.5]non-2-yl)-6-(2,2,2-trifluoroethyl) pyrido[2,3-d]pyrimidine (P39)

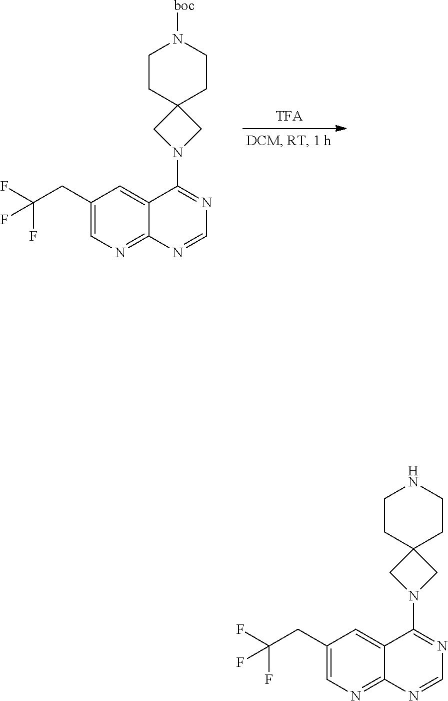

A solution (P38) (39 mg, 0.09 mmol) and TFA (3 ml) in DCM (1 ml) was stirred at rt for 1 h. The reaction mixture was evaporated under reduced pressure, then aqua solution of potassium carbonate was added and extracted with DCM (2×10 ml), dried over sodium sulfate, filtered, and concentrated to give the title compound (P39) as a white solid (25 mg, 83%). ¹H NMR (400 MHz, CDCl₃), δ: 8.94 (d, J=1.8 Hz, 1H), 8.77 (s, 1H), 8.14-8.08 (m, 1H), 4.27 (s, 4H), 3.58-3.49 (m, 2H), 3.17 (d, J=3.0 Hz, 1H), 2.89 (s, 4H), 1.93-1.86 (m, 4H).

### Synthesis of 4-(2,7-Diazaspiro[3.5]non-2-yl)-6-(2,2,2-trifluoroethyl) cinnoline (P49)

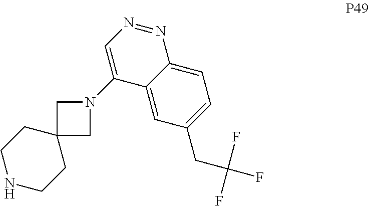 [SMILES: FC(F)(F)Cc1ccc2nncc(N3CC4(CCNCC4)C3)c2c1]

### Preparation 45. tert-Butyl 2-(6-bromocinnolin-4-yl)-2,7-diazaspiro[3.5]nonane-7-carboxylate (P45)

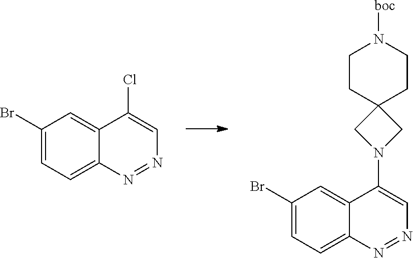

To a solution of crude 6-bromo-4-chlorocinnoline (prepared according to the procedure described in US2015/259331) (1.55 g, 6.37 mmol) in dichloroethane (40 ml) was added DIPEA (7.9 g, 61 mmol) and tert-butyl 2,7-diazaspiro[3.5]nonane-7-carboxylate hydrochloride (1.67 g, 6.37 mmol). Reaction solution was stirred at rt for 18 h (LCMS control). The product was purified by column chromatography on silica with ethylacetate-methanol (10:1) to yield the title compound P45 (1.3 g, 47%) as a white solid. LCMS(ESI) [MH]⁺: 434.

### Preparation 46. tert-Butyl 2-(6-vinilcinnolin-4-yl)-2,7-diazaspiro[3.5]nonane-7-carboxylate (P46)

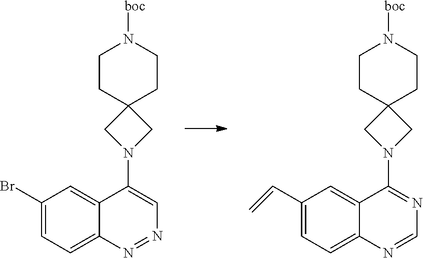

To a solution of tert-butyl 2-(6-bromocinnolin-4-yl)-2,7-diazaspiro[3.5]nonane-7-carboxylate (P45, 0.30 g, 0.69 mmol) in EtOH (10 mL) water (0.5 ml) and TEA (140 mg, 1.39 mmol) were added. Then potassium vinyltrifluoroborate (140 mg, 1.03 mmol) and dichloro[1,1′-bis(diphenylphosphino)ferrocene]palladium (H) dichloromethane adduct (0.17 g, 0.21 mmol) were added, and the reaction mixture was stirred at 80° C. for 24 hours. The reaction mixture was worked up with EtOAc and H₂O, and the layers were separated. The organic layer was washed with brine, dried over MgSO₄, filtered and concentrated. The crude material was purified on silica (gradient elution, 0-40% EtOAc/hexanes) to yield the title compound P46 as white solid (210 mg, 80%). LCMS(ESI) [MH]⁺: 381.

### Preparation 47. tert-Butyl 2-(6-fopmylcinnolin-4-yl)-2,7-diazaspiro[3.5]nonane-7-carboxylate (P47)

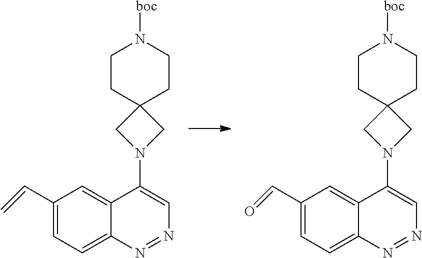

To a solution of tert-butyl 2-(6-vinilcinnolin-4-yl)-2,7-diazaspiro[3.5]nonane-7-carboxylate (P46, 210 mg, 0.55 mmol) in a mixture of dioxane (2 ml) and water (0.42 ml) 2,6-lutidine (113 mg, 1.1 mmol), solution of osmium tetraoxide (10 mg) in tert-BuOH (0.5 ml) and sodium periodate (354 mg, 1.65 mmol) were added. The reaction mixture was stirred at rt for 24 h, after reaction completed, the solution cooled and added to ice-cold water solution of sodium bicarbonate. The mixture was stirred at 15° C. for 30 min, then extracted with DCM (2×50 ml), DCM was evaporated, residue was purified by silica gel column chromatography (DCM-ethylacetate-1:1) to give the title product P47 (145 mg, 65%) as a brown solid. LCMS(ESI) [MH]⁺: 383.

### Preparation 48. tert-Butyl 2-(6-(2,2,2-trifluoroethyl) cinnolin-4-yl)-2,7-diazaspiro[3.5]nonane-7-carboxylate (P48)

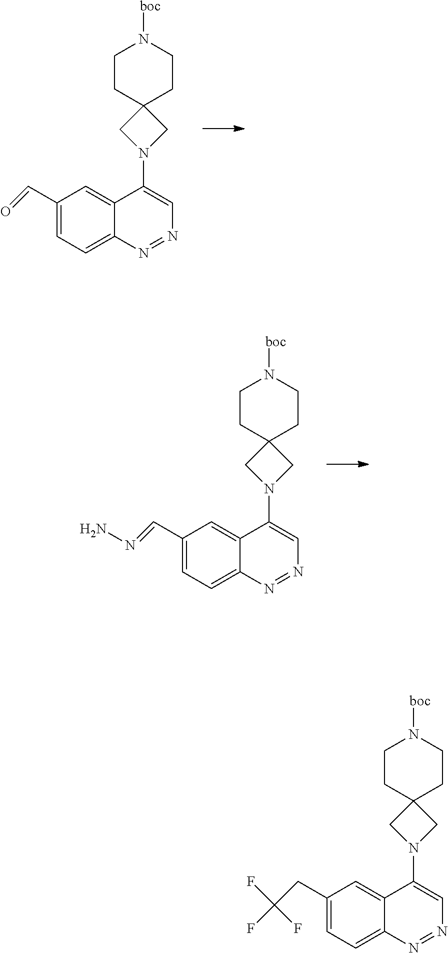

To a solution of tert-butyl 2-(6-fopmylcinnolin-4-yl)-2,7-diazaspiro[3.5]nonane-7-carboxylate (P47, 145 mg, 0.38 mmol) in MeOH (1 ml) hydrazine hydrate (38 mg, 0.76 mmol) was added. The reaction mixture was stirred at rt for 2 h (TLC control), then reaction mixture was evaporated to dryness yield crude hydrazone (150 mg) used in the next step without purification. The hydrazone (150 mg, 0.38 mmol) was dissolved in DMSO (1 ml) under argon atmosphere, Togni's reagent (144 mg, 0.456 mmol) and TFA (43 mg, 0.38 mmol) were added. The resulting mixture was stirred for 24 h at 50° C. After cooling to rt, the mixture was extracted with ethylacetate (3×25 ml), and the combined organic layers were washed with water (3×60 ml) and brine (25 ml) and dried under Na₂SO₄. The organic layers were concentrated, and the residue separated by HPLC (silica C-18, MeCN—H₂O) to yield P48 (75 mg, 45%). LCMS(ESI) [MH]⁺: 437. ¹H NMR (400 MHZ, CDCl₃), δ: 8.35 (s, 1H), 8.29 (d, J=5.2 Hz, 1H), 7.80 (s, 1H), 7.62 (d, J=9.4 Hz, 1H), 4.29-4.17 (m, 4H), 3.60-3.43 (m, 6H), 1.92-1.85 (m, 4H), 1.49 (s, 9H).

### Preparation 49. 4-(2,7-Diazaspiro[3.5]non-2-yl)-6-(2,2,2-trifluoroethyl) cinnoline (P49)

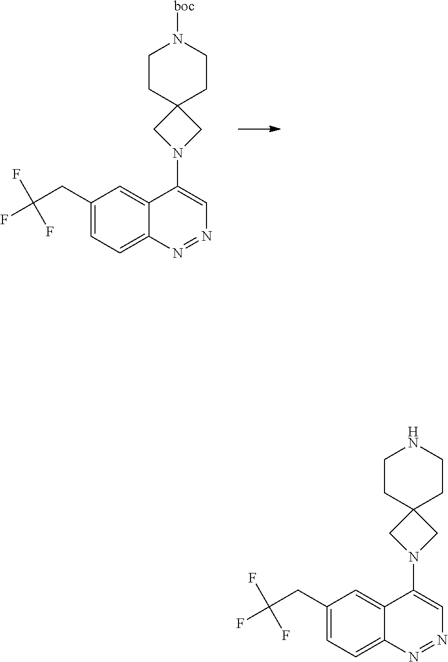

To tert-butyl 2-(6-(2,2,2-trifluoroethyl) cinnolin-4-yl)-2,7-diazaspiro[3.5]nonane-7-carboxylate (P48, 75 mg, 0.17 mmol) was added TFA (1 ml). The solution was stirred at rt for 2 h (LCMS control), then evaporated to dryness. A water solution of sodium bicarbonate (5 ml) was added to the residue, product was extracted with DCM (3×5 ml). The extract was dried with sodium sulphate, concentrated to dryness yield 4-(2,7-Diazaspiro[3.5]non-2-yl)-6-(2,2,2-trifluoroethyl) cinnoline P49 (57 mg, 100%). LCMS(ESI) [MH]⁺: 337. ¹H NMR (400 MHZ, DMSO-d₆), δ: 8.35 (s, 1H), 8.14-8.07 (m, 1H), 8.03 (s, 1H), 7.73-7.66 (m, 1H), 4.25-4.14 (m, 4H), 4.09-3.82 (m, 6H), 2.10-1.94 (m, 4H).

### Synthesis of 4-(2,7-diazaspiro[3.5]non-2-yl)-6-(2,2,2-trifluoroethyl) pyrido[3,2-d]pyrimidine (P61)

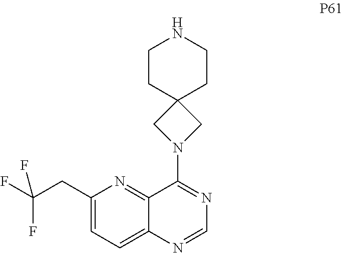 [SMILES: FC(F)(F)Cc1ccc2ncnc(N3CC4(CCNCC4)C3)c2n1]

### Preparation 50. Methyl 3-aminopicolinate (P50)

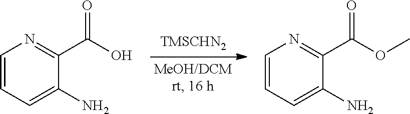

To a solution of 3-aminopicolinic acid (1.00 g, 7.24 mmol, 1.0 eq) in methanol (4.0 mL) and DCM (16 mL) was added trimethylsilyldiazomethane (7.23 mL, 14.5 mmol, 2.0 eq) at rt. Then the mixture solution was stirred at rt for 16 h, the solution was treated with water and extracted with EtOAc. The organic layers were collected, washed with brine, dried over MgSO₄(s), filtered, and concentrated in vacuo to afford P50 (555 mg, 50%) as an orange solid. ¹H NMR (400 MHZ, CDCl₃), δ: 8.06 (dd, J=4.0, 1.4 Hz, 1H), 7.22 (dd, J=8.4, 4.0 Hz, 1H), 7.05 (dd, J=8.4, 1.4 Hz, 1H), 5.73 (br. s, 2H), 3.97 (s, 3H).

### Preparation 51. Methyl 3-amino-6-bromopicolinate (P51)

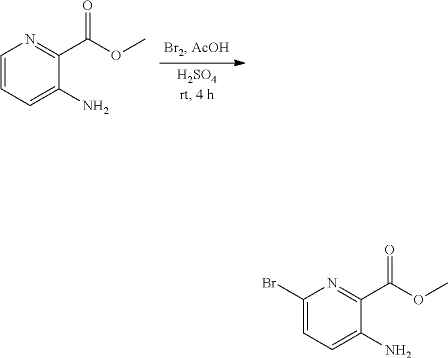

To a solution of P50 (5.00 g, 32.8 mmol, 1.0 eq) in 2M of sulfuric acid (101 mL) was added a solution of bromine (1.68 mL, 32.8 mmol, 1.0 eq) in acetic acid (12.6 mL) at rt. After the mixture solution was stirred at rt for 4 h, the solution was treated with Na₂S₂O₃ (aq) and extracted with EtOAc. The organic layers were collected, washed with brine, dried over MgSO₄(s), filtered, and concentrated in vacuo. The residue was purified by flash column chromatography (EtOAc: n-hexane=1:3) to afford P51 (5.56 g, 73%) as an orange solid. ¹H NMR (400 MHZ, CDCl₃), δ: 7.34 (d, J=8.6 Hz, 1H), 6.94 (d, J=8.6 Hz, 1H), 5.82 (br. s, 2H), 3.95 (s, 3H).

### Preparation 52. Methyl 3-amino-6-vinylpicolinate (P52)

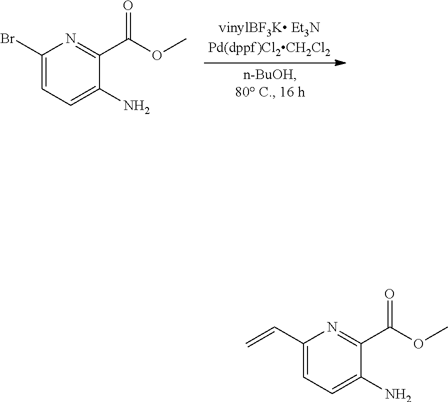

To a stirred solution of P51 (500 mg, 2.16 mmol, 1.0 eq) in n-butanol (10 mL) was added potassium vinyltrifluoroborate (435 mg, 3.25 mmol, 1.5 eq) and trimethylamine (1.51 mL, 10.8 mmol, 5.0 eq) at rt. The solution was degassed with argon, and then was added by 1,1′-bis(diphenylphosphino)ferrocene-palladium (II) dichloride dichloromethane complex. Then the reaction mixture was stirred at 80° C. for 16 h, it was cooled to rt and filtered through with a pad of celite. The solution was treated with water and extracted with EtOAc. The organic layers were collected, washed with brine, dried over MgSO₄(s), filtered, and concentrated in vacuo. The residue was purified by flash column chromatography (EtOAc: n-hexane=1:2) to afford P52 (230 mg, 60%) as a yellow solid. ¹H NMR (400 MHZ, CDCl₃), δ: 7.47 (d, J=8.8 Hz, 1H), 7.04 (d, J=8.8 Hz, 1H), 6.81 (dd, J=17.6, 10.8 Hz, 1H), 5.86 (dd, J=17.6, 0.8 Hz, 1H), 5.79 (br. s, 2H), 5.36 (dd, J=10.8, 0.8 Hz, 1H), 3.97 (s, 3H); LCMS(ESI) [MH]⁺: 179.1.

### Preparation 53. Methyl 3-(di-tert-butoxycarbonylamino)-6-vinylpicolinate (P53)

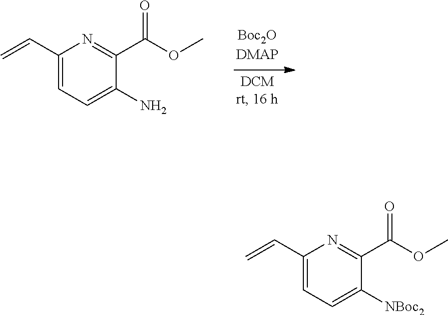

To a solution of P52 (230 mg, 1.29 mmol, 1.0 eq) in DCM (10 mL) was added di-tert-butyl dicarbonate (0.890 mL, 3.87 mol, 3.0 eq) and 4-dimethylaminopyridine (15.7 mg, 0.129 mmol, 0.1 eq) at rt. After the reaction mixture was stirred at rt for 16 h, it was concentrated in vacuo. The residue was purified by flash column chromatography (EtOAc: n-hexane=1:3) to afford P53 (432 mg, 88%) as a white solid. ¹H NMR (400 MHZ, CDCl₃), δ: 7.54 (m, 2H), 6.91 (dd, J=17.6, 11.2 Hz, 1H), 6.22 (d, J=17.6 Hz, 1H), 5.60 (d, J=11.2 Hz, 1H), 3.94 (s, 3H), 1.38 (s, 18H); LCMS(ESI) [MH]⁺: 379.2.

### Preparation 54. Methyl 3-(di-tert-butoxycarbonylamino)-6-formylpicolinate (P54)

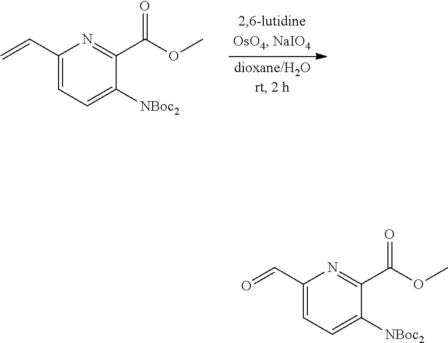

To a solution of P53 (432 mg, 1.14 mmol, 1.00 eq) in 1,4-dioxane (10 mL) and water (2.5 mL) was added 2.5% osmium tetroxide in tert-butanol (0.226 mL, 0.0228 mmol, 0.02 eq), sodium periodate (977 mg, 4.56 mmol, 4.00 eq), 2,6-lutidine (265 mL, 2.28 mmol, 2.00 eq) at room temperature and stirred at room temperature for 2 h. The mixture solution was filtered through celite and extracted with EtOAc. The organic layers were collected, washed with brine, dried over MgSO₄(s), filtered, and concentrated in vacuo. The residue was purified by flash column chromatography (EtOAc: dichloromethane=1:4) to afford P54 (402 mg, 93%) as a yellow solid. ¹H NMR (400 MHz, CDCl₃) δ 10.15 (s, 1H), 8.13 (d, J=8.0 Hz, 1H), 7.77 (dd, J=8.0 Hz, 1H), 4.00 (s, 3H), 1.39 (s, 18H).

### Preparation 55. methyl (E)-3-(di-tert-butoxycarbonylamino)-6-(hydrazineylidenemethyl)-picolinate (P55)

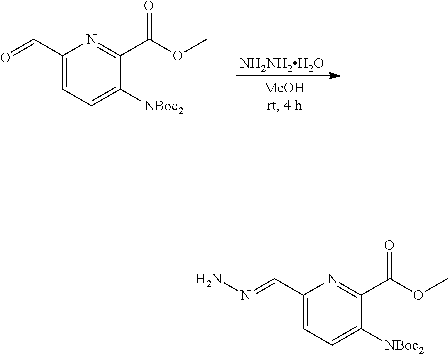

To a solution of P54 (402 mg, 1.06 mmol, 1.0 eq) in methanol (9 mL) was added hydrazine monohydrate (63.5 mg, 1.27 mol, 1.2 eq) at rt and the mixture solution was stirred at room temperature for 4 h. The solution was concentrated in vacuo to afford P55 (428 mg) as a yellow solid which was used in next step without purification.

### Preparation 56. Methyl 3-(di-tert-butoxycarbonylamino)-6-(2,2,2-trifluoroethyl) picolinate (P56)

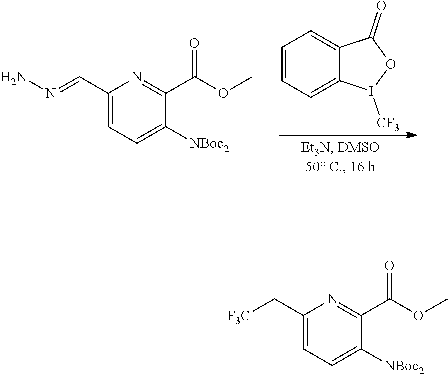

A solution of P55 (428 mg, 1.08 mmol, 1.0 eq) in DMSO (10 mL) was added 1-trifluoromethyl-1,2-benziodoxol-3-(1H)-one (377 mg, 1.19 mmol, 1.1 eq) and the mixture was stirred for 10 min. Triethylamine (0.197 mL, 1.41 mmol, 1.3 eq) was added to the reaction mixture and the solution was stirred at 50° C. for 16 h. After the solution was cooled to rt, it was diluted with water, and extracted with EtOAc. The organic layers were washed with brine, dried over MgSO₄(s), filtered, and concentrated in vacuo. The residue was purified by flash column chromatography (EtOAc: n-hexane=1:2) to afford P56 (258 mg, crude) as a yellow solid. ¹H NMR (400 MHZ, CDCl₃), δ: 7.60 (d, J=8.0 Hz, 1H), 7.53 (d, J=8.0 Hz, 1H), 3.95 (s, 3H), 2.80 (q, J=10.4 Hz, 2H), 1.38 (s, 18H); LRMS(ESI) [MH]⁺: 435.1.

### Preparation 57. Methyl 3-amino-6-(2,2,2-trifluoroethyl) picolinate (P57)

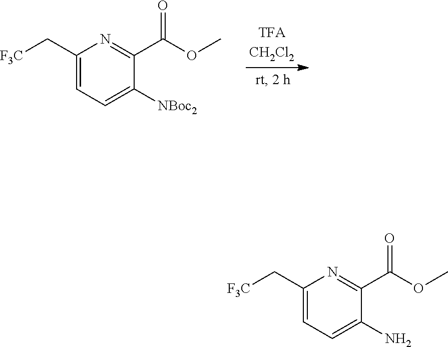

To a solution of P56 (3.6 g, 8.29 mmol, 1.0 eq) in DCM (20 mL) was added trifluoroacetic acid (10 mL) and the mixture was stirred at rt for 2 h. The solution was concentrated in vacuo and purified by flash column chromatography (EtOAc: n-hexane=1:1) to afford P57 (1.9 g, 98%) as a yellow solid. ¹H NMR (400 MHZ, CDCl₃), δ: 7.28 (d, J=8.6 Hz, 1H), 7.07 (d, J=8.6 Hz, 1H), 3.97 (s, 3H), 3.57 (q, J=10.8 Hz, 2H); LCMS(ESI) [MH]⁺: 235.1.

### Preparation 117. 3-Amino-6-(2,2,2-trifluoroethyl) picolinamide (P58)

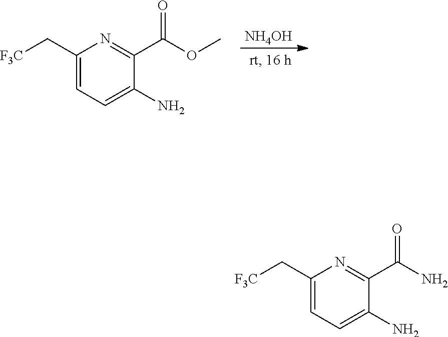

A solution of P57 (1.90 g, 8.11 mmol, 1.0 eq) in 28% of ammonia in water (50 mL) was stirred at rt for 16 h. The solution was diluted with water and extracted with EtOAc. The organic layers were collected, washed with brine, dried over MgSO₄(s), filtered, and concentrated in vacuo to afford P58 (1.45 g, 81%) as a yellow solid. ¹H NMR (400 MHZ, CDCl₃), δ: 7.82 (br. s, 1H), 7.19 (d, J=8.4 Hz, 1H), 7.00 (d, J=8.4 Hz, 1H), 5.98 (br. s, 2H), 5.42 (br. s, 1H), 3.45 (q, J=10.8 Hz, 2H).

### Preparation 59. 6-(2,2,2-trifluoroethyl) pyrido[3,2-d]pyrimidin-4 (3H)-one (P59)

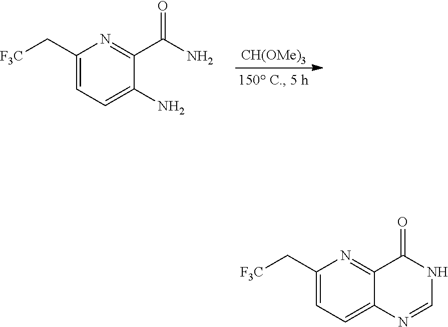

A solution of P58 (50.0 mg, 0.114 mmol, 1.0 eq) in trimethyl orthoformate (5.0 mL) in sealed tube was stirred at 150° C. for 5 h. After the solution was cooled to rt, it was diluted with water and extracted with EtOAc. The organic layers were collected, washed with brine, dried over MgSO₄(s), filtered, and concentrated in vacuo. The residue was purified by flash column chromatography (methanol: DCM=1:10) to afford P59 (38 mg, 73%) as a pale-yellow solid. ¹H NMR (400 MHZ, CDCl₃), δ: 12.30 (br. s, 1H), 8.23 (s, 1H), 8.17 (d, J=8.4 Hz, 1H), 7.82 (d, J=8.4 Hz, 1H), 3.91 (q, J=10.4 Hz, 2H); LCMS(ESI) [MH]⁺: 230.1.

### Preparation 60. tert-Butyl 2-(6-(2,2,2-trifluoroethyl) pyrido[3,2-d]pyrimidin-4-yl)-2,7-diazaspiro[3.5]nonane-7-carboxylate (P60)

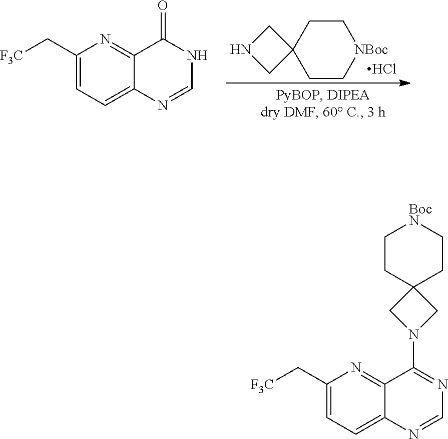 [SMILES: O=c1[nH]cnc2ccc(CC(F)(F)F)nc12 ; CC(C)(C)OC(=O)N1CCC2(CC1)CNC2 ; Cl ; CC(C)(C)OC(=O)N1CCC2(CC1)CN(c1ncnc3ccc(CC(F)(F)F)nc13)C2]

To a solution of P59 (410 mg, 1.79 mmol, 1.0 eq) in anhydrous N,N-dimethylformamide (10 mL) was added tert-butyl2,7-diazaspiro[3.5]nonane-7-carboxylatehydrochloride (564 mg, 2.15 mmol, 1.2 eq), benzotriazol-1-yl-oxytripyrrolidinophosphonium hexafluorophosphate (1.86 g, 3.58 mmol, 2.0 eq) and N,N-diisopropylethylamine (0.934 mg, 5.36 mmol, 3.0 eq). After the reaction mixture was stirred at 60° C. for 3 h, it was cooled to rt and directly purified by C18 flash column chromatography (0-100% methanol in H₂O) to afford P60 (580 mg, 74%) as an orange solid. ¹H NMR (400 MHZ, CDCl₃), δ: 8.55 (s, 1H), 8.05 (d, J=8.6 Hz, 1H), 7.58 (d, J=8.6 Hz, 1H), 4.61 (s, 2H), 4.08 (s, 2H), 3.69 (q, J=10.4 Hz, 2H), 3.53-3.48 (m, 2H), 3.41-3.36 (m, 2H), 1.85-1.83 (m, 4H), 1.47 (s, 9H); LCMS(ESI) [MH]⁺: 438.2.

### Preparation 61. 4-(2,7-diazaspiro[3.5]non-2-yl)-6-(2,2,2-trifluoroethyl)pyrido[3,2-d]pyrimidine (P61)

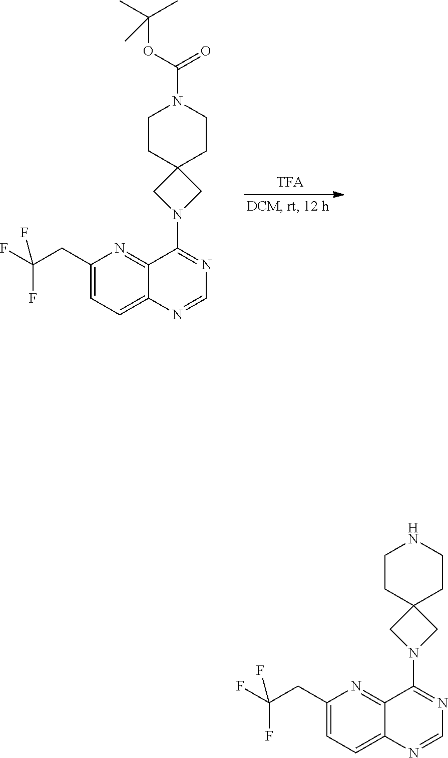 [SMILES: CC(C)(C)OC(=O)N1CCC2(CC1)CN(c1ncnc3ccc(CC(F)(F)F)nc13)C2 ; FC(F)(F)Cc1ccc2ncnc(N3CC4(CCNCC4)C3)c2n1]

To a stirred solution of P60 (330 mg, 0.68 mmol) in DCM (3 ml) was added TFA (0.860 g, 10 eq). After the reaction was stirred at rt overnight and the solution was treated with sat. NaHCO₃ and extracted with EtOAc. The combined organic layers were dried over with MgSO₄(s), filtered, and concentrated to give the product P61 as yellow solid (240 mg, 94%). LCMS(ESI) [MH]⁺: 338.4.

### Synthesis of 4-(2,7-diazaspiro[3.5]non-2-yl)-6-(2,2,2-trifluoroethyl) pteridine (P71)

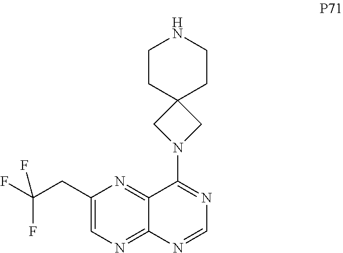 [SMILES: FC(F)(F)Cc1cnc2ncnc(N3CC4(CCNCC4)C3)c2n1]

### Preparation 62. Methyl 3-amino-6-vinyl-pyrazine-2-carboxylate (P62)

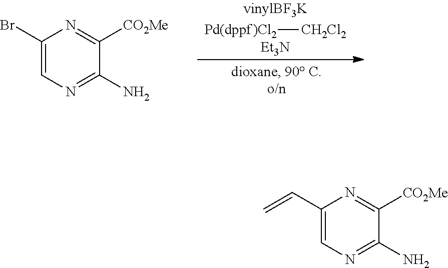

To a stirred solution of methyl 3-amino-6-bromopyrazine-2-carboxylate (12.0 g, 51.72 mmol) in dioxane (120 mL) was added potassium vinyltrifluoroborate (10.4 g, 77.6 mmol) and triethylamine (37.7 mL, 259 mmol). The solution was purged with argon for 30 min, and then [1,1′-bis(diphenylphosphino)ferrocene]dichloropalladium(II) dichloromethane complex (4.23 g, 5.17 mmol) was added and purged again. The mixture was stirred at reflux for 16 h. Monitoring by TLC showed the reaction was completed. The solution was treated with ethyl acetate and washed with NaHCO₃(aq) and water. The organic phase was dried over with MgSO₄(s), filtered, and concentrated in vacuo to give a crude product. The residue was purified by silica gel column chromatography (25% EtOAc in n-hexane) to give methyl 3-amino-6-vinylpyrazine-2-carboxylate (P62, 7.5 g, 75% yield) as a yellow solid.

### Preparation 63. Methyl 3-(di(tert-butoxycarbonyl)amino)-6-vinylpyrazine-2-carboxylate (P63)

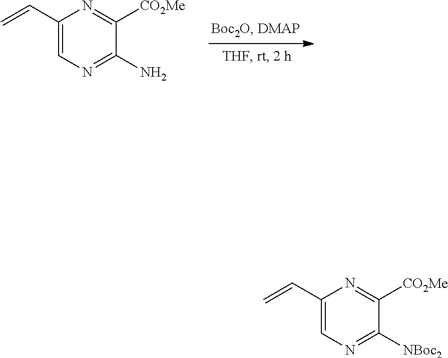

To a solution of P62 (7.5 g, 41.9 mmol) in tetrahydrofuran (100 mL) was added di-tert-butyl dicarbonate (48.2 mL, 209.5 mmol) and 4-dimethylaminopyridine (512 mg, 4.19 mmol). The reaction was stirred at rt for overnight. It showed starting materials was consumed. It was concentrated to remove solvent. The mixture was extracted with EtOAc/Water and purified by silica-gel column chromatography (25% EtOAc in n-Hexane) to give P63 (14.57 g, 92% yield) as a yellow solid. ¹H NMR (400 MHZ, CDCl₃), δ: 8.67 (s, 1H), 6.92 (dd, J=17.6 Hz, 11.0 Hz, 1H), 6.41 (d, J=17.6 Hz, 1H), 5.75 (d, J=11.0 Hz, 1H), 3.98 (s, 3H), 1.39 (s, 18H).

### Preparation 64. Methyl 3-(di(tert-butoxycarbonyl)amino)-6-formylpyrazine-2-carboxylate (P64)

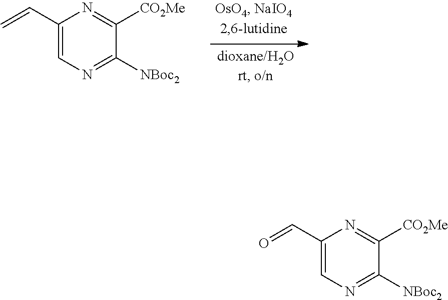

To a stirred solution of P63 (14.0 g, 36.9 mmol) in dioxane (400 mL) and H₂O 100 mL) 2,6-lutidine (8.6 mL, 73.8 mmol), a solution of 2.5% osmium tetroxide in tert-butanol (188 mg, 0.74 mmol), and sodium periodate (31.59 g, 147.6 mmol) were added at rt. The mixture was stirred at rt for 16 h. Monitoring by TLC showed the reaction was completed. The resulting mixture was filtered, and the filtrate was treated with water and extracted with EtOAc. The organic layer was dried over with MgSO₄(s), filtered and concentrated in vacuo to give a crude product. The residue was purified by silica gel column chromatography (25% EtOAc in n-Hexane) to give P64 (9.96 g, 71% yield) as a brown solid. ¹H NMR (400 MHZ, CDCl₃), δ: 10.23 (s, 1H), 9.18 (s, 1H), 4.03 (s, 3H), 1.42 (s, 18H).

### Preparation 65. Methyl (E)-3-(di(tert-butoxycarbonyl)amino)-6-(hydrazineylidenemethyl) pyrazine-2-carboxylate (P65)

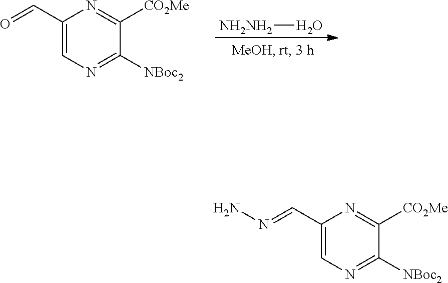

To a stirred solution of P64 (9.96 g, 26.1 mmol) in methanol (50 mL) was added hydrazine monohydrate (1.52 mL, 31.3 mmol) at 0° C. and stirred for 10 min. The solution was warmed to rt and stirred for another 5 h. The solution was concentrated to give P65 as a crude product which was used in next step without further purification. ¹H NMR (400 MHZ, CDCl₃), δ: 9.11 (s, 1H), 7.86 (s, 1H), 6.11 (br. s, 2H), 3.98 (s, 3H), 1.39 (s, 18H).

### Preparation 66. Methyl 3-(di(tert-butoxycarbonyl)amino)-6-(2,2,2-trifluoroethyl) pyrazine-2-carboxylate (P66)

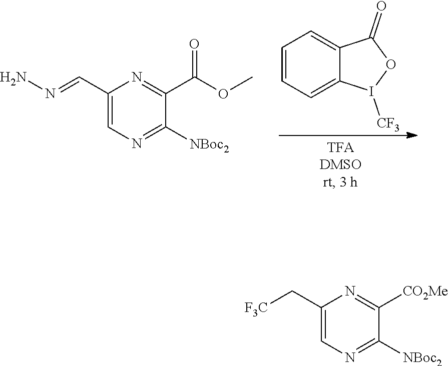

To a stirred solution of P65 (26.1 mmol) in DMSO (70 mL) was added 1-(trifluoromethyl)-1,2-benziodoxol-3 (1H)-one (1.52 mL, 31.3 mmol) and TFA (2.0 mL) at 0° C. and stirred for 10 min. The solution was warmed to rt and stirred for 3 h. The mixture was treated with water and extracted with EtOAc. The combined organic layers were dried over with MgSO₄(s), filtered, and concentrated to give a residue. The residue was purified by silica-gel column chromatography (20-33% EtOAc in n-hexane) to give P66 (4.54 g, 40% yield) as a white solid. ¹H NMR (400 MHZ, CDCl₃), δ: 8.68 (s, 1H), 3.99 (s, 3H), 3.80 (q, J=10.4 Hz, 2H), 1.40 (s, 18H).

### Preparation 67. Methyl 3-amino-6-(2,2,2-trifluoroethyl) pyrazine-2-carboxylate (P67)

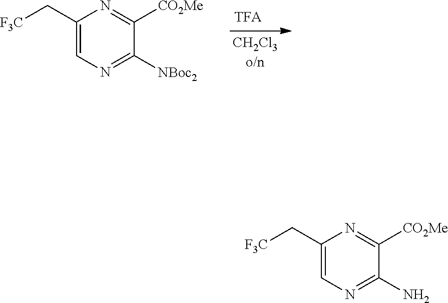

To a stirred solution of P66 (4.54 g, 10.43 mmol) in DCM (20 mL) was added trifluoroacetic acid (10.0 mL) at rt and stirred for overnight. The mixture was concentrated to give P67 as a product which was used in next step without further purification. ¹H NMR (400 MHZ, DMSO-d₆), δ: 8.30 (s, 1H), 7.43 (br. s, 2H), 3.84 (s, 3H), 3.71 (q, J=11.2 Hz, 2H).

### Preparation 68. 3-Amino-6-(2,2,2-trifluoroethyl) pyrazine-2-carboxamide (P68)

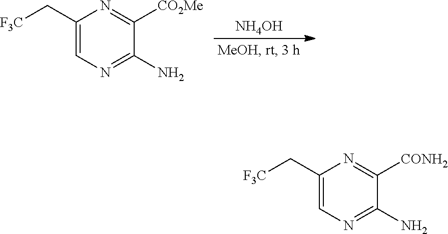 [SMILES: COC(=O)c1nc(CC(F)(F)F)cnc1N ; "NH4OH" ; NC(=O)c1nc(CC(F)(F)F)cnc1N]

To a stirred solution of P67 (11.28 mmol) in methanol (40 mL) 33% ammonium hydroxide solution (10 mL) was added at rt and stirred for 3 h. The solution was concentrated to give P68 as a yellow solid which was used in next step without further purification. ¹H NMR (400 MHZ, DMSO-d₆), δ: 8.23 (s, 1H), 7.89 (br. s, 1H), 7.68 (br. s, 1H), 3.68 (q, J=11.2 Hz, 2H).

### Preparation 69. 6-(2,2,2-trifluoroethyl) pteridin-4 (3H)-one (P69)

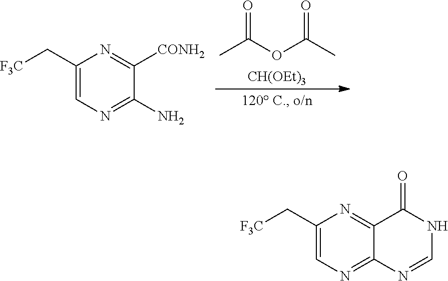

To a stirred solution of P68 (11.28 mmol) in triethyl orthoformate (24.4 mL) acetic anhydride (12.3 mL) was added at rt and heated to 120° C. After the mixture was stirred for 3 h at 120° C., the solution was cooled to rt and concentrated. The crude product was treated with isopropanol. The resulting precipitate was collected by filtration and dried under air to give P69 (1.96 g, 76%) as an apricot solid. ¹H NMR (400 MHZ, DMSO-de), δ: 12.89 (br. s, 1H), 9.04 (s, 1H), 8.37 (s, 1H), 4.12 (q, J=11.4 Hz, 2H); HPLC purity: 100.0%, Rt=8.434 min.

### Preparation 70. tert-Butyl 2-(6-(2,2,2-trifluoroethyl) pteridin-4-yl)-2,7-diazaspiro[3.5]nonane-7-carboxylate (P70)

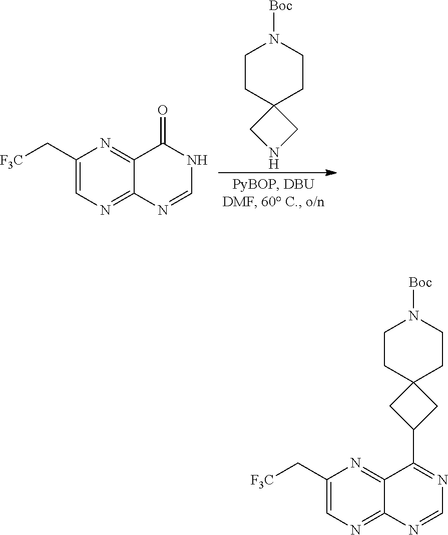 [SMILES: O=c1[nH]cnc2ncc(CC(F)(F)F)nc12 ; CC(C)(C)OC(=O)N1CCC2(CC1)CNC2 ; CC(C)(C)OC(=O)N1CCC2(CC1)CC(c1ncnc3ncc(CC(F)(F)F)nc13)C2]

To a stirred solution of P69 (800 mg, 3.48 mmol) in N,N-dimethylformamide (100 mL) was added tert-butyl 2,7-diazaspiro[3.5]nonane-7-carboxylate hydrochloride (1.094 g, 4.18 mmol), N,N-diisopropylethylamine (1.82 mL) and (benzotriazol-1-yloxy)tripyrrolidinophosphonium hexafluorophosphate (5.44 g, 10.44 mmol). After the reaction was stirred at 60° C. for overnight and cooled to rt, the solution was treated with water and extracted with EtOAc. The combined organic layers were dried over with MgSO₄(s), filtered, and concentrated to give a residue. The residue was purified by C18 reverse phase column (0-75% MeOH in water) to give a crude solid. The solid was suspended in a solution (10% EtOAc in n-hexane). The resulting precipitate was collected by filtration to afford P70 (799 mg, 52% yield) as a yellow solid. ¹H NMR (400 MHZ, DMSO-d₆), δ: 8.92 (s, 1H), 8.71 (s, 1H), 4.57 (s, 2H), 4.13 (s, 2H), 3.76 (q, J=10.4 Hz, 2H), 3.53-3.48 (m, 2H), 3.42-3.35 (m, 2H), 1.86-1.84 (m, 4H), 1.47 (s, 9H); LCMS(ESI) [MH]⁺: 439.2; HPLC purity: 98.7%, Rt=20.806 min.

### Preparation 71. 4-(2,7-diazaspiro[3.5]non-2-yl)-6-(2,2,2-trifluoroethyl) pteridine (P71)

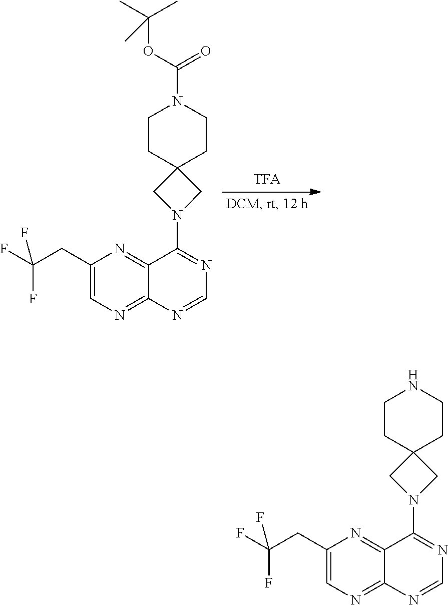 [SMILES: CC(C)(C)OC(=O)N1CCC2(CC1)CN(c1ncnc3ncc(CC(F)(F)F)nc13)C2 ; FC(F)(F)Cc1cnc2ncnc(N3CC4(CCNCC4)C3)c2n1]

To a stirred solution of P70 (300 mg, 0.68 mmol) in DCM (3 ml) was added TFA (0.78 g, 10 eq). After the reaction was stirred at rt overnight and the solution was treated with sat. NaHCO₃ and extracted with EtOAc. The combined organic layers were dried over with MgSO₄(s), filtered, and concentrated to give the product P71 as yellow solid (224 mg, 97%). LCMS(ESI) [MH]⁺: 339.2.

### Synthesis of 4-(2,7-diazaspiro[3.5]non-2-yl)-6-(2,2,2-trifluoroethyl)pyrido[3,4-d]pyrimidine (P85)

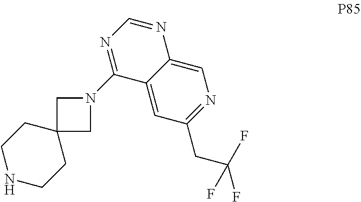 [SMILES: FC(F)(F)Cc1cc2c(N3CC4(CCNCC4)C3)ncnc2cn1]

### Preparation 72. 2-Bromo-5-nitroisonicotinic acid (P72)

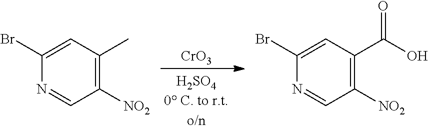

To a mixture of 2-bromo-4-methyl-5-nitro-pyridine (10.0 g, 46.5 mmol) and sulfuric acid (100 mL) was added chromium trioxide (15.5 g, 153 mmol) slowly in ice bath. The mixture was stirred at rt for overnight. The mixture was quenched by water slowly in ice bath. The resulting solid was collected by filtration and washed with water to afford P72 (9.15 g, 80%) as a white solid. ¹H NMR (400 MHZ, DMSO-d₆), δ: 9.10 (s, 1H), 8.13 (s, 1H), 4.59 (br. s, 1H).

### Preparation 73. Methyl 2-bromo-5-nitroisonicotinate (P73)

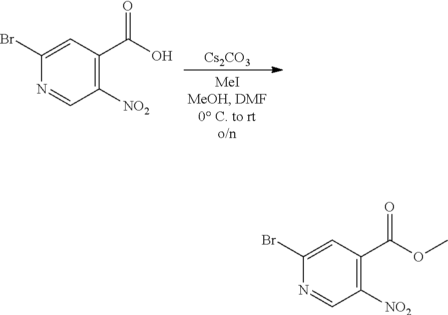

A mixture of P72 (9.15 g, 37.2 mmol) and cesium carbonate (6.04 g, 18.6 mmol) in MeOH (75 mL) was stirred for 30 min, and then the solution was concentrated. The mixture was treated with DMF (75 mL) and methyl iodide (2.80 mL, 44.6 mmol) in ice bath. The reaction was stirred at rt for overnight. The mixture was quenched by water slowly in ice bath. The resulting solid was collected by filtration and washed with water to afford P73 (9.20 g, 95%) as a white solid. ¹H NMR (400 MHZ, DMSO-d₆), δ: 9.17 (d, J=0.4 Hz, 1H), 8.22 (d, J=0.4 Hz, 1H), 3.90 (s, 3H).

### Preparation 74. Methyl 5-amino-2-bromoisonicotinate (P74)

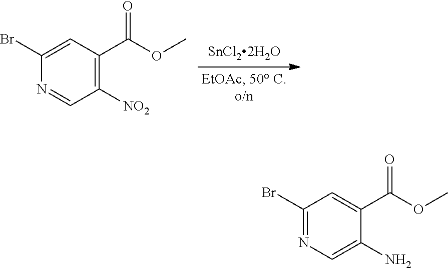

A mixture of P73 (9.20 g, 35.4 mmol) and tin (II) chloride dihydrate (39.91 g, 176.9 mmol) in EtOAc (118 mL) was stirred at 50° C. for overnight. The mixture was quenched by NaHCO₃(aq) in ice bath. The solution was filtered through a pad of celite and washed with EtOAc. The filtrate was extracted with EtOAc. The combined organic layers were dried over MgSO₄(s), filtered, and concentrated under reduced pressure to afford P74 (8.00 g, 98%) as a yellow solid which was used in next step without further purification. ¹H NMR (400 MHZ, DMSO-d₆), δ: 8.04 (s, 1H), 7.59 (s, 1H), 6.79 (s, 2H, NH₂), 3.28 (s, 3H).

### Preparation 75. Methyl 5-amino-2-vinylisonicotinate (P75)

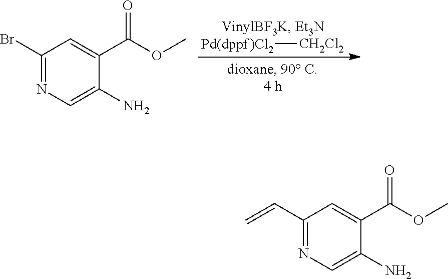

A mixture of P74 (8.00 g, 30.7 mmol), potassium vinyltrifluoroborate (6.16 g, 46.0 mmol) and triethylamine (21.4 mL, 15.3 mmol) in dioxane (123 mL) was degassed with argon for 15 min. The mixture was treated with 1,1′-bis(diphenylphosphino)ferrocene-palladium (II) dichloride dichloromethane complex (2.48 g, 3.07 mmol). The reaction mixture was stirred at 90° C. for 4 h. The mixture was filtered with a pad of celite and concentrated under reduced pressure. The residue was purified by silica gel column chromatography (0-50% EtOAc in n-hexane) to afford P75 (5.8 g, 94%) as a yellow solid. ¹H NMR (400 MHZ, CDCl₃), δ: 8.16 (s, 1H), 7.64 (s, 1H), 6.70 (dd, J=17.6, 10.8 Hz, 1H), 5.98 (dd, J=17.4, 1.2 Hz, 1H), 5.68 (s, 2H), 5.26 (dd, J=10.8, 1.2 Hz, 1H), 3.91 (s, 3H).

### Preparation 76. Methyl 5-(di-tert-butoxycarbonylamino)-2-vinylisonicotinate (P76)

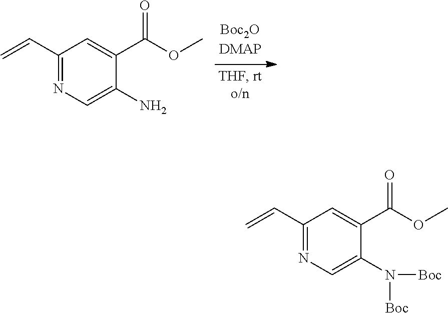 [SMILES: C=Cc1cc(C(=O)OC)c(N)cn1 ; C=Cc1cc(C(=O)OC)c(N(C(=O)OC(C)(C)C)C(=O)OC(C)(C)C)cn1]

A mixture of P75 (5.80 g, 32.6 mmol), di-tert-butyl dicarbonate (28.4 g, 130 mmol) and DMAP (0.40 g, 3.3 mmol) in THF (129 mL) was stirred at rt for overnight. The mixture was concentrated under reduced pressure. The mixture was treated with water and extracted with EtOAc. The combined organic layers were dried over MgSO₄(s), filtered, and concentrated under reduced pressure. The residue was purified by silica gel column chromatography (0-50% EtOAc in n-hexane) to afford P76 (8.3 g, 69%) as a yellow solid. ¹H NMR (400 MHZ, CDCl₃), δ: 8.42 (s, 1H), 7.81 (s, 1H), 6.83 (dd, J=17.4, 10.8 Hz, 1H), 6.29 (dd, J=17.4, 1.0 Hz, 1H), 5.58 (dd, J=10.8, 1.0 Hz, 1H), 3.90 (s, 3H), 1.36 (s, 18H).

### Preparation 77. Methyl 5-(di-tert-butoxycarbonylamino)-2-formylisonicotinate (P77)

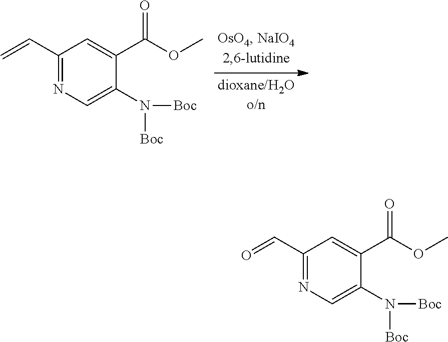 [SMILES: C=Cc1cc(C(=O)OC)c(N(C(=O)OC(C)(C)C)C(=O)OC(C)(C)C)cn1 ; COC(=O)c1cc(C=O)ncc1N(C(=O)OC(C)(C)C)C(=O)OC(C)(C)C]

To a mixture of P76 (8.30 g, 22.5 mmol) in dioxane (180 mL) and water (45 mL) was added sodium periodate (19.2 g, 89.8 mmol), 2,6-lutidine (5.2 mL, 44.92 mmol) and osmium tetroxide (2.5% in tert-BuOH, 4.5 mL, 0.45 mmol). After the solution was stirred at rt for overnight, the mixture was filtered and washed with EtOAc. The organic layer was dried over MgSO₄(s), filtered, and concentrated under reduced pressure. The residue was purified by silica gel column chromatography (0-35% EtOAc in n-hexane) to afford P77 (6.23 g, 74%) as a yellow solid. ¹H NMR (400 MHZ, DMSO-d₆): § 10.02 (s, 1H), 8.92 (d, J=0.8 Hz, 1H), 8.23 (d, J=0.8 Hz, 1H), 3.87 (s, 3H), 1.31 (s, 18H); LCMS(ESI) [MH]⁺: 381.2.

### Preparation 78. Methyl 5-(di-tert-butoxycarbonylamino)-(E)-2-(hydrazonomethyl)isonicotinate (P78)

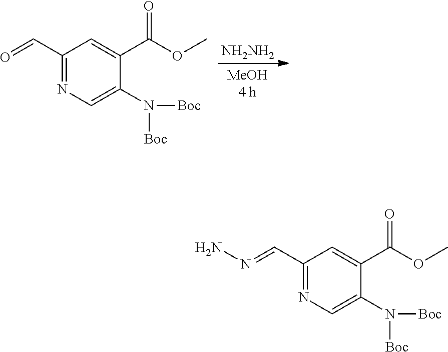

A solution of P77 (6.23 g, 16.4 mmol) and hydrazine (0.90 mL, 20 mmol) in MeOH (126 mL) was stirred at rt for 4 h. The solution was concentrated under reduced pressure to afford P78 (6.50 g, crude) as a yellow oil which was used in next step without further purification. ¹H NMR (400 MHZ, DMSO-d₆), δ: 8.41 (s, 1H), 8.05 (s, 1H), 7.72 (s, 1H), 7.58 (s, 2H), 3.82 (s, 3H), 1.31 (s, 18H).

### Preparation 79. Methyl 5-(di-tert-butoxycarbonylamino)-2-(2,2,2-trifluoroethyl)isonicotinate (P79)

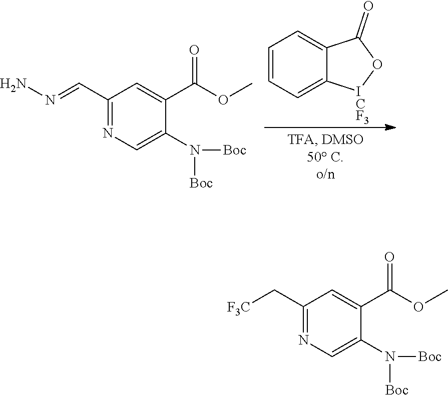

To a solution of P78 (6.50 g, 16.4 mmol) and TFA (1.20 mL, 16.44 mmol) in DMSO (63.5 mL) was added 1-(trifluoromethyl)-1,2 benziodaoxol-3 (1H)-one (5.69 g, 18.0 mmol) in ice bath. After the reaction was stirred at rt for overnight, the mixture was treated with NaHCO₃ (aq.) and extracted with EtOAc. The combined organic layers were dried over MgSO₄(s), filtered, and concentrated under reduced pressure. The residue was purified by silica gel column chromatography (0-50% EtOAc in n-hexane) to afford P79 (4.3 g, 60%) as a yellow solid. ¹H NMR (400 MHz, CDCl₃), δ: 8.48 (s, 1H), 7.83 (s, 1H), 3.91 (s, 3H), 3.68 (q, J=10.4 Hz, 2H), 1.37 (s, 18H).

### Preparation 80. Methyl 5-amino-2-(2,2,2-trifluoroethyl)isonicotinate (P80)

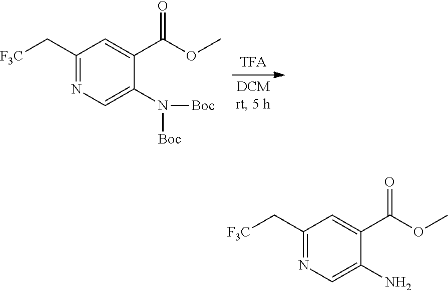 [SMILES: COC(=O)c1cc(CC(F)(F)F)ncc1N(C(=O)OC(C)(C)C)C(=O)OC(C)(C)C ; COC(=O)c1cc(CC(F)(F)F)ncc1N]

A mixture of P79 (4.30 g, 16.4 mmol) and TFA (16.5 mL) in DCM (33 mL) was stirred at rt for 5 h. The mixture was treated NaHCO₃(aq) and extracted with DCM. The combined organic layers were dried over MgSO₄(s), filtered, and concentrated under reduced pressure to afford P80 (2.18 g, crude) as a yellow oil which was used in next step without further purification. ¹H NMR (400 MHZ, DMSO-d₆), δ: 8.21 (s, 1H), 7.55 (s, 1H), 6.74 (s, 2H), 3.83 (s, 3H), 3.61 (q, J=11.6 Hz, 2H).

### Preparation 81. 5-amino-2-(2,2,2-trifluoroethyl) isonicotinic acid (P81)

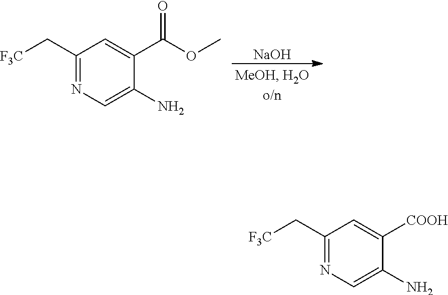

A solution of P80 (2.18 g, 9.31 mmol) and sodium hydroxide (1M solution in water, 25.9 mL, 25.9 mmol) in MeOH (26 mL) was stirred at rt for overnight. Dowax H⁺ was added to the mixture. The solution was filtered, washed with EtOAc and MeOH, and concentrated under reduced pressure to afford P81 (1.94 g) as a yellow oil which was used in next step without further purification. ¹H NMR (400 MHZ, DMSO-d₆), δ: 8.03 (s, 1H), 7.56 (s, 1H), 3.54 (q, J=11.6 Hz, 2H); LRMS(ESI) [MH]⁺: 221.1.

### Preparation 82. 5-Amino-2-(2,2,2-trifluoroethyl) isonicotinamide (P82)

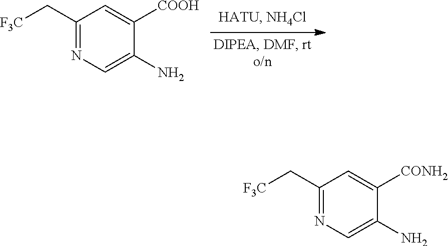 [SMILES: Nc1cnc(CC(F)(F)F)cc1C(=O)O ; NC(=O)c1cc(CC(F)(F)F)ncc1N]

A mixture of P81 (1.94 g, 8.82 mmol), ammonium chloride (1.40 g, 26.45 mmol), HATU (6.70 g, 17.64 mmol), and DIPEA (3.1 mL, 18 mmol) in DMF (38 mL) was stirred at rt for overnight. The mixture was treated with water and extracted with EtOAc. The combined organic layers were dried over MgSO₄(s), filtered, and concentrated under reduced pressure. The residue was purified by silica gel column chromatography (0-75% EtOAc in n-hexane containing 1% of triethylamine) to afford P82 (1.0 g, 51%) as a yellow solid. ¹H NMR (400 MHZ, DMSO-d₆), δ: 8.09 (s, 1H), 7.99 (br. s, 1H), 7.45 (s, 2H), 6.62 (s, 2H), 3.50 (q, J=11.2 Hz, 2H).

### Preparation 83. 6-(2,2,2-Trifluoroethyl)pyrido[3,4-d]pyrimidin-4 (3H)-one (P83)

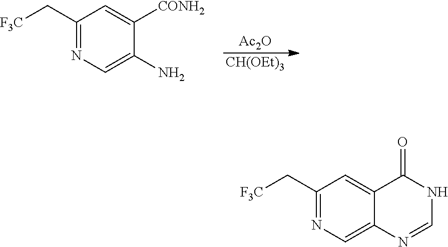 [SMILES: "Ac2O" ; NC(=O)c1cc(CC(F)(F)F)ncc1N ; O=c1[nH]cnc2cnc(CC(F)(F)F)cc12 ; "CH(OEt)3"]

A mixture of P82 (1.0 g, 4.7 mmol) and acetic anhydride (6.5 mL, 69 mmol) in triethyl orthoformate (13 mL) was stirred at 120° C. for 6 h. The mixture was concentrated under reduced pressure. The residue was purified by silica gel column chromatography (0-100% EtOAc in n-hexane with 1% of triethylamine) to afford P83 (0.80 g, 74%) as a yellow solid. ¹H NMR (400 MHZ, DMSO-d₆), δ: 9.03 (s, 1H), 8.24 (s, 1H), 8.05 (s, 1H), 3.98 (q, J=11.2 Hz, 2H); LRMS(ESI) [MH]⁺: 230.1; HPLC purity: 91.5%, t_{R}=11.00 min.

### Preparation 84. tert-Butyl 2-(6-(2,2,2-trifluoroethyl)pyrido[3,4-d]pyrimidin-4-yl)-2,7-diazaspiro[3.5]nonane-7-carboxylate (P84)

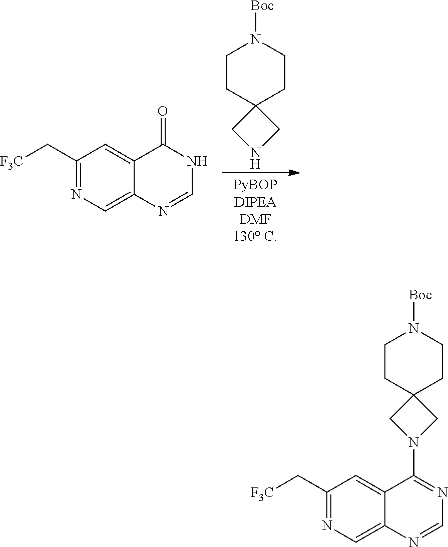 [SMILES: O=c1[nH]cnc2cnc(CC(F)(F)F)cc12 ; CC(C)(C)OC(=O)N1CCC2(CC1)CNC2 ; CC(C)(C)OC(=O)N1CCC2(CC1)CN(c1ncnc3cnc(CC(F)(F)F)cc13)C2]

A mixture of P83 (0.30 g, 1.3 mmol), tert-butyl 2,7-diazaspiro[3.5]nonane-7-carboxylate (549 mg, 2.10 mmol), benzotriazol-1-yloxytripyrrolidinophosphonium hexafluorophosphate (1.09 g, 2.10 mmol) and DIPEA (1.2 mL, 6.5 mmol) in DMF (6.0 mL) was stirred at 130° C. for 6 h. The mixture was treated with water and extracted with EtOAc. The organic layers were washed with brine, dried over Na₂SO₄ (s), filtered, and concentrated under reduced pressure. The residue was purified by silica gel column chromatography (0-100% EtOAc in n-hexane with 1% of triethylamine) to afford P84 (169 mg, 30%) as a yellow solid. ¹H NMR (400 MHZ, CDCl₃), δ: 9. 23 (s, 1H), 8.69 (s, 1H), 7.62 (s, 1H), 4.29 (s, 4H), 3.74 (q, J=10.6 Hz, 2H), 3.46 (s, 4H), 1.88-1.85 (m, 4H), 1.48 (s, 9H); LRMS(ESI) [MH]⁺: 438.2; HPLC purity: 97.07%, t_{R}=19.55 min.

### Preparation 85. 4-(2,7-diazaspiro[3.5]non-2-yl)-6-(2,2,2-trifluoroethyl)pyrido[3,4-d]pyrimidine (P85)

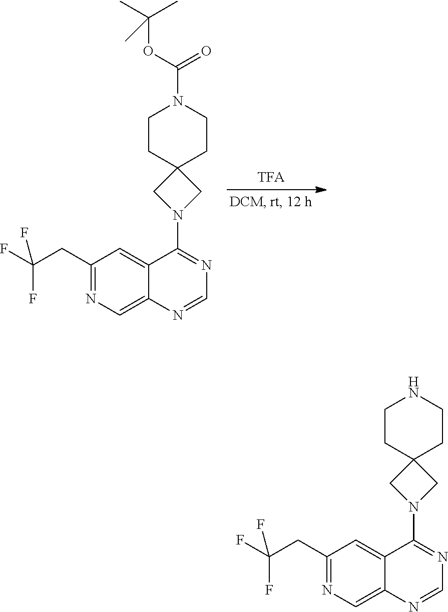 [SMILES: CC(C)(C)OC(=O)N1CCC2(CC1)CN(c1ncnc3cnc(CC(F)(F)F)cc13)C2 ; FC(F)(F)Cc1cc2c(N3CC4(CCNCC4)C3)ncnc2cn1]

To a stirred solution of P84 (150 mg, 0.34 mmol) in DCM (3 ml) was added TFA (0.391 g, 10 eq). After the reaction was stirred at rt overnight and the solution was treated with sat. NaHCO₃ and extracted with EtOAc. The combined organic layers were dried over with MgSO₄(s), filtered, and concentrated to give the product P85 as yellow solid (85 mg, 73%). LCMS(ESI) [MH]⁺: 338.3.

### Synthesis of 2-Trityl-3,4-dihydro-1H-isoquinoline-6-carbaldehyde (P87)

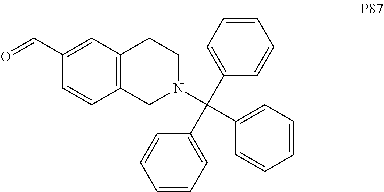 [SMILES: O=Cc1ccc2c(c1)CCN(C(c1ccccc1)(c1ccccc1)c1ccccc1)C2]

### Preparation 86. 6-Bromo-2-trityl-3,4-dihydro-1H-isoquinoline (P86)

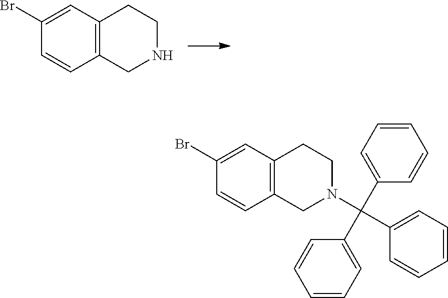 [SMILES: Brc1ccc2c(c1)CCNC2 ; Brc1ccc2c(c1)CCN(C(c1ccccc1)(c1ccccc1)c1ccccc1)C2]

To a stirred solution of 6-bromo-1,2,3,4-tetrahydroisoquinoline (1 g, 4.71 mmol) in DCM (10 mL) was added triethylamine (0.525 g, 0.723 mL, 5.18 mmol), and the solution was stirred in an ice bath. 4-(Dimethylamino)pyridine (0.057 g, 0.47 mmol) and triphenylmethyl chloride (1.972 g, 7.06 mmol) were added in one portion, and the reaction mixture was stirred vigorously at room temperature for 18 h, quenched with 1 N NaOH, and extracted with DCM (thrice), and the combined organic extract was dried over anhydrous Na₂SO₄ and evaporated. The residue was purified by column chromatography on silica with hexane-ethylacetate (100:2) to yield 6-bromo-2-trityl-3,4-dihydro-1H-isoquinoline (P86, 954 mg, 45%) as a white solid. ¹H NMR (400 MHZ, DMSO-d₆), δ: 7.45 (d, J=7.8 Hz, 6H), 7.36-7.28 (m, 7H), 7.30-7.21 (m, 1H), 7.24-7.15 (m, 4H), 6.95 (d, J=8.2 Hz, 1H), 3.27 (s, 2H), 2.97 (s, 2H), 2.38 (s, 2H).

### Preparation 87. 2-Trityl-3,4-dihydro-1H-isoquinoline-6-carbaldehyde (P87)

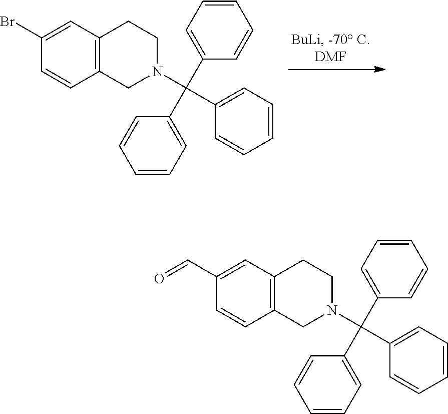 [SMILES: Brc1ccc2c(c1)CCN(C(c1ccccc1)(c1ccccc1)c1ccccc1)C2 ; O=Cc1ccc2c(c1)CCN(C(c1ccccc1)(c1ccccc1)c1ccccc1)C2]

To the solution of 6-bromo-2-trityl-3,4-dihydro-1H-isoquinoline P86 (940 mg, 2.07 mmol) in THF (5 mL) was added dropwise n-BuLi (1.3 mL, 2.5 mol/L, 3.1 mmol) while maintaining the temperature between −75 to −70° C. After 30 min, DMF (756 mg, 10.35 mmol) was added dropwise and the resulting mixture was stirred at −75° C. for 30 min, then allowed to warm to rt. The reaction mixture was quenched with saturated NH₄Cl solution, extracted with EtOAc (3×15 mL), washed with brine, dried over Na₂SO₄, filtered and concentrated. The residue was purified by flash chromatography to give the target compound P87 (512 mg, 61%) as an oil. ¹H-NMR (400 MHZ, DMSO-d₆), δ: 9.93 (s, 1H), 7.68 (s, 1H), 7.60 (d, J=7.9 Hz, 1H), 7.47 (d, J=7.7 Hz, 6H), 7.33 (td, J=7.8, 3.1 Hz, 7H), 7.21 (dt, J=9.5, 5.0 Hz, 5H), 3.42 (s, 2H), 3.07 (s, 2H), 2.43 (s, 2H).

### Synthesis of tert-Butyl 5-formylisoindoline-2-carboxylate (P88)

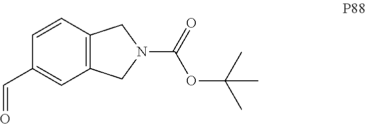 [SMILES: CC(C)(C)OC(=O)N1Cc2ccc(C=O)cc2C1]

### Preparation 88. tert-Butyl 5-formylisoindoline-2-carboxylate (P88)

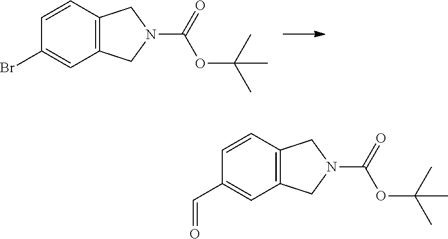

To a solution of the tert-butyl 5-bromoisoindoline-2-carboxylate (510 mg, 1.7 mmol) in THF (5 mL) was added n-BuLi (1 mL, 2.5 mol/L, 2.5 mmol) dropwise maintaining the temperature between −75 and −70° C. After 30 min, DMF (625 mg, 8.55 mmol) was added dropwise and the resulting mixture was stirred at −75° C. for additional 30 min, then the temperature was allowed to increase to room temperature. The reaction was quenched with saturated NH₄Cl solution, extracted with EtOAc (3×15 mL), washed with brine, dried over Na₂SO₄, filtered, and concentrated. The residue was purified by flash chromatography to give the target compound (P88, 110 mg, 26%) as oil. ¹H NMR (400 MHZ, DMSO-d₆), δ: 9.99 (s, 1H), 7.95-7.74 (m, 2H), 7.56 (dd, J=7.9, 5.0 Hz, 1H), 4.66 (d, J=8.6 Hz, 4H), 1.46 (d, J=1.1 Hz, 9H).

### Synthesis of N-(trans-4-Formylcyclohexyl)ethanesulfonamide (P133)

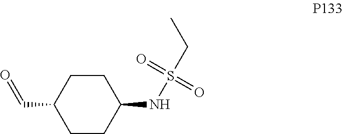

### Preparation 129. {trans-4-[(tert-Butoxycarbonyl)amino]cyclohexyl}methyl acetate (P129)

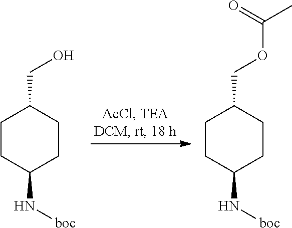

To a solution of tert-butyl [trans-4-(hydroxymethyl)cyclohexyl]carbamate (5 g, 21.8 mmol) and triethylamine (6 g, 59 mmol) in DCM (50 ml) with stirring, at rt was added acetyl chloride (1.8 g, 22 mmol). The solution was stirred for 18 h, then was washed with aq. potassium hydrosulfate up to pH=3 and extracted with chloroform (50 ml). Organic extract was concentrated to give the product (P129, 5.9 g, 99%). ¹H NMR (400 MHZ, DMSO-d₆), δ: 6.70 (s, 1H), 3.95-3.84 (m, 1H), 3.84-3.73 (m, 2H), 3.37-3.24 (m, 1H), 2.07-1.88 (m, 3H), 1.88-1.56 (m, 4H), 1.34 (s, 9H), 1.19-0.86 (m, 4H).

### Preparation 130. (trans-4-Aminocyclohexyl)methyl acetate (P130)

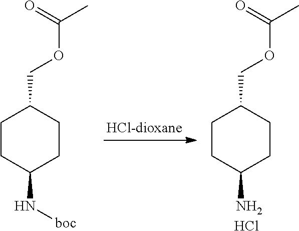

A solution of HCl in dioxan (3 M, 100 ml) was added to {trans-4-[(tert-butoxycarbonyl)amino]cyclohexyl}methyl acetate (P129, 5.9 g, 21.5 mmol). The reaction mixture was stirred at room temperature for 2 h (NMR control), then concentrated to dryness to give the product P130 (4.4 g, 99%). ¹H NMR (400 MHZ, DMSO-d₆), δ: 8.17 (s, 3H), 3.82 (d, J=6 Hz, 2H), 2.05-1.88 (m, 5H), 1.79-1.67 (m, 2H), 1.60-1.44 (m, 1H), 1.40-1.22 (m, 2H), 1.11-0.93 (m, 2H).

### Preparation 131. {trans-4-[(Ethylsulfonyl)amino]cyclohexyl}methyl acetate (P131)

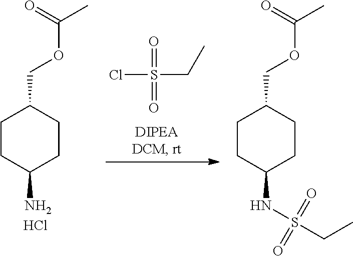

To a suspension of (trans-4-aminocyclohexyl)methyl acetate (P130, 4.4 g, 21.5 mmol) in chloroform (70 ml) DIPEA (1.39 g, 108 mmol) and ethanesulfonyl chloride (4.06 g, 32 mmol). The obtained solution was stirred at rt for 24 h (LCMS control), then washed with aq. potassium hydrosulfate up to pH=3, extracted with chloroform (50 ml) and concentrated. Residue was purified by silica gel column chromatography (ethyl acetate-DCM 1:10) to give the product P131 (4.52 g, 80%).′ H NMR (400 MHZ, DMSO-d₆), δ: 6.99 (d, J=6.8 Hz, 1H), 3.82 (d, J=6.3 Hz, 2H), 3.07-2.84 (m, 3H), 2.00 (d, J=7.6 Hz, 3H), 1.87 (d, J=7.8 Hz, 2H), 1.69 (d, J=10.1 Hz, 2H), 1.48 (s, 1H), 1.34-1.11 (m, 6H), 1.01 (d, J=11.8 Hz, 2H).

### Preparation 132. N-[trans-4-(Hydroxymethyl)cyclohexyl]ethanesulfonamide (P132)

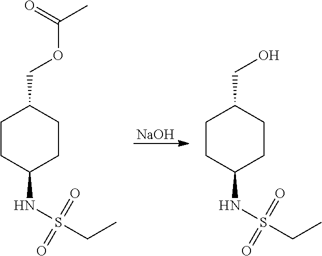 [SMILES: CCS(=O)(=O)N[C@H]1CC[C@H](COC(C)=O)CC1 ; "NaOH" ; CCS(=O)(=O)N[C@H]1CC[C@H](CO)CC1]

P131 (4.52 g, 17.2 mmol) was dissolved in methanol (100 ml), and solution of sodium hydroxide (2 g, 50 mmol) in water (10 ml) was added. Reaction mixture was stirred at rt for 2 h (TLC control), then concentrated to dryness. Residue was dissolved in water (100 ml), and concentrated HCl was added up to pH=3. Product P132 was extracted with dichloromethane (3×100 ml) and concentrated to dryness (3.65 g, 90%). ¹H NMR (400 MHZ, DMSO-d₆), δ: 6. 98 (d, J=8.1 Hz, 1H), 4.36 (t, J=5.1 Hz, 1H), 3.18 (t, J=5.6 Hz), 3.03-2.89 (m, 2H), 1.94-1.80 (m, 2H), 1.77-1.65 (m, 2H), 1,30-1.11 (m, 6H), 0.98-0.82 (m, 2H).

### Preparation 133. N-(trans-4-Formylcyclohexyl)ethanesulfonamide (P133)

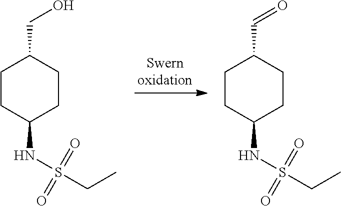

To a solution of oxalyl chloride (2.51 g, 19.7 mmol) in dichloromethane (25 ml) with stirring at −70° C., under argon atmosphere was added a solution of dimethyl sulfoxide (3.35 g) in dichloromethane (5 ml). The mixture was stirred for 30 min at −70° C., then solution of N-[trans-4-(hydroxymethyl)cyclohexyl]ethanesulfonamide (P132, 3.65 g, 16.5 mmol) in dichloromethane (10 ml) was added. The resulting mixture was stirred at −75° C. for additional 90 min, and triethylamine (10.1 g, 0.1 mol) was added at −70° C. After the temperature of the reaction mixture reached rt, water (20 ml) was added, and mixture stirred for additional 10 min. Organic phase was separated, water phase extracted with dichloromethane (50 ml). Combined organic phase was concentrated to dryness to give a product (P133, 3.5 g, 95%). ¹H NMR (400 MHz, DMSO-d₆), δ: 9.54 (d, J=1.0 Hz, 1H), 7.05 (d, J=8.4 Hz, 1H), 3.07-2.93 (m, 3H), 2.52-2.47 (m, 2H), 2.22-2.12 (m, 1H), 1.98-1.83 (m, 4H), 1.27-1.13 (m, 5H).

### Synthesis of N-[4-(Formyl)phenyl]ethanesulfonamide (P137)

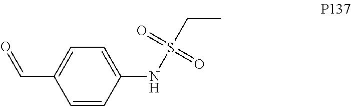

### Preparation 134. 1-(Dimethoxymethyl)-4-nitrobenzene (P134)

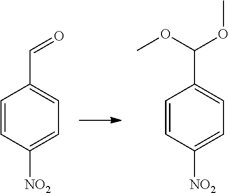

4-Formyl-nitrobenzene (5.5 g, 36.4 mmol) was dissolved in dry methanol (150 ml), then TsOH (0.5 g) was added, and mixture stirred at 60° C. for 5 h. Then the reaction mixture was cooled down to rt, concentrated in vacuo, and residue was dissolved in dichloromethane (100 ml). This solution was stirred with water solution of sodium bicarbonate. Organic layer was separated, water layer was extracted with dichloromethane (100 ml). Combined dichloromethane solution was evaporated to dryness to give pure 1-(dimethoxymethyl)-4-nitrobenzene (P134, 7,1 g, 99%). ¹H NMR (400 MHZ, CDCl₃), δ: 8.20 (d, J=6.2 Hz, 2H), 7.63 (d, J=6.6 Hz, 2H), 3.40-3.27 (m, 6H).

### Preparation 135. 1-(Dimethoxymethyl)-4-aminobenzene (P135)

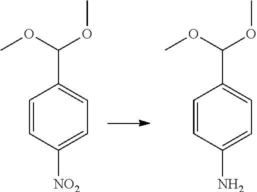

A solution of 1-(dimethoxymethyl)-4-nitrobenzene (P134, 7,1 g, 36 mmol) in methanol (100 ml) was stirred with 5 g of Raney Nickel under hydrogen atmosphere at rt for 1 h. After the reaction was completed, the solution was decanted, filtered throw celite and evaporated to dryness to crude 1-(dimethoxymethyl)-4-aminobenzene (P135, 4.8 g, 80%). ¹H NMR (400 MHz, CDCl₃), δ: 7.23 (d, J=9.2 Hz, 2H), 6.67 (d, J=5.6 Hz, 2H), 5.33-5.28 (m, 1H), 3.37-3.26 (m, 6H).

### Preparation 136. N-[4-(Dimethoxymethyl)phenyl]ethanesulfonamide (P136)

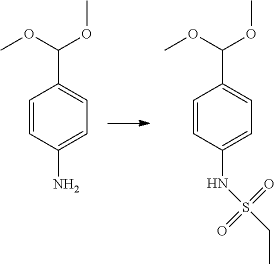

Ethanesulfonyl chloride (8.6 g, 66.4 mmol) slowly added with stirring at rt to a mixture of 1-(dimethoxymethyl)-4-aminobenzene (P135, 4.8 g, 28.7 mmol), sodium bicarbonate (12.5 g), dioxane (60 g) and water (50 ml). The mixture was stirred at rt for 18 h, then extracted with DCM (200 ml). Extract was concentrated in vacuo to dryness, a residue was purified by silica column chromatography (ethyl acetate-dichloromethane 5:95) to give a product (P136, 3 g, 40%). ¹H NMR (400 MHZ, CDCl₃), δ: 7.42 (d, J=7.4 Hz, 2H), 7.24 (d, J=9.0 Hz, 2H), 5.36 (s, 1H), 3.34 (s, 6H), 3.19-3.10 (m, 2H), 1.41-1.28 (m, 3H).

### Preparation 137. N-[4-(Formyl)phenyl]ethanesulfonamide (P137)

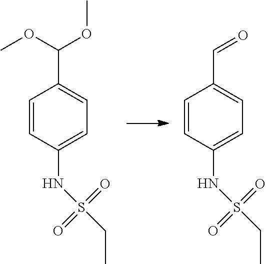 [SMILES: CCS(=O)(=O)Nc1ccc(C(OC)OC)cc1 ; CCS(=O)(=O)Nc1ccc(C=O)cc1]

To a solution of N-[4-(dimethoxymethyl)phenyl]ethanesulfonamide (P136, 3 g, 11.7 mmol) in THF (50 ml) water (5 ml) and TFA (4 g, 35 mmol) were added. The mixture stirred at rt for 5 h (TLC control). Then the reaction mixture was evaporated to dryness to give the product P137 (2.5 g, 100%). ¹H NMR (400 MHZ, DMSO-d₆), δ: 10.46 (s, 1H), 9.87 (d, J=3.1 Hz, 1H), 7.86 (d, J=9.8 Hz, 2H), 7.37 (d, J=7.3 Hz, 2H), 3.30-2.15 (m, 2H), 1.35-0.89 (m, 3H).

### Synthesis of N-[3-(Formyl)phenyl]ethanesulfonamide (P141)

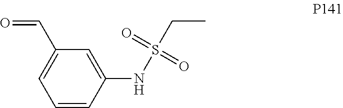

### Preparation 138. 1-(Dimethoxymethyl)-3-nitrobenzene (P138)

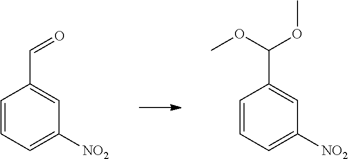

3-Formyl-nitrobenzene (5.5 g, 36.4 mmol) was dissolved in dry methanol (150 ml). TsOH (0.5 g) was added, and mixture stirred at 60° C. for 5 h. After the reaction mixture was cooled down to rt, concentrated under reduce pressure, and dissolved in DCM (100 ml). Dichloromethane solution was washed with water solution of sodium bicarbonate and organic phase was separated. Water phase was extracted with DCM (100 ml). Combined DCM solution was evaporated to dryness to give crude 1-(dimethoxymethyl)-3-nitrobenzene-P138 (7,1 g).

### Preparation 139. 1-(Dimethoxymethyl)-3-aminobenzene (P139)

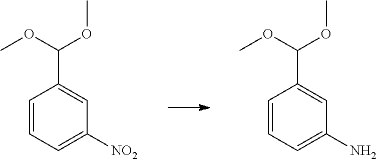

A solution of 1-(dimethoxymethyl)-3-nitrobenzene (P138, 7,1 g, 36 mmol) in methanol (100 ml) was stirred with 5 g of Raney Nickel under hydrogen atmosphere at rt for 1 h. The solution was decanted, filtered with celite, and evaporated to dryness to yield crude 1-(dimethoxymethyl)-3-aminobenzene (P139, 4.8 g).

### Preparation 140. N-[3-(Dimethoxymethyl)phenyl]ethanesulfonamide (P140)

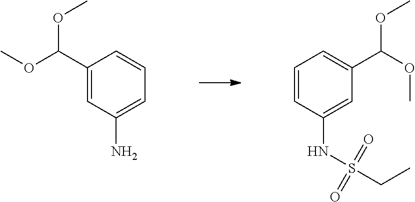 [SMILES: COC(OC)c1cccc(N)c1 ; CCS(=O)(=O)Nc1cccc(C(OC)OC)c1]

Ethanesulfonyl chloride (8.6 g, 66.4 mmol) was slowly added with generous stirring at rt to a mixture of 1-(dimethoxymethyl)-3-aminobenzene (P139, 4.8 g, 28.7 mmol), sodium bicarbonate (12.5 g), dioxane (60 g) and water (50 ml). The reaction mixture was stirred at rt for additional 18 h, then extracted with dichloromethane (200 ml). The extract was concentrated to dryness to yield the crude product P140 (3 g).

### Preparation 141. N-[3-(Formyl)phenyl]ethanesulfonamide (P141)

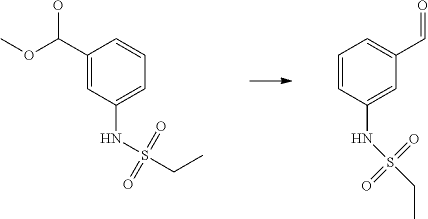 [SMILES: CCS(=O)(=O)Nc1cccc(C(O)OC)c1 ; CCS(=O)(=O)Nc1cccc(C=O)c1]

Water (5 ml) and TFA (4 g, 35 mmol) were added to solution of N-[3-(dimethoxymethyl)phenyl]ethanesulfonamide (P140, 3 g, 11.7 mmol) in THF (50 ml). The mixture was stirred at rt for 5 h (TLC control), after reaction completed the solvents were evaporated to dryness to yield 1.12 g, 50% of the product P141. ¹H NMR (400 MHZ, DMSO-d₆), δ: 10.46 (s, 1H), 9.87 (d, J=3.1 Hz, 1H), 7.73-7.70 (m, 1H), 7.64 (d, J=9 Hz, 1H), 7.57 (t, J=7.3 Hz, 1H), 3.30-2.15 (m, 2H), 1.35-0.89 (m, 3H).

### Synthesis of 7-(2,7-diazaspiro[3.5]nonan-7-ylmethyl)-4H-1,4-benzoxazin-3-one (P145)

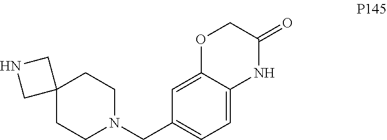 [SMILES: O=C1COc2cc(CN3CCC4(CC3)CNC4)ccc2N1]

### Preparation 142. 2-(5-Formyl-2-nitro-phenoxy) acetamide (P142)

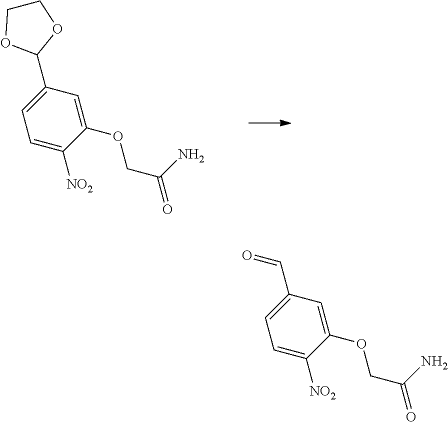 [SMILES: NC(=O)COc1cc(C2OCCO2)ccc1[N+](=O)[O-] ; NC(=O)COc1cc(C=O)ccc1[N+](=O)[O-]]

The solution of 2-[5-(1,3-dioxolan-2-yl)-2-nitrophenoxy]acetamide (350 mg, 1.2 mmol) in AcOH—H₂O (1 ml, 1:1) was stirred at 80° C. for 16 h (TLC control). After reaction completed the solvents were evaporated to dryness to yield P142 (280 mg, 100%). LCMS(ESI) [MH]⁺: 225.

### Preparation 143. tert-Butyl 7-[[3-(2-amino-2-oxo-ethoxy)-4-nitro-phenyl]methyl]-2,7-diazaspiro[3.5]nonane-2-carboxylate (P143)

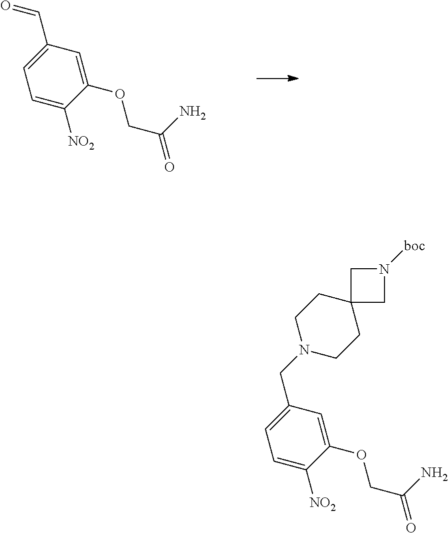

Mixture of tert-butyl 2,7-diazaspiro[3.5]nonane-2-carboxylate hydrochloride (320 mg, 1.2 mmol), 2-(5-formyl-2-nitrophenoxy) acetamide (P142, 280 mg, 1.2 mmol) and sodium triacethoxyborohydride (1.6 g, 7.5 mmol) in DCM (5 ml) was stirred at rt for 5 h (LCMS control). After the reaction was completed, water solution of sodium bicarbonate was added, and mixture stirred for additional 30 min. After extraction with dichloromethane (20 ml), concentration of the solvent obtained residue was purified by silica chromatography (dichloromethane-ethylacetate (50:50) to yield tert-butyl 7-[3-(2-amino-2-oxoethoxy)-4-nitrobenzyl]-2,7-diazaspiro[3.5]nonane-2-carboxylate (P143, 450 mg, 83%). LCMS(ESI) [MH]⁺: 435.

### Preparation 144. tert-Butyl 7-[(3-oxo-4H-1,4-benzoxazin-7-yl)methyl]-2,7-diazaspiro[3.5]nonane-2-carboxylate (P144)

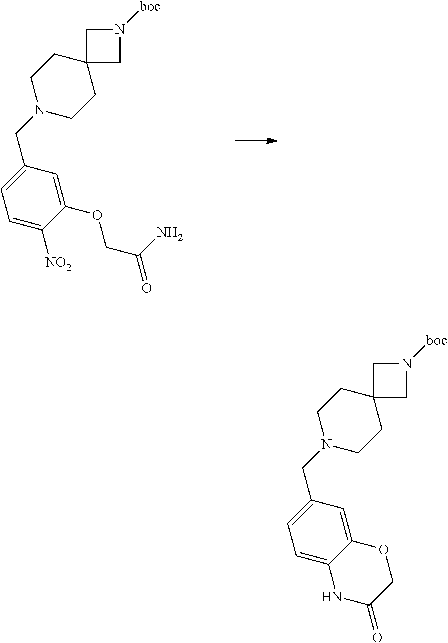

To the solution of tert-butyl 7-[3-(2-amino-2-oxoethoxy)-4-nitrobenzyl]-2,7-diazaspiro[3.5]nonane-2-carboxylate (P143, 450 mg, 1.0 mmol) in THF (5 ml) acetic acid (620 mg, 10.0 mmol) and Zn dust (1.3 g, 20.0 mmol) were added. The mixture was stirred at rt 18 h (LCMS control). After the reaction was completed, the mixture was filtered off from Zn, concentrated, washed with water solution of sodium bicarbonate, and extracted with DCM (2×20 ml). Evaporation the extract to dryness to yield the product P144 (50 mg, 15%). LCMS (ESI) [MH]⁺: 388.

### Preparation 145. 7-(2,7-diazaspiro[3.5]nonan-7-ylmethyl)-4H-1,4-benzoxazin-3-one (P145)

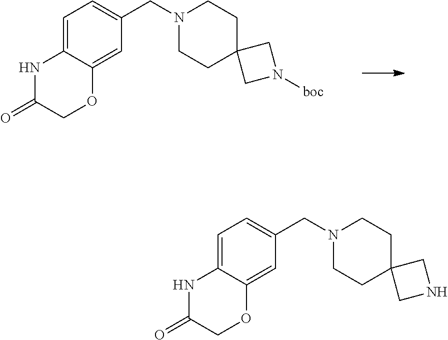

tert-Butyl 7-[(3-oxo-3,4-dihydro-2H-1,4-benzoxazin-7-yl)methyl]-2,7-diazaspiro[3.5]nonane-2-carboxylate (P144, 50 mg, 0.13 mmol) was added to HCl/dioxane solution (3M, 0.2 ml). The reaction mixture stirred at rt for 3 h (LCMS control), then evaporated under vacuum to dryness to yield 7-(2,7-diazaspiro[3.5]non-7-ylmethyl)-2H-1,4-benzoxazin-3 (4H)-one hydrochloride (P145, 42 mg, 100%) as a white solid. LCMS(ESI) [MH]⁺: 288.

### Synthesis of 4-(2,7-Diazaspiro[3.5]non-2-yl)-6-(2-phenylethyl)quinazoline (P148)

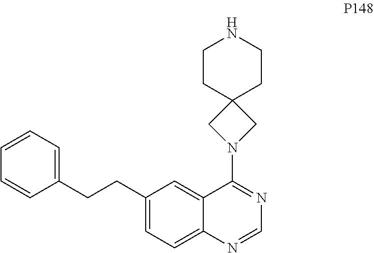 [SMILES: c1ccc(CCc2ccc3ncnc(N4CC5(CCNCC5)C4)c3c2)cc1]

### Preparation 146. tert-Butyl 2-(6-bromoquinazolin-4-yl)-2,7-diazaspiro[3.5]nonane-7-carboxylate (P146)

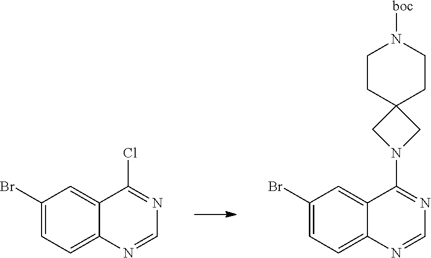

To a solution 6-bromo-4-chloroquinazoline prepared according to WO2013/57711, 2013, A1 (3 g, 12.3 mmol) in DCM (60 ml) were added DIPEA (7.9 g, 61 mmol) and tert-butyl 2,7-diazaspiro[3.5]nonane-7-carboxylate hydrochloride (3.2 g, 12.2 mmol). The reaction solution was stirred at rt for 18 h (LCMS control). Then obtained product was purified by column chromatography on silica with ethylacetate-methanol (10:1) to yield 5.1 g (96%) of P146 as a white solid. LCMS(ESI) [MH]⁺: 434.

### Preparation 147. of tert-Butyl 2-[6-(2-phenylethyl)quinazolin-4-yl]-2,7-diazaspiro[3.5]nonane-7-carboxylate (P147)

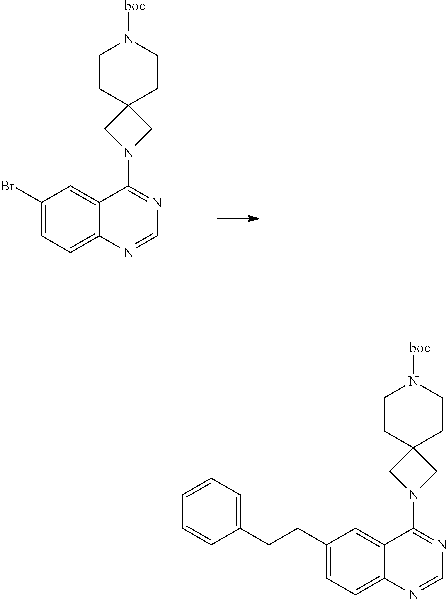

To a solution of tert-butyl 2-(6-bromoquinazolin-4-yl)-2,7-diazaspiro[3.5]nonane-7-carboxylate (P146, 200 mg 0.46 mmol) in DMAA (2 ml) under argon atmosphere were added TEA (186 mg, 1.84 mmol), styrene (144 mg, 1.48 mmol) and Pd(PPh₃)₄ (20 mg). The mixture was stirred at 90° C. for 16 h (LCMS control). After reaction completed Pd—C(30 mg) was added and the mixture was stirred at rt under hydrogen atmosphere until reaction completed (LCMS control). Then the mixture was filtered, washed with brine (40 ml) and extracted with ethylacetate (2×5 ml), concentrated and residue was purified by silica gel column chromatography (dichloromethane-ethylacetate-1:1) to give the title product P147 (145 mg, 69%). LCMS(ESI) [MH]⁺: 459.

### Preparation 148. 4-(2,7-Diazaspiro[3.5]non-2-yl)-6-(2-phenylethyl)quinazoline (P148)

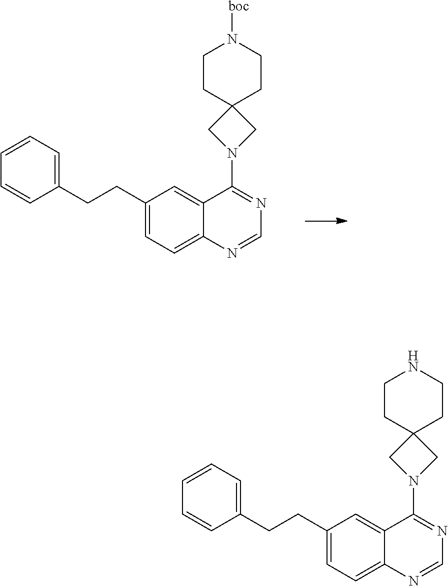

To tert-butyl 2-[6-(2-phenylethyl)quinazolin-4-yl]-2,7-diazaspiro[3.5]nonane-7-carboxylate (P147, 145 mg, 0.316 mmol) was added TFA (1 ml). The solution was stirred at rt for 2 h (LCMS control), then the reaction mixture was evaporated to dryness, water solution of sodium bicarbonate (5 ml) was added, product extracted with DCM (3×5 ml). Combined organic extract was dried with sodium sulphate, concentrated to dryness to yield the title product P148 (113 mg, 100%). LCMS(ESI) [MH]⁺: 359.

### Synthesis of 4-(2,7-Diazaspiro[3.5]non-2-yl)-6-(2-pyridin-3-ylethyl)quinazoline (P150)

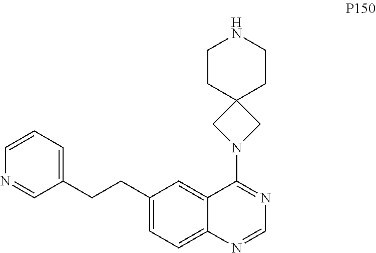 [SMILES: c1cncc(CCc2ccc3ncnc(N4CC5(CCNCC5)C4)c3c2)c1]

### Preparation 149. tert-Butyl 2-[6-(2-pyridin-3-ylethyl)quinazolin-4-yl]-2,7-diazaspiro[3.5]nonane-7-carboxylate (P149)

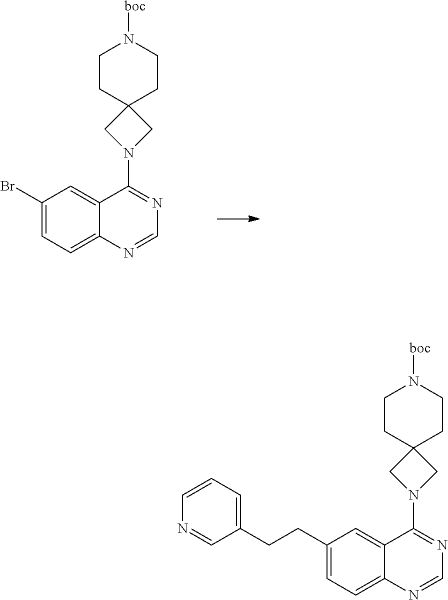

To a solution of tert-butyl 2-(6-bromoquinazolin-4-yl)-2,7-diazaspiro[3.5]nonane-7-carboxylate (P146, 200 mg 0.46 mmol) in DMAA (2 ml) TEA (186 mg, 1.84 mmol), 3-vinylpyridine (144 mg, 1.48 mmol) and Pd(PPh₃)₄ (20 mg) were added under argon atmosphere. The mixture was stirred at 90° C. for 16 h (LCMS control). After reaction completed Pd—C(30 mg) was added and mixture was stirred at rt under hydrogen atmosphere until reaction complete (LCMS control). Then the mixture was filtered, washed with brine (40 ml) and extracted with ethylacetate (2×5 ml), concentrated and residue was purified by silica gel column chromatography (DCM-ethylacetate-1:1) to give the title product P149 (145 mg, 69%). LCMS(ESI) [MH]⁺: 460.

### Preparation 150. 4-(2,7-Diazaspiro[3.5]non-2-yl)-6-(2-pyridin-3-ylethyl)quinazoline (P150)

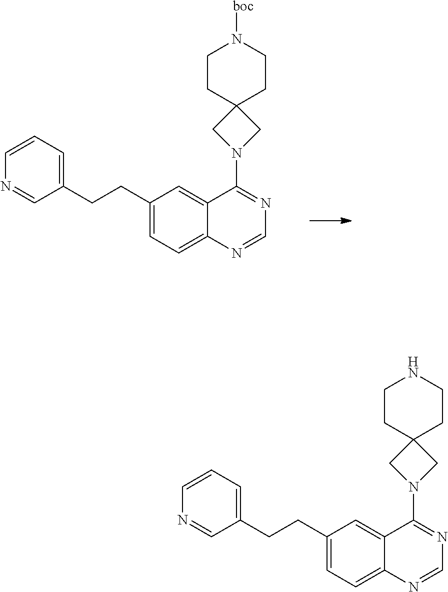

To tert-butyl 2-[6-(2-pyridin-3-ylethyl)quinazolin-4-yl]-2,7-diazaspiro[3.5]nonane-7-carboxylate (P149, 145 mg, 0.316 mmol) was added TFA (1 ml). The solution was stirred at rt for 2 h (LCMS control), then evaporated to dryness, water solution of sodium bicarbonate (5 ml) was added, product was extracted with DCM (3×5 ml). Organic phase dried with sodium sulphate, concentrated to dryness yield title product P150 113 mg, 100%. LCMS(ESI) [MH]⁺: 360.

### Synthesis of 4-(2,7-Diazaspiro[3.5]non-2-yl)-6-(2-pyridin-4-ylethyl)quinazoline (P152)

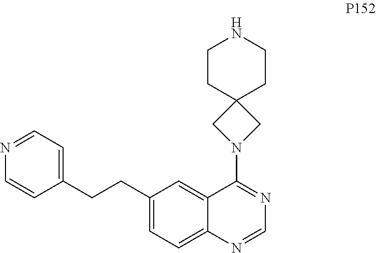 [SMILES: c1cc(CCc2ccc3ncnc(N4CC5(CCNCC5)C4)c3c2)ccn1]

### Preparation 151. tert-Butyl 2-[6-(2-pyridin-4-ylethyl)quinazolin-4-yl]-2,7-diazaspiro[3.5]nonane-7-carboxylate (P151)

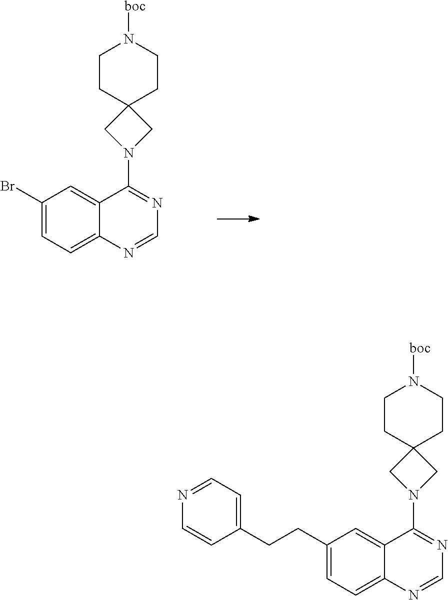

To a solution of tert-butyl 2-(6-bromoquinazolin-4-yl)-2,7-diazaspiro[3.5]nonane-7-carboxylate (P146, 200 mg 0.46 mmol) in DMAA (2 ml) TEA (186 mg, 1.84 mmol), 4-vinylpyridine (144 mg, 1.48 mmol) and Pd(PPh₃)₄ (20 mg) were added under argon atmosphere. The mixture was stirred at 90° C. for 16 h (LCMS control). After reaction completed Pd/C (30 mg) was added and the mixture was stirred at rt under hydrogen atmosphere until reaction was completed (LCMS control). Then mixture was filtered, washed with brine (40 ml) and extracted with ethylacetate (2×5 ml), concentrated and residue was purified by silica gel column chromatography (dichloromethane-ethylacetate-1:1) to give title product P151 (145 mg, 69%). LCMS(EST) [MH]⁺: 460.

### Preparation 152. 4-(2,7-Diazaspiro[3.5]non-2-yl)-6-(2-pyridin-4-ylethyl)quinazoline (P152)

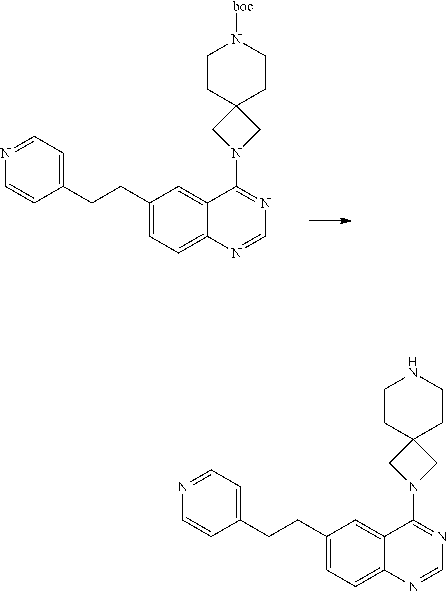

To tert-butyl 2-[6-(2-pyridin-4-ylethyl)quinazolin-4-yl]-2,7-diazaspiro[3.5]nonane-7-carboxylate (P151, 145 mg, 0.316 mmol) was added TFA (1 ml). The solution was stirred at rt for 2 h (LCMS control), then evaporated to dryness, water solution of sodium bicarbonate (5 ml) was added, the product was extracted with DCM (3×5 ml). Organic phase was dried with sodium sulphate, concentrated to dryness to yield the title compound P152 (113 mg, 100%). LCMS(ESI) [MH]⁺: 360

### Synthesis of 4-(2,7-diazaspiro[3.5]non-2-yl)-6-(phenylethynyl)quinazoline (P154)

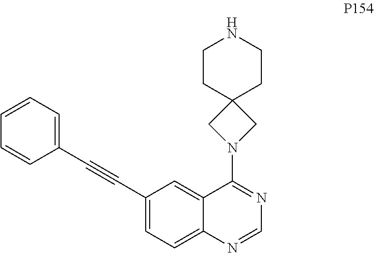 [SMILES: C(#Cc1ccc2ncnc(N3CC4(CCNCC4)C3)c2c1)c1ccccc1]

### Preparation 153. tert-Butyl 2-[6-(phenylethynyl)quinazolin-4-yl]-2,7-diazaspiro[3.5]nonane-7-carboxylate (P153)

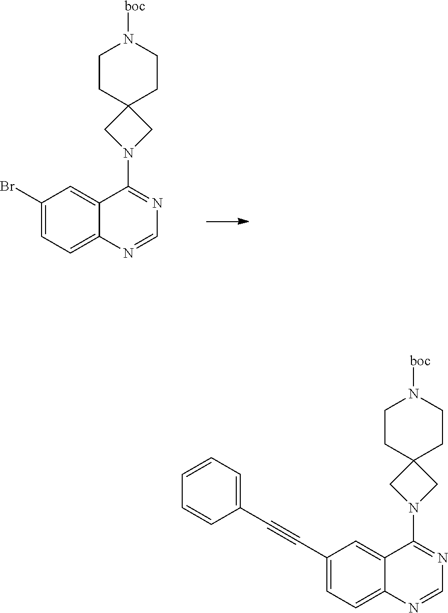

tert-Butyl 2-(6-bromoquinazolin-4-yl)-2,7-diazaspiro[3.5]nonane-7-carboxylate (P146, 80 mg, 0.185 mmol) was dissolved in DMF (1 mL) and purged with N₂ on an oil bath at 80° C. for 10 minutes. Then bis(triphenylphosphine) palladium (II) dichloride (11 mg, 0.016 mmol), triphenylphosphine (10 mg, 0.037 mmol) and copper iodide (7 mg, 0.037 mmol) were added. After 5 minutes of purging with N₂, diethylamine (0.5 mL, 5 mmol) was added followed by the addition of phenylacetylene (0.03 mL, 0.27 mmol). The vessel was closed, and the reaction stirred at 80° C. for 16 hours. The reaction mixture was poured into ice water, and the precipitate was isolated by filtration, washed with water, and dried under vacuum. The product was stirred in DCM for 30 minutes. The precipitate was isolated by filtration, washed with DCM and diisopropyl ether and dried under vacuo at 50° C. to obtain tert-butyl 2-[6-(phenylethynyl)quinazolin-4-yl]-2,7-diazaspiro[3.5]nonane-7-carboxylate (P153, 56 mg, 70%). LCMS(ESI) [MH]⁺: 455.

### Preparation 154. 4-(2,7-diazaspiro[3.5]non-2-yl)-6-(phenylethynyl)quinazoline (P154)

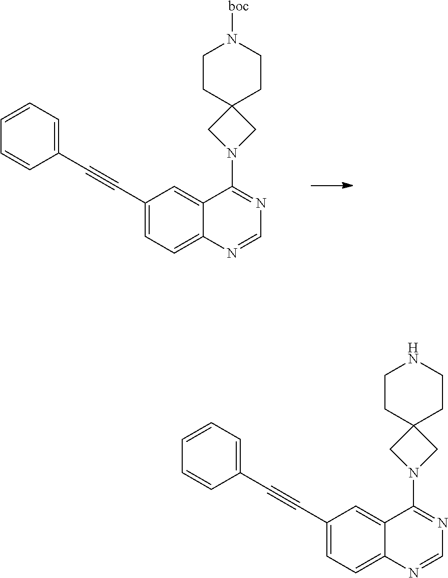

To tert-butyl 2-[6-(phenylethynyl)quinazolin-4-yl]-2,7-diazaspiro[3.5]nonane-7-carboxylate (P153, 56 mg, 0.123 mmol) was added TFA (1 ml). The solution stirred at rt for 2 h (LCMS control), then evaporated to dryness, water solution of sodium bicarbonate (5 ml) was added, product extracted with DCM (3×5 ml). Organic phase dried with sodium sulphate, concentrated to dryness yield 6-(phenylethynyl)-4-(2,7-diazaspiro[3.5]nonan-2-yl)quinazoline (P154, 44 mg, 100%). LCMS(ESI) [MH]⁺: 355.

### Synthesis of 4-(2,7-Diazaspiro[3.5]non-2-yl)quinazoline (P156)

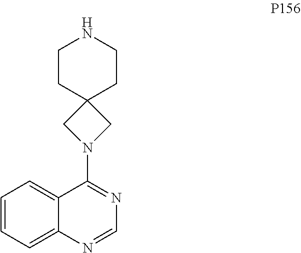 [SMILES: c1ccc2c(N3CC4(CCNCC4)C3)ncnc2c1]

### Preparation 155. tert-Butyl 2-(quinazolin-4-yl)-2,7-diazaspiro[3.5]nonane-7-carboxylate (P155)

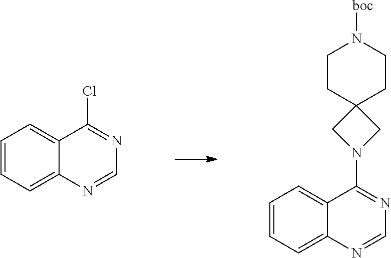

To a solution 4-chloroquinazoline prepared according to WO2007/38387, 2007, A2 (3 g, 18.2 mmol) in DCM (60 ml) was added DIPEA (7/9 g, 61 mmol) and tert-butyl 2,7-diazaspiro[3.5]nonane-7-carboxylate hydrochloride (4.8 g, 18.2 mmol). The reaction solution was stirred at rt for 18 h (LCMS control). Then product was purified by column chromatography on silica with ethylacetate-methanol (10:1) to yield P155 (6.2 g, 96%) as a white solid. LCMS(ESI) [MH]⁺: 355.

### Preparation 156. 4-(2,7-Diazaspiro[3.5]non-2-yl)quinazoline (P156)

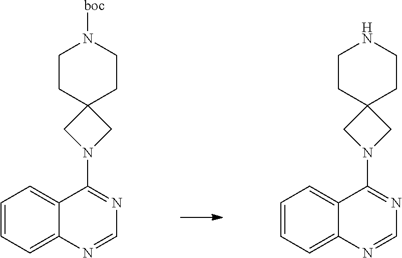

To tert-butyl 2-(quinazolin-4-yl)-2,7-diazaspiro[3.5]nonane-7-carboxylate (6.2 g, 17.5 mmol) was added TFA (60 ml). The solution was stirred at rt for 2 h (LCMS control), then evaporated to dryness, water solution of sodium bicarbonate (100 ml) was added, product was extracted with DCM (3×50 ml). Organic phase was dried with sodium sulphate and concentrated to dryness to yield the title compound P156 (4.44 g, 100%). LCMS(ESI) [MH]⁺: 255.

### Synthesis of 4-(2,7-diazaspiro[3.5]non-2-yl)pyrido[2,3-d]pyrimidine (P158)

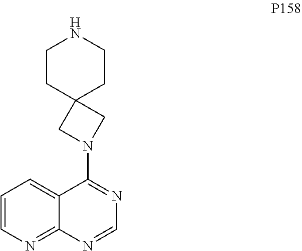 [SMILES: c1cnc2ncnc(N3CC4(CCNCC4)C3)c2c1]

### Preparation 157. tert-Butyl 2-pyrido[2,3-d]pyrimidin-4-yl-2,7-diazaspiro[3.5]nonane-7-carboxylate (P157)

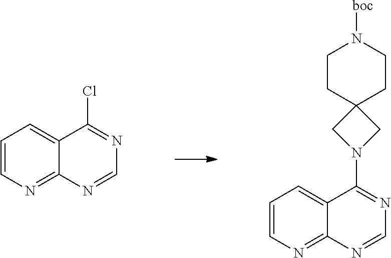

To a solution of 4-chloro-pyrido[2,3-d]pyrimidine prepared according to the procedure described by Robins; Hitchings [Journal of the American Chemical Society, 1955, vol. 77, p. 2256,2259] (3 g, 18.2 mmol) in dichloroethane (60 ml) was added DIPEA (7.9 g, 61 mmol) and tert-butyl 2,7-diazaspiro[3.5]nonane-7-carboxylate hydrochloride (4.8 g, 18.2 mmol). The reaction solution was stirred at rt for 18 h (LCMS control). After evaporation of the solvent the product was purified by column chromatography on silica with ethylacetate-methanol (10:1) to yield P157 (6.2 g, 96%) as a white solid. LCMS(ESI) [MH]⁺: 356.

### Preparation 158. 4-(2,7-Diazaspiro[3.5]non-2-yl)pyrido[2,3-d]pyrimidine (P158)

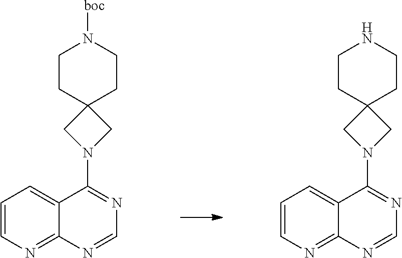

To tert-butyl 2-pyrido[2,3-d]pyrimidin-4-yl-2,7-diazaspiro[3.5]nonane-7-carboxylate (P157, 6.2 g, 17.5 mmol) was added TFA (60 ml). The solution was stirred at rt for 2 h (LCMS control), then evaporated to dryness, water solution of sodium bicarbonate (100 ml) was added, product was extracted with DCM (3×50 ml). Organic phase dried with sodium sulphate, concentrated to dryness to yield the title compound P158 (4.44 g, 100%). LCMS(ESI) [MH]⁺: 256.

### Synthesis of 4-chloro-7-(2,2,2-trifluoroethyl)quinazoline (P165)

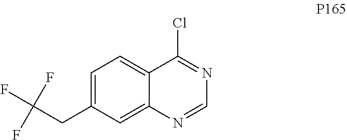 [SMILES: FC(F)(F)Cc1ccc2c(Cl)ncnc2c1]

### Preparation 159. Methyl 4-bromo-2-[(tert-butoxycarbonyl)amino]benzoate (P159)

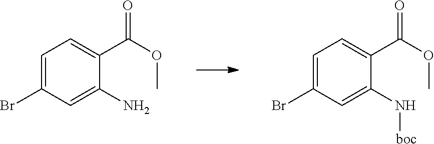

To a solution of methyl 2-amino-4-bromobenzoate (25 g, 0.109 mol) in DCM (250 ml) Boc₂O (26.5 g, 0.122 mol), triethylamine (62 g, 0.61 mol) and DMAP (4 g, 33 mmol) were added. The solution was stirred at ambient temperature for 18 h, then water (250 ml) was added and with stirring potassium hydrosulfate was added small portions up to pH=3. Water phase was extracted with DCM (200 ml), organic solution was concentrated, and residue was purified with silica gel column chromatography (30% DCM in hexane) to yield methyl 4-bromo-2-[(tert-butoxycarbonyl)amino]benzoate P159 as white solid (14.3 g, 40%). LCMS(ESI) [MH]⁺: 331.

### Preparation 160. Methyl 2-[(tert-butoxycarbonyl)amino]-4-(4,4,5,5-tetramethyl-1,3,2-dioxaborolan-2-yl)benzoate (P160)

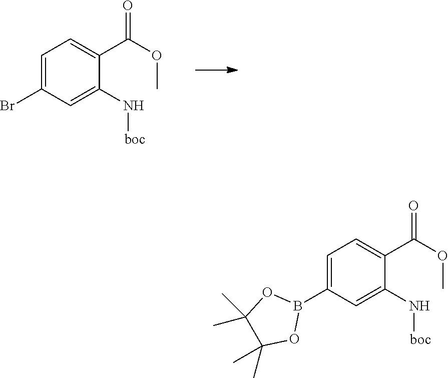

Methyl 4-bromo-2-[(tert-butoxycarbonyl)amino]benzoate (P159, 14.3 g, 43 mmol), and bis(pinacolato)diboron (22 g, 87 mmol) were dissolved in dioxane (120 ml), then potassium acetate (12.6 g, 0.129 mol) was added. The mixture was stirred for 30 min at 70° C. under argon atmosphere, Pd(dppf)Cl₂ (3.2 g) was added, and reaction mixture was stirred at 100° C. for 3 h (TLC control), the mixture was cooled and filtered through a pad of celite. The filtrate was then concentrated. The residue was purified by silica gel column chromatography (DCM) to give the title product P160 (16 g, 99%) as a white solid. LCMS(ESI) [MH]⁺: 378. Preparation 161. Methyl 2-[(tert-butoxycarbonyl)amino]-4-(2,2,2-trifluoroethyl)benzoate (P161)

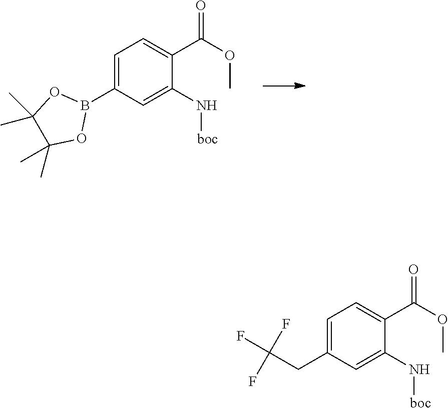

Methyl 2-[(tert-butoxycarbonyl)amino]-4-(4,4,5,5-tetramethyl-1,3,2-dioxaborolan-2-yl)benzoate (P160, 11.2 g, 30 mmol) was dissolved in dioxane (100 ml), cesium carbonate (32.6 g, 118 mmol) and water (8 ml) were added. The mixture was stirred for 30 min at 70° C. under argon atmosphere, then iodotrifluoroethane (18.6 g, 89 mmol), Pd₂(dba)₃ (2.6 g), and Xantphos (2.6 g) were added. Reaction mixture was stirred at 82° C. for 20 h, then the mixture was cooled and filtered through a pad of celite. The filtrate was concentrated. The residue was purified by silica gel column chromatography (DCM-Hexane-1:1) to give the title product P161 (5.85 g 59%) as a white solid. LCMS(ESI) [MH]⁺: 334.

### Preparation 162. Methyl 2-amino-4-(2,2,2-trifluoroethyl)benzoate (P162)

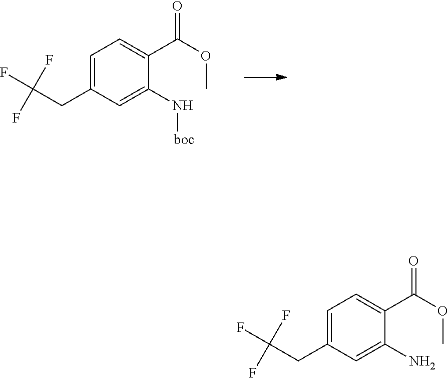

To methyl 2-[(tert-butoxycarbonyl)amino]-4-(2,2,2-trifluoroethyl)benzoate (P161, 5.85 g, 17.6 mmol) solution 3 M HCl in dioxane was added. The reaction mixture was stirred at rt for 2 h (NMR control) and concentrated to dryness to yield the title product P162 (4.6 g, 100%) as hydrochloride. LCMS(ESI) [MH]⁺: 234.

### Preparation 163. 2-Amino-4-(2,2,2-trifluoroethyl)benzoic acid (P163)

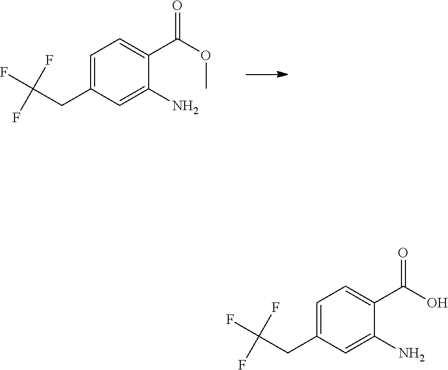 [SMILES: COC(=O)c1ccc(CC(F)(F)F)cc1N ; Nc1cc(CC(F)(F)F)ccc1C(=O)O]

To a solution of methyl 2-amino-4-(2,2,2-trifluoroethyl)benzoate (P162, 4.6 g, 17.6 mmol) in methanol (100 ml) lithium hydroxide (2 g, 83 mmol) and water (35 ml) were added. the mixture was stirred at 50° C. for 1h (TLC control) then cooled, concentrated to dryness, and water (50 ml) was added. To the solution with stirring and cooling with cold water concentrated HCl was added up to pH=3, precipitate was filtered off, washed with water (15 ml) and dried to yield the title product P163 (3 g, 78%) as a white solid. LCMS(ESI) [MH]⁺: 220.

### Preparation 164. 7-(2,2,2-trifluoroethyl)quinazolin-4(3H)-one (P164)

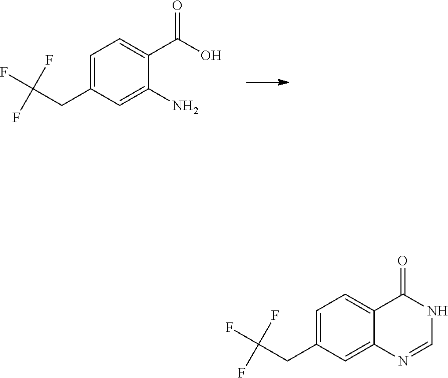 [SMILES: Nc1cc(CC(F)(F)F)ccc1C(=O)O ; O=c1[nH]cnc2cc(CC(F)(F)F)ccc12]

A mixture of 2-amino-4-(2,2,2-trifluoroethyl)benzoic acid (P163, 3 g, 13.7 mmol) and formamide (2.2 g, 49 mmol) were stirred in vial at 155° C. for 1.5 h, and at 165° C. for 40 min, cooled, and saturated solution of sodium bicarbonate (50 ml) was added. The mixture was stirred at rt for 30 min. Product was filtered off, washed with water (55 ml) and dried to yield the title product P164 (2.52 g, 85%) as a white solid. LCMS(ESI) [MH]⁺: 229.

### Preparation 165. 4-chloro-7-(2,2,2-trifluoroethyl)quinazoline (P165)

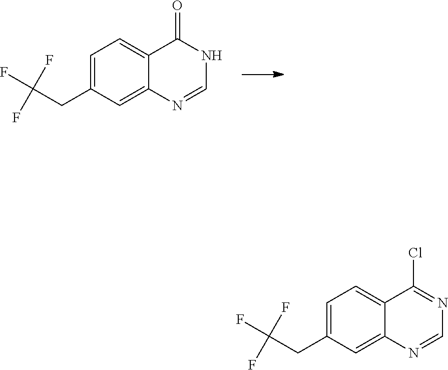 [SMILES: O=c1[nH]cnc2cc(CC(F)(F)F)ccc12 ; FC(F)(F)Cc1ccc2c(Cl)ncnc2c1]

To 7-(2,2,2-trifluoroethyl)quinazolin-4(3H)-one (P164, 2.52 g, 11 mmol) POCl₃ (35 ml) was added, the reaction mixture was stirred and refluxed for 45 min (TLC control), after the reaction completed, the solution was cooled and added to ice-cold water solution of sodium bicarbonate. The mixture was stirred at 15° C. for 30 min, then extracted with DCM (2×50 ml), DCM was concentrated, residue was purified by silica gel column chromatography (dichloromethane-ethylacetate-10:1) to give the title product P165 (1.9 g 70%) as a brown solid. LCMS(ESI) [MH]⁺: 247.

### Synthesis of 4-(2,7-diazaspiro[3.5]non-2-yl)-7-(2,2,2-trifluoroethyl)quinazoline (P167)

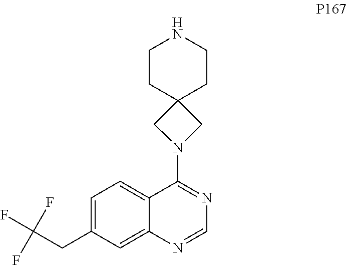 [SMILES: FC(F)(F)Cc1ccc2c(N3CC4(CCNCC4)C3)ncnc2c1]

### Preparation 166. tert-Butyl 2-[7-(2,2,2-trifluoroethyl)quinazolin-4-yl]-2,7-diazaspiro[3.5]nonane-7-carboxylate (P166)

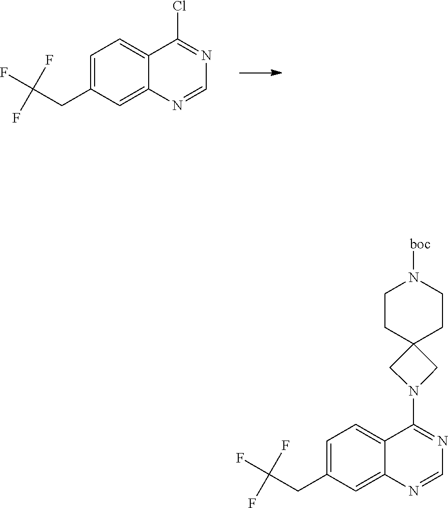

To a solution of 4-chloro-7-(2,2,2-trifluoroethyl)quinazoline (P165, 3 g, 12.2 mmol) in dichloroethane (60 ml) was added DIPEA (7.9 g, 61 mmol) and tert-butyl 2,7-diazaspiro[3.5]nonane-7-carboxylate hydrochloride (3.2 g, 12.2 mmol). The reaction solution was stirred at rt for 18 h (LCMS control). The product was purified by column chromatography on silica with ethylacetate-methanol (10:1) to yield the title product P166 (5.1 g, 96%) as a white solid. LCMS(ESI) [MH]⁺: 437.

### Preparation 167. 4-(2,7-Diazaspiro[3.5]non-2-yl)-7-(2,2,2-trifluoroethyl)quinazoline (P167)

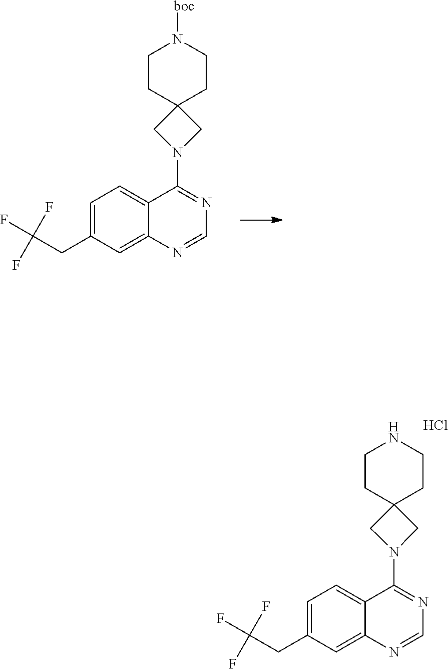

To tert-butyl 2-[7-(2,2,2-trifluoroethyl)quinazolin-4-yl]-2,7-diazaspiro[3.5]nonane-7-carboxylate (P166, 5.1 g, 11.7 mmol) solution HCl in dioxan (3M, 120 ml) was added. The reaction mixture was stirred at rt for 3 h (LCMS control), then evaporated under vacuum to dryness to yield 4-(2,7-diazaspiro[3.5]non-2-yl)-7-(2,2,2-trifluoroethyl)quinazoline hydrochloride (P167, 4.4 g, 100%) as a white solid. LCMS(ESI) [MH]⁺: 337.

### Synthesis of 3-[7-(2,2,2-trifluoroethyl)quinazolin-4-yl]-3,9-diazaspiro[5.5]undecane (P169)

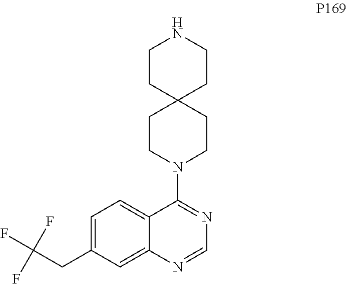 [SMILES: FC(F)(F)Cc1ccc2c(N3CCC4(CCNCC4)CC3)ncnc2c1]

### Preparation 168. tert-butyl 9-[7-(2,2,2-trifluoroethyl)quinazolin-4-yl]-3,9-diazaspiro[5.5]undecane-3-carboxylate (P168)

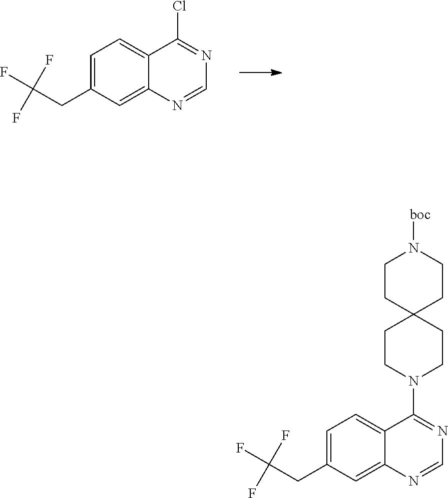

To a solution of 4-chloro-7-(2,2,2-trifluoroethyl)quinazoline (P165, 3 g, 12.2 mmol) in dichloroethane (60 ml) was added DIPEA (7.9 g, 61 mmol) and tert-butyl 3,9-diazaspiro[5.5]undecane-3-carboxylate (3.2 g, 12.0 mmol). The reaction solution was stirred at rt for 18 h (LCMS control). Then product was purified by column chromatography on silica with ethylacetate-methanol (10:1) to yield the title product P168 (5.1 g, 93%) as a white solid. LCMS(ESI) [MH]⁺: 465.

### Preparation 169. 3-[7-(2,2,2-trifluoroethyl)quinazolin-4-yl]-3,9-diazaspiro[5.5]undecane (P169)

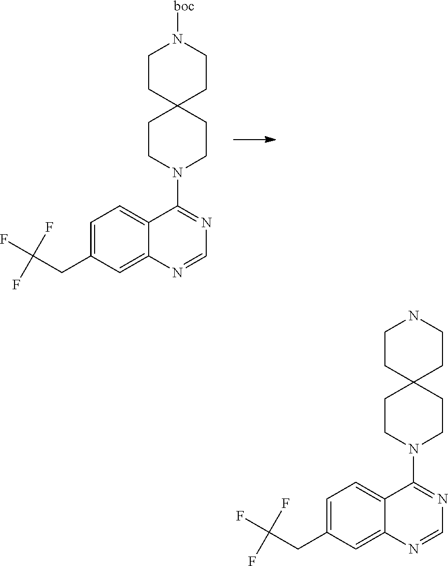

To a solution of tert-butyl 9-[7-(2,2,2-trifluoroethyl)quinazolin-4-yl]-3,9-diazaspiro[5.5]undecane-3-carboxylate (P168, 5.1 g, 11 mmol) in 50 ml of DCM TFA (60 ml) was added. The solution was stirred at rt for 2 h (LCMS control), then evaporated to dryness, water solution of sodium bicarbonate (100 ml) was added, product was extracted with DCM (3×50 ml). Organic phase was dried with sodium sulphate, concentrated to dryness to yield the title compound P169 (4.0 g, 100%). LCMS(EST) [MH]⁺: 365.

### Synthesis of N-piperidin-4-yl-7-(2,2,2-trifluoroethyl)quinazolin-4-amine (P171)

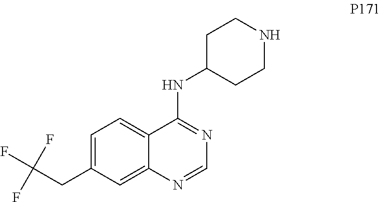 [SMILES: FC(F)(F)Cc1ccc2c(NC3CCNCC3)ncnc2c1]

### Preparation 170. tert-butyl 4-{[7-(2,2,2-trifluoroethyl)quinazolin-4-yl]amino}piperidine-1-carboxylate (P170)

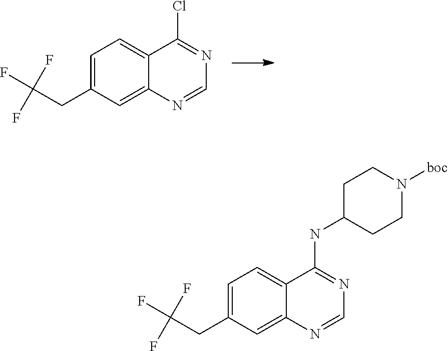

To a solution of 4-chloro-7-(2,2,2-trifluoroethyl)quinazoline (P165, 3 g, 12.2 mmol) in dichloroethane (60 ml) DIPEA (7.9 g, 61 mmol) and N-tert-butyl 4-aminopiperidynecarboxylate (2.4 g, 12.0 mmol) were added. The reaction solution was stirred at rt for 18 h (LCMS control). The product was purified by column chromatography on silica with ethylacetate-methanol (10:1) to yield the title compound P170 (4.53 g, 92%) as a white solid. LCMS(ESI) [MH]⁺: 411.

### Preparation 171. N-piperidin-4-yl-7-(2,2,2-trifluoroethyl)quinazolin-4-amine (P171)

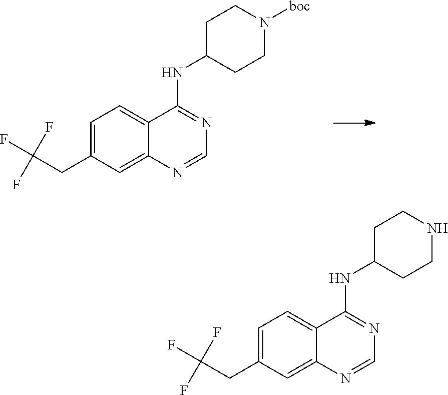

To solution of tert-butyl 4-{[7-(2,2,2-trifluoroethyl)quinazolin-4-yl]amino}piperidine-1-carboxylate (P170, 4.53 g, 11 mmol) in 50 ml of DCM TFA (60 ml) was added. The solution was stirred at rt for 2 h (LCMS control), then evaporated to dryness, water solution of sodium bicarbonate (100 ml) was added, product was extracted with DCM (3×50 ml). Organic phase dried with sodium sulphate, concentrated to dryness to yield the title compound P171 (3.41 g, 100%). LCMS(ESI) [MH]⁺: 365.

### Synthesis of 4-(2,7-diazaspiro[3.5]non-2-yl)-6-[2-(3-methyl-1,2,4-oxadiazol-5-yl)ethyl]quinazoline (P175)

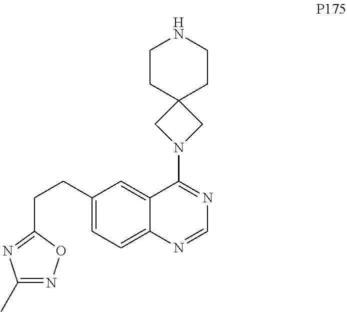 [SMILES: Cc1noc(CCc2ccc3ncnc(N4CC5(CCNCC5)C4)c3c2)n1]

### Preparation 172. tert-butyl 2-[6-(3-ethoxy-3-oxopropyl)quinazolin-4-yl]-2,7-diazaspiro[3.5]nonane-7-carboxylate (P172)

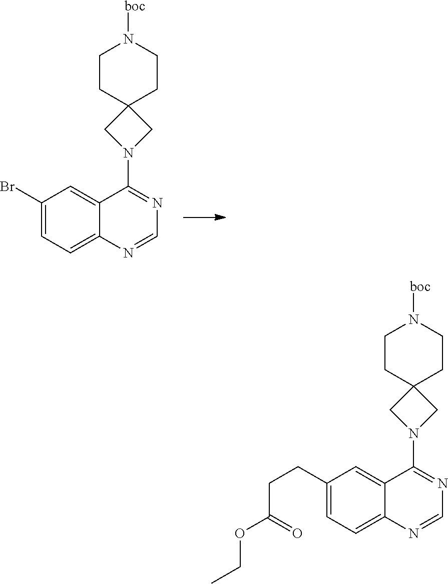

To a solution of tert-butyl 2-(6-bromoquinazolin-4-yl)-2,7-diazaspiro[3.5]nonane-7-carboxylate (P146, 200 mg 0.46 mmol) in DMAA (2 ml) under argon atmosphere TEA (186 mg, 1.84 mmol), ethyl acrylate (148 mg, 1.48 mmol) and Pd(PPh₃)₄ (20 mg) were added. The mixture was stirred at 90° C. for 16 h (LCMS control). After reaction completed Pd/C (30 mg) was added and the mixture was stirred at rt under hydrogen atmosphere until reaction completed (LCMS control). Then the mixture was filtered, washed with brine (40 ml) and extracted with ethylacetate (2×5 ml), concentrated and residue was purified by silica gel column chromatography (DCM-ethylacetate-1:1) to give title product P172 (145 mg, 69%). LCMS(ESI) [MH]⁺: 455.

### Preparation 173. 3-{4-[7-(tert-butoxycarbonyl)-2,7-diazaspiro[3.5]non-2-yl]quinazolin-6-yl}propanoic acid (P173)

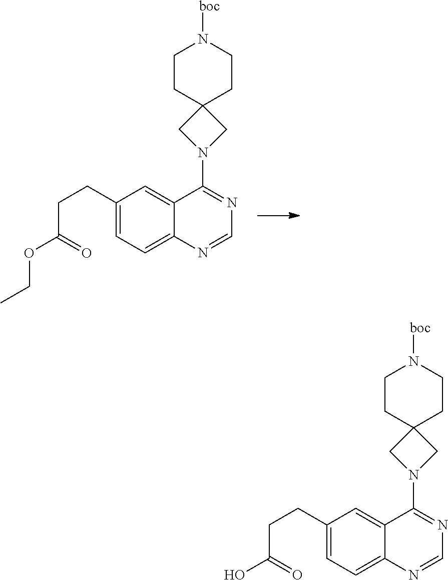

To a solution of tert-butyl 2-[6-(3-ethoxy-3-oxopropyl)quinazolin-4-yl]-2,7-diazaspiro[3.5]nonane-7-carboxylate (P172, 145 mg, 0.53 mmol) in MeOH (10 ml) solution of NaOH (64 mg, 1.6 mmol) in water (1 ml) was added. The mixture stirred at rt for 4 h (TLC control), then concentrated, dissolved in water (8 ml), HCl was added up to pH=4, water phase was extracted with DCM (3×30 ml). Organic phase was evaporated to dryness to yield the title compound P173 (115 mg, 84%). LCMS(ESI) [MH]⁺: 427.

### Preparation 174. tert-butyl 2-{6-[2-(3-methyl-1,2,4-oxadiazol-5-yl)ethyl]quinazolin-4-yl}-2,7-diazaspiro[3.5]nonane-7-carboxylate (P174)

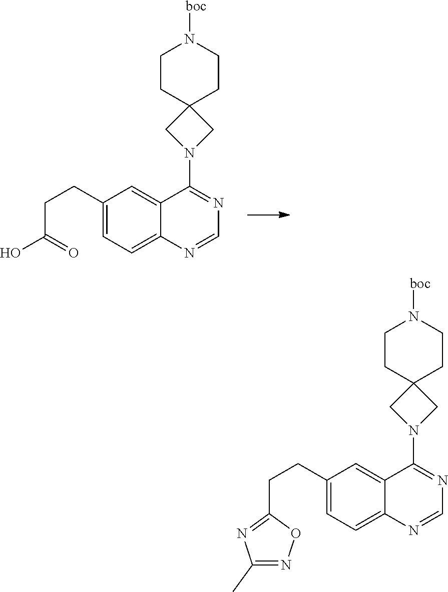

The acid prepared on the previous stage (P173, 115 mg, 0.27 mmol) was dissolved in DMAA (1 ml), CDI (66 mg, 0.41 mmol) was added, the mixture was stirred at 50° C. for 4 h, then N-hydroxyethanimidamide (25 mg, 0.41 mmol) was added and the mixture was stirred at 90° C. for 24 h. After reaction completed (LCMS control) the reaction mixture cooled, brine (20 ml) was added, product was extracted with ethylacetate (2×5 ml). Organic phase evaporated to dryness and residue was separated by silica gel column chromatography (dichloromethane-ethylacetate-1:1) to give title product P174 (81 mg, 65%). LCMS(ESI) [MH]⁺: 465.

### Preparation 175. 4-(2,7-diazaspiro[3.5]non-2-yl)-6-[2-(3-methyl-1,2,4-oxadiazol-5-yl)ethyl]quinazoline (P175)

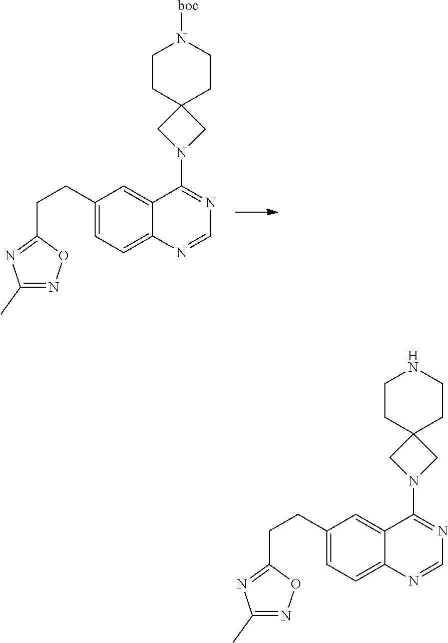

A solution of tert-butyl 2-{6-[2-(3-methyl-1,2,4-oxadiazol-5-yl)ethyl]quinazolin-4-yl}-2,7-diazaspiro[3.5]nonane-7-carboxylate (P174, 81 mg, 17.6 mmol) in TFA (1 ml) was stirred at rt for 2 h (LCMS control), then evaporated to dryness, water solution of sodium bicarbonate (10 ml) was added, product was extracted with DCM (3×5 ml). Organic phase was dried with sodium sulphate, concentrated to dryness to yield the title compound P175 (64 mg, 100%). LCMS(ESI) [MH]⁺: 365.

Table 2 presents certain examples of key intermediates in accordance with the present disclosure.

TABLE 2
Certain examples of key intermediates.

| | | | [MH]⁺ | [MH]⁺ |
|---|---|---|---|---|
| # | Structure | IUPAC Name | Calc. | Found |
| P7 | 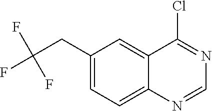 [SMILES: FC(F)(F)Cc1ccc2ncnc(Cl)c2c1] | 4-chloro-6-(2,2,2- trifluoroethyl)quinazoline | 247.025 | 247 |
| P9 | 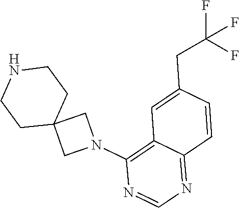 [SMILES: FC(F)(F)Cc1ccc2ncnc(N3CC4(CCNCC4)C3)c2c1] | 4-(2,7- diazaspiro[3.5]nonan-2- yl)-6-(2,2,2- trifluoroethyl)quinazoline | 337.164 | 337 |
| P11 | 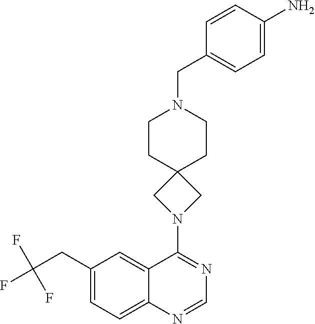 [SMILES: Nc1ccc(CN2CCC3(CC2)CN(c2ncnc4ccc(CC(F)(F)F)cc24)C3)cc1] | [4-({2-[6-(2,2,2- trifluoroethyl)quinazolin-4- yl]-2,7-diazaspiro[3.5]non- 7-yl}methyl)phenyl]amine | 442.222 | 442 |
| P13 | 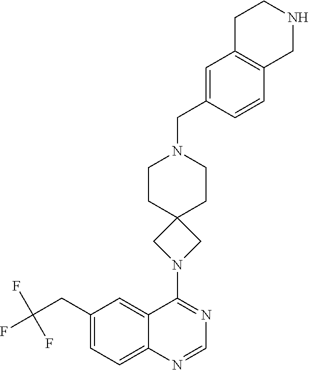 [SMILES: FC(F)(F)Cc1ccc2ncnc(N3CC4(CCN(Cc5ccc6c(c5)CCNC6)CC4)C3)c2c1] | 4-[7-(1,2,3,4- tetrahydroisoquinolin-6- ylmethyl)-2,7- diazaspiro[3.5]nonan-2- yl]-6-(2,2,2- trifluoroethyl)quinazoline | 482.253 | 482 |
| P15 | 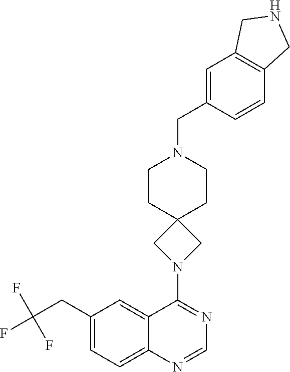 [SMILES: FC(F)(F)Cc1ccc2ncnc(N3CC4(CCN(Cc5ccc6c(c5)CNC6)CC4)C3)c2c1] | 4-[7-(isoindolin-5- ylmethyl)-2,7- diazaspiro[3.5]nonan-2- yl]-6-(2,2,2- trifluoroethyl)quinazoline | 468.238 | 468 |
| P28 | 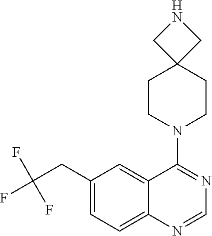 [SMILES: FC(F)(F)Cc1ccc2ncnc(N3CCC4(CC3)CNC4)c2c1] | 4-(2,7- diazaspiro[3.5]nonan-7- yl)-6-(2,2,2- trifluoroethyl)quinazoline | 337.164 | 337 |
| P30 | 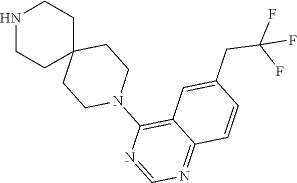 [SMILES: FC(F)(F)Cc1ccc2ncnc(N3CCC4(CCNCC4)CC3)c2c1] | 3-(6-(2,2,2- trifluoroethyl)quinazolin-4- yl)-3,9- diazaspiro[5.5]undecane | 365.1953 | 365 |
| P39 | 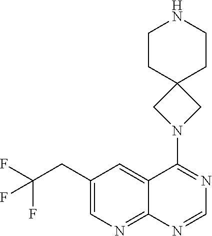 [SMILES: FC(F)(F)Cc1cnc2ncnc(N3CC4(CCNCC4)C3)c2c1] | 4-(2,7- diazaspiro[3.5]nonan-2- yl)-6-(2,2,2- trifluoroethyl)pyrido[2,3- d]pyrimidine | 338.159 | 338 |
| P49 | 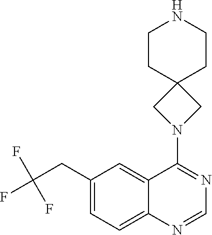 [SMILES: FC(F)(F)Cc1ccc2ncnc(N3CC4(CCNCC4)C3)c2c1] | 4-(2,7- diazaspiro[3.5]nonan-2- yl)-6-(2,2,2- trifluoroethyl)cinnoline | 337.164 | 337 |
| P60 | 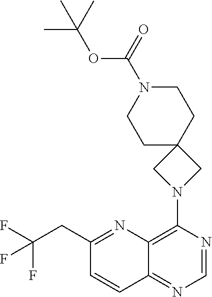 [SMILES: CC(C)(C)OC(=O)N1CCC2(CC1)CN(c1ncnc3ccc(CC(F)(F)F)nc13)C2] | tert-butyl 2-[6-(2,2,2- trifluoroethyl)pyrido[3,2- d]pyrimidin-4-yl]-2,7- diazaspiro[3.5]nonane-7- carboxylate | 438.2117 | 438 |
| P61 | 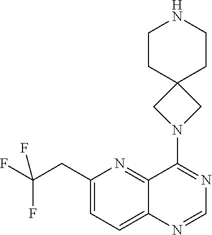 [SMILES: FC(F)(F)Cc1ccc2ncnc(N3CC4(CCNCC4)C3)c2n1] | 4-(2,7- diazaspiro[3.5]nonan-2- yl)-6-(2,2,2- trifluoroethyl)pyrido[3,2- d]pyrimidine | 338.159 | 338 |
| P70 | 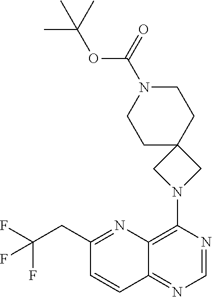 [SMILES: CC(C)(C)OC(=O)N1CCC2(CC1)CN(c1ncnc3ccc(CC(F)(F)F)nc13)C2] | tert-butyl 2-[6-(2,2,2- trifluoroethyl)pteridin-4- yl]-2,7- diazaspiro[3.5]nonane-7- carboxylate | 439.2069 | 439 |
| P71 | 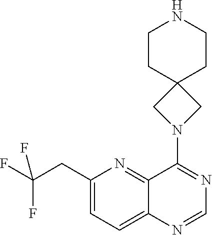 [SMILES: FC(F)(F)Cc1ccc2ncnc(N3CC4(CCNCC4)C3)c2n1] | 4-(2,7- diazaspiro[3.5]nonan-2- yl)-6-(2,2,2- trifluoroethyl)pteridine | 339.155 | 339 |
| P84 | 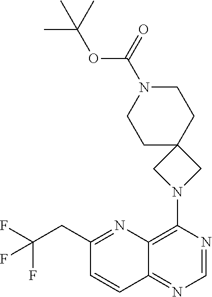 [SMILES: CC(C)(C)OC(=O)N1CCC2(CC1)CN(c1ncnc3ccc(CC(F)(F)F)nc13)C2] | tert-butyl 2-[6-(2,2,2- trifluoroethyl)pyrido[3,4- d]pyrimidin-4-yl]-2,7- diazaspiro[3.5]nonane-7- carboxylate | 438.2117 | 438 |
| P85 | 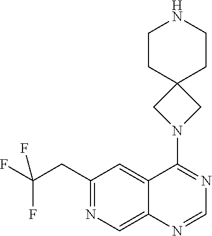 [SMILES: FC(F)(F)Cc1cc2c(N3CC4(CCNCC4)C3)ncnc2cn1] | 4-(2,7- diazaspiro[3.5]nonan-2- yl)-6-(2,2,2- trifluoroethyl)pyrido[3,4- d]pyrimidine | 338.159 | 338 |
| P87 | 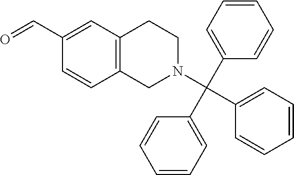 [SMILES: O=Cc1ccc2c(c1)CCN(C(c1ccccc1)(c1ccccc1)c1ccccc1)C2] | 2-trityl-3,4-dihydro-1H- isoquinoline-6- carbaldehyde | 404.201 | 404 |
| P88 | 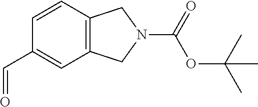 [SMILES: CC(C)(C)OC(=O)N1Cc2ccc(C=O)cc2C1] | tert-butyl 5- formylisoindoline-2- carboxylate | 248.129 | 248 |
| P101 | 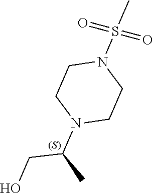 | (S)-2-(4- (Methylsulfonyl)piperazin- 1-yl)propan-1-ol | 223.112 | 223.1 |
| P107 | 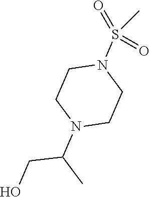 | 2-(4- methylsulfonylpiperazin-1- yl)propan-1-ol | 223.112 | 223 |
| P133 | 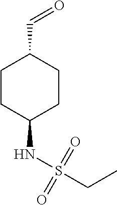 | N-(trans-4- Formylcyclohexyl)ethane- sulfonamide | 220.101 | 220 |
| P137 | 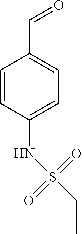 | N-(4-formylphenyl) ethanesulfonamide | 214.054 | 214 |
| P141 | 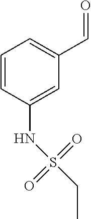 | N-(3-formylphenyl) ethanesulfonamide | 214.054 | 214 |
| P145 | 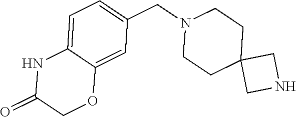 [SMILES: O=C1COc2cc(CN3CCC4(CC3)CNC4)ccc2N1] | 7-(2,7- diazaspiro[3.5]nonan-7- ylmethyl)-4H-1,4- benzoxazin-3-one | 288.177 | 288 |
| P148 | 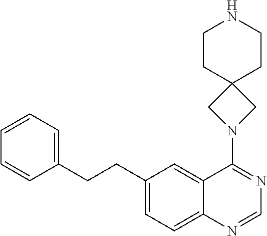 [SMILES: c1ccc(CCc2ccc3ncnc(N4CC5(CCNCC5)C4)c3c2)cc1] | 4-(2,7-Diazaspiro[3.5]non- 2-yl)-6-(2- phenylethyl)quinazoline | 359.224 | 359 |
| P150 | 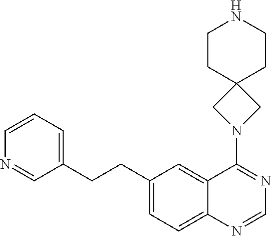 [SMILES: c1cncc(CCc2ccc3ncnc(N4CC5(CCNCC5)C4)c3c2)c1] | 4-(2,7-Diazaspiro[3.5]non- 2-yl)-6-(2-pyridin-3- ylethyl)quinazoline | 360.219 | 360 |
| P152 | 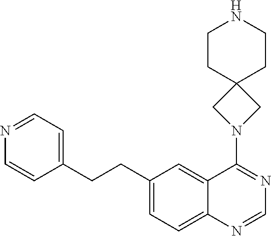 [SMILES: c1cc(CCc2ccc3ncnc(N4CC5(CCNCC5)C4)c3c2)ccn1] | 4-(2,7-Diazaspiro[3.5]non- 2-yl)-6-(2-pyridin-4- ylethyl)quinazoline | 360.129 | 360 |
| P154 | 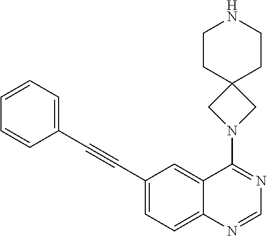 [SMILES: C(#Cc1ccc2ncnc(N3CC4(CCNCC4)C3)c2c1)c1ccccc1] | 4-(2,7-diazaspiro[3.5]non- 2-yl)-6- (phenylethynyl)quinazoline | 355.192 | 355 |
| P156 | 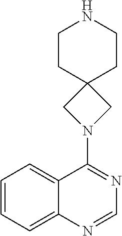 [SMILES: c1ccc2c(N3CC4(CCNCC4)C3)ncnc2c1] | 4-(2,7-Diazaspiro[3.5]non- 2-yl)quinazoline | 255.161 | 255 |
| P158 | 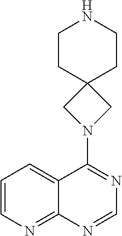 [SMILES: c1cnc2ncnc(N3CC4(CCNCC4)C3)c2c1] | 4-(2,7-diazaspiro[3.5]non- 2-yl)pyrido[2,3- d]pyrimidine | 256.156 | 256 |
| P165 | 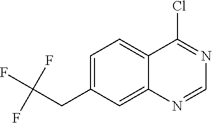 [SMILES: FC(F)(F)Cc1ccc2c(Cl)ncnc2c1] | 4-chloro-7-(2,2,2- trifluoroethyl)quinazoline | 247.025 | 247 |
| P167 | 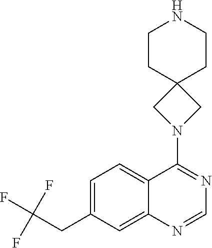 [SMILES: FC(F)(F)Cc1ccc2c(N3CC4(CCNCC4)C3)ncnc2c1] | 4-(2,7-diazaspiro[3.5]non- 2-yl)-7-(2,2,2- trifluoroethyl)quinazoline | 337.164 | 337 |
| P169 | 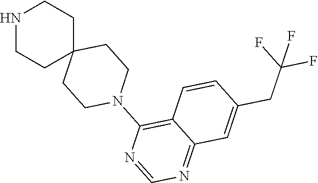 [SMILES: FC(F)(F)Cc1ccc2c(N3CCC4(CCNCC4)CC3)ncnc2c1] | 3-(7-(2,2,2- trifluoroethyl)quinazolin-4- yl)-3,9- diazaspiro[5.5]undecane | 365.1953 | 365 |
| P171 | 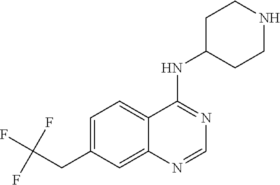 [SMILES: FC(F)(F)Cc1ccc2c(NC3CCNCC3)ncnc2c1] | N-piperidin-4-yl-7-(2,2,2- trifluoroethyl)quinazolin-4- amine | 311.148 | 311 |
| P175 | 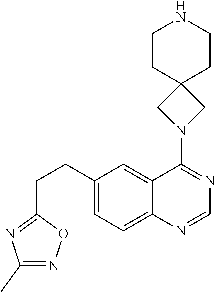 [SMILES: Cc1noc(CCc2ccc3ncnc(N4CC5(CCNCC5)C4)c3c2)n1] | 4-(2,7-diazaspiro[3.5]non- 2-yl)-6-[2-(3-methyl-1,2,4- oxadiazol-5- yl)ethyl]quinazoline | 365.209 | 365 |

### Examples of the Final Compound

Table 3 presents certain non-limiting examples of the compound of Formula (I).

TABLE 3
Selected examples of the compound of Formula (I)

| | | | [MH]⁺ | [MH]⁺ |
|---|---|---|---|---|
| # | Structure | IUPAC Name | Calc. | Found |
| 1 | 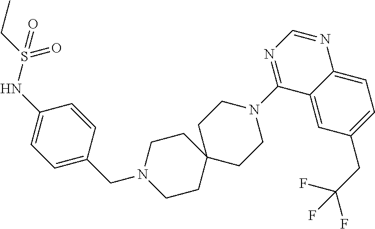 [SMILES: CCS(=O)(=O)Nc1ccc(CN2CCC3(CC2)CCN(c2ncnc4ccc(CC(F)(F)F)cc24)CC3)cc1] | N-[4-[3-[6-(2,2,2-trifluoroethyl) quinazolin-4- yl]-3,9-diazaspiro [5.5]undecan-9- yl]methyl]phenyl] ethanesulfonamide | 562.2464 | 562 |
| 2 | 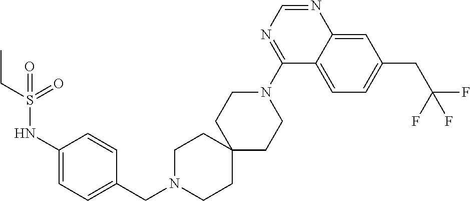 [SMILES: CCS(=O)(=O)Nc1ccc(CN2CCC3(CC2)CCN(c2ncnc4cc(CC(F)(F)F)ccc24)CC3)cc1] | N-[4-[[3-[7-(2,2,2-trifluoroethyl) quinazolin-4- yl]-3,9-diazaspiro [5.5]undecan-9- yl]methyl]phenyl] ethanesulfonamide | 562.2464 | 562 |
| 3 | 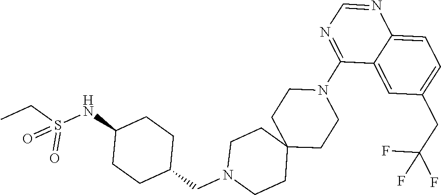 [SMILES: CCS(=O)(=O)N[C@H]1CC[C@H](CN2CCC3(CC2)CCN(c2ncnc4ccc(CC(F)(F)F)cc24)CC3)CC1] | N-((1R,4R)-4-((9-(6-(2,2,2- trifluoroethyl) quinazolin-4-yl)-3,9- diazaspiro[5.5]undecan-3- yl)methyl)cyclohexyl) ethanesulfonamide | 568.2933 | 568 |
| 4 | 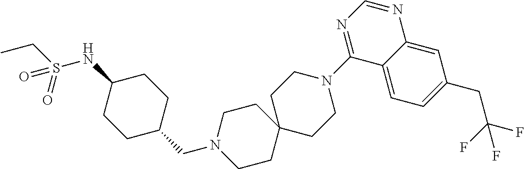 [SMILES: CCS(=O)(=O)N[C@H]1CC[C@H](CN2CCC3(CC2)CCN(c2ncnc4cc(CC(F)(F)F)ccc24)CC3)CC1] | N-((1R,4R)-4-((9-(7-(2,2,2- trifluoroethyl) quinazolin-4-yl)-3,9- diazaspiro[5.5]undecan-3- yl)methyl)cyclohexyl) ethanesulfonamide | 568.2933 | 568 |
| 5 | 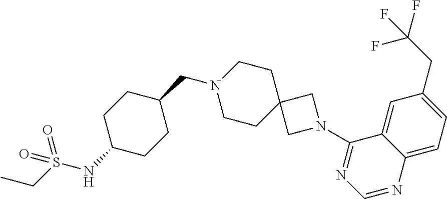 [SMILES: CCS(=O)(=O)N[C@H]1CC[C@H](CN2CCC3(CC2)CN(c2ncnc4ccc(CC(F)(F)F)cc24)C3)CC1] | N-((1R,4R)-4-((2-(6-(2,2,2- trifluoroethyl) quinazolin-4-yl)-2,7- diazaspiro[3.5]nonan-7- yl)methyl)cyclohexyl) ethanesulfonamide | 540.262 | 540 |
| 6 | 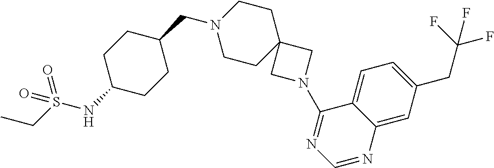 [SMILES: CCS(=O)(=O)N[C@H]1CC[C@H](CN2CCC3(CC2)CN(c2ncnc4cc(CC(F)(F)F)ccc24)C3)CC1] | N-((1R,4R)-4-((2-(7-(2,2,2- trifluoroethyl) quinazolin-4-yl)-2,7- diazaspiro[3.5]nonan-7- yl)methyl)cyclohexyl) ethanesulfonamide | 540.262 | 540 |
| 7 | 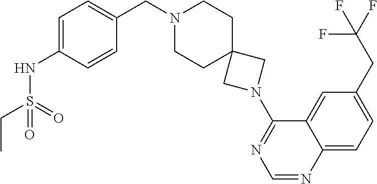 [SMILES: CCS(=O)(=O)Nc1ccc(CN2CCC3(CC2)CN(c2ncnc4ccc(CC(F)(F)F)cc24)C3)cc1] | N-(4-((2-(6-(2,2,2- trifluoroethyl) quinazolin-4- yl)-2,7-diazaspiro [3.5]nonan-7- yl)methyl)phenyl) ethanesulfonamide | 534.215 | 534 |
| 8 | 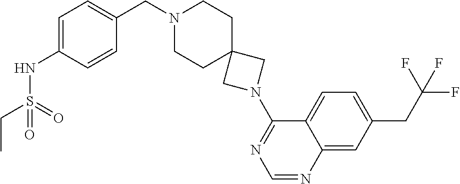 [SMILES: CCS(=O)(=O)Nc1ccc(CN2CCC3(CC2)CN(c2ncnc4cc(CC(F)(F)F)ccc24)C3)cc1] | N-(4-((2-(7-(2,2,2- trifluoroethyl) quinazolin-4- yl)-2,7-diazaspiro [3.5]nonan-7- yl)methyl)phenyl) ethanesulfonamide | 534.215 | 534 |
| 9 | 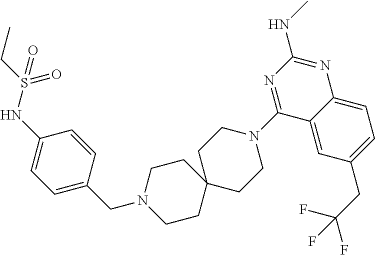 [SMILES: CCS(=O)(=O)Nc1ccc(CN2CCC3(CC2)CCN(c2nc(NC)nc4ccc(CC(F)(F)F)cc24)CC3)cc1] | N-(4-((9-(2- (methylamino)-6-(2,2,2- trifluoroethyl) quinazolin-4-yl)-3,9- diazaspiro[5.5]undecan-3- yl)methyl)phenyl) ethanesulfonamide | 591.2729 | 591 |
| 10 | 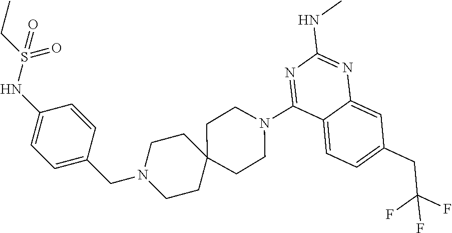 [SMILES: CCS(=O)(=O)Nc1ccc(CN2CCC3(CC2)CCN(c2nc(NC)nc4cc(CC(F)(F)F)ccc24)CC3)cc1] | N-(4-((9-(2- (methylamino)-7-(2,2,2- trifluoroethyl) quinazolin-4-yl)-3,9- diazaspiro[5.5]undecan-3- yl)methyl)phenyl) ethanesulfonamide | 591.2729 | 591 |
| 11 | 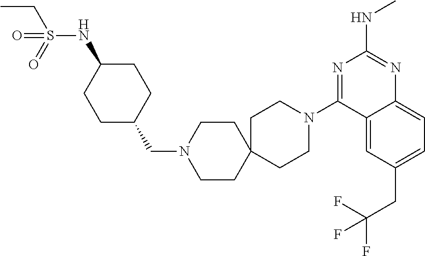 [SMILES: CCS(=O)(=O)N[C@H]1CC[C@H](CN2CCC3(CC2)CCN(c2nc(NC)nc4ccc(CC(F)(F)F)cc24)CC3)CC1] | N-((1R,4R)-4-((9-(2- (methylamino)-6-(2,2,2- trifluoroethyl) quinazolin-4-yl)-3,9- diazaspiro[5.5]undecan-3- yl)methyl)cyclohexyl) ethanesulfonamide | 597.3198 | 597 |
| 12 | 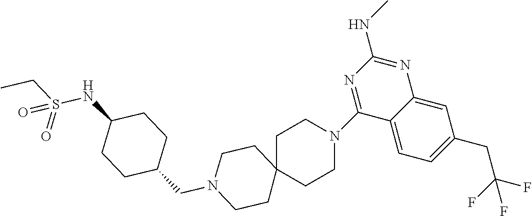 [SMILES: CCS(=O)(=O)N[C@H]1CC[C@H](CN2CCC3(CC2)CCN(c2nc(NC)nc4cc(CC(F)(F)F)ccc24)CC3)CC1] | N-((1R,4R)-4-((9-(2- (methylamino)-7-(2,2,2- trifluoroethyl) quinazolin-4-yl)-3,9- diazaspiro[5.5]undecan-3- yl)methyl)cyclohexyl) ethanesulfonamide | 597.3198 | 597 |
| 13 | 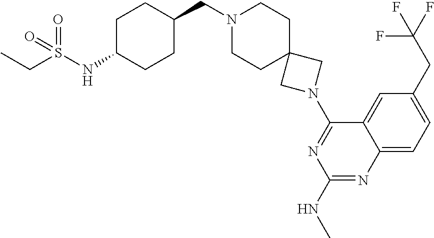 [SMILES: CCS(=O)(=O)N[C@H]1CC[C@H](CN2CCC3(CC2)CN(c2nc(NC)nc4ccc(CC(F)(F)F)cc24)C3)CC1] | N-((1R,4R)-4-((2-(2- (methylamino)-6-(2,2,2- trifluoroethyl) quinazolin-4-yl)-2,7- diazaspiro[3.5]nonan-7- yl)methyl)cyclohexyl) ethanesulfonamide | 569.2885 | 569 |
| 14 | 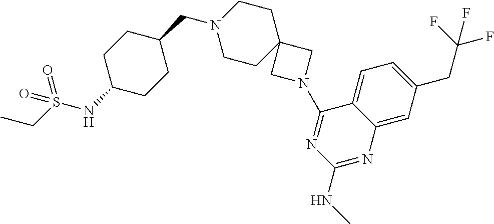 [SMILES: CCS(=O)(=O)N[C@H]1CC[C@H](CN2CCC3(CC2)CN(c2nc(NC)nc4cc(CC(F)(F)F)ccc24)C3)CC1] | N-((1R,4R)-4-((2-(2- (methylamino)-7-(2,2,2- trifluoroethyl) quinazolin-4-yl)-2,7- diazaspiro[3.5]nonan-7- yl)methyl)cyclohexyl) ethanesulfonamide | 569.2885 | 569 |
| 15 | 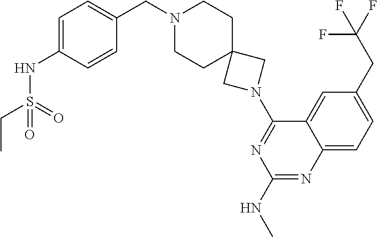 [SMILES: CCS(=O)(=O)Nc1ccc(CN2CCC3(CC2)CN(c2nc(NC)nc4ccc(CC(F)(F)F)cc24)C3)cc1] | N-(4-((2-(2- (methylamino)-6-(2,2,2- trifluoroethyl) quinazolin-4-yl)-2,7- diazaspiro[3.5]nonan-7- yl)methyl)phenyl) ethanesulfonamide | 563.2416 | 563 |
| 16 | 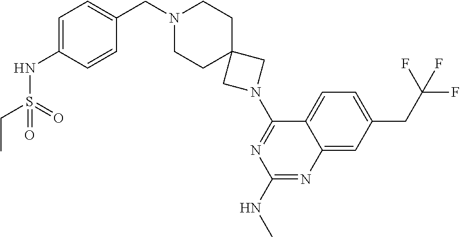 [SMILES: CCS(=O)(=O)Nc1ccc(CN2CCC3(CC2)CN(c2nc(NC)nc4cc(CC(F)(F)F)ccc24)C3)cc1] | N-(4-((2-(2- (methylamino)-7-(2,2,2- trifluoroethyl) quinazolin-4-yl)-2,7- diazaspiro[3.5]nonan-7- yl)methyl)phenyl) ethanesulfonamide | 563.2416 | 563 |
| 21 | 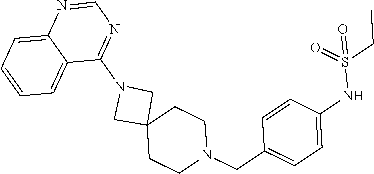 [SMILES: CCS(=O)(=O)Nc1ccc(CN2CCC3(CC2)CN(c2ncnc4ccccc24)C3)cc1] | N-(4-((2-(quinazolin- 4-yl)-2,7- diazaspiro[3.5]nonan-7- yl)methyl)phenyl) ethanesulfonamide | 452.212 | 452 |
| 22 | 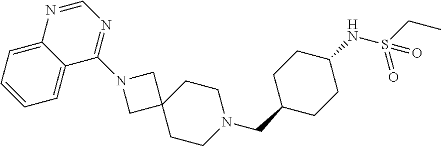 [SMILES: CCS(=O)(=O)N[C@H]1CC[C@H](CN2CCC3(CC2)CN(c2ncnc4ccccc24)C3)CC1] | N-((1R,4R)-4-((2- (quinazolin-4-yl)-2,7- diazaspiro[3.5]nonan-7- yl)methyl)cyclohexyl) ethanesulfonamide | 458.259 | 458 |
| 23 | 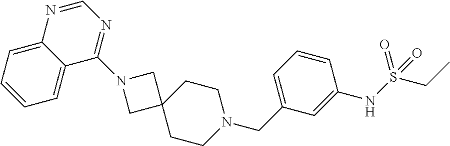 [SMILES: CCS(=O)(=O)Nc1cccc(CN2CCC3(CC2)CN(c2ncnc4ccccc24)C3)c1] | N-(3-((2-(quinazolin- 4-yl)-2,7- diazaspiro[3.5]nonan-7- yl)methyl)phenyl) ethanesulfonamide | 452.212 | 452 |
| 24 | 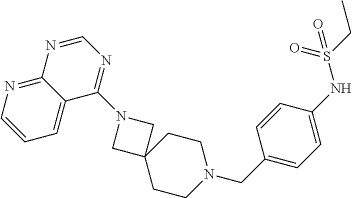 [SMILES: CCS(=O)(=O)Nc1ccc(CN2CCC3(CC2)CN(c2ncnc4ncccc24)C3)cc1] | N-[4-[(2-pyrido[2,3-d] pyrimidin-4-yl-2,7- diazaspiro[3.5]nonan-7- yl)methyl]phenyl] ethanesulfonamide | 453.2073 | 453 |
| 25 | 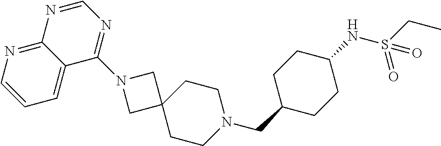 [SMILES: CCS(=O)(=O)N[C@H]1CC[C@H](CN2CCC3(CC2)CN(c2ncnc4ncccc24)C3)CC1] | N-[(1R,4R)-4-[(2-pyrido [2,3-d]pyrimidin-4-yl- 2,7-diazaspiro[3.5]nonan-7- yl)methyl]cyclohexyl] ethanesulfonamide | 459.2542 | 459 |
| 26 | 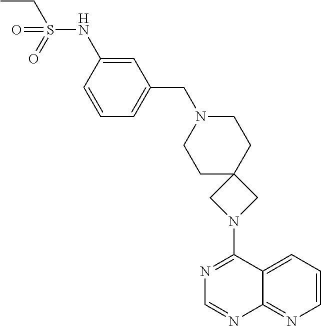 [SMILES: CCS(=O)(=O)Nc1cccc(CN2CCC3(CC2)CN(c2ncnc4ncccc24)C3)c1] | N-[3-[(2-pyrido[2,3-d] pyrimidin-4-yl-2,7- diazaspiro[3.5]nonan-7- yl)methyl]phenyl] ethanesulfonamide | 453.2073 | 453 |
| 27 | 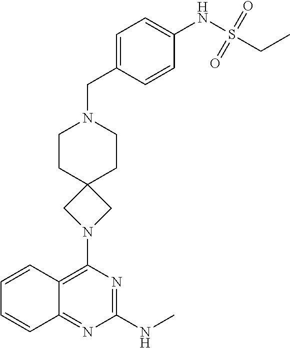 [SMILES: CCS(=O)(=O)Nc1ccc(CN2CCC3(CC2)CN(c2nc(NC)nc4ccccc24)C3)cc1] | N-(4-((2-(2-(methylamino) quinazolin-4-yl)-2,7- diazaspiro[3.5]nonan-7- yl)methyl)phenyl) ethanesulfonamide | 481.2386 | 481 |
| 28 | 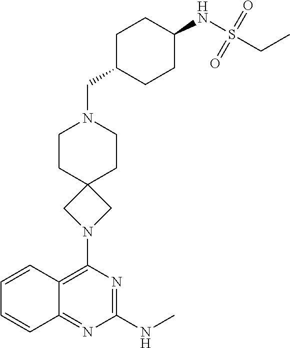 [SMILES: CCS(=O)(=O)N[C@H]1CC[C@H](CN2CCC3(CC2)CN(c2nc(NC)nc4ccccc24)C3)CC1] | N-((1R,4R)-4-((2-(2- (methylamino)quinazolin- 4-yl)-2,7-diazaspiro [3.5]nonan-7- yl)methyl)cyclohexyl) ethanesulfonamide | 487.2855 | 487 |
| 29 | 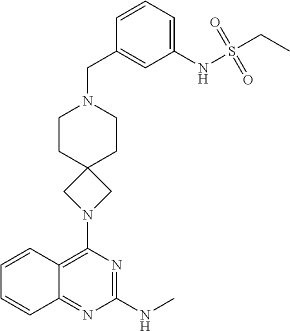 [SMILES: CCS(=O)(=O)Nc1cccc(CN2CCC3(CC2)CN(c2nc(NC)nc4ccccc24)C3)c1] | N-(3-((2-(2-(methylamino) quinazolin-4-yl)-2,7- diazaspiro[3.5]nonan-7- yl)methyl)phenyl) ethanesulfonamide | 481.2386 | 481 |
| 30 | 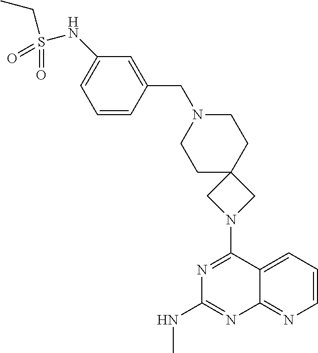 [SMILES: CCS(=O)(=O)Nc1cccc(CN2CCC3(CC2)CN(c2nc(NC)nc4ncccc24)C3)c1] | N-[3-[[2-[2-(methylamino) pyrido[2,3- d]pyrimidin-4-yl]-2,7- diazaspiro[3.5]nonan-7- yl]methyl]phenyl] ethanesulfonamide | 482.2338 | 482 |
| 31 | 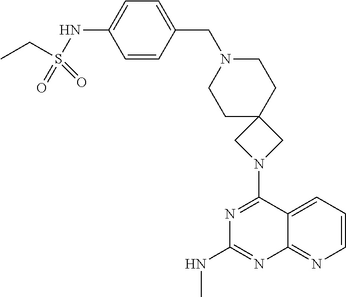 [SMILES: CCS(=O)(=O)Nc1ccc(CN2CCC3(CC2)CN(c2nc(NC)nc4ncccc24)C3)cc1] | N-[4-[[2-[2-(methylamino) pyrido[2,3- d]pyrimidin-4-yl]-2,7- diazaspiro[3.5]nonan-7- yl]methyl]phenyl] ethanesulfonamide | 482.2338 | 482 |
| 32 | 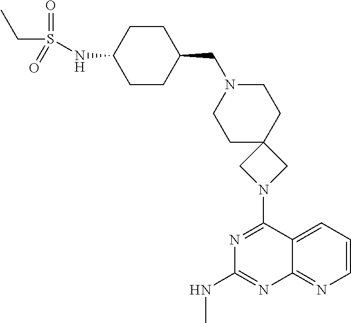 [SMILES: CCS(=O)(=O)N[C@H]1CC[C@H](CN2CCC3(CC2)CN(c2nc(NC)nc4ncccc24)C3)CC1] | N-(1R,4R)-[4-[[2-[2- (methylamino)pyrido[2,3- d]pyrimidin-4-yl]-2,7- diazaspiro[3.5]nonan-7- yl]methyl]cyclohexyl] ethanesulfonamide | 488.2808 | 488 |
| 33 | 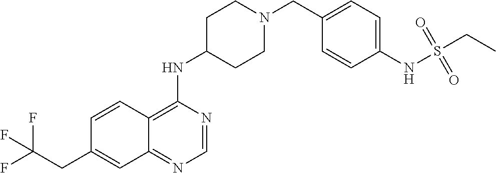 [SMILES: CCS(=O)(=O)Nc1ccc(CN2CCC(Nc3ncnc4cc(CC(F)(F)F)ccc34)CC2)cc1] | N-(4-((4-((7-(2,2,2- trifluoroethyl)quinazolin-4- yl)amino)piperidin-1- yl)methyl)phenyl) ethanesulfonamide | 508.1994 | 508 |
| 34 | 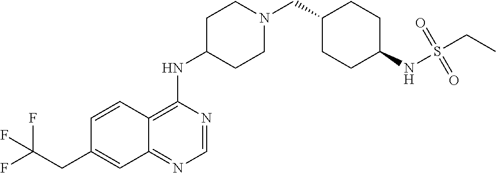 [SMILES: CCS(=O)(=O)N[C@H]1CC[C@H](CN2CCC(Nc3ncnc4cc(CC(F)(F)F)ccc34)CC2)CC1] | N-((1R,4R)-4-((4-((7-(2,2,2- trifluoroethyl)quinazolin- 4-yl)amino)piperidin- 1-yl)methyl)cyclohexyl) ethanesulfonamide | 514.2464 | 514 |
| 35 | 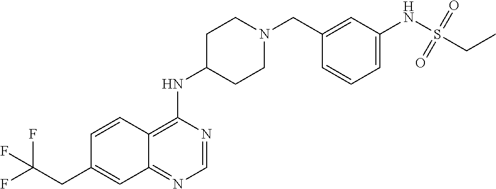 [SMILES: CCS(=O)(=O)Nc1cccc(CN2CCC(Nc3ncnc4cc(CC(F)(F)F)ccc34)CC2)c1] | N-(3-((4-((7-(2,2,2- trifluoroethyl)quinazolin-4- yl)amino)piperidin-1- yl)methyl)phenyl) ethanesulfonamide | 508.1994 | 508 |
| 36 | 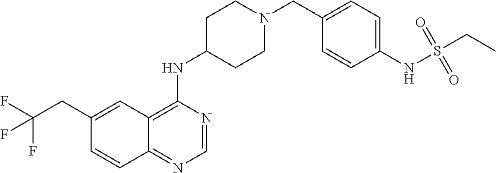 [SMILES: CCS(=O)(=O)Nc1ccc(CN2CCC(Nc3ncnc4ccc(CC(F)(F)F)cc34)CC2)cc1] | N-(4-((4-((6-(2,2,2- trifluoroethyl)quinazolin-4- yl)amino)piperidin-1- yl)methyl)phenyl) ethanesulfonamide | 508.1994 | 508 |
| 37 | 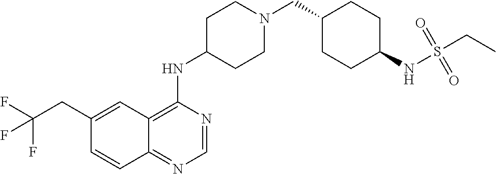 [SMILES: CCS(=O)(=O)N[C@H]1CC[C@H](CN2CCC(Nc3ncnc4ccc(CC(F)(F)F)cc34)CC2)CC1] | N-((1R,4R)-4-((4-((6-(2,2,2- trifluoroethyl)quinazolin- 4-yl)amino)piperidin- 1-yl)methyl)cyclohexyl) ethanesulfonamide | 514.2464 | 514 |
| 38 | 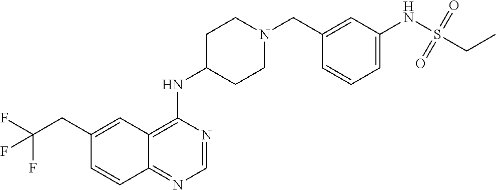 [SMILES: CCS(=O)(=O)Nc1cccc(CN2CCC(Nc3ncnc4ccc(CC(F)(F)F)cc34)CC2)c1] | N-(3-((4-((6-(2,2,2- trifluoroethyl)quinazolin-4- yl)amino)piperidin-1- yl)methyl)phenyl) ethanesulfonamide | 508.1994 | 508 |
| 39 | 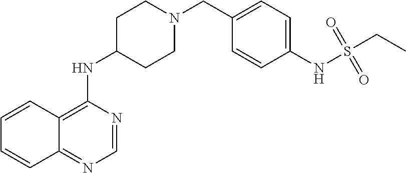 [SMILES: CCS(=O)(=O)Nc1ccc(CN2CCC(Nc3ncnc4ccccc34)CC2)cc1] | N-(4-((4-(quinazolin- 4-ylamino)piperidin-1- yl)methyl)phenyl) ethanesulfonamide | 426.1964 | 426 |
| 40 | 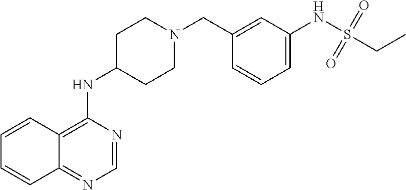 [SMILES: CCS(=O)(=O)Nc1cccc(CN2CCC(Nc3ncnc4ccccc34)CC2)c1] | N-(3-((4-(quinazolin- 4-ylamino)piperidin-1- yl)methyl)phenyl) ethanesulfonamide | 426.1964 | 426 |
| 41 | 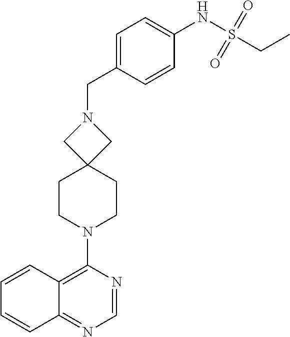 [SMILES: CCS(=O)(=O)Nc1ccc(CN2CC3(CCN(c4ncnc5ccccc45)CC3)C2)cc1] | N-(4-((7-(quinazolin- 4-yl)-2,7- diazaspiro[3.5]nonan-2- yl)methyl)phenyl) ethanesulfonamide | 452.212 | 452 |
| 42 | 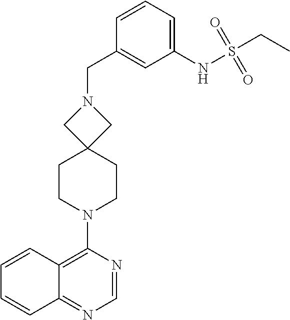 [SMILES: CCS(=O)(=O)Nc1cccc(CN2CC3(CCN(c4ncnc5ccccc45)CC3)C2)c1] | N-(3-((7-(quinazolin- 4-yl)-2,7- diazaspiro[3.5]nonan-2- yl)methyl)phenyl) ethanesulfonamide | 452.212 | 452 |
| 43 | 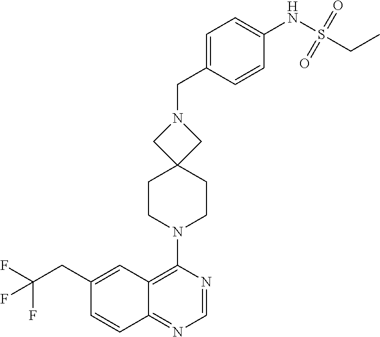 [SMILES: CCS(=O)(=O)Nc1ccc(CN2CC3(CCN(c4ncnc5ccc(CC(F)(F)F)cc45)CC3)C2)cc1] | N-(4-((7-(6-(2,2,2- trifluoroethyl)quinazolin-4- yl)-2,7-diazaspiro [3.5]nonan-2- yl)methyl)phenyl) ethanesulfonamide | 534.215 | 534 |
| 44 | 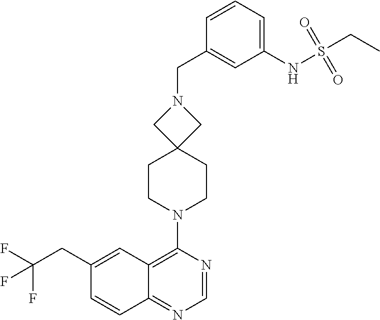 [SMILES: CCS(=O)(=O)Nc1cccc(CN2CC3(CCN(c4ncnc5ccc(CC(F)(F)F)cc45)CC3)C2)c1] | N-(3-((7-(6-(2,2,2- trifluoroethyl)quinazolin-4- yl)-2,7-diazaspiro [3.5]nonan-2- yl)methyl)phenyl) ethanesulfonamide | 534.215 | 534 |
| 45 | 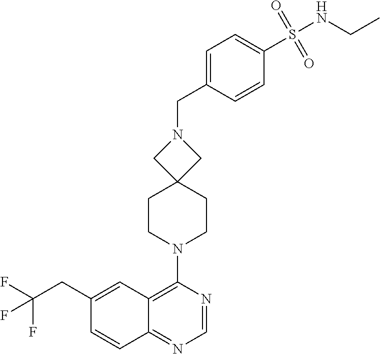 [SMILES: CCNS(=O)(=O)c1ccc(CN2CC3(CCN(c4ncnc5ccc(CC(F)(F)F)cc45)CC3)C2)cc1] | N-ethyl-4-((7-(6-(2,2,2- trifluoroethyl) quinazolin-4-yl)-2,7- diazaspiro[3.5]nonan-2- yl)methyl) benzenesulfonamide | 534.215 | 534 |
| 46 | 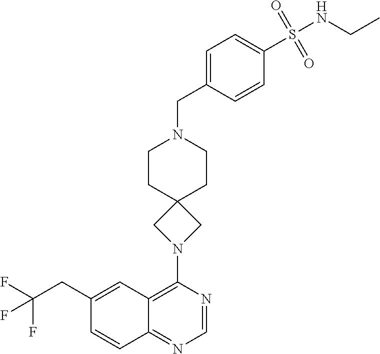 [SMILES: CCNS(=O)(=O)c1ccc(CN2CCC3(CC2)CN(c2ncnc4ccc(CC(F)(F)F)cc24)C3)cc1] | N-ethyl-4-((2-(6-(2,2,2- trifluoroethyl) quinazolin-4-yl)-2,7- diazaspiro[3.5]nonan-7- yl)methyl) benzenesulfonamide | 534.215 | 534 |
| 48 | 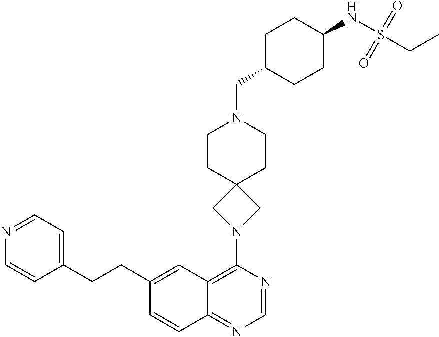 [SMILES: CCS(=O)(=O)N[C@H]1CC[C@H](CN2CCC3(CC2)CN(c2ncnc4ccc(CCc5ccncc5)cc24)C3)CC1] | N-((1r,4r)-4-((2- (6-(2-(pyridin-4- yl)ethyl)quinazolin-4-yl)-2,7- diazaspiro[3.5]nonan-7- yl)methyl)cyclohexyl) ethanesulfonamide | 563.3168 | 563 |
| 49 | 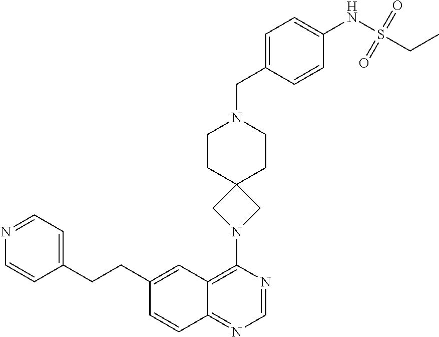 [SMILES: CCS(=O)(=O)Nc1ccc(CN2CCC3(CC2)CN(c2ncnc4ccc(CCc5ccncc5)cc24)C3)cc1] | N-(4-((2-(6-(2-(pyridin-4- yl)ethyl)quinazolin-4- yl)-2,7-diazaspiro [3.5]nonan-7- yl)methyl)phenyl) ethanesulfonamide | 557.2698 | 557 |
| 51 | 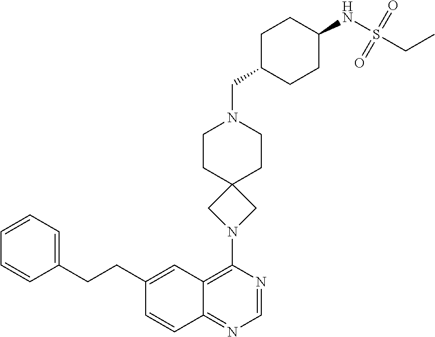 [SMILES: CCS(=O)(=O)N[C@H]1CC[C@H](CN2CCC3(CC2)CN(c2ncnc4ccc(CCc5ccccc5)cc24)C3)CC1] | N-((1R,4R)-4-((2-(6- phenethylquinazolin-4-yl)- 2,7-diazaspiro[3.5]nonan-7- yl)methyl)cyclohexyl) ethanesulfonamide | 562.3215 | 562 |
| 52 | 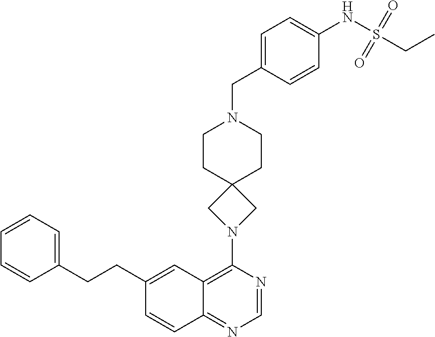 [SMILES: CCS(=O)(=O)Nc1ccc(CN2CCC3(CC2)CN(c2ncnc4ccc(CCc5ccccc5)cc24)C3)cc1] | N-(4-((2-(6- phenethylquinazolin-4- yl)-2,7- diazaspiro[3.5]nonan-7- yl)methyl)phenyl) ethanesulfonamide | 556.2746 | 556 |
| 53 | 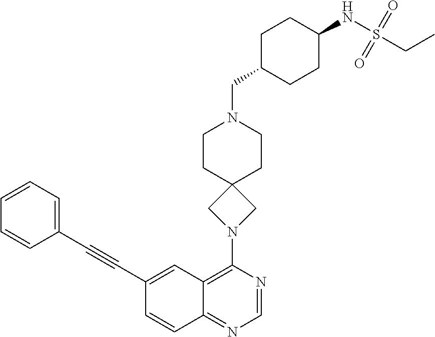 [SMILES: CCS(=O)(=O)N[C@H]1CC[C@H](CN2CCC3(CC2)CN(c2ncnc4ccc(C#Cc5ccccc5)cc24)C3)CC1] | N-((1R,4R)-4-((2-(6- (phenylethynyl)quinazolin- 4-yl)-2,7-diazaspiro [3.5]nonan-7- yl)methyl)cyclohexyl) ethanesulfonamide | 558.2903 | 558 |
| 54 | 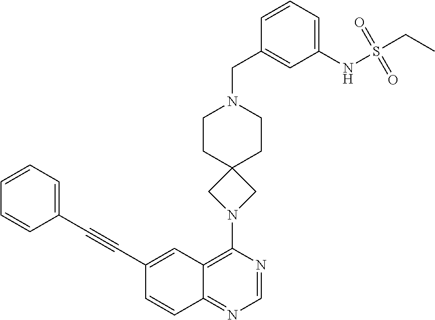 [SMILES: CCS(=O)(=O)Nc1cccc(CN2CCC3(CC2)CN(c2ncnc4ccc(C#Cc5ccccc5)cc24)C3)c1] | N-(3-((2-(6-(phenylethynyl) quinazolin-4-yl)- 2,7-diazaspiro[3.5]nonan-7- yl)methyl)phenyl) ethanesulfonamide | 552.2433 | 552 |
| 55 | 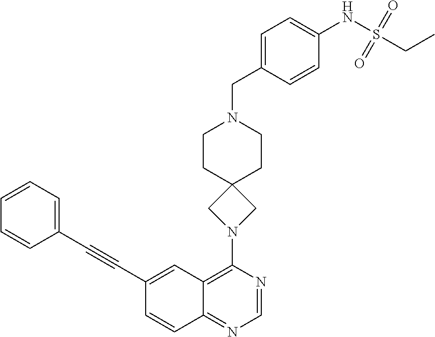 [SMILES: CCS(=O)(=O)Nc1ccc(CN2CCC3(CC2)CN(c2ncnc4ccc(C#Cc5ccccc5)cc24)C3)cc1] | N-(4-((2-(6-(phenylethynyl) quinazolin-4-yl)- 2,7-diazaspiro[3.5]nonan-7- yl)methyl)phenyl) ethanesulfonamide | 552.2433 | 552 |
| 56 | 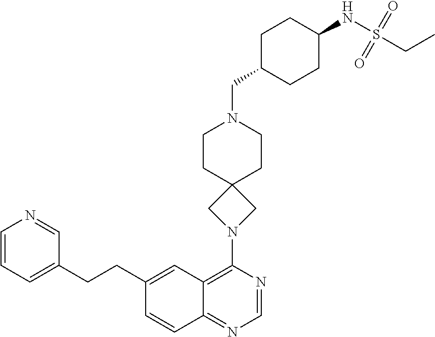 [SMILES: CCS(=O)(=O)N[C@H]1CC[C@H](CN2CCC3(CC2)CN(c2ncnc4ccc(CCc5cccnc5)cc24)C3)CC1] | N-((1R,4R)-4- ((2-(6-(2-(pyridin-3- yl)ethyl)quinazolin-4-yl)-2,7- diazaspiro[3.5]nonan-7- yl)methyl)cyclohexyl) ethanesulfonamide | 563.3168 | 563 |
| 57 | 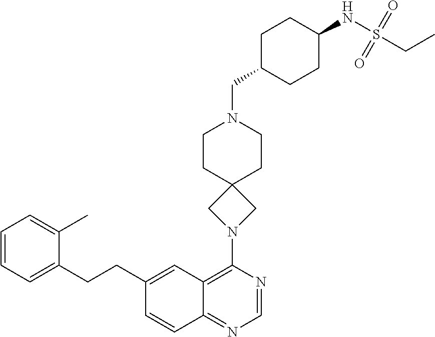 [SMILES: CCS(=O)(=O)N[C@H]1CC[C@H](CN2CCC3(CC2)CN(c2ncnc4ccc(CCc5ccccc5C)cc24)C3)CC1] | N-((1R,4R)-4-((2-(6-(2- methylphenethyl) quinazolin-4-yl)-2,7- diazaspiro[3.5]nonan-7- yl)methyl)cyclohexyl) ethanesulfonamide | 576.3372 | 576 |
| 58 | 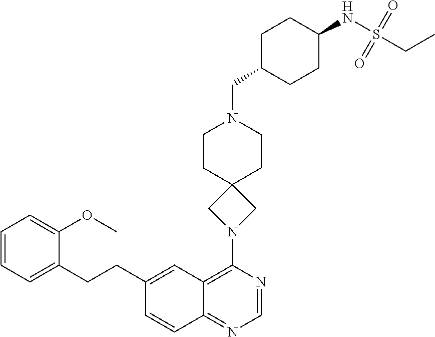 [SMILES: CCS(=O)(=O)N[C@H]1CC[C@H](CN2CCC3(CC2)CN(c2ncnc4ccc(CCc5ccccc5OC)cc24)C3)CC1] | N-((1R,4R)-4-((2-(6-(2- methoxyphenethyl) quinazolin-4-yl)-2,7- diazaspiro[3.5]nonan-7- yl)methyl)cyclohexyl) ethanesulfonamide | 592.3321 | 592 |
| 59 | 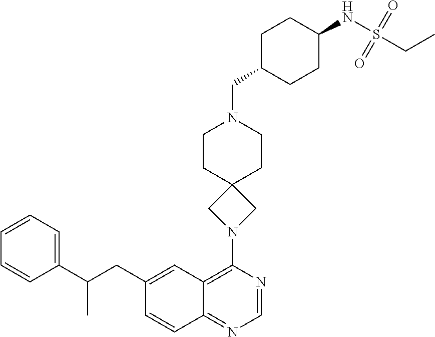 [SMILES: CCS(=O)(=O)N[C@H]1CC[C@H](CN2CCC3(CC2)CN(c2ncnc4ccc(CC(C)c5ccccc5)cc24)C3)CC1] | N-((1R,4R)-4-((2-(6-((RS)-2- phenylpropyl) quinazolin-4-yl)-2,7- diazaspiro[3.5]nonan-7- yl)methyl)cyclohexyl) ethanesulfonamide | 576.3372 | 576 |
| 60 | 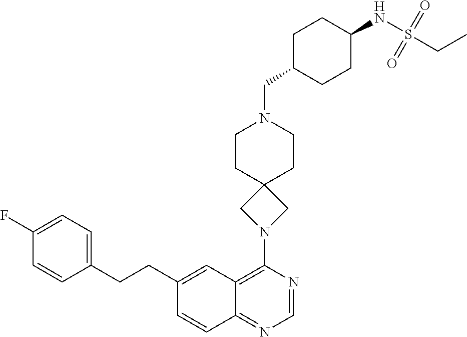 [SMILES: CCS(=O)(=O)N[C@H]1CC[C@H](CN2CCC3(CC2)CN(c2ncnc4ccc(CCc5ccc(F)cc5)cc24)C3)CC1] | N-((1R,4R)-4-((2-(6-(4- fluorophenethyl) quinazolin-4-yl)-2,7- diazaspiro[3.5]nonan-7- yl)methyl)cyclohexyl) ethanesulfonamide | 580.3121 | 580 |
| 61 | 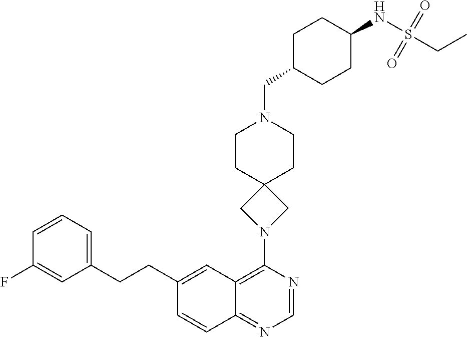 [SMILES: CCS(=O)(=O)N[C@H]1CC[C@H](CN2CCC3(CC2)CN(c2ncnc4ccc(CCc5cccc(F)c5)cc24)C3)CC1] | N-((1R,4R)-4-((2-(6-(3- fluorophenethyl) quinazolin-4-yl)-2,7- diazaspiro[3.5]nonan-7- yl)methyl)cyclohexyl) ethanesulfonamide | 580.3121 | 580 |
| 62 | 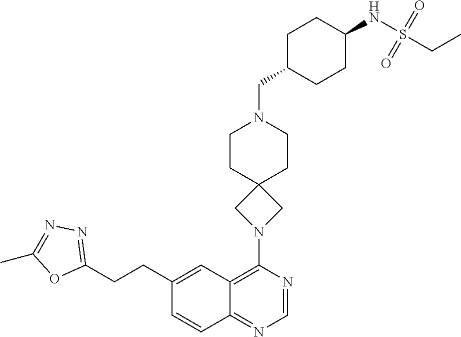 [SMILES: CCS(=O)(=O)N[C@H]1CC[C@H](CN2CCC3(CC2)CN(c2ncnc4ccc(CCc5nnc(C)o5)cc24)C3)CC1] | N-((1R,4R)-4-((2- (6-(2-(5-methyl-1,3,4- oxadiazol-2-yl)ethyl) quinazolin-4-yl)-2,7- diazaspiro[3.5]nonan-7- yl)methyl)cyclohexyl) ethanesulfonamide | 568.307 | 568 |
| 63 | 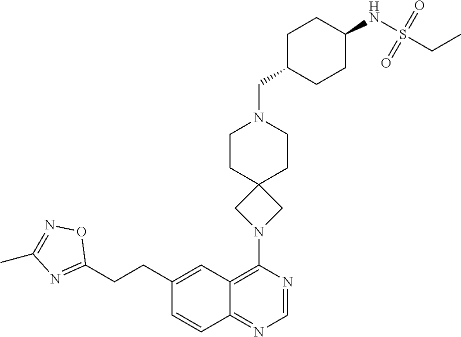 [SMILES: CCS(=O)(=O)N[C@H]1CC[C@H](CN2CCC3(CC2)CN(c2ncnc4ccc(CCc5nc(C)no5)cc24)C3)CC1] | N-((1R,4R)-4-((2- (6-(2-(3-methyl-1,2,4- oxadiazol-5-yl)ethyl) quinazolin-4-yl)-2,7- diazaspiro[3.5]nonan-7- yl)methyl)cyclohexyl) ethanesulfonamide | 568.307 | 568 |
| 64 | 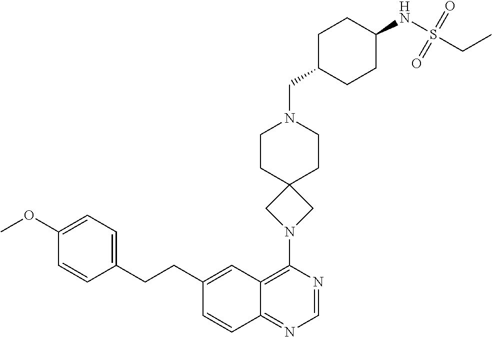 [SMILES: CCS(=O)(=O)N[C@H]1CC[C@H](CN2CCC3(CC2)CN(c2ncnc4ccc(CCc5ccc(OC)cc5)cc24)C3)CC1] | N-((1R,4R)-4-((2-(6-(4- methoxyphenethyl) quinazolin-4-yl)-2,7- diazaspiro[3.5]nonan-7- yl)methyl)cyclohexyl) ethanesulfonamide | 592.3321 | 592 |
| 65 | 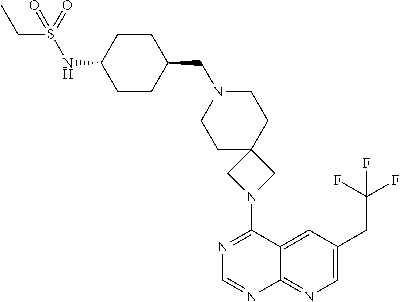 [SMILES: CCS(=O)(=O)N[C@H]1CC[C@H](CN2CCC3(CC2)CN(c2ncnc4ncc(CC(F)(F)F)cc24)C3)CC1] | N-(1R,4R)-[4-[[2-[6-(2,2,2- trifluoroethyl)pyrido[2,3-d] pyrimidin-4-yl]-2,7- diazaspiro[3.5]nonan-7- yl]methyl]cyclohexyl] ethanesulfonamide | 541.2573 | 541 |
| 66 | 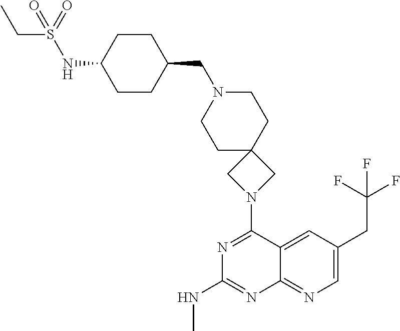 [SMILES: CCS(=O)(=O)N[C@H]1CC[C@H](CN2CCC3(CC2)CN(c2nc(NC)nc4ncc(CC(F)(F)F)cc24)C3)CC1] | N-(1R,4R)-[4-[[2-[2- (methylamino)-6-(2,2,2- trifluoroethyl)pyrido[2,3-d] pyrimidin-4-yl]-2,7- diazaspiro[3.5]nonan-7- yl]methyl]cyclohexyl] ethanesulfonamide | 570.2838 | 570 |
| 69 | 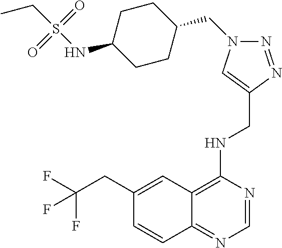 [SMILES: CCS(=O)(=O)N[C@H]1CC[C@H](Cn2cc(CNc3ncnc4ccc(CC(F)(F)F)cc34)nn2)CC1] | N-((1R,4R)-4-((4-(((6-(2,2,2- trifluoroethyl)quinazolin- 4-yl)amino)methyl)- 1H-1,2,3-triazol-1- yl)methyl)cyclohexyl) ethanesulfonamide | 512.2055 | 512 |
| 70 | 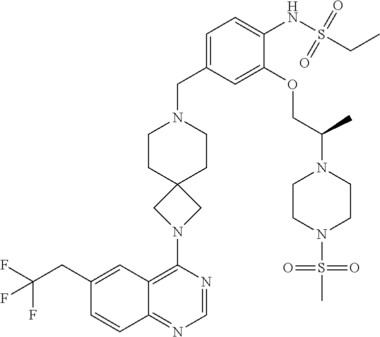 [SMILES: CCS(=O)(=O)Nc1ccc(CN2CCC3(CC2)CN(c2ncnc4ccc(CC(F)(F)F)cc24)C3)cc1OC[C@@H](C)N1CCN(S(C)(=O)=O)CC1] | rac-(R)-N-(2-(2-(4- (methylsulfonyl)piperazin-1- yl)propoxy)-4-((2-(6-(2,2,2- trifluoroethyl) quinazolin-4-yl)-2,7- diazaspiro[3.5]nonan-7- yl)methyl)phenyl) ethanesulfonamide | 754.3032 | 754 |
| 71 | 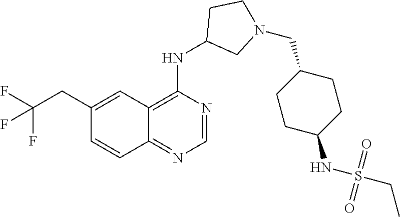 [SMILES: CCS(=O)(=O)N[C@H]1CC[C@H](CN2CCC(Nc3ncnc4ccc(CC(F)(F)F)cc34)C2)CC1] | N-((1R,4R)-4- (((RS)-3-((6-(2,2,2- trifluoroethyl)quinazolin- 4-yl)amino)pyrrolidin- 1-yl)methyl)cyclohexyl) ethanesulfonamide | 500.2307 | 500 |
| 72 | 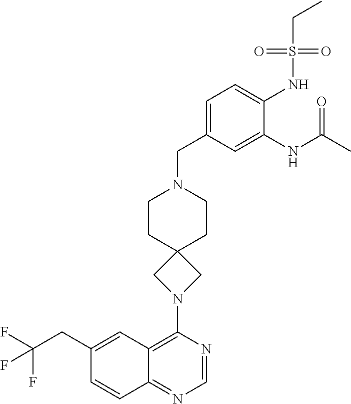 [SMILES: CCS(=O)(=O)Nc1ccc(CN2CCC3(CC2)CN(c2ncnc4ccc(CC(F)(F)F)cc24)C3)cc1NC(C)=O] | N-(2-(ethylsulfonamido)- 5-((2-(6-(2,2,2- trifluoroethyl) quinazolin-4-yl)-2,7- diazaspiro[3.5]nonan-7- yl)methyl)phenyl)acetamide | 591.2365 | 591 |
| 73 | 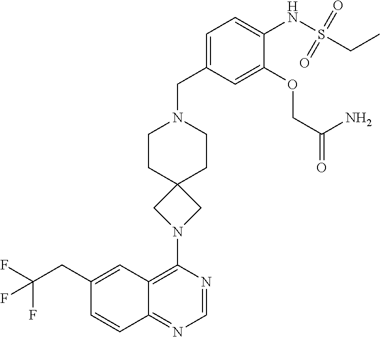 [SMILES: CCS(=O)(=O)Nc1ccc(CN2CCC3(CC2)CN(c2ncnc4ccc(CC(F)(F)F)cc24)C3)cc1OCC(N)=O] | 2-(2-(ethylsulfonamido)- 5-((2-(6-(2,2,2- trifluoroethyl) quinazolin-4-yl)-2,7- diazaspiro[3.5]nonan-7- yl)methyl)phenoxy)acetamide | 607.2315 | 607 |
| 74 | 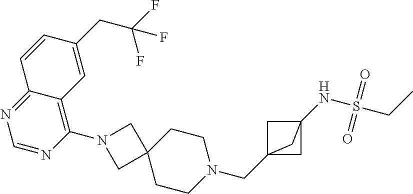 [SMILES: CCS(=O)(=O)NC12CC(CN3CCC4(CC3)CN(c3ncnc5ccc(CC(F)(F)F)cc35)C4)(C1)C2] | N-(3-((2-(6-(2,2,2- trifluoroethyl)quinazolin-4- yl)-2,7-diazaspiro[3.5]nonan-7- yl)methyl)bicyclo [1.1.1]pentan-1- yl)ethanesulfonamide | 524.2307 | 524 |
| 75 | 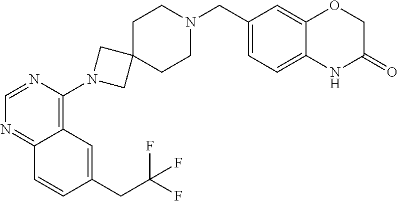 [SMILES: O=C1COc2cc(CN3CCC4(CC3)CN(c3ncnc5ccc(CC(F)(F)F)cc35)C4)ccc2N1] | 7-((2-(6-(2,2,2- trifluoroethyl) quinazolin-4-yl)- 2,7-diazaspiro[3.5]nonan- 7-yl)methyl)-2H- benzo[b][1,4]oxazin- 3(4H)-one | 498.2117 | 498 |
| 77 | 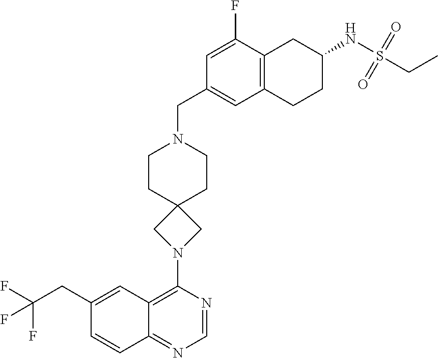 [SMILES: CCS(=O)(=O)N[C@@H]1CCc2cc(CN3CCC4(CC3)CN(c3ncnc5ccc(CC(F)(F)F)cc35)C4)cc(F)c2C1] | rac-(R)-N-(8-fluoro- 6-((2-(6-(2,2,2- trifluoroethyl) quinazolin-4-yl)-2,7- diazaspiro[3.5]nonan- 7-yl)methyl)-1,2,3,4- tetrahydronaphthalen-2- yl)ethanesulfonamide | 606.2526 | 606 |
| 78 | 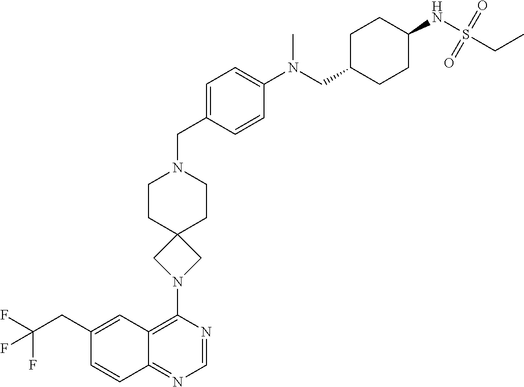 [SMILES: CCS(=O)(=O)N[C@H]1CC[C@H](CN(C)c2ccc(CN3CCC4(CC3)CN(c3ncnc5ccc(CC(F)(F)F)cc35)C4)cc2)CC1] | N-((1R,4R)-4- ((methyl(4-((2-(6-(2,2,2- trifluoroethyl) quinazolin-4-yl)-2,7- diazaspiro[3.5]nonan-7- yl)methyl)phenyl)amino) methyl)cyclohexyl) ethanesulfonamide | 659.3355 | 659 |
| 79 | 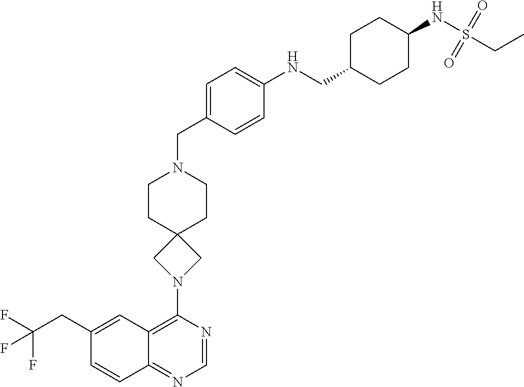 [SMILES: CCS(=O)(=O)N[C@H]1CC[C@H](CNc2ccc(CN3CCC4(CC3)CN(c3ncnc5ccc(CC(F)(F)F)cc35)C4)cc2)CC1] | N-((1R,4R)-4- (((4-((2-(6-(2,2,2- trifluoroethyl) quinazolin-4-yl)-2,7- diazaspiro[3.5]nonan-7- yl)methyl)phenyl)amino) methyl)cyclohexyl) ethanesulfonamide | 645.3198 | 645 |
| 80 | 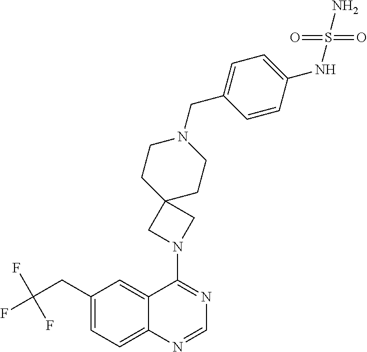 [SMILES: NS(=O)(=O)Nc1ccc(CN2CCC3(CC2)CN(c2ncnc4ccc(CC(F)(F)F)cc24)C3)cc1] | 4-[7-[[4-(sulfamoylamino) phenyl]methyl]-2,7- diazaspiro[3.5]nonan- 2-yl]-6-(2,2,2- trifluoroethyl)quinazoline | 521.1947 | 521 |
| 81 | 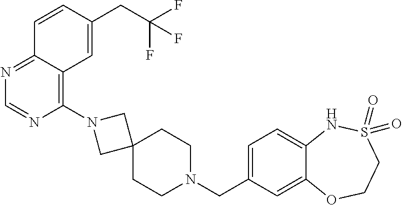 [SMILES: O=S1(=O)CCOc2cc(CN3CCC4(CC3)CN(c3ncnc5ccc(CC(F)(F)F)cc35)C4)ccc2N1] | 7-((2-(6-(2,2,2- trifluoroethyl) quinazolin-4-yl)- 2,7-diazaspiro[3.5]nonau- 7-yl)methyl)-3,4- dihydro-1H-benzo[f][1,4,5] oxathiazepine 2,2- dioxide | 548.1943 | 548 |
| 82 | 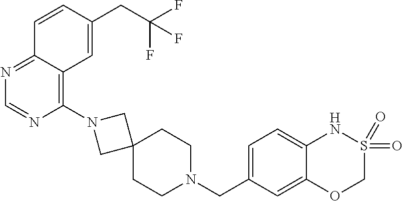 [SMILES: O=S1(=O)COc2cc(CN3CCC4(CC3)CN(c3ncnc5ccc(CC(F)(F)F)cc35)C4)ccc2N1] | 6-((2-(6-(2,2,2-trifluoroethyl) quinazolin-4-yl)- 2,7-diazaspiro[3.5]nonan- 7-yl)methyl)-1H,3H- benzo[e][1,3,4]oxathiazine 2,2-dioxide | 534.1787 | 534 |
| 83 | 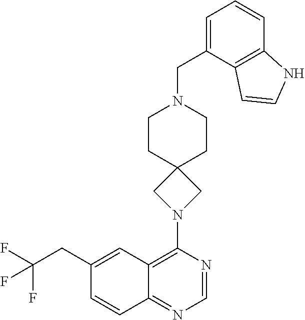 [SMILES: FC(F)(F)Cc1ccc2ncnc(N3CC4(CCN(Cc5cccc6[nH]ccc56)CC4)C3)c2c1] | 4-(7-((1H-indol- 4-yl)methyl)-2,7- diazaspiro[3.5]nonan- 2-yl)-6-(2,2,2- trifluoroethyl)quinazoline | 466.2218 | 466 |
| 84 | 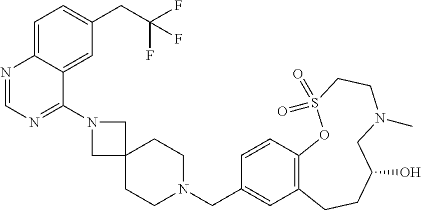 [SMILES: CN1CCS(=O)(=O)Oc2ccc(CN3CCC4(CC3)CN(c3ncnc5ccc(CC(F)(F)F)cc35)C4)cc2CC[C@@H](O)C1] | rac-(R)-7-hydroxy-5- methyl-11-((2-(6-(2,2,2- trifluoroethyl) quinazolin-4-yl)-2,7- diazaspiro[3.5]nonan-7- yl)methyl)-3,4,5,6,7,8- hexahydro-1H- benzo[b][1]oxa[5]thia[4,8] diazacycloundecine 2,2-dioxide | 635.2627 | 635 |
| 85 | 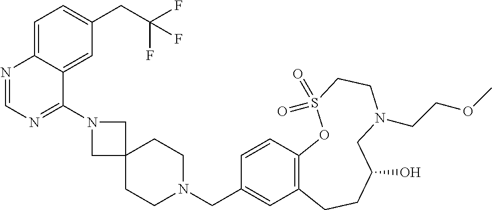 [SMILES: COCCN1CCS(=O)(=O)Oc2ccc(CN3CCC4(CC3)CN(c3ncnc5ccc(CC(F)(F)F)cc35)C4)cc2CC[C@@H](O)C1] | rac-(R)-7-hydroxy-5- (2-methoxyethyl)-11-((2- (6-(2,2,2-trifluoroethyl) quinazolin-4-yl)-2,7- diazaspiro[3.5]nonan-7- yl)methyl)-3,4,5,6,7,8- hexahydro-1H- benzo[b][1]oxa[5]thia[4,8] diazacycloundecine 2,2-dioxide | 679.289 | 679 |
| 86 | 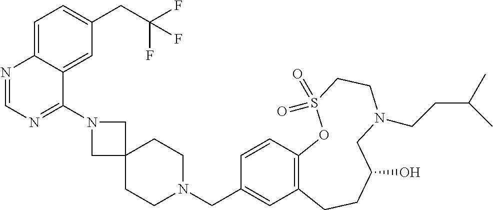 [SMILES: CC(C)CCN1CCS(=O)(=O)Oc2ccc(CN3CCC4(CC3)CN(c3ncnc5ccc(CC(F)(F)F)cc35)C4)cc2CC[C@@H](O)C1] | rac-(R)-7-hydroxy-5- isopentyl-11-((2-(6-(2,2,2- trifluoroethyl) quinazolin-4-yl)-2,7- diazaspiro[3.5]nonan-7- yl)methyl)-3,4,5,6,7,8- hexahydro-1H- benzo[b][1]oxa[5]thia[4,8] diazacyclo-undecine 2,2-dioxide | 691.3253 | 691 |
| 87 | 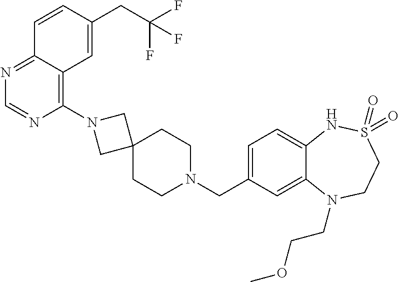 [SMILES: COCCN1CCS(=O)(=O)Nc2ccc(CN3CCC4(CC3)CN(c3ncnc5ccc(CC(F)(F)F)cc35)C4)cc21] | 5-(2-methoxyethyl)- 7-((2-(6-(2,2,2- trifluoroethyl) quinazolin-4-yl)-2,7- diazaspiro[3.5]nonan- 7-yl)methyl)-1,3,4,5- tetrahydrobenzo[c][1,2,5] thiadiazepine 2,2- dioxide | 605.2521 | 605 |
| 88 | 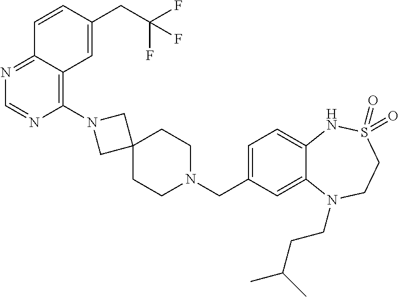 [SMILES: CC(C)CCN1CCS(=O)(=O)Nc2ccc(CN3CCC4(CC3)CN(c3ncnc5ccc(CC(F)(F)F)cc35)C4)cc21] | 5-isopentyl-7-((2-(6-(2,2,2- trifluoroethyl) quinazolin-4-yl)-2,7- diazaspiro[3.5]nonan-7- yl)methyl)-1,3,4,5- tetrahydrobenzo[c][1,2,5] thiadiazepine 2,2- dioxide | 617.2885 | 617 |
| 89 | 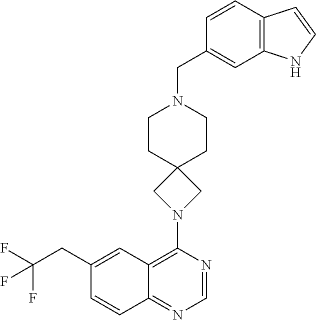 [SMILES: FC(F)(F)Cc1ccc2ncnc(N3CC4(CCN(Cc5ccc6cc[nH]c6c5)CC4)C3)c2c1] | 4-(7-((1H-indol-6- yl)methyl)-2,7- diazaspiro[3.5]nonan- 2-yl)-6-(2,2,2- trifluoroethyl)quinazoline | 466.2218 | 466 |
| 90 | 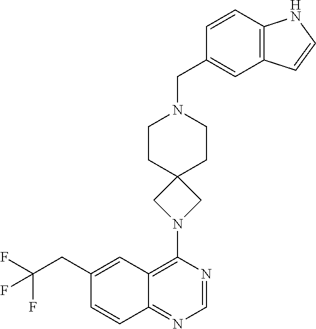 [SMILES: FC(F)(F)Cc1ccc2ncnc(N3CC4(CCN(Cc5ccc6[nH]ccc6c5)CC4)C3)c2c1] | 4-(7-((1H-indol-5- yl)methyl)-2,7- diazaspiro[3.5]nonan- 2-yl)-6-(2,2,2- trifluoroethyl)quinazoline | 466.2218 | 466 |
| 91 | 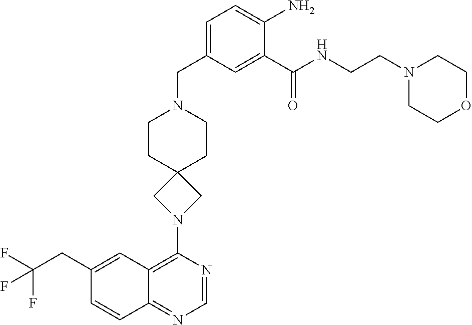 [SMILES: Nc1ccc(CN2CCC3(CC2)CN(c2ncnc4ccc(CC(F)(F)F)cc24)C3)cc1C(=O)NCCN1CCOCC1] | 2-amino-N-(2- morpholinoethyl)- 5-((2-(6-(2,2,2- trifluoroethyl) quinazolin-4-yl)-2,7- diazaspiro[3.5]nonan-7- yl)methyl)benzamide | 598.3117 | 598 |
| 92 | 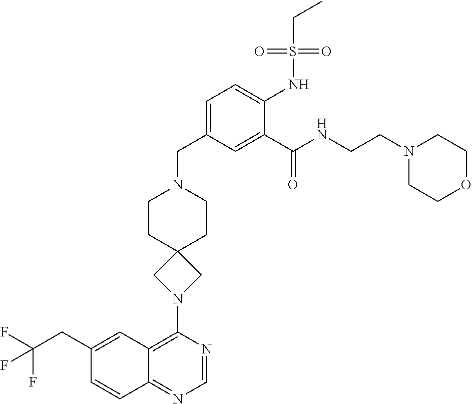 [SMILES: CCS(=O)(=O)Nc1ccc(CN2CCC3(CC2)CN(c2ncnc4ccc(CC(F)(F)F)cc24)C3)cc1C(=O)NCCN1CCOCC1] | 2-(ethylsulfonamido)-N- (2-morpholinoethyl)-5- ((2-(6-(2,2,2-trifluoroethyl) quinazolin-4-yl)-2,7- diazaspiro[3.5]nonan-7-yl) methyl)benzamide | 690.3049 | 690 |
| 93 | 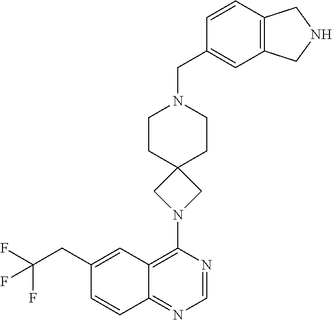 [SMILES: FC(F)(F)Cc1ccc2ncnc(N3CC4(CCN(Cc5ccc6c(c5)CNC6)CC4)C3)c2c1] | 4-(7-(isoindolin- 5-ylmethyl)-2,7- diazaspiro[3.5]nonan- 2-yl)-6-(2,2,2- trifluoroethyl)quinazoline | 468.2375 | 468 |
| 94 | 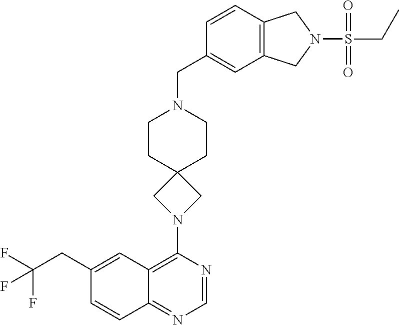 [SMILES: CCS(=O)(=O)N1Cc2ccc(CN3CCC4(CC3)CN(c3ncnc5ccc(CC(F)(F)F)cc35)C4)cc2C1] | 4-(7-((2-(ethylsulfonyl) isoindolin-5-yl)methyl)- 2,7-diazaspiro[3.5]nonan- 2-yl)-6-(2,2,2- trifluoroethyl)quinazoline | 560.2307 | 560 |
| 95 | 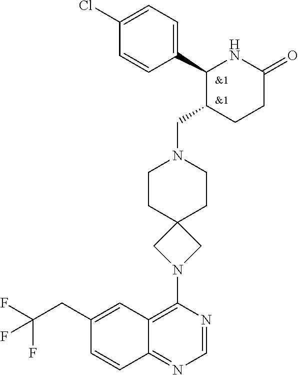 [SMILES: O=C1CC[C@H](CN2CCC3(CC2)CN(c2ncnc4ccc(CC(F)(F)F)cc24)C3)[C@@H](c2ccc(Cl)cc2)N1] | rac-(5R,6S)-6- (4-chlorophenyl)- 5-((2-(6-(2,2,2- trifluoroethyl) quinazolin-4-yl)-2,7- diazaspiro[3.5]nonan-7- yl)methyl)piperidin-2- one | 558.2247 | 558 |
| 100 | 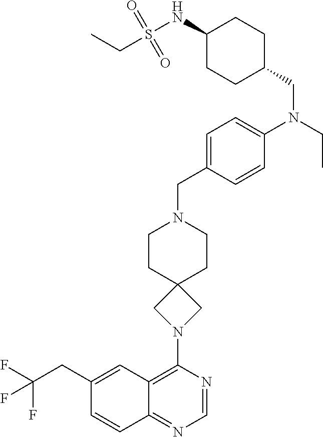 [SMILES: CCN(C[C@H]1CC[C@H](NS(=O)(=O)CC)CC1)c1ccc(CN2CCC3(CC2)CN(c2ncnc4ccc(CC(F)(F)F)cc24)C3)cc1] | N-((1R,4R)-4-((ethyl (4-((2-(6-(2,2,2- trifluoroethyl) quinazolin-4-yl)-2,7- diazaspiro[3.5]nonan-7- yl)methyl)phenyl)amino) methyl)cyclohexyl) ethanesulfonamide | 673.3511 | 673 |
| 101 | 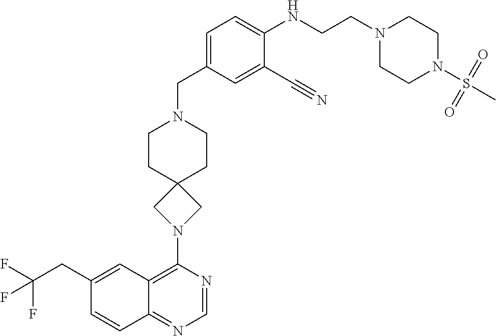 [SMILES: CS(=O)(=O)N1CCN(CCNc2ccc(CN3CCC4(CC3)CN(c3ncnc5ccc(CC(F)(F)F)cc35)C4)cc2C#N)CC1] | 2-((2-(4-(methylsulfonyl) piperazin-1- yl)ethyl)amino)- 5-((2-(6-(2,2,2- trifluoroethyl) quinazolin-4-yl)-2,7- diazaspiro[3.5]nonan-7- yl)methyl)benzonitrile | 657.2947 | 657 |
| 102 | 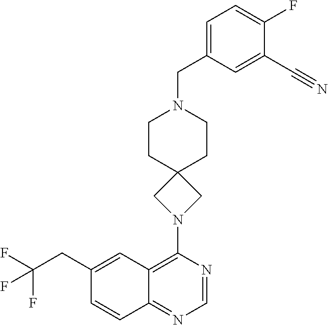 [SMILES: N#Cc1cc(CN2CCC3(CC2)CN(c2ncnc4ccc(CC(F)(F)F)cc24)C3)ccc1F] | 2-fluoro-5-((2-(6-(2,2,2- trifluoroethyl) quinazolin-4-yl)-2,7- diazaspiro[3.5]nonan-7- yl)methyl)benzonitrile | 470.1968 | 470 |
| 110 | 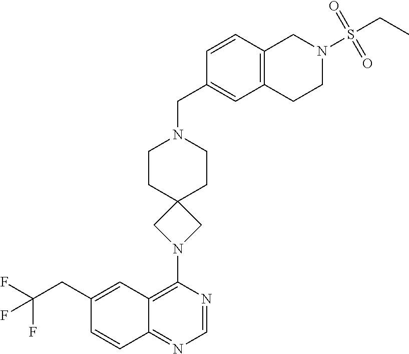 [SMILES: CCS(=O)(=O)N1CCc2cc(CN3CCC4(CC3)CN(c3ncnc5ccc(CC(F)(F)F)cc35)C4)ccc2C1] | 4-(7-((2- (ethylsulfonyl)-1,2,3,4- tetrahydroisoquinolin- 6-yl)methyl)-2,7- diazaspiro[3.5]nonan- 2-yl)-6-(2,2,2- trifluoroethyl)quinazoline | 574.2464 | 574 |
| 111 | 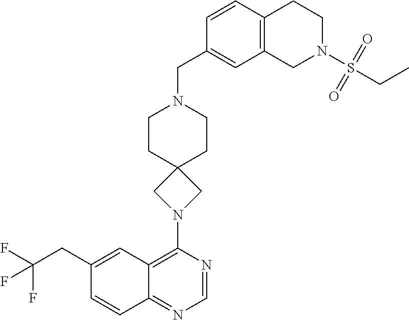 [SMILES: CCS(=O)(=O)N1CCc2ccc(CN3CCC4(CC3)CN(c3ncnc5ccc(CC(F)(F)F)cc35)C4)cc2C1] | 4-(7-((2- (ethylsulfonyl)-1,2,3,4- tetrahydroisoquinolin- 7-yl)methyl)-2,7- diazaspiro[3.5]nonan- 2-yl)-6-(2,2,2- trifluoroethyl)quinazoline | 574.2464 | 574 |
| 138 | 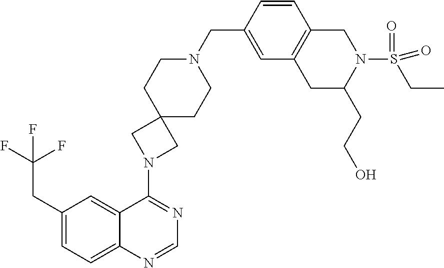 [SMILES: CCS(=O)(=O)N1Cc2ccc(CN3CCC4(CC3)CN(c3ncnc5ccc(CC(F)(F)F)cc35)C4)cc2CC1CCO] | 2-[2-ethylsulfonyl- 6-[[2-[6-(2,2,2- trifluoroethyl) quinazolin-4-yl]-2,7- diazaspiro[3.5]nonan-7-yl] methyl]-3,4-dihydro- 1H-isoquinolin-3-yl]ethanol | 618.2726 | 618 |

### Synthesis of the Representative Examples of the Compound

### Example 3. 4-(7-{[2-(Ethylsulfonyl)-1,2,3,4-tetrahydroisoquinolin-6-yl]methyl}-2,7-diazaspiro[3.5]non-2-yl)-6-(2,2,2-trifluoroethyl)quinazoline (Compound 110)

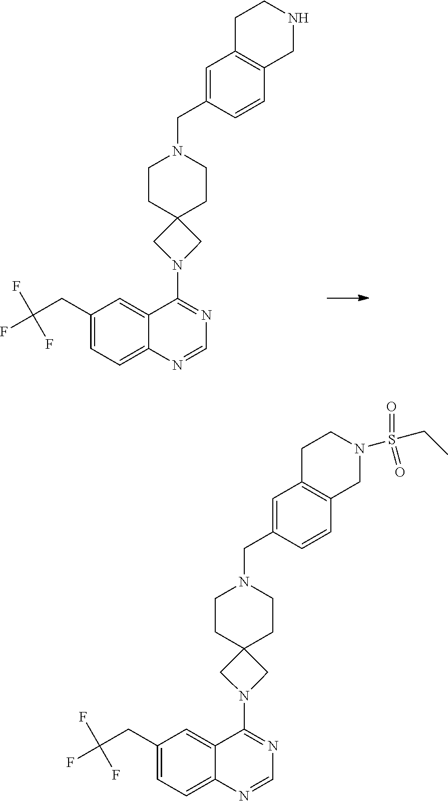 [SMILES: FC(F)(F)Cc1ccc2ncnc(N3CC4(CCN(Cc5ccc6c(c5)CCNC6)CC4)C3)c2c1 ; CCS(=O)(=O)N1CCc2cc(CN3CCC4(CC3)CN(c3ncnc5ccc(CC(F)(F)F)cc35)C4)ccc2C1]

The solution of 4-[7-(1,2,3,4-tetrahydroisoquinolin-6-ylmethyl)-2,7-diazaspiro[3.5]non-2-yl]-6-(2,2,2-trifluoroethyl)quinazoline (P36, 31 mg, 0.060 mmol) and ethanesulfonyl chloride (11 mg, 0.084 mmol) and DIPEA (97 mg, 0.75 mmol) in DCM (5 ml) was stirred at rt for 24 h (LCMS control). Then a water solution of sodium bicarbonate was added to the reaction mixture with stirring. The mixture was stirred for 30 min and layers were separated. The water phase was extracted with DCM (100 ml). Combined DCM extract was concentrated, and residue purified by column chromatography on silica with ethylacetate-methanol (10:2) to yield the target compound (Compound 110, 14 mg, 40%). ¹H NMR (400 MHZ, DMSO-d₆), δ: 8.44 (s, 1H), 7.96 (s, 1H), 7.77-7.66 (m, 2H), 17.30-7.11 (m, 3H), 4.39 (s, 2H), 4.45-4.02 (m, 4H), 3.84 (q, J=11.3 Hz, 2H), 3.48 (t, J=5.6 Hz, 2H), 3.46-3.38 (m, 2H), 3.12 (q, J=7.3 Hz, 2H), 2.87 (t, J=5.4 Hz, 2H), 2.40-2.26 (m, 4H), 1.95-1.72 (m, 4H), 1.20 (t, J=7.3 Hz, 3H). LCMS (ESI) [MH]⁺: 574.

### Example 4. 4-(7-{[2-(methylsulfonyl)-2,3-dihydro-1H-isoindol-5-yl]methyl}-2,7-diazaspiro[3.5]non-2-yl)-6-(2,2,2-trifluoroethyl)quinazoline (Compound 94)

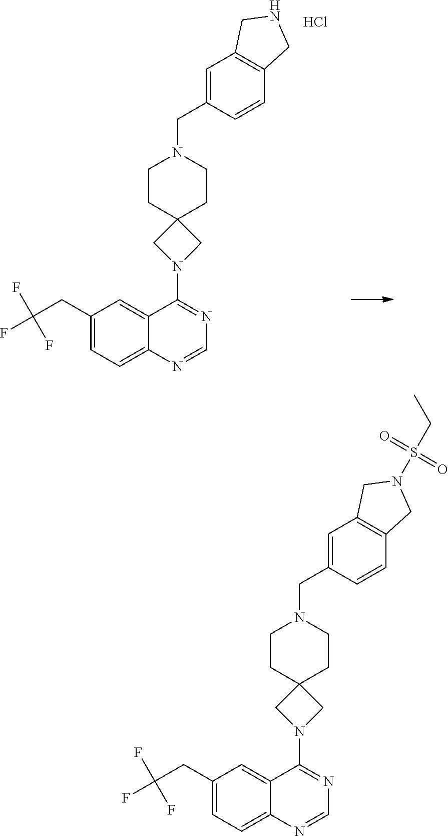 [SMILES: FC(F)(F)Cc1ccc2ncnc(N3CC4(CCN(Cc5ccc6c(c5)CNC6)CC4)C3)c2c1 ; Cl ; CCS(=O)(=O)N1Cc2ccc(CN3CCC4(CC3)CN(c3ncnc5ccc(CC(F)(F)F)cc35)C4)cc2C1]

To the solution of 4-[7-(2,3-dihydro-1H-isoindol-5-ylmethyl)-2,7-diazaspiro[3.5]non-2-yl]-6-(2,2,2-trifluoroethyl)quinazoline (P38, 28 mg, 0.054 mmol) in DCM (5 ml) were added DIPEA (35 mg, 0.27 mmol) and ethanesulfonyl chloride (11 mg, 0.081 mmol). The mixture was stirred at room temperature for 24 h (LCMS control). Then a water solution of sodium bicarbonate was added to the reaction mixture with stirring. The mixture was stirred for 30 min and layers were separated. The water phase was extracted with DCM (100 ml). Combined DCM extract was concentrated, and residue purified by column chromatography on silica with ethylacetate-methanol (10:2) to yield the target compound (Compound 94, 9 mg, 12%). LCMS(ESI) [MH]⁺: 560.

### Example 5. N-[trans-4-({9-[6-(2,2,2-trifluoroethyl)quinazolin-4-yl]-3,9-diazaspiro[5.5]undec-3-yl}methyl)cyclohexyl]ethanesulfonamide (Compound 3)

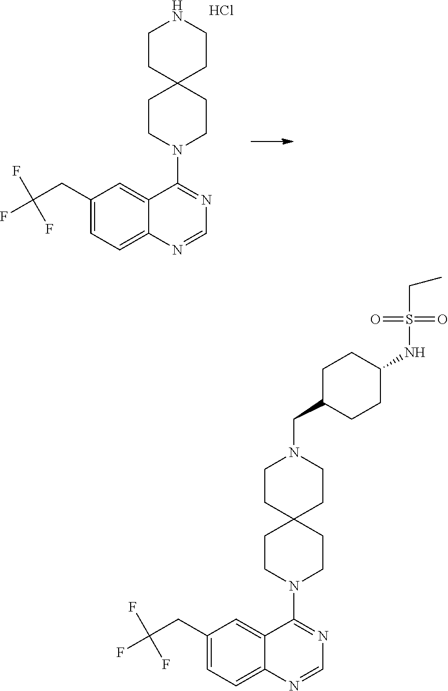

The mixture of 3-[6-(2,2,2-trifluoroethyl)quinazolin-4-yl]-3,9-diazaspiro[5.5]undecane (P28, 54 mg, 0.13 mmol), N-(trans-4-formylcyclohexyl)ethanesulfonamide (P133, 37 mg, 0.2 mmol), sodium triacethoxyborohydride (114 mg, 0.54 mmol) in DCM (3 ml) was stirred at rt for 24 h (LCMS control). Then a water solution of sodium bicarbonate was added to the reaction mixture with stirring. The mixture was stirred for 30 min and layers were separated. The water phase was extracted with DCM (100 ml). Combined DCM extract was concentrated, and residue purified by column chromatography on silica with ethylacetate-methanol (10:2) to yield the target compound (Compound 3, 16 mg, 29%). ¹H NMR. (400 MHZ, DMSO-d₆), δ: 8.57 (s, 1H), 7.95 (s, 1H), 7.80-7.70 (m, 2H), 6.99 (d, J=7.8 Hz, 1H), 3.88 (q, J=11.7 Hz, 2H), 3.87-3.78 (m, 4H), 3.00-2.92 (m, 3H), 2.50-2.25 (m, 4H), 2.20-1.95 (m, 2H), 1.80-1.70 (m, 2H), 1.70-1.61 (m, 2H), 1.60 (s, 4H), 1.37 (s, 1H), 1.17 (t, J=7.3 Hz, 3H), 0.85 (q, J=11.2 Hz, 2H). LCMS(EST) [MH]⁺: 568.

### Example 6. N-[3-({9-[6-(2,2,2-trifluoroethyl)quinazolin-4-yl]-3,9-diazaspiro[5.5]undec-3-yl}methyl)phenyl]ethanesulfonamide (Compound 44)

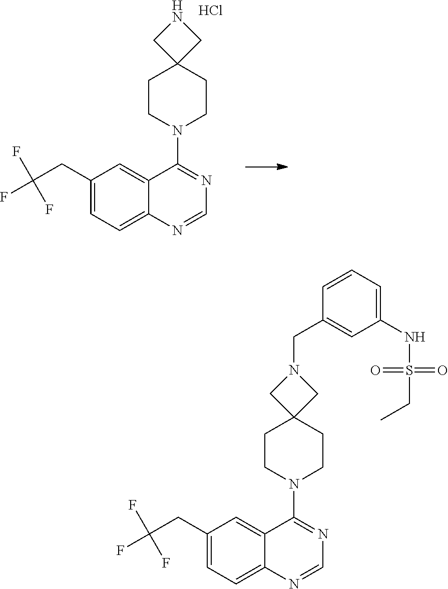 [SMILES: Cl ; FC(F)(F)Cc1ccc2ncnc(N3CCC4(CC3)CNC4)c2c1 ; CCS(=O)(=O)Nc1cccc(CN2CC3(CCN(c4ncnc5ccc(CC(F)(F)F)cc45)CC3)C2)c1]

The mixture of 4-(2,7-diazaspiro[3.5]non-7-yl)-6-(2,2,2-trifluoroethyl)quinazoline (P26, 50 mg, 0.13 mmol), N-[3-(formyl)phenyl]ethanesulfonamide (P22, 36 mg, 0.2 mmol) and sodium triacethoxyborohydride (114 mg, 0.54 mmol) in DCM (3 ml) was stirred at rt for 24 h (LCMS control). Then a water solution of sodium bicarbonate was added to the reaction mixture with stirring. The mixture was stirred for 30 min and layers were separated. The water phase was extracted with DCM (100 ml). Combined DCM extract was concentrated, and residue purified by column chromatography on silica with ethylacetate-methanol (10:2) to yield the target compound (Compound 44, 18 mg, 33%). ¹H NMR (400 MHZ, DMSO-d₆), δ: 9.74 (s, 1H), 8.59 (s, 1H), 7.93 (s, 1H), 8.81-7.78 (m, 2H), 7.25 (t, J=7.8 Hz, 1H), 7.26-7.15 (m, 1H), 7.10 (d, J=7.8 Hz, 1H), 7.00 (d, J=7.3 Hz, 1H), 3.89 (q, J=11.7 Hz, 2H), 3.75-3.40 (m, 5H), 3.38-3.25 (m, 4H), 3.06 (q, J=7.3 Hz, 2H), 2.05-1.80 (m, 4H), 1.18 (t, J=7.3 Hz, 3H). LCMS(ESI) [MH]⁺: 534.

### Example 7. N-[4-({9-[6-(2,2,2-trifluoroethyl)quinazolin-4-yl]-3,9-diazaspiro[5.5]undec-3-yl}methyl)phenyl]ethanesulfonamide (Compound 7)

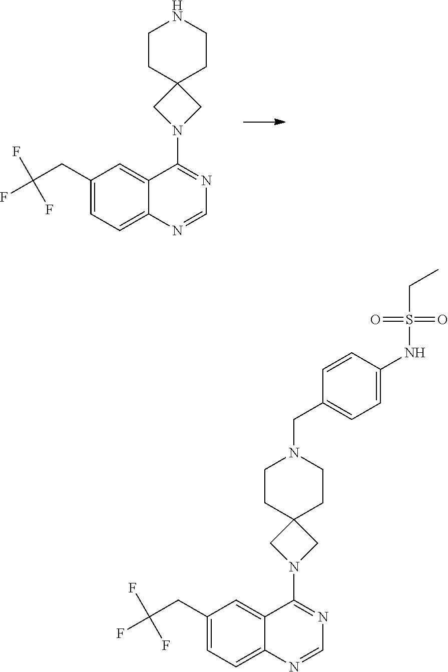 [SMILES: FC(F)(F)Cc1ccc2ncnc(N3CC4(CCNCC4)C3)c2c1 ; CCS(=O)(=O)Nc1ccc(CN2CCC3(CC2)CN(c2ncnc4ccc(CC(F)(F)F)cc24)C3)cc1]

Compound 7 was prepared using the procedure described in Example 6 and appropriate substrates: 4-(2,7-diazaspiro[3.5]non-2-yl)-6-(2,2,2-trifluoroethyl)quinazoline (P9) and N-[4-(formyl)phenyl]ethanesulfonamide (P18). Yield of the Compound 7 was 51%. ¹H NMR (400 MHz, DMSO-d₆), δ: 9.70 (s, 1H), 8.43 (s, 1H), 7.96 (s, 1H), 7.80-7.71 (m, 2H), 7.23 (d, J=8.3 Hz, 2H), 7.17 (d, J=8.8 Hz, 2H), 4.41-4.15 (m, 3H), 3.86 (q, J=11.7 Hz, 2H), 3.39 (s, 2H), 3.05 (q, J=7.3 Hz, 2H), 2.40-2.25 (m, 3H), 1.80-1.74 (m, 4H), 1.18 (t, J=7.3 Hz, 3H). LCMS(ESI) [MH]⁺: 534.

### Example 8. N-[trans-4-({[4-({9-[6-(2,2,2-trifluoroethyl)quinazolin-4-yl]-3,9-diazaspiro[5.5]undec-3-yl}methyl)phenyl]amino}methyl)cyclohexyl]ethanesulfonamide (Compound 79)

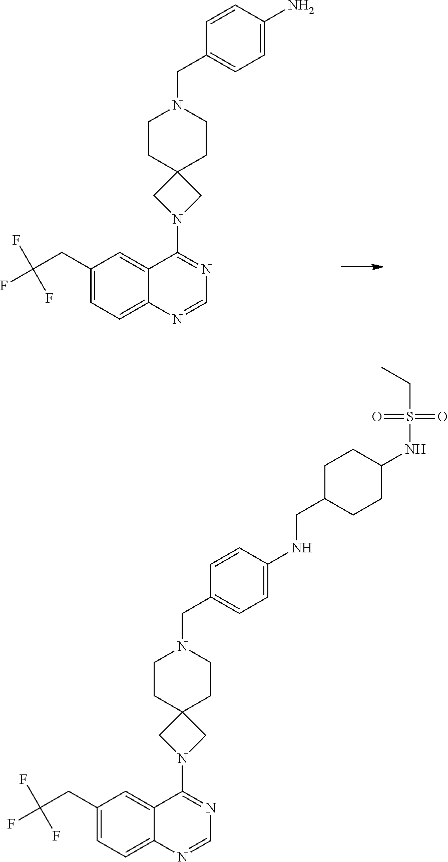 [SMILES: Nc1ccc(CN2CCC3(CC2)CN(c2ncnc4ccc(CC(F)(F)F)cc24)C3)cc1 ; CCS(=O)(=O)NC1CCC(CNc2ccc(CN3CCC4(CC3)CN(c3ncnc5ccc(CC(F)(F)F)cc35)C4)cc2)CC1]

Compound 79 was prepared using the procedure described in Example 6 and appropriate substrates: [4-({2-[6-(2,2,2-trifluoroethyl)quinazolin-4-yl]-2,7-diazaspiro[3.5]non-7-yl}methyl)phenyl]amine (P24) and N-(trans-4-formylcyclohexyl)ethanesulfonamide (P133). Yield 65%. ¹H NMR (400 MHz, DMSO-d₆), δ: 10.41 (s, 1H), 8.84 (s, 1H), 8.08-7.99 (m, 2H), 7.90 (t, J=8.3 Hz, 1H), 7.25 (d, J=7.8 Hz, 2H), 6.98 (d, J=7.8 Hz, 1H), 6.60 (d, J=8.3 Hz, 2H), 4.85 (s, 1H), 4.77 (s, 1H), 4.33-4.26 (m, 6H), 4.18-4.02 (m, 2H), 3.93 (q, J=11.7 Hz, 2H), 3.43-3.21 (m, 2H), 3.04-2.91 (m, 4H), 2.85 (d, J=6.4 Hz, 2H), 2.28-2.18 (m, 2H), 2.16-2.05 (m, 1H), 1.92-1.77 (m, 3H), 1.50-1.31 (m, 1H), 1.26-1.15 (m, 1H), 1.17 (t, J=6.7 Hz, 3H), 1.06-0.98 (m, 2H). LCMS(ESI) [MH]⁺: 645.

### Example 11. 4-[7-(1H-indol-6-ylmethyl)-2,7-diazaspiro[3.5]non-2-yl]-6-(2,2,2-trifluoroethyl)quinazoline (Compound 89)

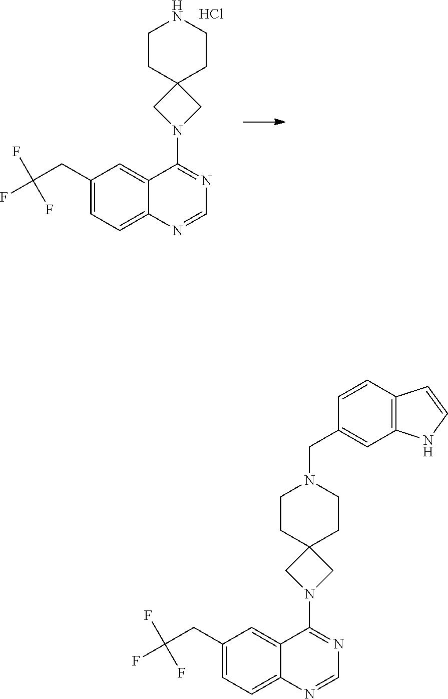 [SMILES: Cl ; FC(F)(F)Cc1ccc2ncnc(N3CC4(CCNCC4)C3)c2c1 ; FC(F)(F)Cc1ccc2ncnc(N3CC4(CCN(Cc5ccc6cc[nH]c6c5)CC4)C3)c2c1]

Compound 89 was prepared using the procedure described in Example 6 and appropriate substrates: 6-formylindole and 4-(2,7-diazaspiro[3.5]non-2-yl)-6-(2,2,2-trifluoroethyl)quinazoline hydrochloride (P9). Yield 35%, ¹H NMR (400 MHZ, DMSO-d₆), δ: 10.97 (s, 1H), 8.43 (s, 1H), 7.96 (s, 1H), 7.70 (m, 2H), 7.45 (d, J=8.3 Hz, 1H), 7.85-7.75 (m, 2H), 6.96 (d, J=8.3 Hz, 1H), 6.37 (s, 1H), 4.40-4.00 (m, 4H), 3.86 (q, J=11.3 Hz, 2H), 3.54 (s, 2H), 2.60-2.25 (m, 4H), 1.87-1.71 (m, 4H). LCMS(ESI) [MH]⁺: 466.

### Example 12. 4-[7-(1H-indol-4-ylmethyl)-2,7-diazaspiro[3.5]non-2-yl]-6-(2,2,2-trifluoroethyl)quinazoline (Compound 83)

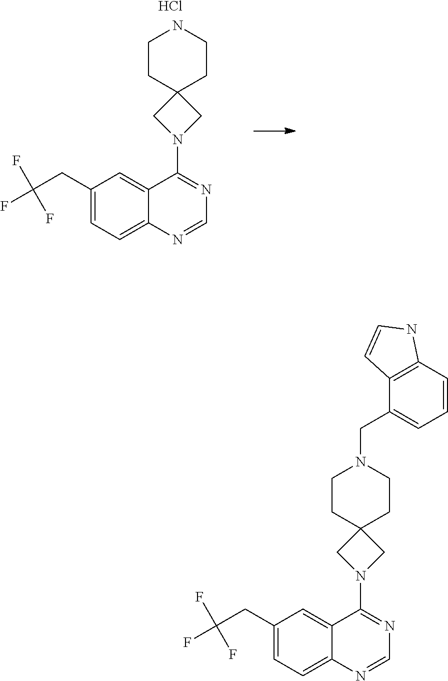 [SMILES: Cl ; FC(F)(F)Cc1ccc2ncnc(N3CC4(CCNCC4)C3)c2c1 ; FC(F)(F)Cc1ccc2ncnc(N3CC4(CCN(Cc5cccc6[nH]ccc56)CC4)C3)c2c1]

Compound 83 was prepared using the procedure described in Example 6 and appropriate substrates: 4-formylindole and 4-(2,7-diazaspiro[3.5]non-2-yl)-6-(2,2,2-trifluoroethyl)quinazoline hydrochloride (P9). Yield 35%. ¹H NMR (400 MHZ, DMSO-d₆), δ: 11.04 (s, 1H), 8.43 (s, 1H), 7.96 (s, 1H), 7.79-7.74 (m, 2H), 7.40-7.24 (m, 2H), 7.03 (t, J=7.8 Hz, 1H), 6.94 (d, J=6.9 Hz, 1H), 6.58 (s, 1H), 4.41-4.00 (m, 4H), 3.84 (q, J=11.7 Hz, 2H), 3.70 (s, 2H), 2.48-2.25 (m, 4H), 1.90-1.75 (m, 4H). LCMS(ESI) [MH]⁺: 466.

### Example 4-[7-(1H-indol-5-ylmethyl)-2,7-diazaspiro[3.5]non-2-yl]-6-(2,2,2-trifluoroethyl)quinazoline (Compound 90)

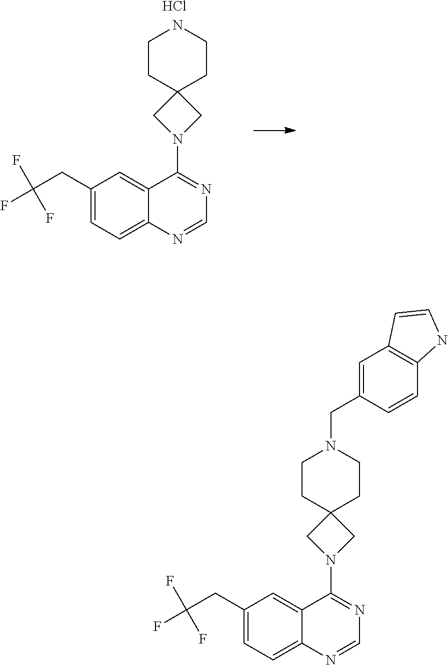 [SMILES: Cl ; FC(F)(F)Cc1ccc2ncnc(N3CC4(CCNCC4)C3)c2c1 ; FC(F)(F)Cc1ccc2ncnc(N3CC4(CCN(Cc5ccc6[nH]ccc6c5)CC4)C3)c2c1]

Compound 90 was prepared using the procedure described in Example 6 and appropriate substrates: 5-formylindole and 4-(2,7-diazaspiro[3.5]non-2-yl)-6-(2,2,2-trifluoroethyl)quinazoline hydrochloride (P9). Yield 26%. ¹H NMR (400 MHZ, DMSO-d₆), δ: 11.01 (s, 1H), 8.43 (s, 1H), 7.96 (s, 1H), 7.86-7.55 (m, 2H), 7.50-7.38 (m, 1H), 7.25-7.35 (m, 2H), 7.05 (d, J=8.3 Hz, 1H), 6.37 (s, 1H), 4.50-4.00 (m, 4H), 3.86 (q, J=11.7 Hz, 2H), 3.51 (s, 2H), 2.65-2.21 (m, 4H), 2.06 (s, 3H), 1.87-1.75 (m, 4H). LCMS(ESI) [MH]⁺: 466.

### Example 14. N-[trans-4-({2-[6-(2,2,2-trifluoroethyl)quinazolin-4-yl]-2,7-diazaspiro[3.5]non-7-yl}methyl)cyclohexyl]ethanesulfonamide (Compound 5)

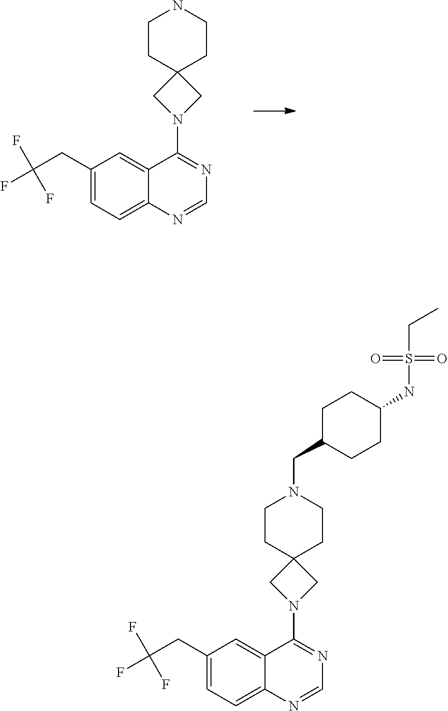

Compound 5 was prepared using the procedure described in Example 6 and appropriate substrates: N-(trans-4-formylcyclohexyl)ethanesulfonamide (P133) and 4-(2,7-diazaspiro[3.5]non-2-yl)-6-(2,2,2-trifluoroethyl)quinazoline hydrochloride (P9). Yield 40%. ¹H NMR (400 MHZ, DMSO-d₆), δ: 8.58 (s, 1H), 7.79 (d, J=8.8 Hz, 1H), 7.76 (s, 1H), 7.62 (d, J=8.3 Hz, 1H), 4.30-4.21 (m, 4H), 4.06 (d, J=7.8 Hz, 1H), 3.49 (q, J=10.8 Hz, 2H), 3.32-3.21 (m, 1H), 3.02 (q, J=7.3 Hz, 2H), 2.50-2.30 (m, 4H), 2.13-2.03 (m, 4H), 1.92-1.81 (m, 6H), 1.81-1.73 (m, 1H), 1.47-1.31 (m, 4H), 1.24 (m, 2H), 1.20-0.88 (m, 2H). LCMS(ESI) [MH]⁺: 540.

### Example 16. 7-({2-[6-(2,2,2-trifluoroethyl)quinazolin-4-yl]-2,7-diazaspiro[3.5]non-7-yl}methyl)-2H-1,4-benzoxazin-3 (4H)-one (Compound 75)

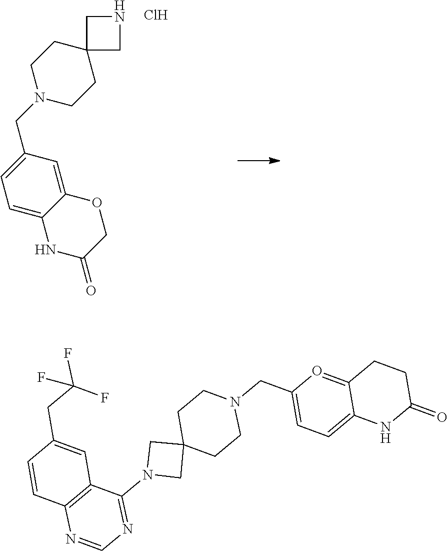

7-(2,7-Diazaspiro[3.5]non-7-ylmethyl)-2H-1,4-benzoxazin-3 (4H)-one hydrochloride (P42, 42 mg, 0.13 mmol) and DIPEA (35 μl, 0.19 mmol) were added to a solution of 4-chloro-6-(2,2,2-trifluoroethyl)quinazoline (P7, 35 mg, 0.13 mmol) in DCM (1 ml). The reaction mixture was stirred at rt for 18 h (LCMS control). The product was purified by column chromatography on silica with ethylacetate-methanol (9:1). 7-({2-[6-(2,2,2-Trifluoroethyl)quinazolin-4-yl]-2,7-diazaspiro[3.5]non-7-yl}methyl)-2H-1,4-benzoxazin-3 (4H)-one (Compound 75) was obtained as a white solid (15 mg, 24%). ¹H NMR (400 MHz, DMSO-d₆), δ: 10.66 (s, 1H), 8.44 (s, 1H), 7.97 (s, 1H), 7.76-7.65 (m, 2H), 6.91-6.80 (m, 3H), 4.55 (s, 2H), 4.19 (s, 4H), 3.86 (q, J=11.3 Hz, 2H), 2.52 (s, 2H), 2.33 (s, 4H), 1.80 (s, 4H). LCMS(ESI) [MH]⁺: 498.

### Example 35. N-[trans-4-({2-[6-(2-Phenylethyl)quinazolin-4-yl]-2,7-diazaspiro[3.5]non-7-yl}methyl)cyclohexyl]ethanesulfonamide (Compound 51)

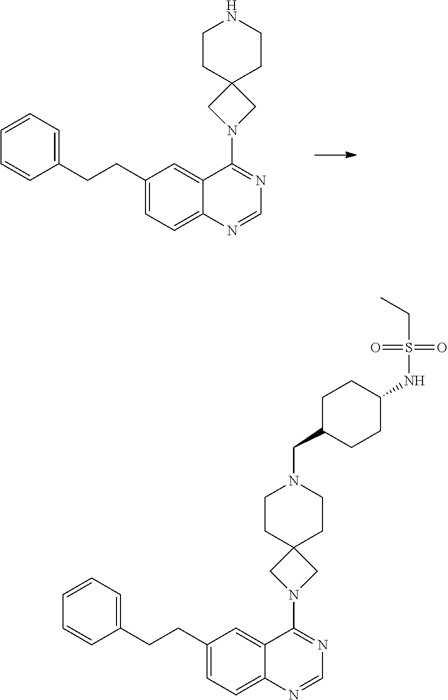

To a solution of 4-(2,7-diazaspiro[3.5]non-2-yl)-6-(2-phenylethyl)quinazoline (P148, 55 mg, 0.154 mmol) in DCM (5 ml) were added N-(trans-4-formylcyclohexyl)ethanesulfonamide (P133, 39 mg, 0.183 mmol) and triacethoxyborohydride (114 mg, 0.54 mmol). The mixture was stirred at rt for 24 h (LCMS control), then with stirring a water solution of sodium bicarbonate was added. The mixture was stirred for 30 min, and extracted with DCM (2×30 ml), DCM was evaporated, and residue purified by column chromatography on silica with ethylacetate-methanol (10:2) to yield the Compound 51 (25 mg, 29%) as a white solid. ¹H NMR (400 MHZ, DMSO-d₆), δ: 8.37 (s, 1H), 7.66 (dd, J=8.6, 1.5 Hz, 1H), 7.61 (d, J=8.6 Hz, 1H), 7.56 (s, 1H), 7.23 (m, 5H), 7.01 (d, J=7.7 Hz, 1H), 4.05 (bs, 4H), 3.05 (t, J=7.5 Hz, 2H), 2.95 (m, 5H), 2.31 (bs, 4H), 2.02 (bs, 2H), 1.87 (d, J=12.5 Hz, 2H), 1.76 (s, 6H), 1.40 (s, 1H), 1.18 (m, 5H), 0.89 (m, 2H). LCMS(ESI) [MH]⁺: 562.

### Example 36. N-[4-({2-[6-(2-Phenylethyl)quinazolin-4-yl]-2,7-diazaspiro[3.5]non-7-yl}methyl)phenyl]ethanesulfonamide (Compound 52)

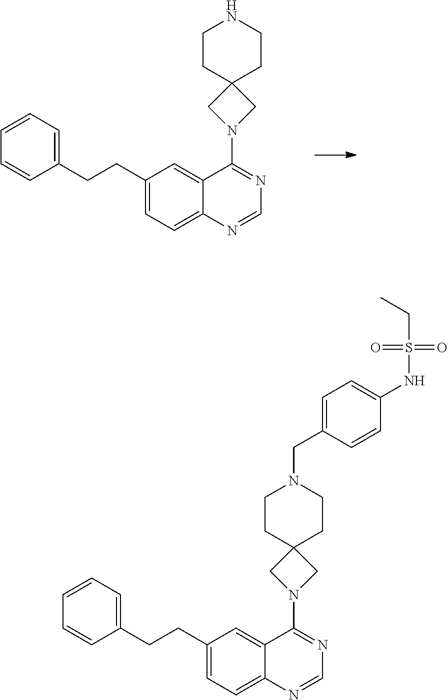 [SMILES: c1ccc(CCc2ccc3ncnc(N4CC5(CCNCC5)C4)c3c2)cc1 ; CCS(=O)(=O)Nc1ccc(CN2CCC3(CC2)CN(c2ncnc4ccc(CCc5ccccc5)cc24)C3)cc1]

To a solution of 4-(2,7-diazaspiro[3.5]non-2-yl)-6-(2-phenylethyl)quinazoline (P148, 55 mg, 0.154 mmol) in DCM (5 ml) were added N-4-formylphenylethanesulfonamide (P137, 40 mg, 0.183 mmol), triacethoxyborohydride (114 mg, 0.54 mmol). The mixture was stirred at rt for 24 h (LCMS control), then water solution of sodium bicarbonate was added with stirring. The mixture was stirred for 30 min, water phase was extracted with DCM (2×30 ml), DCM was evaporated, and residue purified by column chromatography on silica with ethylacetate-methanol (10:2) to yield the Compound 52 (25 mg, 29%) as a white solid. ¹H NMR (400 MHZ, DMSO-d₆), δ: 9.74 (s, 1H), 8.38 (s, 1H), 7.66 (dd, J=8.5, 1.8 Hz, 1H), 7.61 (d, J=8.5 Hz, 1H), 7.55 (s, 1H), 7.21 (m, 9H), 4.05 (bs, 4H), 3.43 (bs, 2H), 3.06 (m, 4H), 2.93 (t, J=7.5 Hz, 2H), 2.34 (bs, 4H), 1.77 (bs, 4H), 1.19 (t, J=7.3 Hz, 3H). LCMS(ESI) [MH]⁺: 556.

### Example 37. N-[trans-4-({2-[6-(2-pyridin-3-ylethyl)quinazolin-4-yl]-2,7-diazaspiro[3.5]non-7-yl}methyl)cyclohexyl]ethanesulfonamide (Compound 56)

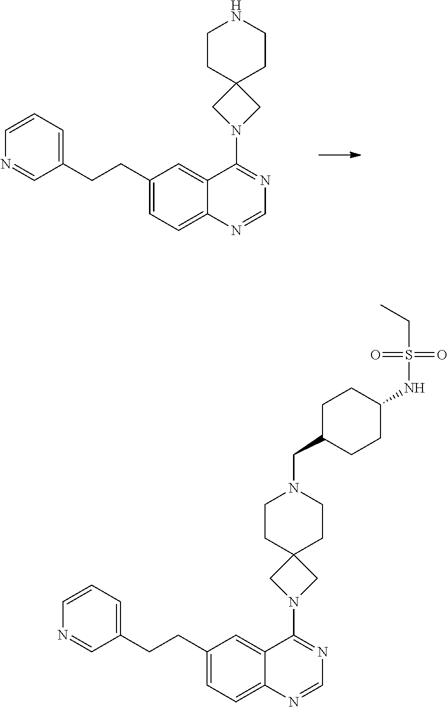

To a solution of 4-(2,7-diazaspiro[3.5]non-2-yl)-6-(2-pyridin-3-ylethyl)quinazoline (P150, 55 mg, 0.154 mmol) in DCM (5 ml)N-(trans-4-formylcyclohexyl)ethanesulfonamide (44 mg, 0.20 mmol), triacethoxyborohydride (114 mg, 0.54 mmol) were added. The mixture was stirred at rt for 24 h (LCMS control), then water solution of sodium bicarbonate was added with stirring. The mixture was stirred for 30 min, water phase was extracted with DCM (2×30 ml), DCM was removed under vacuum and residue was purified by column chromatography on silica with ethylacetate-methanol (10:2) to yield the Compound 56 (25 mg, 29%) as a white solid. ¹H NMR (400 MHZ, DMSO-d₆), δ: 8.38 (m, 3H), 7.63 (m, 3H), 7.58 (d, J=1.7 Hz, 1H), 7.29 (dd, J=7.8, 4.8 Hz, 1H), 7.00 (d, J=7.7 Hz, 1H), 4.06 (bs, 4H), 3.07 (t, J=7.5 Hz, 2H), 2.97 (m, 5H), 2.29 (bs, 4H), 2.05 (d, J=7.1 Hz, 2H), 1.87 (m, 2H), 1.75 (m, 6H), 1.39 (s, 1H), 1.18 (m, 5H), 0.89 (q, J=12.0 Hz, 2H). LCMS(ESI) [MH]⁺: 563.

### Example 38. N-[trans-4-({2-[6-(2-Pyridin-4-ylethyl)quinazolin-4-yl]-2,7-diazaspiro[3.5]non-7-yl}methyl)cyclohexyl]ethanesulfonamide (Compound 48)

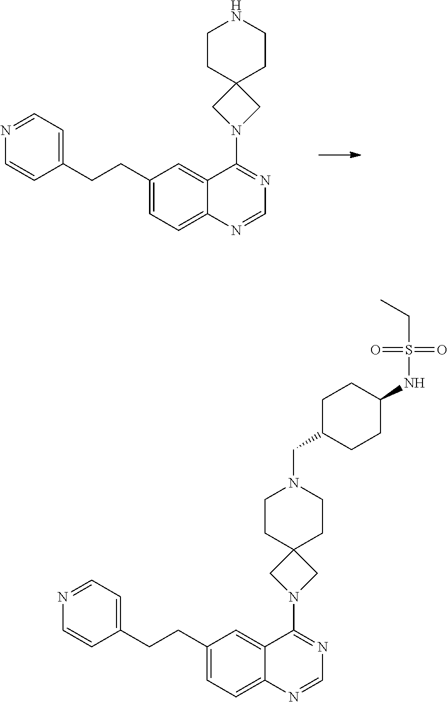

To a solution of 4-(2,7-diazaspiro[3.5]non-2-yl)-6-(2-pyridin-4-ylethyl)quinazoline (P152, 55 mg, 0.154 mmol) in DCM (5 ml)N-(trans-4-formylcyclohexyl)ethanesulfonamide (P133, 44 mg, 0.20 mmol), and triacethoxyborohydride (114 mg, 0.54 mmol) were added. The mixture was stirred at rt for 24 h (LCMS control), then a water solution of sodium bicarbonate was added with stirring. The mixture was stirred for 30 min, water phase extracted with DCM (2×30 ml), DCM was concentrated, and residue was purified by column chromatography on silica with ethylacetate-methanol (10:2) to yield the Compound 48 (25 mg, 29%) as a white solid. ¹H NMR (400 MHz, DMSO-d₆), δ: 8.44 (d, J=6.1 Hz, 2H), 8.38 (s, 1H), 7.63 (m, 3H), 7.25 (d, J=6.1 Hz, 2H), 7.00 (d, J=7.7 Hz, 1H), 4.06 (bs, 4H), 3.09 (t, J=7.6 Hz, 2H), 2.97 (m, 5H), 2.29 (bs, 4H), 2.06 (d, J=7.1 Hz, 2H), 1.87 (m, 2H), 1.74 (m, 6H), 1.39 (m, 1H), 1.18 (m, 5H), 0.88 (q, J=12.4 Hz, 2H). LCMS(ESI) [MH]⁺: 563.

### Example 39. N-[trans-4-({2-[6-(phenylethynyl)quinazolin-4-yl]-2,7-diazaspiro[3.5]non-7-yl}methyl)cyclohexyl]ethanesulfonamide (Compound 53)

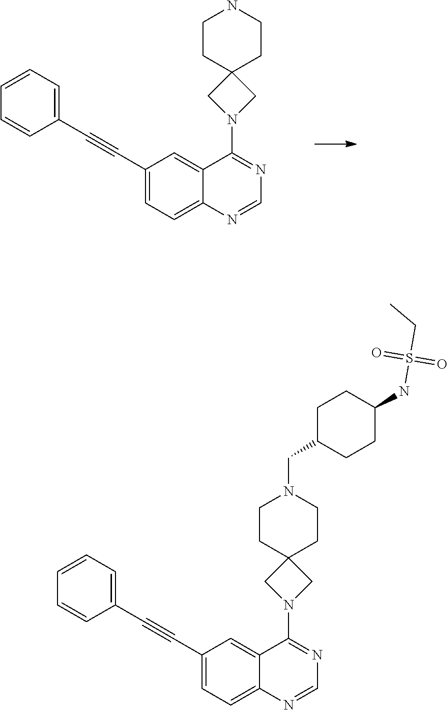

To a solution of P154 (44 mg, 0.097 mmol) in DCM (5 ml)N-(trans-4-formylcyclohexyl)ethanesulfonamide (P13333 mg, 0.15 mmol), triacethoxyborohydride (114 mg, 0.54 mmol) were added. The mixture stirred at rt for 24 h (LCMS control), then water solution of sodium bicarbonate was added with stirring. The mixture was stirred for 30 min, water phase was extracted with DCM (2×30 ml), DCM was evaporated, and residue was purified by column chromatography on silica with ethylacetate-methanol (10:2) to yield the Compound 53 (25 mg, 34%) as a white solid. ¹H NMR (400 MHZ, DMSO-d₆), δ: 8.45 (s, 1H), 8.08 (s, 1H), 7.87 (d, J=8.6 Hz, 1H), 7.70 (d, J=8.6 Hz, 1H), 7.61 (m, 2H), 7.45 (m, 3H), 7.00 (d, J=7.7 Hz, 1H), 4.24 (bs, 4H), 2.96 (m, 3H), 2.31 (bs, 4H), 2.06 (bs, 2H), 1.80 (m, 8H), 1.39 (s, 1H), 1.19 (m, 5H), 0.88 (q, J=12.1, 10.6 Hz, 2H). LCMS(EST) [MH]⁺: 558.

### Example 40. N-{3-[(2-quinazolin-4-yl-2,7-diazaspiro[3.5]non-7-yl)methyl]phenyl}ethanesulfonamide (Compound 23)

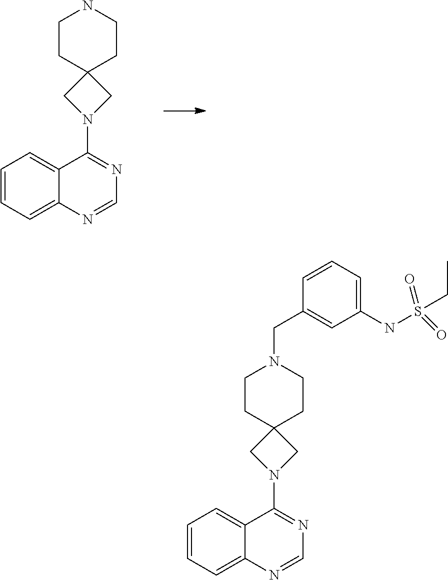 [SMILES: c1ccc2c(N3CC4(CCNCC4)C3)ncnc2c1 ; CCS(=O)(=O)Nc1cccc(CN2CCC3(CC2)CN(c2ncnc4ccccc24)C3)c1]

To a solution of 4-(2,7-diazaspiro[3.5]non-2-yl)quinazoline (P156, 51 mg, 0.2 mmol) in DCM (8 ml)N-(3-formylphenyl)ethanesulfonamide (P141, 43 mg, 0.4 mmol), and triacethoxyborohydride (228 mg, 1.08 mmol) were added. The mixture was stirred at rt for 24 h (LCMS control), then water solution of sodium bicarbonate was added with stirring. The mixture was stirred for 30 min, water phase was extracted with DCM (2×30 ml), DCM was concentrated, and residue was purified by column chromatography on silica with ethylacetate-methanol (10:2) to yield the Compound 23 (48 mg, 53%) as a white solid. ¹H NMR (400 MHZ, DMSO-d₆), δ: 8.43 (s, 1H), 7.99 (d, J=8.3 Hz, 1H), 7.73 (m, 4H), 7.50 (m, 4H), 4.21 (s, 4H), 3.32 (s, 2H), 2.77 (q, J=7.2 Hz, 2H), 2.38 (bs, 4H), 1.83 (bs, 4H), 0.96 (t, J=7.2 Hz, 3H). LCMS(ESI) [MH]⁺: 452.

### Example 41. N-{4-[(2-pyrido[2,3-d]pyrimidin-4-yl-2,7-diazaspiro[3.5]non-7-yl)methyl]phenyl}methanesulfonamide (Compound 25)

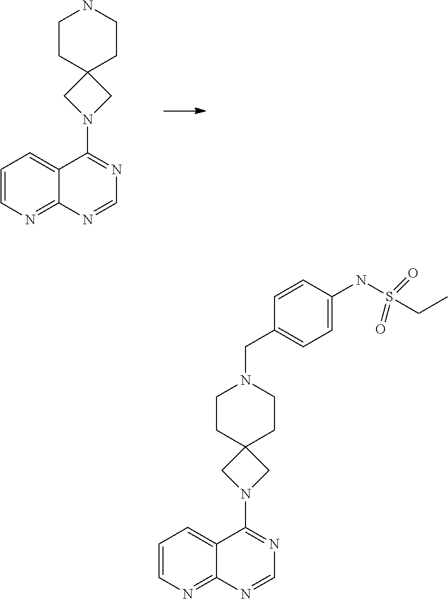 [SMILES: c1cnc2ncnc(N3CC4(CCNCC4)C3)c2c1 ; CCS(=O)(=O)Nc1ccc(CN2CCC3(CC2)CN(c2ncnc4ncccc24)C3)cc1]

To a solution of 4-(2,7-diazaspiro[3.5]non-2-yl)pyrido[2,3-d]pyrimidine (P158, 51 mg, 0.2 mmol) in DCM (8 ml)N-(4-formylphenyl)ethanesulfonamide (P137, 43 mg, 0.4 mmol), and triacethoxyborohydride (228 mg, 1.08 mmol) were added. The mixture was stirred at rt for 24 h (LCMS control), then water solution of sodium bicarbonate was added with stirring. The mixture was stirred for 30 min, water phase was extracted with DCM (2×30 ml), DCM was concentrated, and residue was purified by column chromatography on silica with ethylacetate-methanol (10:2) to yield the Compound 25 (48 mg, 53%) as a white solid. ¹H NMR (400 MHZ, DMSO-d₆), δ: 9.71 (s, 1H), 8.95 (dd, J=4.4, 1.7 Hz, 1H), 8.56 (s, 1H), 8.40 (dd, J=8.4, 1.7 Hz, 1H), 7.45 (dd, J=8.4, 4.4 Hz, 1H), 7.24 (d, J=8.2 Hz, 2H), 7.16 (d, J=8.2 Hz, 2H), 4.08 (bs, 4H), 3.39 (s, 2H), 3.06 (q, J=7.3 Hz, 2H), 2.33 (bs, 4H), 1.80 (bs, 4H), 1.19 (t, J=7.2 Hz, 3H). LCMS(ESI) [MH]⁺: 453.

### Example 42. N-[4-({2-[7-(2,2,2-trifluoroethyl)quinazolin-4-yl]-2,7-diazaspiro[3.5]non-7-yl}methyl)phenyl]ethanesulfonamide (Compound 8)

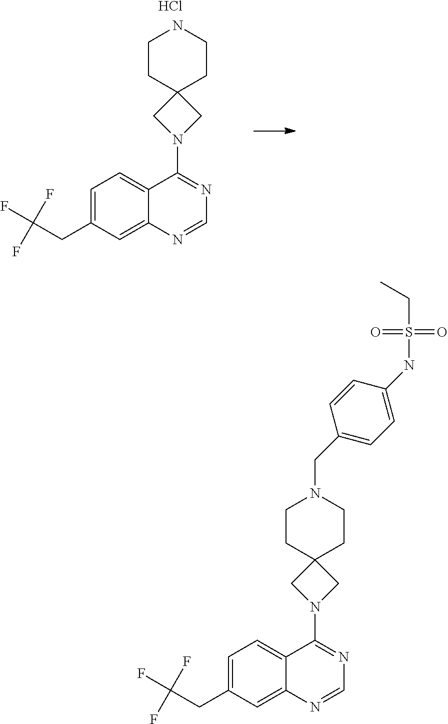 [SMILES: Cl ; FC(F)(F)Cc1ccc2c(N3CC4(CCNCC4)C3)ncnc2c1 ; CCS(=O)(=O)Nc1ccc(CN2CCC3(CC2)CN(c2ncnc4cc(CC(F)(F)F)ccc24)C3)cc1]

To a mixture of 4-(2,7-diazaspiro[3.5]non-2-yl)-7-(2,2,2-trifluoroethyl)quinazoline hydrochloride (P167, 60 mg, 0.16 mmol) in DCM (8 ml)N-(4-formylphenyl)ethanesulfonamide (P137, 43 mg, 0.4 mmol), triacethoxyborohydride (228 mg, 1.08 mmol) were added. The mixture was stirred at rt for 24 h (LCMS control), then with stirring water solution of sodium bicarbonate was added. The mixture was stirred for 30 min, water phase was extracted with DCM (2×30 ml), DCM was evaporated, and residue purified by column chromatography on silica with ethylacetate-methanol (10:2) to yield the Compound 8 (48 mg, 56%) as a white solid. ¹H NMR (400 MHZ, DMSO-d₆), δ: 9.72 (s, 1H), 8.44 (s, 1H), 7.99 (d, J=8.6 Hz, 1H), 7.70 (s, 1H), 7.43 (d, J=8.6 Hz, 1H), 7.25 (d, J=8.0 Hz, 2H), 7.17 (d, J=8.0 Hz, 2H), 4.20 (bs, 4H), 3.86 (m, 2H), 3.39 (bs, 2H), 3.06 (q, J=7.3 Hz, 2H), 2.33 (bs, 4H), 1.80 (bs, 4H), 1.19 (t, J=7.3 Hz, 3H). LCMS(ESI) [MH]⁺: 534.

### Example 43. N-[4-({9-[7-(2,2,2-trifluoroethyl)quinazolin-4-yl]-3,9-diazaspiro[5.5]undec-3-yl}methyl)phenyl]ethanesulfonamide (Compound 2)

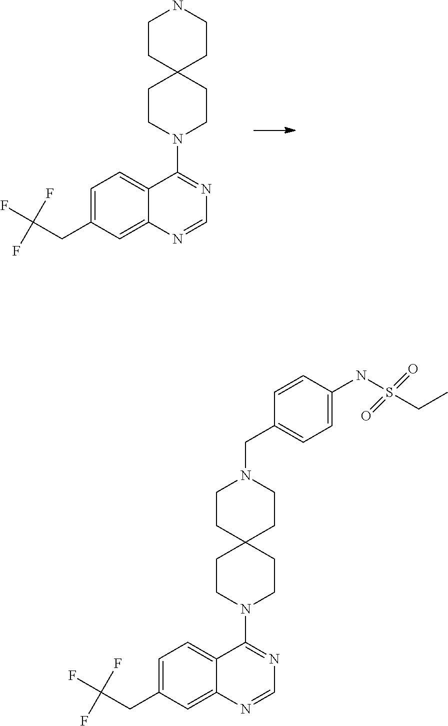 [SMILES: FC(F)(F)Cc1ccc2c(N3CCC4(CCNCC4)CC3)ncnc2c1 ; CCS(=O)(=O)Nc1ccc(CN2CCC3(CC2)CCN(c2ncnc4cc(CC(F)(F)F)ccc24)CC3)cc1]

To a mixture of 3-[7-(2,2,2-trifluoroethyl)quinazolin-4-yl]-3,9-diazaspiro[5.5]undecane (P169, 60 mg, 0.16 mmol) in DCM (8 ml)N-(4-formylphenyl)ethanesulfonamide (P137, 43 mg, 0.4 mmol), and triacethoxyborohydride (228 mg, 1.08 mmol) were added. The mixture was stirred at rt for 24 h (LCMS control), then with stirring a water solution of sodium bicarbonate was added. The mixture was stirred for additional 30 min, water phase was extracted with DCM (2×30 ml), DCM was evaporated, and residue was purified by column chromatography on silica with ethylacetate-methanol (10:2) to yield the Compound 2 (48 mg, 53%) as a white solid. ¹H NMR (400 MHZ, DMSO-d₆), δ: 9.71 (s, 1H), 8.56 (s, 1H), 7.97 (d, J=8.6 Hz, 1H), 7.78 (s, 1H), 7.47 (d, J=8.6 Hz, 1H), 7.23 (d, J=8.2 Hz, 2H), 7.15 (d, J=8.2 Hz, 2H), 3.88 (m, 2H), 3.72 (t, J=5.7 Hz, 4H), 3.41 (bs, 2H), 3.05 (q, J=7.3 Hz, 2H), 2.35 (bs, 4H), 1.60 (bs, 4H), 1.53 (bs, 4H), 1.19 (t, J=7.3 Hz, 3H). LCMS(ESI) [MH]⁺: 562.

### Example 44. N-{4-[(4-{[7-(2,2,2-trifluoroethyl)quinazolin-4-yl]amino}piperidin-1-yl)methyl]phenyl}ethanesulfonamide (Compound 33)

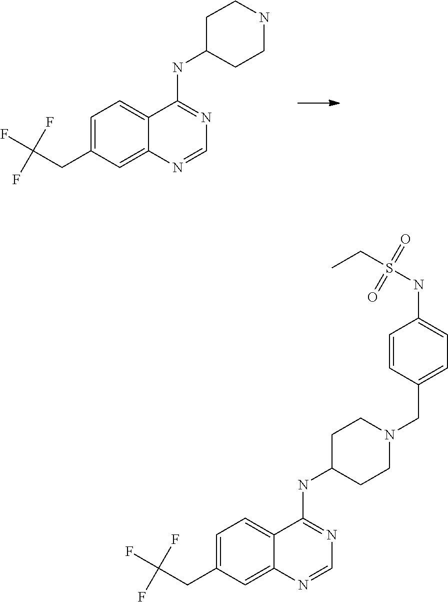 [SMILES: FC(F)(F)Cc1ccc2c(NC3CCNCC3)ncnc2c1 ; CCS(=O)(=O)Nc1ccc(CN2CCC(Nc3ncnc4cc(CC(F)(F)F)ccc34)CC2)cc1]

To mixture of N-piperidin-4-yl-7-(2,2,2-trifluoroethyl)quinazolin-4-amine (P171, 60 mg, 0.194 mmol) in DCM (8 ml)N-(4-formylphenyl)ethanesulfonamide (P137, 43 mg, 0.4 mmol), triacethoxyborohydride (228 mg, 1.08 mmol) were added. The mixture was stirred at rt for 24 b (LCMS control), then with stirring water solution of sodium bicarbonate was added. The mixture was stirred for 30 min, water phase was extracted with DCM (2×30 ml), DCM was concentrated, and residue was purified by column chromatography on silica with ethylacetate-methanol (10:2) to yield the Compound 33 (38 mg, 39%) as a white solid. ¹H NMR (400 MHZ, DMSO-d₆), δ: 9.72 (s, 1H), 8.45 (s, 1H), 8.31 (d, J=8.5 Hz, 1H), 7.95 (d, J=7.6 Hz, 1H), 7.68 (s, 1H), 7.47 (d, J=8.5 Hz, 1H), 7.26 (d, J=8.1 Hz, 2H), 7.17 (d, J=8.1 Hz, 2H), 4.18 (m, 1H), 3.84 (m, 2H), 3.43 (s, 2H), 3.06 (q, J=7.3 Hz, 2H), 2.86 (m, 2H), 2.05 (t, J=11.8 Hz, 2H), 1.90 (d, J=12.2 Hz, 2H), 1.66 (q, J=11.7 Hz, 2H), 1.19 (t, J=7.3 Hz, 3H). LCMS(ESI) [MH]⁺: 508.

### Example 45. N-{trans-4-[(2-{6-[2-(3-methyl-1,2,4-oxadiazol-5-yl)ethyl]quinazolin-4-yl}-2,7-diazaspiro[3.5]non-7-yl)methyl]cyclohexyl}ethane-sulfonamide (Compound 63)

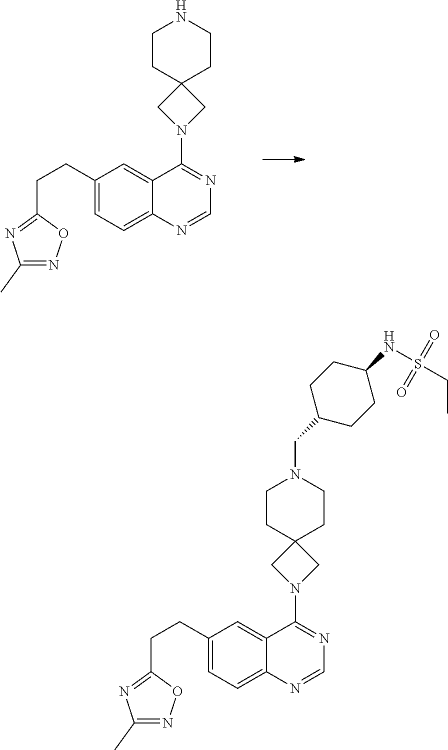

To solution of (2,7-diazaspiro[3.5]non-2-yl)-6-[2-(3-methyl-1,2,4-oxadiazol-5-yl)ethyl]quinazoline (P175, 64 mg, 0.178 mmol) in DCM (5 ml)N-(trans-4-formylcyclohexyl)ethanesulfonamide (P133, 77 mg, 0.35 mmol), triacethoxyborohydride (114 mg, 0.54 mmol) were added. The mixture was stirred at rt for 24 h (LCMS control), then with stirring water solution of sodium bicarbonate was added. The mixture was stirred 30 min, water phase extracted with DCM (2×30 ml), DCM was concentrated, and residue was purified by column chromatography on silica with ethylacetate-methanol (10:2) to yield the Compound 63 (28 mg, 28%) as a white solid. ¹H NMR (400 MHz, DMSO-d₆), δ: 8.39 (s, 1H), 7.77 (d, J=1.8 Hz, 1H), 7.68 (dd, J=8.6, 1.8 Hz, 1H), 7.62 (d, J=8.6 Hz, 1H), 7.00 (d, J=7.7 Hz, 1H), 4.16 (bs, 4H), 3.28 (m, 2H), 3.21 (m, 2H), 2.96 (m, 3H), 2.30 (s, 3H), 2.29 (bs, 4H), 2.05 (d, J=7.1 Hz, 2H), 1.80 (m, 8H), 1.39 (s, 1H), 1.18 (m, 5H), 0.88 (q, J=12.5 Hz, 2H). LCMS(ESI) [MH]⁺: 558.

### Biological Assays

### Example A. Primary Assay Used to Determine Potency of MENI activity Inhibition

Compound activity was determined using recombinant MEN1 protein (Creativebiomart, Cat #MEN1-35H) and a custom fluorescein-labeled MLL4-43 peptide (Eton Bioscience Inc.). Interaction between MEN1 and MLL4-43 in the presence of compounds was determined by fluorescence polarization assay using a Microplate Reader ClarioStar Plus. The reaction was carried out in assay buffer (50 mM TRIS-HCl PH 7.4-7.6, 50 mM NaCl, 1 mM DTT, 0.1 mg/ml BSA). The compounds were dispensed on a 384 well Diamond Well Plate (Axigen, Cat #P-384-120 SQ-C—S) using the Biomek FX liquid handling system at 100× solutions of compounds in DMSO. 2× MEN1 mix (final concentration of MEN1 10 nM) was prepared in Assay buffer and 10 μl of mixture per well was added into 384w white Reaction plate with NBS(Corning, Cat #4513). 10 μl of Assay buffer w/o MENI was used for negative control. Plates were centrifuged for 1 min at 100 g. Next step the Compounds were added to Reaction plate using Biomek station via following steps: 3 μl of 100× compounds (in DMSO) were mixed thoroughly with 27 μl Assay Buffer, then 2 μl of this mixture was added to Reaction plate with 10 μl of MENI mix. Plates were centrifuged for 1 min at 100 g and incubated for 20 min at room temperature. Next 8 μL of MLL4-43 peptide per well was added to final concentration of MLL 0.5 nM. Plates were incubated for 1 hour at room temperature. Then fluorescence polarization was measured using Microplate Reader. The results of this assay are shown in the Table A. The values of EC₅₀ shown as a letters A-E, where: A≤0.075 μM; 0.075 μM<B≤0.5 μM; 0.5 μM<C≤1 μM; 1<D≤5 μM; E>5.

TABLE A
MEN1 Activity Inhibition Assays

| Compound | EC₅₀, |
|---|---|
| Number | uM* |
| 1 | D |
| 2 | E |
| 3 | C |
| 4 | D |
| 5 | A |
| 7 | A |
| 8 | E |
| 21 | E |
| 23 | E |
| 24 | E |
| 31 | E |
| 33 | E |
| 36 | D |
| 43 | D |
| 44 | C |
| 45 | D |
| 48 | C |
| 49 | D |
| 51 | C |
| 52 | C |
| 53 | E |
| 56 | C |
| P148 | E |
| 59 | B |
| 61 | B |
| 63 | D |
| 65 | B |
| 69 | E |
| 70 | E |
| 71 | E |
| 74 | D |
| 75 | B |
| 77 | D |
| 79 | B |
| 83 | A |
| 89 | A |
| 90 | C |
| 91 | D |
| 92 | E |
| 94 | B |
| 100 | D |
| 102 | D |
| 107 | E |
| 110 | B |
| 111 | D |

*EC₅₀: Half maximal effective concentration: the concentration of a compound which induces a response halfway between the baseline and maximum after a specific exposure time; EC₅₀: A ≤ 0.075 μM; 0.075 μM < B ≤ 0.5 μM; 0.5 μM < C ≤ 1 μM; 1 < D ≤ 5 μM; E > 5

### Example B. Cellular Growth Inhibition Assay

HEK293 (Institute of Cytology Russian Academy of Science), MV4-11 (ATCC, CRL-9591), MOLM-13 (AcceGen, ABC-TC517S) were seeded at a density of 500 cells per well (HEK293) and 2000 cells per well (MV4-11, MOLM-13) in a 384-well clear bottom plate (Greiner Cat #781090) in 45 μl total volume of DMEM (PanEco, Cat #C₄₂₀, Russia) or RPMI (PanEco, Cat #C₃₃₀, Russia) with 10% FBS(HyClone Cat #SV30160.03). HEK293 were allowed to adhere overnight at 37° C., 5% CO₂. 500× compounds solutions in DMSO (Sigma Cat #D2650) were prepared into Cmpnds plate (Diamond Well Plate, Axigen, Cat #P-384-120 SQ-C—S) and DMSO only control was included. 1 μl of 500× compounds (Cmpnds plate) was added to 49 μl of culture medium into Dilution plate (Diamond Well Plate, Axigen, Cat #P-384-120 SQ-C—S), mixed and then 5 μl of 10× compounds solutions were transferred to cells followed by centrifugation at 100 g for 1 min. Final DMSO concentration was 0.2%. After 3 days of incubation, 10 μl of 1× compounds were added to cells. After 7 days of incubation, 12 μl of CellTiter-Glo (Promega, CAT #G7572) were added to the cells, plate was centrifuged at 100 g for 1 min and luminescence signal was measured using Microplate Reader (CLARIOStar). The results of these assays are shown in the Tables B1, B2, and B3.

TABLE B1
MV4-11 Cellular Growth Inhibition Assay

| Compound | MV4-11, |
|---|---|
| Number | CC₅₀,* uM |
| 1 | C |
| 2 | E |
| 3 | D |
| 5 | A |
| 7 | B |
| 8 | D |
| 36 | D |
| 43 | D |
| 44 | B |
| 45 | D |
| 48 | E |
| 49 | D |
| 51 | C |
| 52 | B |
| 53 | C |
| 56 | D |
| 59 | A |
| 61 | B |
| 65 | B |
| 69 | E |
| 70 | C |
| 71 | E |
| 74 | E |
| 75 | C |
| 79 | E |
| 83 | B |
| 89 | A |
| 90 | C |
| 91 | C |
| 94 | C |

*CC₅₀: Cytotoxic Concentration; the extract concentration that reduced the cell viability by 50% when compared to untreated controls; MV4-11 CC₅₀: A ≤ 1 μM; 1 μM < B ≤ 2.5 μM; 2.5 μM < C ≤ 5 μM; 5 < D ≤ 10 μM; E > 10

TABLE B2
MOLM-13 Cellular Growth Inhibition Assay

| Compound | MOLM-13 |
|---|---|
| Number | CC₅₀,* μM |
| 1 | C |
| 3 | D |
| 5 | B |
| 7 | B |
| 8 | D |
| 36 | C |
| 43 | C |
| 44 | C |
| 45 | C |
| 48 | E |
| 49 | D |
| 51 | C |
| 52 | B |
| 53 | C |
| 56 | C |
| 59 | A |
| 61 | B |
| 65 | B |
| 69 | C |
| 70 | C |
| 71 | E |
| 74 | D |
| 75 | B |
| 79 | B |
| 83 | C |
| 89 | B |
| 90 | C |
| 91 | D |
| 94 | C |

*CC₅₀: Cytotoxic Concentration: the extract concentration that reduced the cell viability by 50% when compared to untreated controls; MOLM-13 CC₅₀: A ≤ 1.0 μM; 1.0 μM < B ≤ 5 μM; 5 μM < C ≤ 10 μM; 10 < D ≤ 20 μM; E > 20

TABLE B3
HEK293 Cellular Growth Inhibition Assay

| Compound | HEK293, |
|---|---|
| Number | CC₅₀,* μM |
| 1 | B |
| 2 | C |
| 3 | B |
| 5 | B |
| 7 | B |
| 8 | C |
| 36 | C |
| 43 | C |
| 44 | B |
| 45 | C |
| 48 | D |
| 49 | D |
| 51 | B |
| 52 | B |
| 53 | A |
| 56 | C |
| 59 | A |
| 61 | B |
| 65 | E |
| 69 | D |
| 70 | C |
| 71 | E |
| 74 | D |
| 75 | C |
| 79 | A |
| 83 | B |
| 89 | A |
| 90 | B |
| 91 | B |
| 94 | B |

*CC₅₀: Cytotoxic Concentration: the extract concentration that reduced the cell viability by 50% when compared to untreated controls; HEK293 CC₅₀: A ≤ 5 μM; 5 μM < B ≤ 15 μM; 15 μM < C ≤ 30 μM; 30 < D ≤ 50 μM; E > 50

### Equivalents

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A compound of Formula (I): 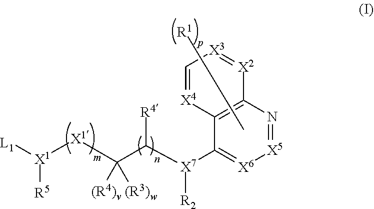
or a pharmaceutically acceptable salt, stereoisomer, solvate, or tautomer thereof, wherein
X¹ is N;
each X¹ is independently selected from CH₂ and N;
each from X², X³, X⁴, X⁵, and X⁶ is independently selected from CH or N;
and at least one from X², X³, X⁴, X⁵, and X⁶ is N;
X⁷ is N;
each R¹ is independently selected from halogen, C₁-C₆ alkyl, C₁-C₆ alkoxy, C₃-C₁₀ cycloalkyl, C₂-C₆ alkenyl, C₂-C₆ alkynyl, heterocycle, aryl, heteroaryl, C₁-C₆ alkyl-aryl, C₁-C₆ alkyl-heteroaryl, C₂-C₆ alkenyl-aryl, C₂-C₆ alkenyl-heteroaryl, C₂-C₆ alkynyl-aryl, C₂-C₆ alkynyl-heteroaryl, and NR¹²R¹³ wherein the alkyl, alkoxy, alkenyl, alkynyl, heterocycle, cycloalkyl, aryl, or heteroaryl is optionally substituted with one or more substituents independently selected from halogen, OH, NH₂, C₁-C₆ alkyl, C₁-C₆ alkoxy, NR¹²R¹³, C₃-C₁₀ cycloalkyl, C₂-C₆ alkenyl, C₂-C₆ alkynyl, heterocycle, aryl, heteroaryl;
R⁴′ is selected from H and C₁-C₆ alkyl;
R² and R³ are each independently selected from H or C₁-C₆ alkyl;
or R² and R³, together with the atoms to which they are bound and any intervening atoms, form a 3-8 membered heterocycle, wherein the heterocycle is optionally substituted with one or more substituents independently selected from halogen, OH, NH₂, C₁-C₆ alkyl, C₁-C₆ alkoxy, and NR¹²R¹³;
R⁴ and R⁵ are each independently selected from H and C₁-C₆ alkyl;
or R⁴ and R⁵, together with the atoms to which they are bound and any intervening atoms, form a 3-8 membered heterocycle or heteroaryl, wherein the heterocycle and heteroaryl are optionally substituted with one or more substituents independently selected from halogen, OH, NH₂, C₁-C₆ alkyl, C₁-C₆ alkoxy, and NR¹²R¹³; or
R⁴′ and R⁵, together with the atoms to which they are bound and any intervening atoms, form a 3-8 membered heterocycle or heteroaryl, wherein the heterocycle and heteroaryl are optionally substituted with one or more substituents independently selected from halogen, OH, NH₂, C₁-C₆ alkyl, C₁-C₆ alkoxy, and NR¹²R¹³;
L¹ is 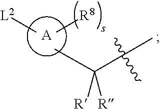
wherein
R′ and R″ are both H;
Rings A is selected from aryl and C₃-C₁₄ cycloalkyl;
L² is independently selected from the group consisting halogen, C₁-C₆ alkyl, NR¹²R¹³, —N(R⁶)S(O)₂R⁷, —N(R⁶)CH₂R⁷, —N(R⁶)C(O)R⁷, —C(O)NR⁶R⁷, —S(O)₂NR⁶R⁷, wherein the alkyl is optionally substituted with one or more substituents independently selected from halogen, oxo, OH, C₁-C₆ alkyl, C₁-C₆ alkoxy, NR¹²R¹³, cycloalkyl, and heterocycle;
R⁶ is selected from H, C₁-C₆ alkyl;
R⁷ is selected from NR¹²R¹³, C₁-C₆ alkyl, C₂-C₆ alkenyl, C₂-C₆ alkynyl, C₃-C₁₀ cycloalkyl, aryl, heteroaryl, or heterocycle, wherein the alkyl, alkenyl, alkynyl, cycloalkyl, aryl, heteroaryl, or heterocycle is optionally substituted with one or more substituents independently selected from halogen, OH, NH₂, C₁-C₆ alkyl, C₁-C₆ alkoxy, —S(O)₂R⁷, N(R⁶)S(O)₂R⁷, and NR¹²R¹³,
each R⁸ is independently selected from halogen, OH, oxo, CN, C₁-C₆ alkyl, C₂-C₆ alkenyl, C₁-C₆ alkoxy, C₁-C₆ alkyl-NR¹²R¹³, —NR¹²R¹³, wherein the alkyl, or alkoxy is optionally substituted with one or more substituents independently selected from halogen, oxo, OH, NH₂, C₁-C₆ alkyl, C₁-C₆ alkoxy, NR¹²R¹³, cycloalkyl, and heterocycle; or
L² and R⁸ together with the atoms to which they are attached and any intervening atoms, form a 3-14 membered cycloalkyl, 5-14 membered heterocycle, or 5-6 membered heteroaryl, wherein the cycloalkyl, heterocycle or heteroaryl is optionally substituted with one or more substituents independently selected from halogen, OH, NH₂, C₁-C₆ alkyl, C₁-C₆ alkoxy, C₁-C₆ alkyl-C₁-C₆ alkoxy, C₁-C₆ alkyl-NHC₁-C₆ alkyl, and NR¹²R¹³, or each R¹² and R¹³ is independently H, C₁-C₆ alkyl, C₁-C₆ alkoxy, C₃-C₁₀ cycloalkyl, C₂-C₆ alkenyl, —S(O)₂R⁷, C₁-C₆ acyl, heterocycle, aryl, or heteroaryl;
or R¹² and R¹³ together with the atoms to which they are attached and any intervening atoms, form a 5-14 membered heterocycle optionally substituted with one or more substitutents independently selected from —S(O)₂R⁷;
m is an integer selected from 0, 1, 2, and 3;
n is an integer selected from 0, 1, 2, and 3;
p is an integer selected from 0, 1 and 2;
s is an integer selected from 0, 1 and 2;
v is 1;
w is an integer selected from 0 and 1, w is 0 when R⁴ is a part of aromatic ring; wherein,
aryl is cyclic, aromatic hydrocarbon groups that have 1 to 3 aromatic rings;
heterocyclyl is saturated or partially unsaturated 3-10 membered monocyclic, 7-12 membered bicyclic (fused, bridged, or spiro rings), or 11-14 membered tricyclic ring system (fused, bridged, or spiro rings) having one or more heteroatoms selected from O, N, S, P, Se, or B;
heteroaryl is a monovalent monocyclic or a polycyclic aromatic radical of 5 to 24 ring atoms, containing one or more ring heteroatoms selected from N, O, S, P, or B, the remaining ring atoms being C.

2. The compound of claim 1, wherein the compound is of Formula (I-A), (I-B), (I-C), (I-D), (I-E), (I-F), or (I-G): 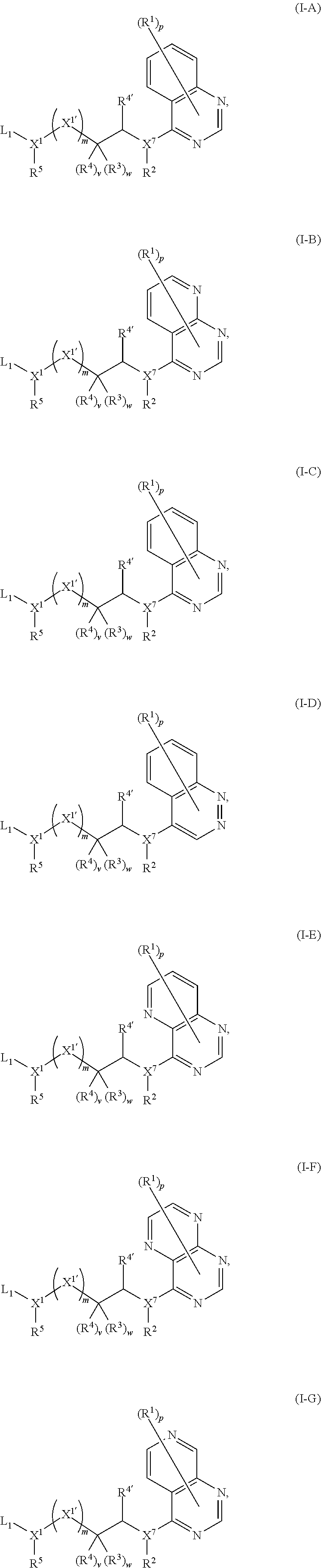
or a pharmaceutically acceptable salt, stereoisomer, solvate, or tautomer thereof.

3. The compound of claim 1, wherein the compound is of Formula (I-A-I), (I-A-II), (I-A-III), (I-A-IV), (I-A-V), or (I-A-VI): 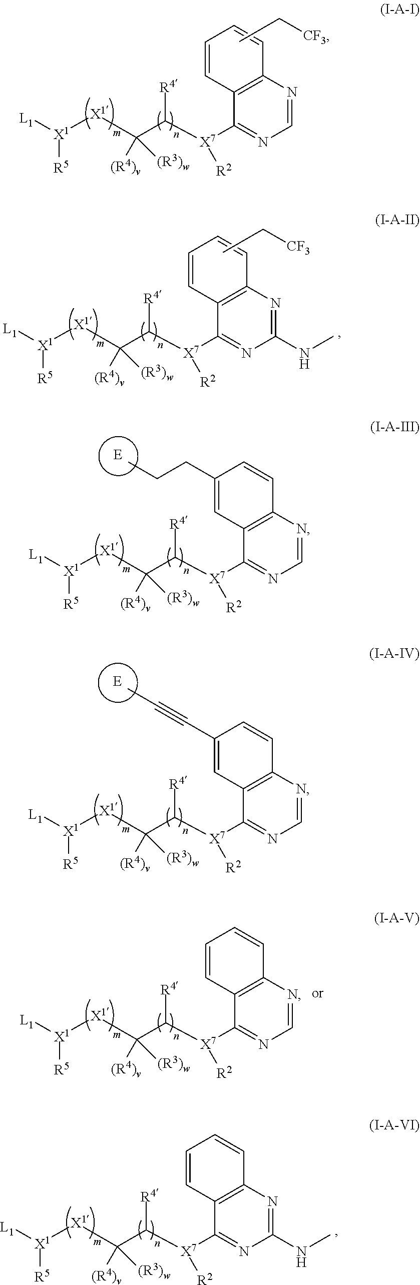
or a pharmaceutically acceptable salt, stereoisomer, solvate, or tautomer thereof, wherein Ring E is aryl or heteroaryl, optionally substituted with one or more substituents independently selected from halogen, OH, NH₂, C₁-C₆ alkyl, C₁-C₆ alkoxy, and NR¹²R¹³.

4. The compound of claim 1, wherein the compound is of Formula (I-1) 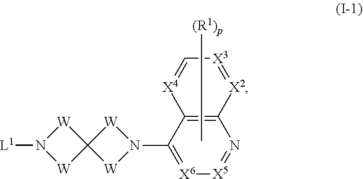
or a pharmaceutically acceptable salt, stereoisomer, solvate, or tautomer thereof, wherein W is independently, at each occurrence —CH₂—, —(CH₂)₂—, or —(CH₂)₃—.

5. The compound of claim 4, wherein the compound is of Formula (I-1-A): 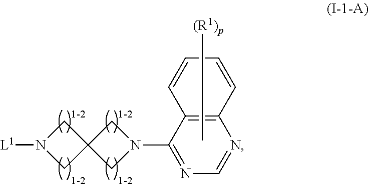
or a pharmaceutically acceptable salt, stereoisomer, solvate, or tautomer thereof.

6. The compound of claim 1, wherein the compound is of Formula (I-1-A-1), (I-1-A-2), (I-1-A-3), or (I-A-VII): 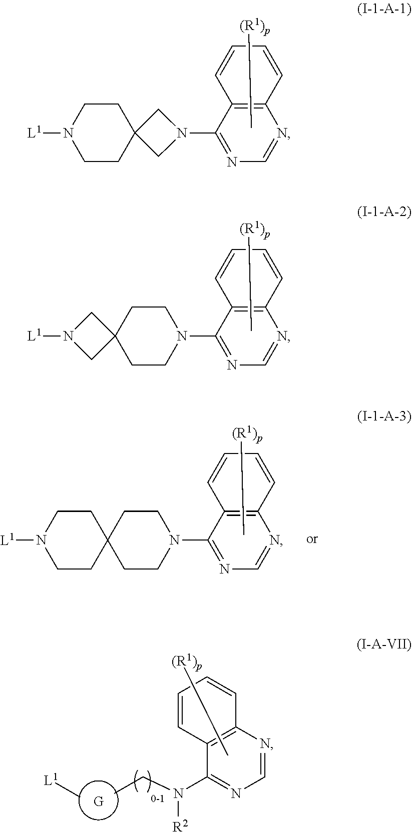
or a pharmaceutically acceptable salt, stereoisomer, solvate, or tautomer thereof, wherein Ring G is a 3-8 membered heterocycle, optionally substituted with one or more substituents independently selected from halogen, OH, NH₂, C₁-C₆ alkyl, C₁-C₆ alkoxy, NR¹²R¹³.

7. The compound of claim 1, wherein the compound is of Formula (I-2-1), (I-2-II), (I-2-IIII), or (I-2-IV): 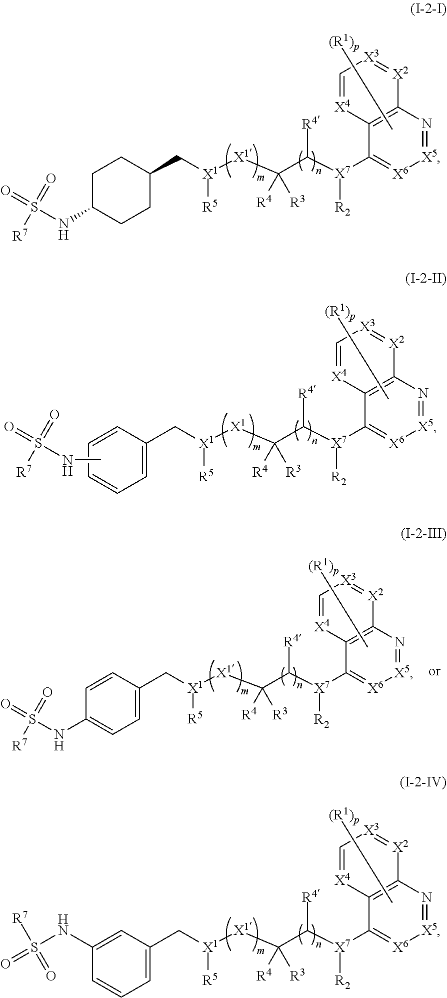
or a pharmaceutically acceptable salt, stereoisomer, solvate, or tautomer thereof.

8. The compound of claim 1, wherein the compound is of Formula (I-2-A-I), (I-2-A-II), (I-2-A-III), or (I-2-A-IV): 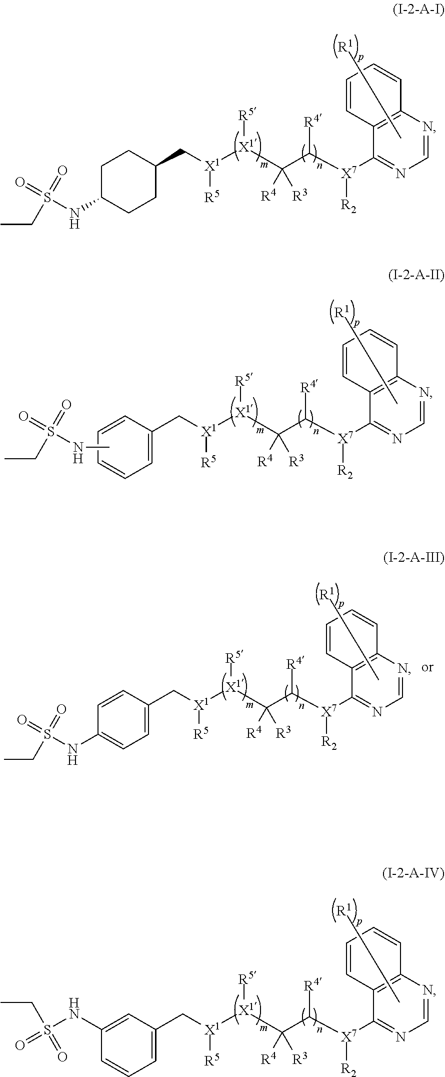
or a pharmaceutically acceptable salt, stereoisomer, solvate, or tautomer thereof.

9. The compound of claim 1, wherein A is selected from

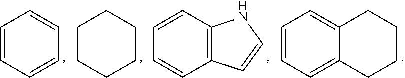

10. The compound of claim 1, wherein L² is N(R⁶)S(O)₂R⁷.

11. The compound of claim 1, wherein L² is

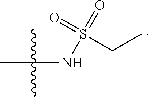 [SMILES: *NS(=O)(=O)CC ; "."]

12. The compound of claim 1, wherein L² is S(O)₂NR⁶R⁷.

13. The compound of claim 1, wherein L² is

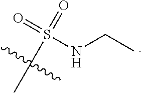 [SMILES: *S(=O)(=O)NCC ; "."]

14. A compound selected from:

| # | Structure | IUPAC Name |
|---|---|---|
| 1 | 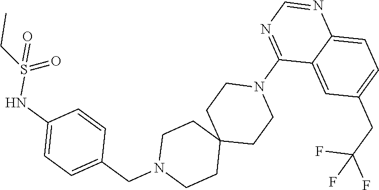 [SMILES: CCS(=O)(=O)Nc1ccc(CN2CCC3(CC2)CCN(c2ncnc4ccc(CC(F)(F)F)cc24)CC3)cc1] | N-[4-[[3-[6-(2,2,2-trifluoroethyl)quinazolin-4-yl]-3,9- diazaspiro[5.5]undecan-9- yl]methyl]phenyl]ethanesulfonamide |
| 2 | 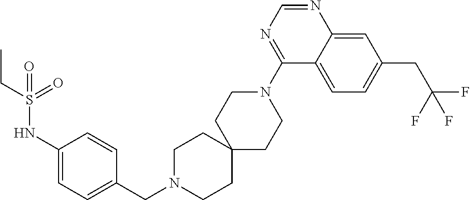 [SMILES: CCS(=O)(=O)Nc1ccc(CN2CCC3(CC2)CCN(c2ncnc4cc(CC(F)(F)F)ccc24)CC3)cc1] | N-[4-[[3-[7-(2,2,2-trifluoroethyl)quinazolin-4-yl]-3,9- diazaspiro[5.5]undecan-9- yl]methyl]phenyl]ethanesulfonamide |
| 3 | 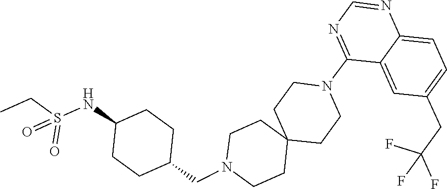 [SMILES: CCS(=O)(=O)N[C@H]1CC[C@H](CN2CCC3(CC2)CCN(c2ncnc4ccc(CC(F)(F)F)cc24)CC3)CC1] | N-((1R,4R)-4-((9-(6-(2,2,2-trifluoroethyl)quinazolin-4-yl)- 3,9-diazaspiro[5.5]undecan-3- yl)methyl)cyclohexyl)ethanesulfonamide |
| 4 | 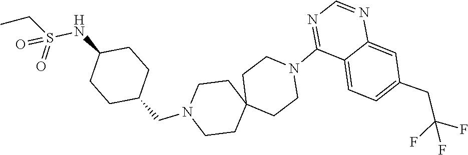 [SMILES: CCS(=O)(=O)N[C@H]1CC[C@H](CN2CCC3(CC2)CCN(c2ncnc4cc(CC(F)(F)F)ccc24)CC3)CC1] | N-((1R,4R)-4-((9-(7-(2,2,2-trifluoroethyl)quinazolin-4-yl)- 3,9-diazaspiro[5.5]undecan-3- yl)methyl)cyclohexyl)ethanesulfonamide |
| 5 | 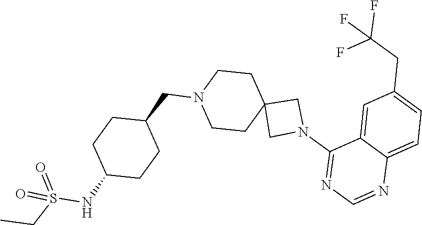 [SMILES: CCS(=O)(=O)N[C@H]1CC[C@H](CN2CCC3(CC2)CN(c2ncnc4ccc(CC(F)(F)F)cc24)C3)CC1] | N-((1R,4R)-4-((2-(6-(2,2,2-trifluoroethyl)quinazolin-4-yl)- 2,7-diazaspiro[3.5]nonan-7- yl)methyl)cyclohexyl)ethanesulfonamide |
| 6 | 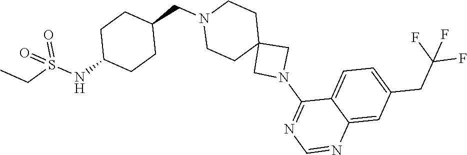 [SMILES: CCS(=O)(=O)N[C@H]1CC[C@H](CN2CCC3(CC2)CN(c2ncnc4cc(CC(F)(F)F)ccc24)C3)CC1] | N-((1R,4R)-4-((2-(7-(2,2,2-trifluoroethyl)quinazolin-4-yl)- 2,7-diazaspiro[3.5]nonan-7- yl)methyl)cyclohexyl)ethanesulfonamide |
| 7 | 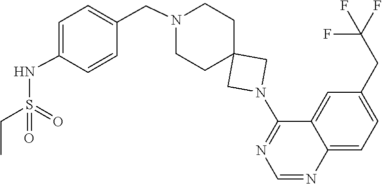 [SMILES: CCS(=O)(=O)Nc1ccc(CN2CCC3(CC2)CN(c2ncnc4ccc(CC(F)(F)F)cc24)C3)cc1] | N-(4-((2-(6-(2,2,2-trifluoroethyl)quinazolin-4-yl)-2,7- diazaspiro[3.5]nonan-7-yl)methyl)phenyl)ethanesulfonamide |
| 8 | 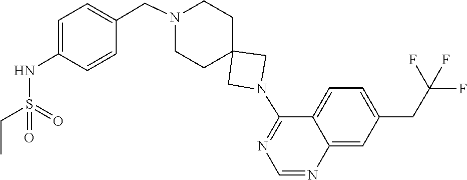 [SMILES: CCS(=O)(=O)Nc1ccc(CN2CCC3(CC2)CN(c2ncnc4cc(CC(F)(F)F)ccc24)C3)cc1] | N-(4-((2-(7-(2,2,2-trifluoroethyl)quinazolin-4-yl)-2,7- diazaspiro[3.5]nonan-7-yl)methyl)phenyl)ethanesulfonamide |
| 9 | 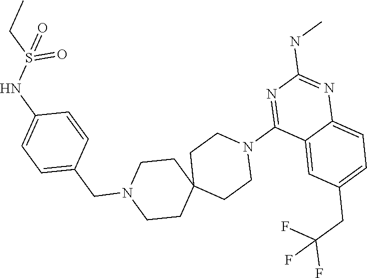 | N-(4-((9-(2-(methylamino)-6-(2,2,2- trifluoroethyl)quinazolin-4-yl)-3,9-diazaspiro[5.5]undecan- 3-yl)methyl)phenyl)ethanesulfonamide |
| 10 | 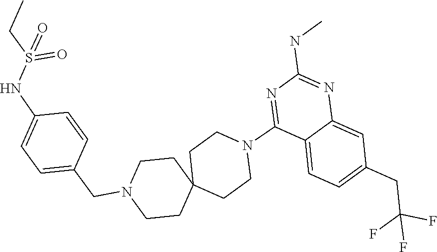 | N-(4-((9-(2-(methylamino)-7-(2,2,2- trifluoroethyl)quinazolin-4-yl)-3,9-diazaspiro[5.5]undecan- 3-yl)methyl)phenyl)ethanesulfonamide |
| 11 | 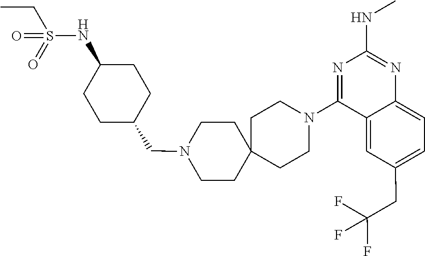 [SMILES: CCS(=O)(=O)N[C@H]1CC[C@H](CN2CCC3(CC2)CCN(c2nc(NC)nc4ccc(CC(F)(F)F)cc24)CC3)CC1] | N-((1R,4R)-4-((9-(2-(methylamino)-6-(2,2,2- trifluoroethyl)quinazolin-4-yl)-3,9-diazaspiro[5.5]undecan- 3-yl)methyl)cyclohexyl)ethanesulfonamide |
| 12 | 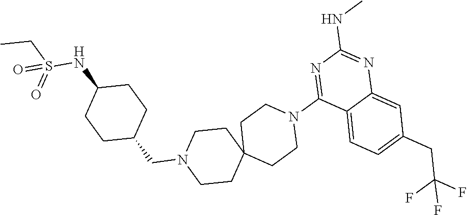 [SMILES: CCS(=O)(=O)N[C@H]1CC[C@H](CN2CCC3(CC2)CCN(c2nc(NC)nc4cc(CC(F)(F)F)ccc24)CC3)CC1] | N-((1R,4R)-4-((9-(2-(methylamino)-7-(2,2,2- trifluoroethyl)quinazolin-4-yl)-3,9-diazaspiro[5.5]undecan- 3-yl)methyl)cyclohexyl)ethanesulfonamide |
| 13 | 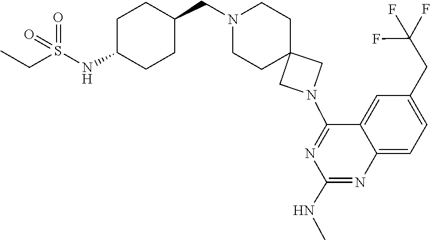 [SMILES: CCS(=O)(=O)N[C@H]1CC[C@H](CN2CCC3(CC2)CN(c2nc(NC)nc4ccc(CC(F)(F)F)cc24)C3)CC1] | N-((1R,4R)-4-((2-(2-(methylamino)-6-(2,2,2- trifluoroethyl)quinazolin-4-yl)-2,7-diazaspiro[3.5]nonan-7- yl)methyl)cyclohexyl)ethanesulfonamide |
| 14 | 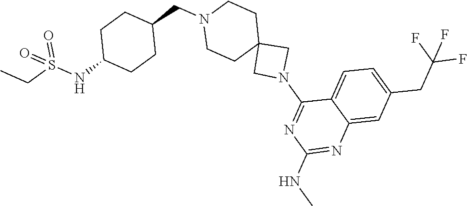 [SMILES: CCS(=O)(=O)N[C@H]1CC[C@H](CN2CCC3(CC2)CN(c2nc(NC)nc4cc(CC(F)(F)F)ccc24)C3)CC1] | N-((1R,4R)-4-((2-(2-(methylamino)-7-(2,2,2- trifluoroethyl)quinazolin-4-yl)-2,7-diazaspiro[3.5]nonan-7- yl)methyl)cyclohexyl)ethanesulfonamide |
| 15 | 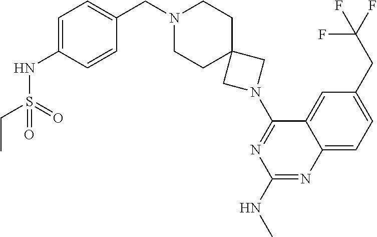 [SMILES: CCS(=O)(=O)Nc1ccc(CN2CCC3(CC2)CN(c2nc(NC)nc4ccc(CC(F)(F)F)cc24)C3)cc1] | N-(4-((2-(2-(methylamino)-6-(2,2,2- trifluoroethyl)quinazolin-4-yl)-2,7-diazaspiro[3.5]nonan-7- yl)methyl)phenyl)ethanesulfonamide |
| 16 | 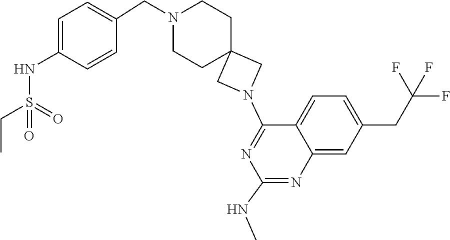 [SMILES: CCS(=O)(=O)Nc1ccc(CN2CCC3(CC2)CN(c2nc(NC)nc4cc(CC(F)(F)F)ccc24)C3)cc1] | N-(4-((2-(2-(methylamino)-7-(2,2,2- trifluoroethyl)quinazolin-4-yl)-2,7-diazaspiro[3.5]nonan-7- yl)methyl)phenyl)ethanesulfonamide |
| 21 | 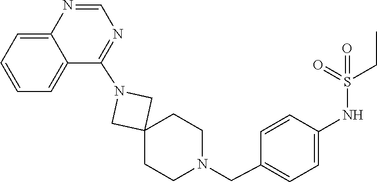 [SMILES: CCS(=O)(=O)Nc1ccc(CN2CCC3(CC2)CN(c2ncnc4ccccc24)C3)cc1] | N-(4-((2-(quinazolin-4-yl)-2,7-diazaspiro[3.5]nonan-7- yl)methyl)phenyl)ethanesulfonamide |
| 22 | 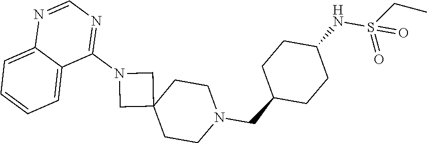 [SMILES: CCS(=O)(=O)N[C@H]1CC[C@H](CN2CCC3(CC2)CN(c2ncnc4ccccc24)C3)CC1] | N-((1R,4R)-4-((2-(quinazolin-4-yl)-2,7- diazaspiro[3.5]nonan-7- yl)methyl)cyclohexyl)ethanesulfonamide |
| 23 | 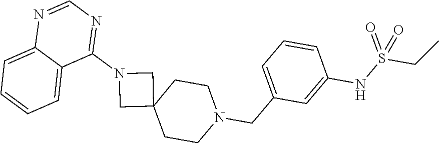 [SMILES: CCS(=O)(=O)Nc1cccc(CN2CCC3(CC2)CN(c2ncnc4ccccc24)C3)c1] | N-(3-((2-(quinazolin-4-yl)-2,7-diazaspiro[3.5]nonan-7- yl)methyl)phenyl)ethanesulfonamide |
| 24 | 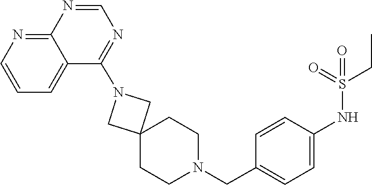 [SMILES: CCS(=O)(=O)Nc1ccc(CN2CCC3(CC2)CN(c2ncnc4ncccc24)C3)cc1] | N-[4-[(2-pyrido[2,3-d]pyrimidin-4-yl-2,7- diazaspiro[3.5]nonan-7-yl)methyl]phenyl]ethanesulfonamide |
| 25 | 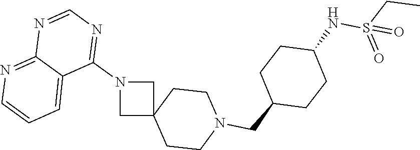 [SMILES: CCS(=O)(=O)N[C@H]1CC[C@H](CN2CCC3(CC2)CN(c2ncnc4ncccc24)C3)CC1] | N-[(1R,4R)-4-[(2-pyrido[2,3-d]pyrimidin-4-yl-2,7- diazaspiro[3.5]nonan-7- yl)methyl]cyclohexyl]ethanesulfonamide |
| 26 | 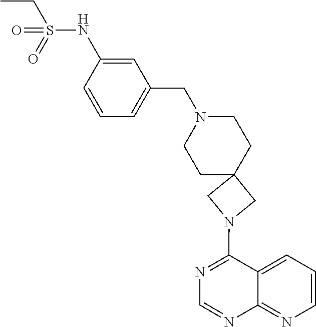 [SMILES: CCS(=O)(=O)Nc1cccc(CN2CCC3(CC2)CN(c2ncnc4ncccc24)C3)c1] | N-[3-[(2-pyrido[2,3-d]pyrimidin-4-yl-2,7- diazaspiro[3.5]nonan-7-yl)methyl]phenyl]ethanesulfonamide |
| 27 | 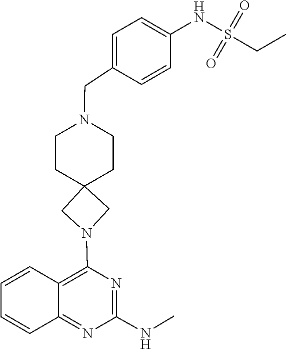 [SMILES: CCS(=O)(=O)Nc1ccc(CN2CCC3(CC2)CN(c2nc(NC)nc4ccccc24)C3)cc1] | N-(4-((2-(2-(methylamino)quinazolin-4-yl)-2,7- diazaspiro[3.5]nonan-7-yl)methyl)phenyl)ethanesulfonamide |
| 28 | 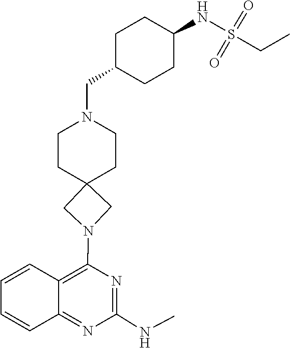 [SMILES: CCS(=O)(=O)N[C@H]1CC[C@H](CN2CCC3(CC2)CN(c2nc(NC)nc4ccccc24)C3)CC1] | N-((1R,4R)-4-((2-(2-(methylamino)quinazolin-4-yl)-2,7- diazaspiro[3.5]nonan-7- yl)methyl)cyclohexyl)ethanesulfonamide |
| 29 | 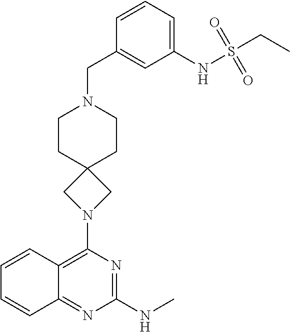 [SMILES: CCS(=O)(=O)Nc1cccc(CN2CCC3(CC2)CN(c2nc(NC)nc4ccccc24)C3)c1] | N-(3-((2-(2-(methylamino)quinazolin-4-yl)-2,7- diazaspiro[3.5]nonan-7-yl)methyl)phenyl)ethanesulfonamide |
| 30 | 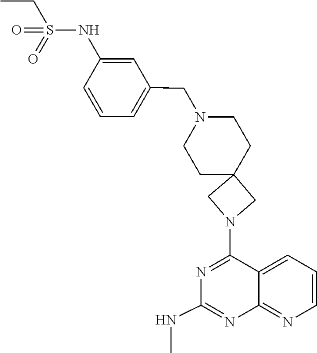 [SMILES: CCS(=O)(=O)Nc1cccc(CN2CCC3(CC2)CN(c2nc(NC)nc4ncccc24)C3)c1] | N-[3-[2-[2-(methylamino)pyrido[2,3-d]pyrimidin-4-yl]-2,7- diazaspiro[3.5]nonan-7-yl]methyl]phenyl]ethanesulfonamide |
| 31 | 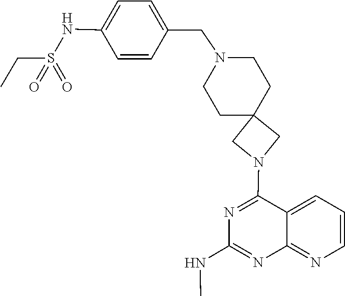 [SMILES: CCS(=O)(=O)Nc1ccc(CN2CCC3(CC2)CN(c2nc(NC)nc4ncccc24)C3)cc1] | N-[4-[2-[2-(methylamino)pyrido[2,3-d]pyrimidin-4-yl]-2,7- diazaspiro[3.5]nonan-7-yl]methyl]phenyl]ethanesulfonamide |
| 32 | 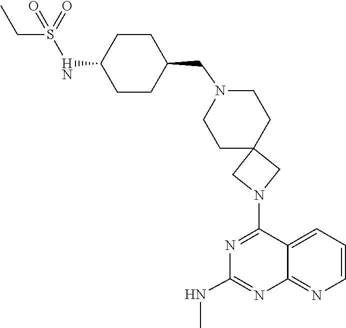 [SMILES: CCS(=O)(=O)N[C@H]1CC[C@H](CN2CCC3(CC2)CN(c2nc(NC)nc4ncccc24)C3)CC1] | N-(1R,4R)-[4-[[2-[2-(methylamino)pyrido[2,3-d]pyrimidin- 4-yl]-2,7-diazaspiro[3.5]nonan-7- yl]methyl]cyclohexyl]ethanesulfonamide |
| 33 | 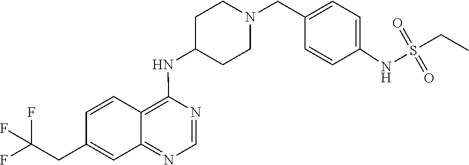 [SMILES: CCS(=O)(=O)Nc1ccc(CN2CCC(Nc3ncnc4cc(CC(F)(F)F)ccc34)CC2)cc1] | N-(4-((4-((7-(2,2,2-trifluoroethyl)quinazolin-4- yl)amino)piperidin-1-yl)methyl)phenyl)ethanesulfonamide |
| 34 | 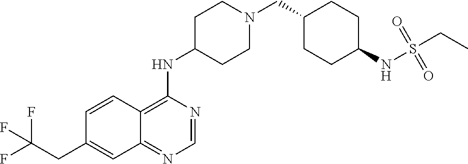 [SMILES: CCS(=O)(=O)N[C@H]1CC[C@H](CN2CCC(Nc3ncnc4cc(CC(F)(F)F)ccc34)CC2)CC1] | N-((1R,4R)-4-((4-((7-(2,2,2-trifluoroethyl)quinazolin-4- yl)amino)piperidin-1- yl)methyl)cyclohexyl)ethanesulfonamide |
| 35 | 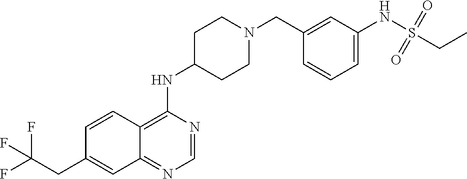 [SMILES: CCS(=O)(=O)Nc1cccc(CN2CCC(Nc3ncnc4cc(CC(F)(F)F)ccc34)CC2)c1] | N-(3-((4-((7-(2,2,2-trifluoroethyl)quinazolin-4- yl)amino)piperidin-1-yl)methyl)phenyl)ethanesulfonamide |
| 36 | 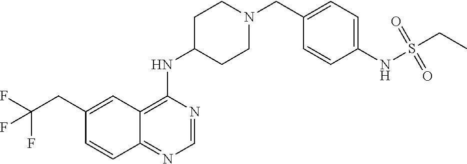 [SMILES: CCS(=O)(=O)Nc1ccc(CN2CCC(Nc3ncnc4ccc(CC(F)(F)F)cc34)CC2)cc1] | N-(4-((4-((6-(2,2,2-trifluoroethyl)quinazolin-4- yl)amino)piperidin-1-yl)methyl)phenyl)ethanesulfonamide |
| 37 | 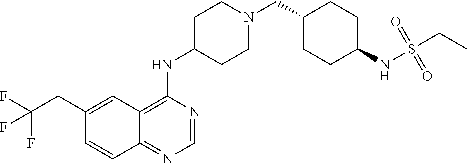 [SMILES: CCS(=O)(=O)N[C@H]1CC[C@H](CN2CCC(Nc3ncnc4ccc(CC(F)(F)F)cc34)CC2)CC1] | N-((1R,4R)-4-((4-((6-(2,2,2-trifluoroethyl)quinazolin-4- yl)amino)piperidin-1- yl)methyl)cyclohexyl)ethanesulfonamide |
| 38 | 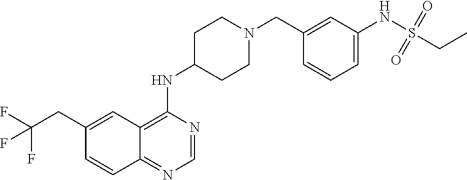 [SMILES: CCS(=O)(=O)Nc1cccc(CN2CCC(Nc3ncnc4ccc(CC(F)(F)F)cc34)CC2)c1] | N-(3-((4-((6-(2,2,2-trifluoroethyl)quinazolin-4- yl)amino)piperidin-1-yl)methyl)phenyl)ethanesulfonamide |
| 39 | 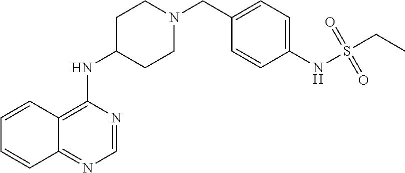 [SMILES: CCS(=O)(=O)Nc1ccc(CN2CCC(Nc3ncnc4ccccc34)CC2)cc1] | N-(4-((4-(quinazolin-4-ylamino)piperidin-1- yl)methyl)phenyl)ethanesulfonamide |
| 40 | 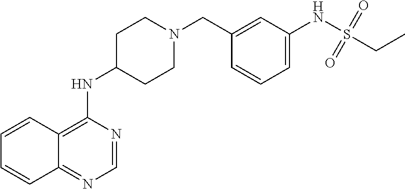 [SMILES: CCS(=O)(=O)Nc1cccc(CN2CCC(Nc3ncnc4ccccc34)CC2)c1] | N-(3-((4-(quinazolin-4-ylamino)piperidin-1- yl)methyl)phenyl)ethanesulfonamide |
| 41 | 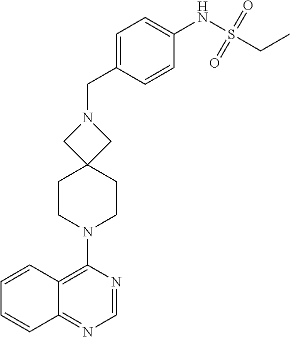 [SMILES: CCS(=O)(=O)Nc1ccc(CN2CC3(CCN(c4ncnc5ccccc45)CC3)C2)cc1] | N-(4-((7-(quinazolin-4-yl)-2,7-diazaspiro[3.5]nonan-2- yl)methyl)phenyl)ethanesulfonamide |
| 42 | 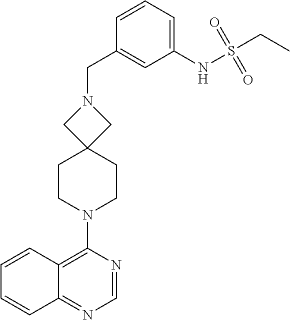 [SMILES: CCS(=O)(=O)Nc1cccc(CN2CC3(CCN(c4ncnc5ccccc45)CC3)C2)c1] | N-(3-((7-(quinazolin-4-yl)-2,7-diazaspiro[3.5]nonan-2- yl)methyl)phenyl)ethanesulfonamide |
| 43 | 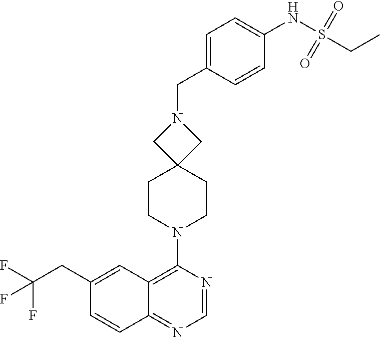 [SMILES: CCS(=O)(=O)Nc1ccc(CN2CC3(CCN(c4ncnc5ccc(CC(F)(F)F)cc45)CC3)C2)cc1] | N-(4-((7-(6-(2,2,2-trifluoroethyl)quinazolin-4-yl)-2,7- diazaspiro[3.5]nonan-2-yl)methyl)phenyl)ethanesulfonamide |
| 44 | 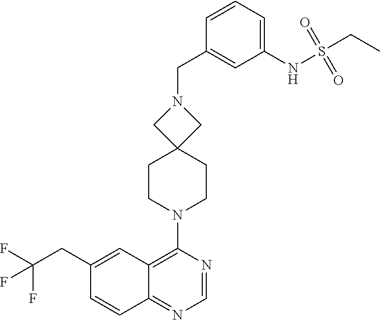 [SMILES: CCS(=O)(=O)Nc1cccc(CN2CC3(CCN(c4ncnc5ccc(CC(F)(F)F)cc45)CC3)C2)c1] | N-(3-((7-(6-(2,2,2-trifluoroethyl)quinazolin-4-yl)-2,7- diazaspiro[3.5]nonan-2-yl)methyl)phenyl)ethanesulfonamide |
| 45 | 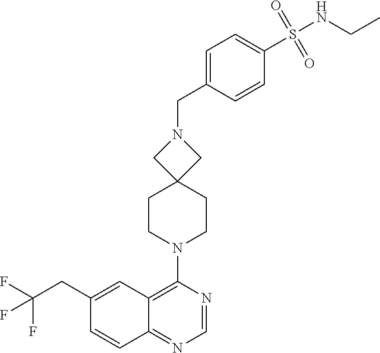 [SMILES: CCNS(=O)(=O)c1ccc(CN2CC3(CCN(c4ncnc5ccc(CC(F)(F)F)cc45)CC3)C2)cc1] | N-ethyl-4-((7-(6-(2,2,2-trifluoroethyl)quinazolin-4-yl)-2,7- diazaspiro[3.5]nonan-2-yl)methyl)benzenesulfonamide |
| 46 | 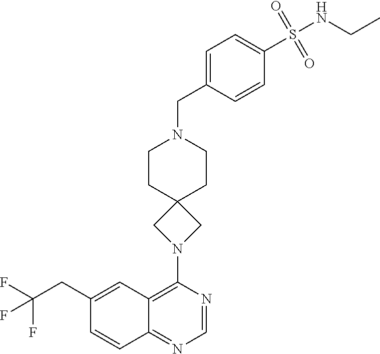 [SMILES: CCNS(=O)(=O)c1ccc(CN2CCC3(CC2)CN(c2ncnc4ccc(CC(F)(F)F)cc24)C3)cc1] | N-ethyl-4-((2-(6-(2,2,2-trifluoroethyl)quinazolin-4-yl)-2,7- diazaspiro[3.5]nonan-7-yl)methyl)benzenesulfonamide |
| 48 | 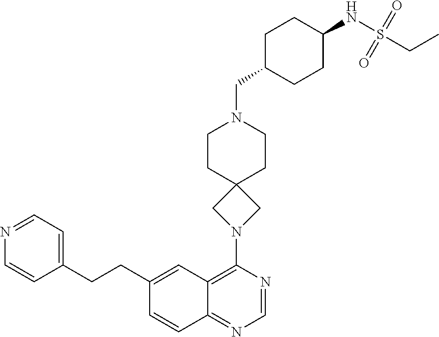 [SMILES: CCS(=O)(=O)N[C@H]1CC[C@H](CN2CCC3(CC2)CN(c2ncnc4ccc(CCc5ccncc5)cc24)C3)CC1] | N-((1R,4R)-4-((2-(6-(2-(pyridin-4-yl)ethyl)quinazolin-4-yl)- 2,7-diazaspiro[3.5]nonan-7- yl)methyl)cyclohexyl)ethanesulfonamide |
| 49 | 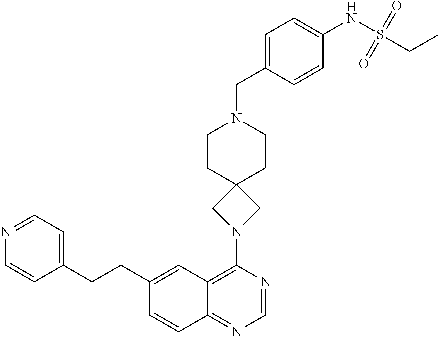 [SMILES: CCS(=O)(=O)Nc1ccc(CN2CCC3(CC2)CN(c2ncnc4ccc(CCc5ccncc5)cc24)C3)cc1] | N-(4-((2-(6-(2-(pyridin-4-yl)ethyl)quinazolin-4-yl)-2,7- diazaspiro[3.5]nonan-7-yl)methyl)phenyl)ethanesulfonamide |
| 51 | 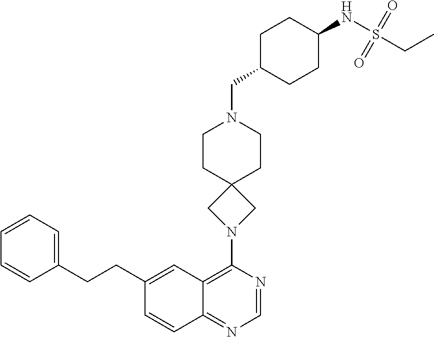 [SMILES: CCS(=O)(=O)N[C@H]1CC[C@H](CN2CCC3(CC2)CN(c2ncnc4ccc(CCc5ccccc5)cc24)C3)CC1] | N-((1R,4R)-4-((2-(6-phenethylquinazolin-4-yl)-2,7- diazaspiro[3.5]nonan-7- yl)methyl)cyclohexyl)ethanesulfonamide |
| 52 | 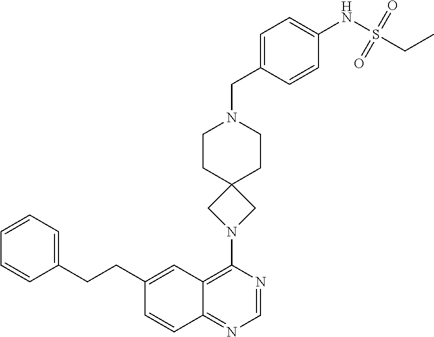 [SMILES: CCS(=O)(=O)Nc1ccc(CN2CCC3(CC2)CN(c2ncnc4ccc(CCc5ccccc5)cc24)C3)cc1] | N-(4-((2-(6-phenethylquinazolin-4-yl)-2,7- diazaspiro[3.5]nonan-7-yl)methyl)phenyl)ethanesulfonamide |
| 53 | 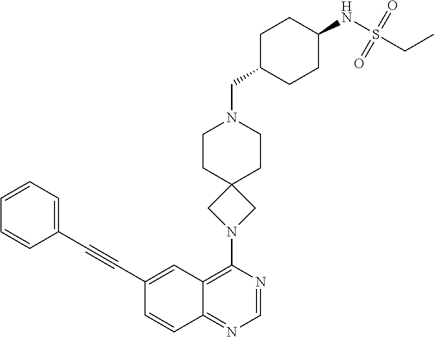 [SMILES: CCS(=O)(=O)N[C@H]1CC[C@H](CN2CCC3(CC2)CN(c2ncnc4ccc(C#Cc5ccccc5)cc24)C3)CC1] | N-((1R,4R)-4-((2-(6-(phenylethynyl)quinazolin-4-yl)-2,7- diazaspiro[3.5]nonan-7- yl)methyl)cyclohexyl)ethanesulfonamide |
| 54 | 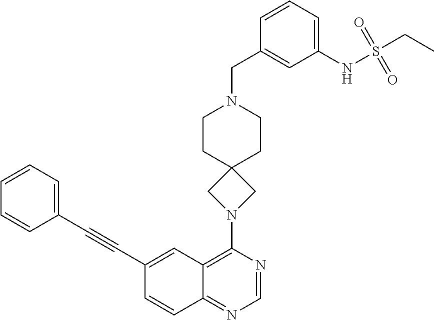 [SMILES: CCS(=O)(=O)Nc1cccc(CN2CCC3(CC2)CN(c2ncnc4ccc(C#Cc5ccccc5)cc24)C3)c1] | N-(3-((2-(6-(phenylethynyl)quinazolin-4-yl)-2,7- diazaspiro[3.5]nonan-7-yl)methyl)phenyl)ethanesulfonamide |
| 55 | 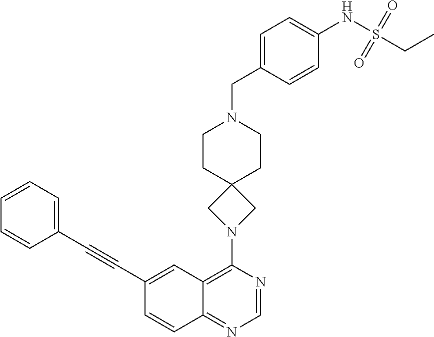 [SMILES: CCS(=O)(=O)Nc1ccc(CN2CCC3(CC2)CN(c2ncnc4ccc(C#Cc5ccccc5)cc24)C3)cc1] | N-(4-((2-(6-(phenylethynyl)quinazolin-4-yl)-2,7- diazaspiro[3.5]nonan-7-yl)methyl)phenyl)ethanesulfonamide |
| 56 | 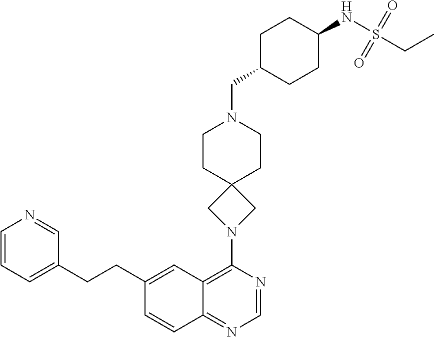 [SMILES: CCS(=O)(=O)N[C@H]1CC[C@H](CN2CCC3(CC2)CN(c2ncnc4ccc(CCc5cccnc5)cc24)C3)CC1] | N-((1R,4R)-4-((2-(6-(2-(pyridin-3-yl)ethyl)quinazolin-4-yl)- 2,7-diazaspiro[3.5]nonan-7- yl)methyl)cyclohexyl)ethanesulfonamide |
| 57 | 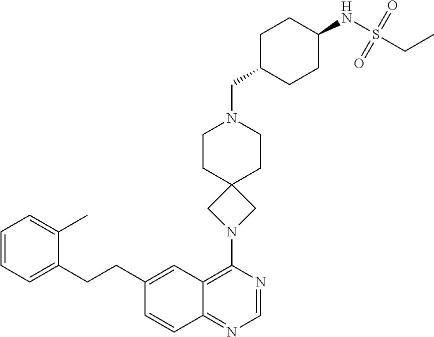 [SMILES: CCS(=O)(=O)N[C@H]1CC[C@H](CN2CCC3(CC2)CN(c2ncnc4ccc(CCc5ccccc5C)cc24)C3)CC1] | N-((1R,4R)-4-((2-(6-(2-methylphenethyl)quinazolin-4-yl)- 2,7-diazaspiro[3.5]nonan-7- yl)methyl)cyclohexyl)ethanesulfonamide |
| 58 | 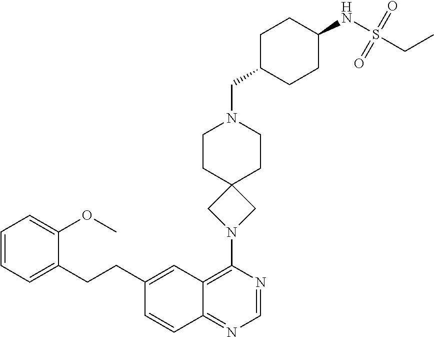 [SMILES: CCS(=O)(=O)N[C@H]1CC[C@H](CN2CCC3(CC2)CN(c2ncnc4ccc(CCc5ccccc5OC)cc24)C3)CC1] | N-((1R,4R)-4-((2-(6-(2-methoxyphenethyl)quinazolin-4-yl)- 2,7-diazaspiro[3.5]nonan-7- yl)methyl)cyclohexyl)ethanesulfonamide |
| 59 | 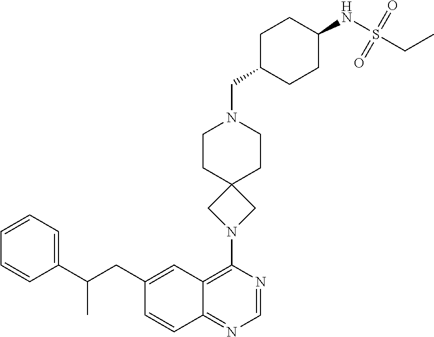 [SMILES: CCS(=O)(=O)N[C@H]1CC[C@H](CN2CCC3(CC2)CN(c2ncnc4ccc(CC(C)c5ccccc5)cc24)C3)CC1] | N-((1R,4R)-4-((2-(6-((RS)-2-phenylpropyl)quinazolin-4-yl)- 2,7-diazaspiro[3.5]nonan-7- yl)methyl)cyclohexyl)ethanesulfonamide |
| 60 | 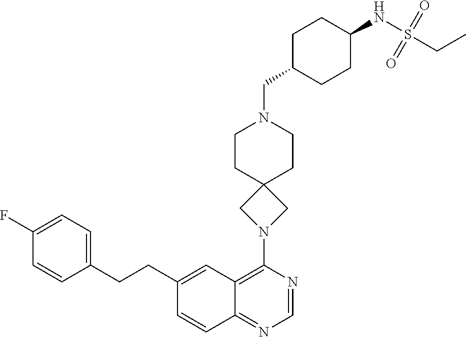 [SMILES: CCS(=O)(=O)N[C@H]1CC[C@H](CN2CCC3(CC2)CN(c2ncnc4ccc(CCc5ccc(F)cc5)cc24)C3)CC1] | N-((1R,4R)-4-((2-(6-(4-fluorophenethyl)quinazolin-4-yl)- 2,7-diazaspiro[3.5]nonan-7- yl)methyl)cyclohexyl)ethanesulfonamide |
| 61 | 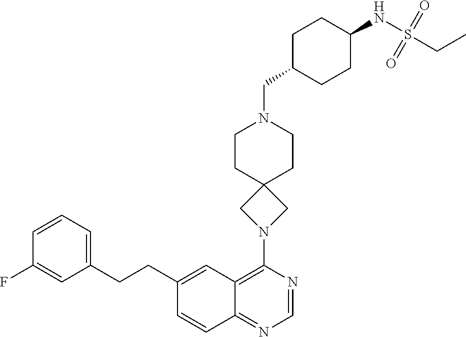 [SMILES: CCS(=O)(=O)N[C@H]1CC[C@H](CN2CCC3(CC2)CN(c2ncnc4ccc(CCc5cccc(F)c5)cc24)C3)CC1] | N-((1R,4R)-4-((2-(6-(3-fluorophenethyl)quinazolin-4-yl)- 2,7-diazaspiro[3.5]nonan-7- yl)methyl)cyclohexyl)ethanesulfonamide |
| 62 | 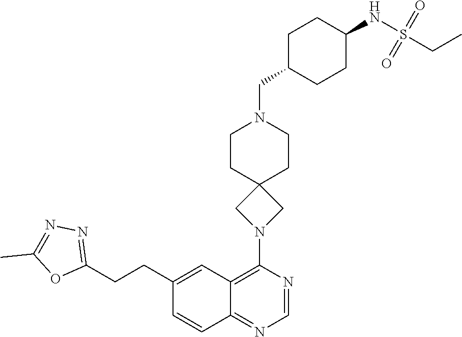 [SMILES: CCS(=O)(=O)N[C@H]1CC[C@H](CN2CCC3(CC2)CN(c2ncnc4ccc(CCc5nnc(C)o5)cc24)C3)CC1] | N-((1R,4R)-4-((2-(6-(2-(5-methyl-1,3,4-oxadiazol-2- yl)ethyl)quinazolin-4-yl)-2,7-diazaspiro[3.5]nonan-7- yl)methyl)cyclohexyl)ethanesulfonamide |
| 63 | 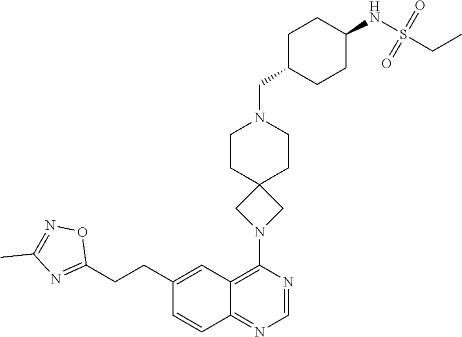 [SMILES: CCS(=O)(=O)N[C@H]1CC[C@H](CN2CCC3(CC2)CN(c2ncnc4ccc(CCc5nc(C)no5)cc24)C3)CC1] | N-((1R,4R)-4-((2-(6-(2-(3-methyl-1,2,4-oxadiazol-5- yl)ethyl)quinazolin-4-yl)-2,7-diazaspiro[3.5]nonan-7- yl)methyl)cyclohexyl)ethanesulfonamide |
| 64 | 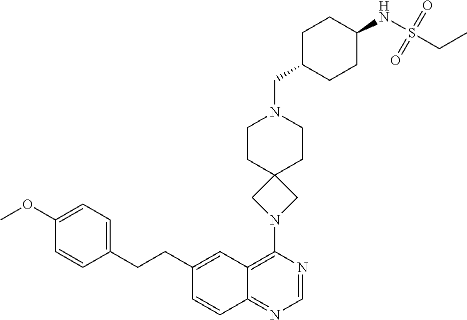 [SMILES: CCS(=O)(=O)N[C@H]1CC[C@H](CN2CCC3(CC2)CN(c2ncnc4ccc(CCc5ccc(OC)cc5)cc24)C3)CC1] | N-((1R,4R)-4-((2-(6-(4-methoxyphenethyl)quinazolin-4-yl)- 2,7-diazaspiro[3.5]nonan-7- yl)methyl)cyclohexyl)ethanesulfonamide |
| 65 | 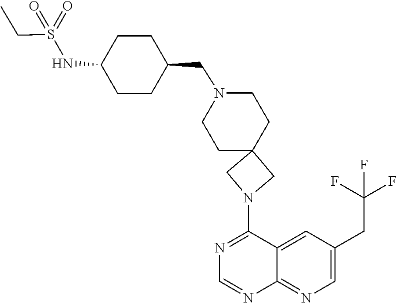 [SMILES: CCS(=O)(=O)N[C@H]1CC[C@H](CN2CCC3(CC2)CN(c2ncnc4ncc(CC(F)(F)F)cc24)C3)CC1] | N-(1R,4R)-[4-[[2-[6-(2,2,2-trifluoroethyl)pyrido[2,3- d]pyrimidin-4-yl]-2,7-diazaspiro[3.5]nonan-7- yl]methyl]cyclohexyl]ethanesulfonamide |
| 66 | 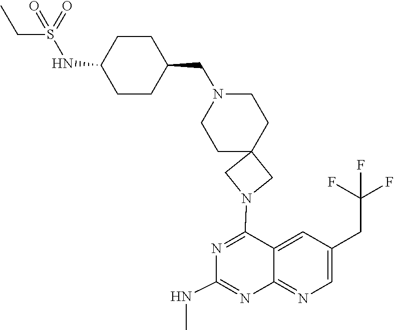 [SMILES: CCS(=O)(=O)N[C@H]1CC[C@H](CN2CCC3(CC2)CN(c2nc(NC)nc4ncc(CC(F)(F)F)cc24)C3)CC1] | N-(1R,4R)-[4-[[2-[2-(methylamino)-6-(2,2,2- trifluoroethyl)pyrido[2,3-d]pyrimidin-4-yl]-2,7- diazaspiro[3.5]nonan-7- yl]methyl]cyclohexyllethanesulfonamide |
| 69 | 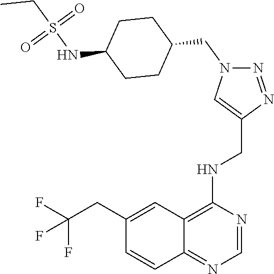 [SMILES: CCS(=O)(=O)N[C@H]1CC[C@H](Cn2cc(CNc3ncnc4ccc(CC(F)(F)F)cc34)nn2)CC1] | N-((1R,4R)-4-((4-(((6-(2,2,2-trifluoroethyl)quinazolin-4- yl)amino)methyl)-1H-1,2,3-triazol-1- yl)methyl)cyclohexyl)ethanesulfonamide |
| 70 | 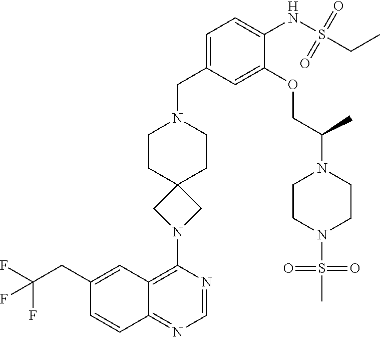 [SMILES: CCS(=O)(=O)Nc1ccc(CN2CCC3(CC2)CN(c2ncnc4ccc(CC(F)(F)F)cc24)C3)cc1OC[C@@H](C)N1CCN(S(C)(=O)=O)CC1] | rac-(R)-N-(2-(2-(4-(methylsulfonyl)piperazin-1-yl)propoxy)- 4-((2-(6-(2,2,2-trifluoroethyl)quinazolin-4-yl)-2,7- diazaspiro[3.5]nonan-7-yl)methyl)phenyl)ethanesulfonamide |
| 71 | 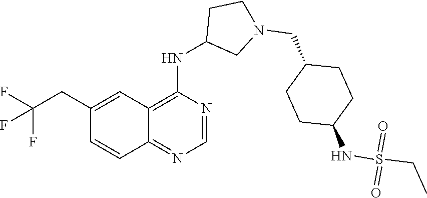 [SMILES: CCS(=O)(=O)N[C@H]1CC[C@H](CN2CCC(Nc3ncnc4ccc(CC(F)(F)F)cc34)C2)CC1] | N-((1R,4R)-4-(((RS)-3-((6-(2,2,2-trifluoroethyl)quinazolin- 4-yl)amino)pyrrolidin-1- yl)methyl)cyclohexyl)ethanesulfonamide |
| 72 | 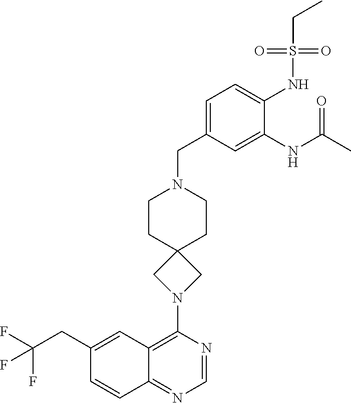 [SMILES: CCS(=O)(=O)Nc1ccc(CN2CCC3(CC2)CN(c2ncnc4ccc(CC(F)(F)F)cc24)C3)cc1NC(C)=O] | N-(2-(ethylsulfonamido)-5-((2-(6-(2,2,2- trifluoroethyl)quinazolin-4-yl)-2,7-diazaspiro[3.5]nonan-7- yl)methyl)phenyl)acetamide |
| 73 | 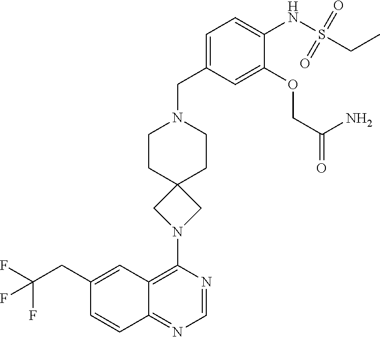 [SMILES: CCS(=O)(=O)Nc1ccc(CN2CCC3(CC2)CN(c2ncnc4ccc(CC(F)(F)F)cc24)C3)cc1OCC(N)=O] | 2-(2-(ethylsulfonamido)-5-((2-(6-(2,2,2- trifluoroethyl)quinazolin-4-yl)-2,7-diazaspiro[3.5]nonan-7- yl)methyl)phenoxy)acetamide |
| 74 | 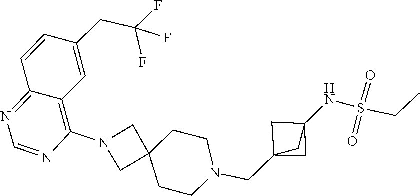 [SMILES: CCS(=O)(=O)NC12CC(CN3CCC4(CC3)CN(c3ncnc5ccc(CC(F)(F)F)cc35)C4)(C1)C2] | N-(3-((2-(6-(2,2,2-trifluoroethyl)quinazolin-4-yl)-2,7- diazaspiro[3.5]nonan-7-yl)methyl)bicyclo[1.1.1]pentan-1- yl)ethanesulfonamide |
| 75 | 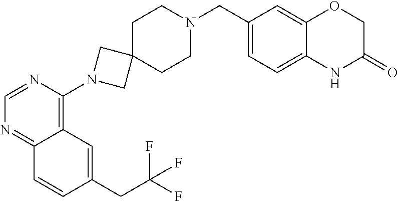 [SMILES: O=C1COc2cc(CN3CCC4(CC3)CN(c3ncnc5ccc(CC(F)(F)F)cc35)C4)ccc2N1] | 7-((2-(6-(2,2,2-trifluoroethyl)quinazolin-4-yl)-2,7- diazaspiro[3.5]nonan-7-yl)methyl)-2H-benzo[b][1,4]oxazin- 3(4H)-one |
| 77 | 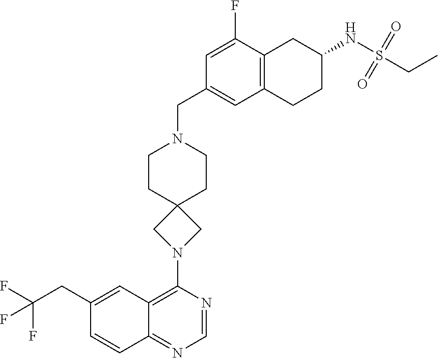 [SMILES: CCS(=O)(=O)N[C@@H]1CCc2cc(CN3CCC4(CC3)CN(c3ncnc5ccc(CC(F)(F)F)cc35)C4)cc(F)c2C1] | rac-(R)-N-(8-fluoro-6-((2-(6-(2,2,2-trifluoroethyl)quinazolin- 4-yl)-2,7-diazaspiro[3.5]nonan-7-yl)methyl)-1,2,3,4- tetrahydronaphthalen-2-yl)ethanesulfonamide |
| 78 | 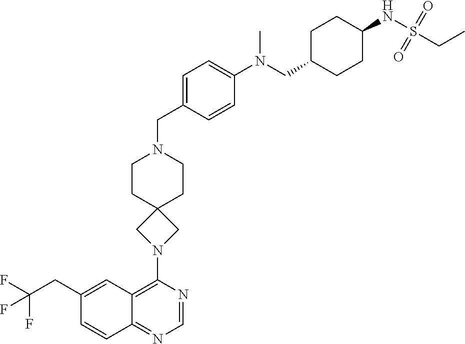 | N-((1R,4R)-4-((methyl(4-((2-(6-(2,2,2- trifluoroethyl)quinazolin-4-yl)-2,7-diazaspiro[3.5]nonan-7- yl)methyl)phenyl)amino)methyl)cyclohexyl)ethanesulfonamide |
| 79 | 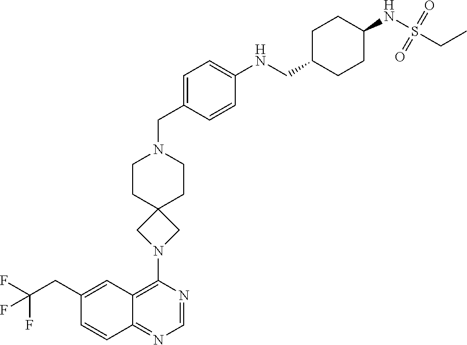 | N-((1R,4R)-4-(((4-((2-(6-(2,2,2-trifluoroethyl)quinazolin-4- yl)-2,7-diazaspiro[3.5]nonan-7- yl)methyl)phenyl)amino)methyl)cyclohexyl)ethanesulfonamide |
| 80 | 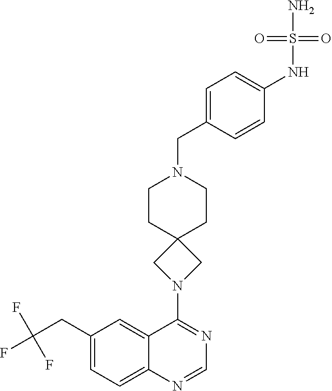 [SMILES: NS(=O)(=O)Nc1ccc(CN2CCC3(CC2)CN(c2ncnc4ccc(CC(F)(F)F)cc24)C3)cc1] | 4-[7-[[4-(sulfamoylamino)phenyl]methyl]-2,7- diazaspiro[3.5]nonan-2-yl]-6-(2,2,2- trifluoroethyl)quinazoline |
| 81 | 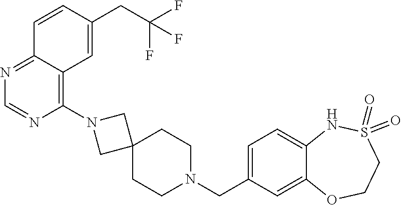 [SMILES: O=S1(=O)CCOc2cc(CN3CCC4(CC3)CN(c3ncnc5ccc(CC(F)(F)F)cc35)C4)ccc2N1] | 7-((2-(6-(2,2,2-trifluoroethyl)quinazolin-4-yl)-2,7- diazaspiro[3.5]nonan-7-yl)methyl)-3,4-dihydro-1H- benzo[f][1,4,5]oxathiazepine 2,2-dioxide |
| 82 | 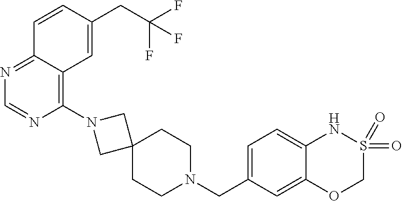 [SMILES: O=S1(=O)COc2cc(CN3CCC4(CC3)CN(c3ncnc5ccc(CC(F)(F)F)cc35)C4)ccc2N1] | 6-((2-(6-(2,2,2-trifluoroethyl)quinazolin-4-yl)-2,7- diazaspiro[3.5]nonan-7-yl)methyl)-1H,3H- benzo[e][1,3,4]oxathiazine 2,2-dioxide |
| 83 | 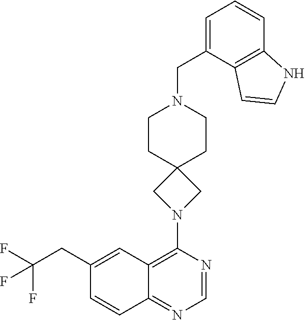 [SMILES: FC(F)(F)Cc1ccc2ncnc(N3CC4(CCN(Cc5cccc6[nH]ccc56)CC4)C3)c2c1] | 4-(7-((1H-indol-4-yl)methyl)-2,7-diazaspiro[3.5]nonan-2- yl)-6-(2,2,2-trifluoroethyl)quinazoline |
| 84 | 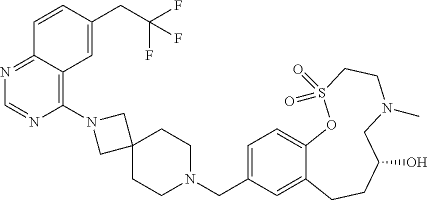 [SMILES: CN1CCS(=O)(=O)Oc2ccc(CN3CCC4(CC3)CN(c3ncnc5ccc(CC(F)(F)F)cc35)C4)cc2CC[C@@H](O)C1] | rac-(R)-7-hydroxy-5-methyl-11-((2-(6-(2,2,2- trifluoroethyl)quinazolin-4-yl)-2,7-diazaspiro[3.5]nonan-7- yl)methyl)-3,4,5,6,7,8-hexahydro-1H- benzo[b][1]oxa[5]thia[4,8]diazacycloundecine 2,2-dioxide |
| 85 | 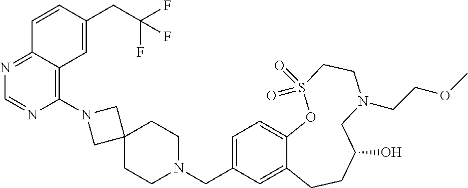 [SMILES: COCCN1CCS(=O)(=O)Oc2ccc(CN3CCC4(CC3)CN(c3ncnc5ccc(CC(F)(F)F)cc35)C4)cc2CC[C@@H](O)C1] | rac-(R)-7-hydroxy-5-(2-methoxyethyl)-11-((2-(6-(2,2,2- trifluoroethyl)quinazolin-4-yl)-2,7-diazaspiro[3.5]nonan-7- yl)methyl)-3,4,5,6,7,8-hexahydro-1H- benzo[b][1]oxa[5]thia[4,8]diazacycloundecine 2,2-dioxide |
| 86 | 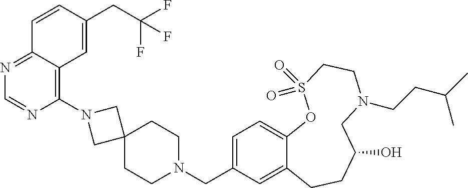 [SMILES: CC(C)CCN1CCS(=O)(=O)Oc2ccc(CN3CCC4(CC3)CN(c3ncnc5ccc(CC(F)(F)F)cc35)C4)cc2CC[C@@H](O)C1] | rac-(R)-7-hydroxy-5-isopentyl-11-((2-(6-(2,2,2- trifluoroethyl)quinazolin-4-yl)-2,7-diazaspiro[3.5]nonan-7- yl)methyl)-3,4,5,6,7,8-hexahydro-1H- benzo[b][1]oxa[5]thia[4,8]diazacycloundecine 2,2-dioxide |
| 87 | 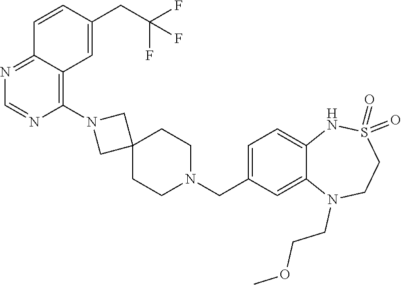 [SMILES: COCCN1CCS(=O)(=O)Nc2ccc(CN3CCC4(CC3)CN(c3ncnc5ccc(CC(F)(F)F)cc35)C4)cc21] | 5-(2-methoxyethyl)-7-((2-(6-(2,2,2-trifluoroethyl)quinazolin- 4-yl)-2,7-diazaspiro[3.5]nonan-7-yl)methyl)-1,3,4,5- tetrahydrobenzo[c][1,2,5]thiadiazepine 2,2-dioxide |
| 88 | 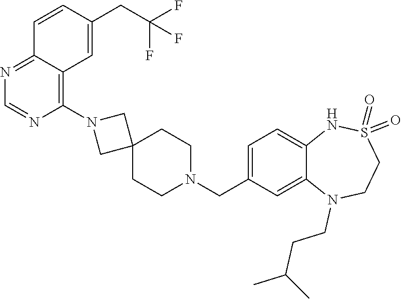 [SMILES: CC(C)CCN1CCS(=O)(=O)Nc2ccc(CN3CCC4(CC3)CN(c3ncnc5ccc(CC(F)(F)F)cc35)C4)cc21] | 5-isopentyl-7-((2-(6-(2,2,2-trifluoroethyl)quinazolin-4-yl)- 2,7-diazaspiro[3.5]nonan-7-yl)methyl)-1,3,4,5- tetrahydrobenzo[c][1,2,5]thiadiazepine 2,2-dioxide |
| 89 | 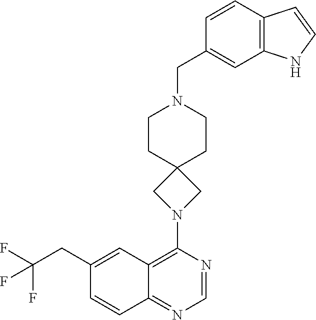 [SMILES: FC(F)(F)Cc1ccc2ncnc(N3CC4(CCN(Cc5ccc6cc[nH]c6c5)CC4)C3)c2c1] | 4-(7-((1H-indol-6-yl)methyl)-2,7-diazaspiro[3.5]nonan-2- yl)-6-(2,2,2-trifluoroethyl)quinazoline |
| 90 | 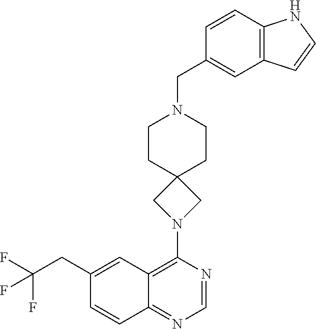 [SMILES: FC(F)(F)Cc1ccc2ncnc(N3CC4(CCN(Cc5ccc6[nH]ccc6c5)CC4)C3)c2c1] | 4-(7-((1H-indol-5-yl)methyl)-2,7-diazaspiro[3.5]nonan-2- yl)-6-(2,2,2-trifluoroethyl)quinazoline |
| 91 | 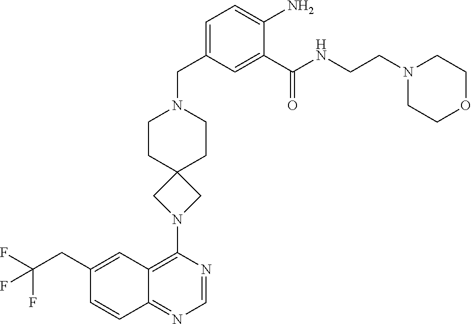 [SMILES: Nc1ccc(CN2CCC3(CC2)CN(c2ncnc4ccc(CC(F)(F)F)cc24)C3)cc1C(=O)NCCN1CCOCC1] | 2-amino-N-(2-morpholinoethyl)-5-((2-(6-(2,2,2- trifluoroethyl)quinazolin-4-yl)-2,7-diazaspiro[3.5]nonan-7- yl)methyl)benzamide |
| 92 | 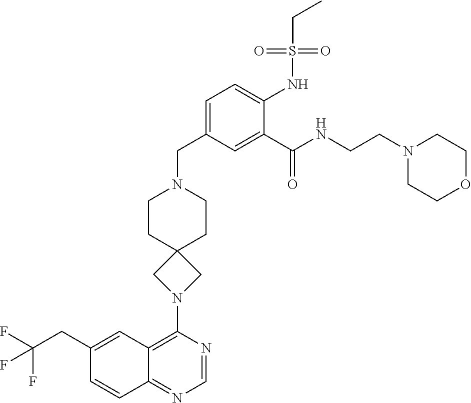 [SMILES: CCS(=O)(=O)Nc1ccc(CN2CCC3(CC2)CN(c2ncnc4ccc(CC(F)(F)F)cc24)C3)cc1C(=O)NCCN1CCOCC1] | 2-(ethylsulfonamido)-N-(2-morpholinoethyl)-5-((2-(6-(2,2,2- trifluoroethyl)quinazolin-4-yl)-2,7-diazaspiro[3.5]nonan-7- yl)methyl)benzamide |
| 93 | 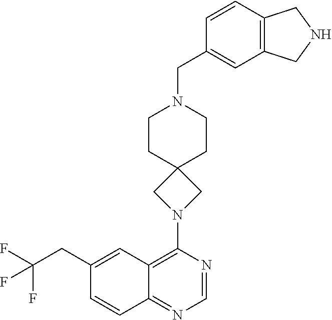 [SMILES: FC(F)(F)Cc1ccc2ncnc(N3CC4(CCN(Cc5ccc6c(c5)CNC6)CC4)C3)c2c1] | 4-(7-(isoindolin-5-ylmethyl)-2,7-diazaspiro[3.5]nonan-2-yl)- 6-(2,2,2-trifluoroethyl)quinazoline |
| 94 | 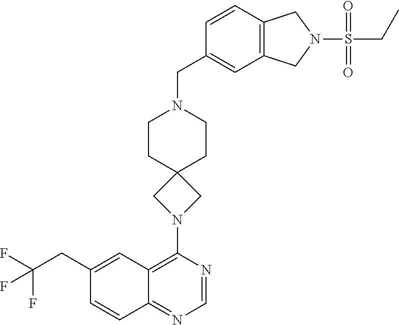 [SMILES: CCS(=O)(=O)N1Cc2ccc(CN3CCC4(CC3)CN(c3ncnc5ccc(CC(F)(F)F)cc35)C4)cc2C1] | 4-(7-((2-(ethylsulfonyl)isoindolin-5-yl)methyl)-2,7- diazaspiro[3.5]nonan-2-yl)-6-(2,2,2- trifluoroethyl)quinazoline |
| 100 | 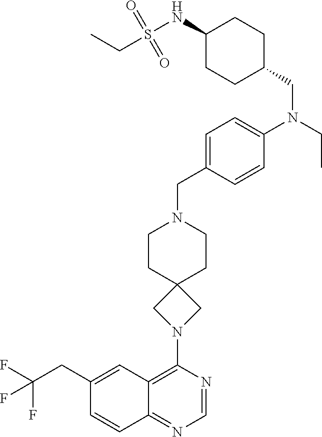 [SMILES: CCN(C[C@H]1CC[C@H](NS(=O)(=O)CC)CC1)c1ccc(CN2CCC3(CC2)CN(c2ncnc4ccc(CC(F)(F)F)cc24)C3)cc1] | N-((1R,4R)-4-((ethyl(4-((2-(6-(2,2,2- trifluoroethyl)quinazolin-4-yl)-2,7-diazaspiro[3.5]nonan-7- yl)methyl)phenyl)amino)methyl)cyclohexyl)ethanesulfonamide |
| 101 | 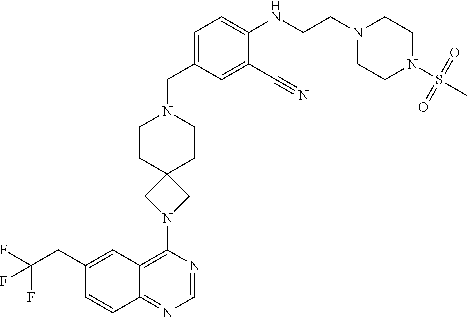 [SMILES: CS(=O)(=O)N1CCN(CCNc2ccc(CN3CCC4(CC3)CN(c3ncnc5ccc(CC(F)(F)F)cc35)C4)cc2C#N)CC1] | 2-((2-(4-(methylsulfonyl)piperazin-1-yl)ethyl)amino)-5-((2- (6-(2,2,2-trifluoroethyl)quinazolin-4-yl)-2,7- diazaspiro[3.5]nonan-7-yl)methyl)benzonitrile |
| 102 | 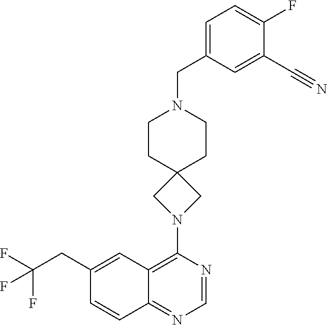 [SMILES: N#Cc1cc(CN2CCC3(CC2)CN(c2ncnc4ccc(CC(F)(F)F)cc24)C3)ccc1F] | 2-fluoro-5-((2-(6-(2,2,2-trifluoroethyl)quinazolin-4-yl)-2,7- diazaspiro[3.5]nonan-7-yl)methyl)benzonitrile |
| 110 | 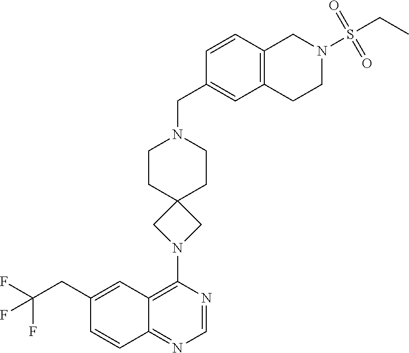 [SMILES: CCS(=O)(=O)N1CCc2cc(CN3CCC4(CC3)CN(c3ncnc5ccc(CC(F)(F)F)cc35)C4)ccc2C1] | 4-(7-((2-(ethylsulfonyl)-1,2,3,4-tetrahydroisoquinolin-6- yl)methyl)-2,7-diazaspiro[3.5]nonan-2-yl)-6-(2,2,2- trifluoroethyl)quinazoline |
| 111 | 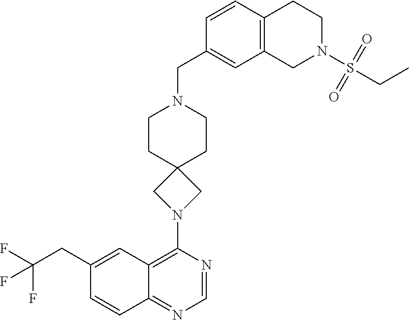 [SMILES: CCS(=O)(=O)N1CCc2ccc(CN3CCC4(CC3)CN(c3ncnc5ccc(CC(F)(F)F)cc35)C4)cc2C1] | 4-(7-((2-(ethylsulfonyl)-1,2,3,4-tetrahydroisoquinolin-7- yl)methyl)-2,7-diazaspiro[3.5]nonan-2-yl)-6-(2,2,2- trifluoroethyl)quinazoline |
| 138 | 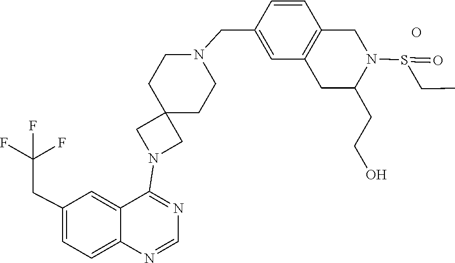 | 2-[2-ethylsulfonyl-6-[2-[6-(2,2,2-trifluoroethyl)quinazolin- 4-yl]-2,7-diazaspiro[3.5]nonan-7-yl]methyl]-3,4-dihydro- 1H-isoquinolin-3-yl]ethanol |

or a pharmaceutically acceptable salt, stereoisomer, solvate, or tautomer thereof.

15. A pharmaceutical composition comprising the compound of claim 1 or a pharmaceutically acceptable salt, stereoisomer, solvate, or tautomer thereof, and a pharmaceutically acceptable carrier.

16. The pharmaceutical composition of claim 15, further comprising one or more additional pharmaceutically active agents.

17. A method of inhibiting the interaction of menin and MILL in a cell, comprising contacting the cell with a compound of claim 1.

18. A method of inhibiting the interaction of menin and MLL1 in a cell, comprising contacting the cell with a compound claim 1.

19. The method of claim 17 or 18, wherein the contacting is in vitro or in vivo.

20. A method for the treatment or prevention of a disease or disorder associated with the inhibition of the interaction of menin and MLL comprising administering to a subject in need thereof a compound of claim 1.

21. The method of claim 20, wherein the disease or disorder is selected from the group consisting of a leukemia, hematologic malignancy, solid tumor cancer, prostate cancer, breast cancer, liver cancer, brain tumor, and diabetes.

22. The method of claim 21, wherein the leukemia is selected from the group consisting of AML, ALL, Mixed Lineage Leukemia, and a leukemia with Partial Tandem Duplications of MLL.

23. The method of any one of claims 17-22, wherein the subject is a mammal.

24. The method of claim 23, wherein the subject is a human.
